(19) United States
(12) Patent Application Publication
(10) Pub. No.: US 2009/0264306 A1
(43) Pub. Date: Oct. 22, 2009

(54) DNA METHYLATION BIOMARKERS IN LYMPHOID AND HEMATOPOIETIC MALIGNANCIES

(75) Inventors: Charles W. Caldwell, Columbia, MO (US); Huidong Shi, Martinez, GA (US); Farahnaz Rahmatpanah, Newport Beach, CA (US); Kristen H. Taylor, Columbia, MO (US); Douglas E. Laux, Iowa City, IA (US); Deiter J. Duff, Columbia, MO (US); Juyuan Guo, Columbia, MO (US)

Correspondence Address: DAVIS WRIGHT TREMAINE, LLP/Seattle, 1201 Third Avenue, Suite 2200, SEATTLE, WA, 98101-3045, US

(73) Assignee: Curators of the University of Missouri, Columbia, MO (US)

(21) Appl. No.: 12/091,718

(22) PCT Filed: Oct. 27, 2006
(86) PCT No.: PCT/US2006/041670

§ 371 Date: Mar. 12, 2009

Related U.S. Application Data
(60) Provisional application No. 60/731,040, filed on Oct. 27, 2005.
(60) Provisional application No. 60/733,648, filed on Nov. 4, 2005.

(51) Int. Cl. C40B 30/04; C12Q 1/68

(52) U.S. Cl. 506/9; 435/6

(57) ABSTRACT

Differential Methylation Hybridization (DMH) was used to identify novel methylation markers and methylation profiles for hematopoieetic malignancies, leukemia, lymphomas, etc. (e.g., non-Hodgkin's lymphomas (NHL), small B-cell lymphomas (SBCL), diffuse large B-cell lymphoma (DLBCL), follicular lymphoma (FL), mantle cell lymphoma (MCL), B-cell chronic lymphocytic leukemia/small lymphocytic lymphoma (B-CLL/SLL), chronic lymphocytic leukemia (CLL), multiple myeloma (MM), acute myelogenous leukemia (AML), acute lymphoblastic leukemia (ALL), etc.). Particular aspects provide novel biomarkers for NHL and subtypes thereof (e.g., MCL, B-CLL/SLL, FL, DLBCL, etc.), AML, ALL and MM, and further provide non-invasive tests (e.g. blood tests) for lymphomas and leukemias. Additional aspects provide markers for diagnosis, prognosis, monitoring responses to therapies, relapse, etc., and further provide targets and methods for therapeutic demethylating treatments. Further aspects provide cancer staging markers, and expression assays and approaches comprising idealized methylation and/or patterns” (IMP and/or IEP) and fusion of gene rankings.

### CROSS-REFERENCE TO RELATED APPLICATIONS

This application claims the benefit of priority to U.S. Provisional Application Ser. Nos. 60/731,040, filed 27 Oct. 2005, and 60/733,648, filed 4 Nov. 2005, both of which are incorporated herein by reference in their entirety.

### STATEMENT REGARDING FEDERALLY-SPONSORED RESEARCH

Aspects of this disclosure were developed with funding from NIH grant CA097880-01. The United States government has certain rights in this invention.

### FIELD OF THE INVENTION

Particular aspects are related generally to DNA methylation and cancer, and more particularly to novel compositions and methods based on novel methylation and/or expression markers having substantial utility for cancer detection, monitoring, diagnosis, prognosis, staging, treatment response prediction/monitoring, etc., where the cancers include hematopoietic malignancies, leukemia, lymphomas, etc., (e.g., non-Hodgkin's lymphomas (NHL), small B-cell lymphomas (SBCL), diffuse large B-cell lymphoma (DLBCL), follicular lymphoma (FL), mantle cell lymphoma (MCL), B-cell chronic lymphocytic leukemia/small lymphocytic lymphoma (B-CLL/SLL), chronic lymphocytic leukemia (CLL), multiple myeloma (MM), acute myelogenous leukemia (AML), acute lymphoblastic leukemia (ALL), etc.).

### SEQUENCE LISTING

A Sequence Listing in paper form (----pages) and comprising SEQ ID NOS:1----- is attached to this application, is part of this application, and is incorporated herein by reference in its entirety.

### BACKGROUND

CpG methylation. Methylation of cytosine residues at CpG dinucleotides is a major epigenetic modification in mammalian genomes and is known to frequently have profound effects on gene expression. This epigenetic event occurs globally in the normal genome, and 70-80% of all CpG dinucleotides are heavily methylated in human cells. However, ˜0.2 to 1-kb long DNA sequence stretches of GC-rich (G+C content: >50-60%) DNA, called CpG islands (CGI), appear to be protected from the modification in somatic cells. CpG islands are frequently located in the promoters and first exon regions of 40 to 50% of all genes. The rest may be located in the intronic or other exonic regions of the genes, or in regions containing no genes. Some of these normally unmethylated promoter CGIs become methylated in cancer cells, and this may result in loss of expression of adjacent genes. As a result, critical genes may be silenced, leading to clonal proliferation of tumor cells.

In cancer cells, patterns of DNA methylation are altered, and promoter (including the first exon) CpG island hypermethylation is a frequent epigenetic event in many types of cancer. This epigenetic process can result in gene silencing via alteration of local chromatin structure in the 5′ end of regulatory regions, preventing normal interaction of the promoters with the transcriptional machinery. If this occurs in genes critical to growth inhibition, the silencing event could promote tumor progression.

Although the list of methylation-repressed genes in Non-Hodgkin's Lymphomas (NHLs) is expanding rapidly, there is a substantial need in the art for identification of novel epigenetic biomarkers to provide for earlier and more accurate diagnoses, and for guiding therapy-related issues.

Non-Hodgkin's Lymphoma. Non-Hodgkin's Lymphoma (NHL) is the 5^{th} most common malignancy in the United States, accounting for approximately 56,390 new cases in year 2005. Unfortunately, the incidence has increased yearly over past decades for unknown reasons, and is one of only two cancers increasing in incidence. Mature B-cell NHL including mantle cell lymphoma (MCL), B-cell chronic lymphocytic lymphoma/small lymphocytic lymphoma (B-CLL/SLL), follicular lymphoma (FL), and diffuse large B-cell lymphoma (DLBCL) comprise >80% of all NHL cases. Together, B-cell chronic lymphocytic leukemia/small lymphocytic lymphoma (B-CLL/SLL), mantle cell lymphoma (MCL), and grades I and II follicular lymphoma (FLI/FLII) comprise one-third of all NHL cases [1]. The NHLs B-CLL/SLL and FLI/FLII are generally thought to be of low aggressiveness, but still exhibit a spectrum of clinical behavior. B-CLL/SLL is a lymphoma of at least 2 subtypes comprising both pre-germinal center and post-germinal center derivation, while MCL is also of pre-germinal center derivation, and FLI/FLII derives from germinal centers of lymphoid tissues. B-CLL/SLL is diverse across different groups of patients. Many B-CLL/SLL and FLI/FLII patients have a relatively good prognosis, with median survival of ˜7-10 years, but usually are not curable in advanced clinical stages. MCL is a pre-germinal center derived malignancy, and FLs are germinal center derived NHLs. MCL is typically more rapidly progressive than these other SBCLs.

Although advances in cancer treatment over the past several decades have improved outcomes for many patients with NHLs, the diseases are still not generally curable. The time from diagnosis to death is variable, ranging from months to many years. Current classification systems are based on clinical staging, chromosomal abnormalities and cell surface antigens, and offer important diagnostic information. Diagnostically, it is usually possible to discern each type of SBCL from the other on the basis of histologic pattern, but, there is still considerable overlap in biology, clinical behavior/disease and genetic and epigenetic alterations among the SBCL subtypes. Indolent SBCL subtypes are B cell malignancies that correlate with different stages of normal B cell differentiation. Biologically, a naive B-cell that has not been stimulated with antigen expresses a different set of genes from antigen-stimulated B-cells.

There is, therefore, a substantial need in the art for novel compositions and methods for distinguishing subtypes, and to provide improvements in therapy, as well as better ways to detect NHL and to monitor responses to therapy.

Multiple Myeloma. A number of individual genes have been reported silenced in multiple myeloma MMs. For example, alteration of p16 and p15 solely by hypermethylation has been detected in high frequencies in MMs, and hypermethylation of p16 has been shown to be associated with plasmablastic disease in primary MM. Moreover, transcriptional silencing of p16 and p 15 has been found to correlate with hypermethylation of these genes in MM-derived cell lines. These results indicate that hypermethylation of p16 and p15 plays an important role in MM tumorigenesis. Hypermethylation of the DAP-kinase (DAPK) CpG island is also a very common alteration in MM. Another example of epigenetic alteration in myeloma is dysregulation of the IL-6/JAK/STAT3 pathway, a signal pathway that is subjected to negative regulation by three families of proteins: the protein inhibitors of activated STATs (PIAS); the suppressor of cytokine signaling (SOCS); and the SH2-cotaining phosphatases (SHP). Frequent hypermethylation of both SHP-1 (79.4%) and SOCS-1 (62.9%) has been reported in multiple myelomas. Therefore, CpG island methylation is likely critical in the genesis and clinical behavior of MMs and may provide useful molecular markers for detection and determining the clinical status of these diseases.

However, because of the limited number of informative genes analyzed so far analyzed, there is a substantial need in the art for additional methylation markers for MM.

Acute myelogenous leukemia (AML). Aberrant DNA methylation is believed to be important in the tumorigenesis of numerous cancers by both silencing transcription of tumor suppressor genes and destabilizing chromatin. Previous studies have demonstrated that several tumor suppressor genes are hypermethylated in AML, suggesting a roll for this epigenetic process during tumorigenesis. However, it is unknown how the genomic methylation profiles differ among AML variants, or even whether AML can be distinguished on this basis from normal bone marrow or other hematologic malignancies.

There is, therefore, a pronounced need in the art for novel compositions and methods for detecting and distinguishing AML.

Acute Lymphoblastic Leukemia (ALL). Acute lymphoblastic leukemia (ALL) arises when B or T cell progenitors are unable to differentiate into mature B or T cells resulting in the rapid proliferation of immature cells. A multitude of factors are known to be responsible for blocking this process including translocations and epigenetic modifications which can nullify the function of a gene or cause a change in the regulation of a gene product. Many non-random translocations are known to occur in ALL resulting in aberrant proliferation, differentiation, apoptosis and gene transcription. Assays to detect these molecular anomalies have been developed and some are currently being used as prognostic markers. However, a major shortcoming of these assays has been the reliance of their detection in specific morphological subtypes of ALL (Faderl et al. 1998) demonstrating the need for alternative prognostic and classification tools in ALL.

There is a pronounced need in the art for novel compositions and methods for detecting and distinguishing ALL and/or its subtypes.

### SUMMARY OF ASPECTS OF THE INVENTION

Differential Methylation Hybridization (DMH) was used to identify novel methylation markers and methylation profiles for hematopoieetic malignancies, leukemia, lymphomas, etc. (e.g., non-Hodgkin's lymphomas (NHL), small B-cell lymphomas (SBCL), diffuse large B-cell lymphoma (DLBCL), follicular lymphoma (FL), mantle cell lymphoma (MCL), B-cell chronic lymphocytic leukemia/small lymphocytic lymphoma (B-CLL/SLL), chronic lymphocytic leukemia (CLL), multiple myeloma (MM), acute myelogenous leukemia (AML), acute lymphoblastic leukemia (ALL), etc.).

According to particular aspects, the use of a quantitative assay for DLC-1 promoter methylation has substantial utility to improve the detection rate of NHL in tissue biopsies, and from blood and/or plasma samples. Moreover, gene promoter methylation of DLC-1 occurred in a differentiation-related manner and has substantial utility as a biomarker in non-Hodgkin's Lymphoma (NHL) (e.g., for distinguishing between and among MCL (mantle cell lymphoma), B-CLL/SLL (B-cell chronic lymphocytic leukemia/small lymphocytic lymphoma), FL (follicular lymphoma), and DLBCL (diffuse large B-cell lymphoma) samples (see Example 1).

Particular aspects therefore provide novel non-invasive blood tests for lymphomas and leukemias (Id).

In further aspects, down-regulation of DLC-1 expression was correlated with NHL compared to normal lymph nodes (Id).

In additional aspects, differential methylation of LHX2, POU3F3, HOX10, NRP2, PRKCE, RAMP, MLLT2, NKX6-1, LPR1B, and ARF4 markers was validated, and demonstrated a preferential methylation pattern in germinal center-derived tumors compared to pre- and post-germinal center tumors. Therefore, in particular embodiments, these markers define distinct sub-types of SBCL that are not recognized by current classification systems, and have substantial utility for detecting and characterizing the biology of these tumors (see Example 2).

Further aspects provide promoter region markers for Non-Hodgkin's Lymphoma (NHL) and NHL subtypes, including markers based on PCDHGB7, EFNA5, CYP27B1, CCND1, DLC-1, NOPE, RPIB9, FLJ39155, PON3 and RARβ2 gene sequences that provide novel methylated gene markers relevant to molecular pathways in NHLs, and that have substantial utility as biomarkers of disease (e.g., cancer, and specific subtypes thereof). Preferably, the NHL and NHL subtype methylation markers include markers based on DLC-1, PCDHGB7, CYP27B1, EFNA5, CCND1 and RARβ2 promoter region sequences (see Example 3).

Additional aspects provide methylation markers for Multiple Myeloma (MM) and subtypes thereof, including markers based on PCDHGB7, CYP27B1, DLC-1, NOPE, FLJ39155, PON3, PITX2, DCC, FTHFD and RARβ2 promoter region sequences. Preferably, the markers for Multiple Myeloma (MM) and subtypes thereof, include markers based on PCDGHB7, CYP27B1, and NOPE promoter region sequences (see Example 4).

Yet additional aspects provide methylation markers for Acute Myelogenous Leukemia (AML) having substantial utility for distinguishing NHL FAB M0-M3 subtypes, based on their methylation profiles. For example, markers are provided that are based on genes not previously associated with abnormal methylation in AML, including the dual-specificity tyrosine phosphorylation regulated kinase 4, structural maintenance of chromosome 2-like-1, and the exportin 5 genes (see Example 5).

Additional aspects provide promoter region markers for Acute Lymphoblastic Leukemia (ALL), including markers based on ABCB1/MDR1, DLC-1, DCC, LRP1B, PCDHGAI2, RPIB9, KCNK2, NOPE, DDX51, SLC2A14, LRP1B and NKX6-1 promoter region sequences (see Example 6).

Further aspects provide for a novel goal oriented approach and algorithm for finding differentially methylated gene markers (e.g., in small B-cell lymphoma) was developed. The inventive gene selection algorithm comprises 3 main steps: array normalization; gene selection (based on idealized methylation patterns, and comprising fused gene rankings); and gene clustering (see Example 7). Variants of this approach, comprising fusion of differential methylation ranking and differential expression ranking are also disclosed.

Therefore, particular aspects of the present invention provide for novel biomarkers for NHL, SBCL and subtypes thereof (e.g., for distinguishing MCL, B-CLL/SLL, FL, DLBCL, etc.), and for AML, ALL and MM. In particular embodiments, these markers have substantial utility in providing for non-invasive tests (e.g. blood tests) for lymphomas and leukemias.

In additional aspects these markers have substantial utility for detection, diagnosis, prognosis, monitoring responses to therapies, detection of relapse patients, and the respective genes provide targets for therapeutic demethylating methods and treatments.

Further aspects provide markers for classification or staging of cancer (e.g., lymphomas and leukemias), based on characteristic methylation profiles.

Yet further aspects provide expression markers and respective methods for detection, diagnosis, prognosis, monitoring responses to therapies, detection of relapse patients.

### BRIEF DESCRIPTION OF THE DRAWINGS

FIG. 1 shows, according to particular aspects, a schematic of the DLC-1 promoter region of interest. Relative positions of CG dinucleotides are illustrated as vertical bars, forward and reverse primers are indicated as mF and mR respectively, and the area covered by the fluorescent probe.

FIG. 2 shows, according to particular aspects, representative MSP gels illustrating cases of follicular lymphoma (FL) and B-CLL/SLL (CLL). Each panel includes (from the left) lanes for water (H₂O), positive (P) and negative (N) controls, and 15 samples each of FL and CLL. The methylated alleles are shown with the M primers and the unmethylated with the U primers.

FIG. 3 shows, according to particular aspects, methylation analysis by real-time MSP from controls (BFH and PB) and samples of NHLs as indicated. All values are normalized to β-actin for each sample.

FIG. 4 shows, according to particular aspects, expression analysis of DLC-1 by real-time RT-PCR from controls (BFH and PB) and samples of NHLs as indicated. All values are normalized to GAPDH for each sample.

FIG. 5 shows, according to particular aspects, standard curves for DLC-1 real-time MSP. The two graphs on the right illustrate results from 1, 5, 10, 50, 100, and 500 ng of input DNA from the RL cell line without any added salmon sperm DNA. The two graphs on the left illustrate results from the same input DNA from the RL cell line, but with addition of 1 μg salmon sperm DNA.

FIG. 6 shows, according to particular aspects, hierarchical clustering analysis of DNA methylation data. The dendrogram on the top lists the patient sample from the small B cell lymphoma subtypes (MCL, B-CLL/SLL, FL) and follicular hyperplasia (HP). This illustrates a measure of the relatedness of DNA methylation across all loci for each sample. Each column represents one sample and each row represents a single CGI clone on the microarray chip. The fluorescence ratios of Cy3/Cy5 are measures of DNA methylation and are depicted as a color intensity (−2.5 to +2.5) in log 2 base scale; yellow indicates hypermethylated CpG loci, blue indicates hypomethylated loci, and black indicates no change. Regions A-D in the left panel illustrate patterns from the overall array. Interesting sub-regions for each of these is expanded in the middle panel, and the labels on the right identify named genes that are candidates for further study.

FIGS. 7A, 7B and 7C show, according to particular aspects, pair-wise hierarchical clustering analysis of FL and MCL (7A, left panel), B-CLL/SLL and MCL (7B, middle panel), and B-CLL with FL (7C, right panel). Regions of each pairing that show preferential methylation of named genes are shown to the right of each set. The fluorescence ratios of Cy3/Cy5 are measures of DNA methylation and are depicted as a color intensity (−2.5 to +2.5) in log 2 base scale; yellow indicates hypermethylated CpG loci, blue indicates hypomethylated loci, and black indicates no change.

FIG. 7D shows a demonstration of class separation of various subtypes of B-cell non-Hodgkin's lymphomas. Shown is the hierarchical clustering of cases from B-cell chronic lymphocytic leukemia/small lymphocytic lymphoma (CLL), mantle cell lymphoma (MCL), grades I and II follicular lymphoma (FL), and diffuse large B-cell lymphoma (DLBCL). Thus, methylation profiling, according to particular aspects, has located many genes that are useful in diagnosis and/or classification and as markers of diagnosis, response to therapy, early relapse, or as therapeutic drug targets.

FIG. 8 shows, according to particular aspects, methylation specific PCR validation of a subset of candidate genes from microarray studies using NHL cell lines. The presence of a visible PCR product is indicated as M (methylated) or U (unmethylated) genes. In some instances, both methylated and unmethylated alleles are present. Normal female (NL1) and male (NL2) peripheral blood lymphocyte DNA was used as negative controls and in vitro methylated DNA using SssI methyltransferase was the positive control.

FIG. 9 shows, according to particular aspects, determination of promoter hypermethylation of 9 genes from microarray findings in SBCL subsets (MCL, B-CLL/SLL and FLI). The left panel shows patterns in the NHL cell lines, while the de novo tumor groups are indicated at the top of each additional panel, with the gene names listed to the left. The methylation status of a given gene in a particular patient is indicated by a filled square.

FIG. 10 shows, according to particular aspects, an illustration of the relationship of B-cell non-Hodgkin's lymphomas in this study to stages of normal B-cell maturation.

FIG. 11 shows, according to particular aspects, DNA methylation analysis of 6 NHL cell lines. Left panel; cluster analysis of the methylation microarray data derived from 6 NHL cell lines using Cluster 3.0 and Treeview™ software. BCL6 expression was measured by real time PCR and CD10 expression by flow cytometry as described in the materials and methods. Right panel; analysis of DNA methylation in 10 methylation-dependent genes in a panel of 6 NHL cell lines. MSP and COBRA were used to determine the methylation status of 10 CpG island loci in lymphoma cell lines. For COBRA assay, genomic DNA (2 μg) was bisulfite-treated and subjected to PCR using primers flanking the interrogating BstUI site(s) in each CpG island locus. PCR products were digested with BstUI and separated on 3% agarose gels. As shown, the digested fragments reflect BstUI methylation within a CpG island. Control DNA was methylated in vitro with the SssI methylase. Primers specific for methylated and unmethylated DNA were used in MSP assay.

FIG. 12 shows, according to particular aspects, expression analysis of four selected genes in 6 NHL cell lines: total RNA (2 μg) isolated from treated (A, DAC; T, TSA; and AT, DAC+TSA;) or untreated (C) cells was used to generate cDNA for real time RT-PCR. cDNA generated from a normal lymph node samples served as a positive control (scored 100). GAPDH was used as a control to normalize the gene expression under different conditions.

FIG. 13 shows, according to particular aspects, confirmation of promoter hypermethylation in clinical NHL cases. Only representative COBRA results are showed. Briefly, genomic DNA (2 μg) was bisulfite-treated and subjected to PCR using primers flanking the interrogating BstUI site(s) in each CpG island locus. PCR products were digested with BstUI and separated on 3% agarose gels. As shown, the digested fragments reflect BstUI methylation within a CpG island. P: positive control DNA methylated in vitro with the Sss I methylase; N: negative control (normal peripheral lymphocyte) DNA.

FIGS. 14A, B and C show, according to particular aspects, comparative analysis of methylated genes across NHL subtypes. FIG. 14A; methylation distribution of 6 genes among 57 clinical NHL cases. Red box: methylated; Green box: unmethylated; Grey box: not determined. FIG. 14B; comparison of frequencies of aberrant methylation in NHL samples. FIG. 14C; comparison of mean methylation indices in NHL subtypes. Frequencies of methylation of two groups were compared using Fisher's exact test. Ps are shown when there was a significant difference between two groups. The methylation index (MI) is defined as the total number of genes methylated divided by the total number of genes analyzed. To compare the extent of methylation for a panel of genes examined, the MIs for each case were calculated and the mean for the different groups was then determined. Mann-Whitney U test was used to compare the mean MIs between two variables.

FIGS. 15A, B and C show, according to particular aspects, quantitative analysis of DLC-1 methylation and expression in primary NHLs. FIG. 15A; Methylation analysis by real-time MSP from controls (BFH and PB) and samples of NHLs as indicated. Each circle represents a unique sample and the solid horizontal bar indicates the median ratio of methylated DLC-1/β-Actin ratios×1000 within a group of patients. FIG. 15B; Expression analysis of DLC-1 by real-time RT-PCR from controls (BFH and PB) and samples of NHLs as indicated. All values are normalized to GAPDH for each sample. FIG. 15C; Methylation analysis by real-time MSP from plasma samples of NHLs.

FIG. 16 shows, according to particular aspects, a scheme of DNA methylation analysis using a CpG island microarray. Genomic DNA is digested with restriction enzyme Mse I. The digested fragments are ligated to linkers that are specific for MseI restriction ends and contain PCR primer sequences. The linker-ligated DNA is then divided into two aliquots. One aliquot is the test sample and is digested with a methylation sensitive restriction enzyme McrBC which only cuts methylated DNA sequences, while the other aliquot is the reference and is not digested with McrBC. These two aliquots are then amplified by PCR, followed by a random labeling step with aa-dUTP. The aa-dUTP labeled DNA from the test and reference samples are coupled with Cy5 and Cy3 and then used for microarray hybridization.

FIG. 17 shows, according to particular aspects, scatter plots A-D of the methylation microarray analysis in multiple myelomoa (MM) cell lines using the 12K CpG island microarray panel. Microarray hybridization was conducted as described herein (e.g., Example 4). Cy5/Cy3 ratios of tumor cells were plotted against sex matched normal control samples. The blue line is a 45 degree angle line (y=x), the pink line is ½ fold line (y=½x), and the yellow line is ¼ fold line (y=¼x). A lower Cy5/cy3 ratio of the cancer cell line as compared to the normal control indicates hypermethylation and a higher Cy5/Cy3 ratio of the cancer cell line indicates hypomethylation.

FIG. 18 shows, according to particular aspects, hierarchical clustering of the DNA methylation data was performed using Cluster software. Analysis of 3,962 CpG island loci that are associated with annotated genes yielded a tree that separates the 18 MM samples into groups. The methylation index ratios used for the cluster analysis are defined as the Cy5/Cy3 ratio from tumor sample divided by the Cy5/Cy3 ratio from a normal control sample. A lower Cy5/cy3 ratio of the tumor cells as compared to the normal control indicates hypermethylation and a higher Cy5/Cy3 ratio of the tumor cells indicates hypomethylation.

FIGS. 19A and B show, according to particular aspects, analysis of DNA methylation in 10 methylation-dependent genes in a panel 4MM cell lines. MSP and COBRA were used to determine the methylation status of 10 CpG island loci in myeloma cell lines. For COBRA assay, genomic DNA (1 μg) was bisulfite-treated and subjected to PCR using primers flanking the interrogating BstUI site(s) in each CpG island locus. PCR products were digested with BstUI and separated on 3% agarose gels. As shown, the digested fragments reflect BstUI methylation within a CpG island. Control DNA was methylated in vitro with the SssI methylase. Primers specific for methylated and unmethylated DNA were used in an MSP assay.

FIGS. 20A and B show, according to particular aspects, the sensitivity of a qMSP assay for DLC-1. The standard curves were generated using serial dilutions of Raji cell DNA before bisulfite treatment. For these purposes, 10, 50, 100 and 500 ng of Raji DNA was bisulfite treated and used for the qMSP assay. The Ct value of each reaction was then plotted against the amount of input DNA used in the bisulfite reaction. The results indicate how much DNA is needed for a positive detection of DLC-1 methylation. It also demonstrated that the quantitative aspect of this assay is not affected by bisulfite treatments.

FIG. 21 shows, according to particular aspects, Real-time methylation specific PCR shows a quantitative difference of DLC-1 promoter methylation between MMs and normal controls. The methylated DLC-1/β-Actin ratios X1000 represents the degree of methylation. The qMSP primers and probe for Actin do not contain the CGs and therefore represent the quantitative estimate of input DNA in the PCR reaction.

FIG. 22 shows, according to particular aspects,

FIGS. 23A and B show, according to particular aspects, cluster analysis of sample methylation features, demonstrating that the FAB M0-M3 subtypes could be discriminated on the basis of their methylation profile patterns (FIG. 23A).

FIG. 23B shows, according to additional aspects, Hierarchical clustering of DNA methylation in AML and ALL. Methylation microarray analysis revealed distinctive methylation patterns in AML and ALL patients from different subtypes: Region “1” illustrates loci hpermethylated in AML; Region “2” shows loci hypermethylated in both AML and ALL; and Region “3” shows loci hypermehtylated in ALL patients.

FIGS. 24A and B show, according to particular aspects, validation of promoter methylation in 10 genes identified in CpG island array analysis. FIG. 24A shows validation in 16 ALL patients. DLC-1 was validated by real-time qMSP assay, LRP1B was validated by MSP and the remaining genes were validated by COBRA. Shaded blocks indicate methylation detected and white blocks indicate no methylation detected. Each column represents an individual gene and each row represents an individual patient.

FIG. 24B shows validation in 4 ALL cell lines: 1) Jurkat; 2) MN-60; 3) NALM-6; 4) SD-1; N) bisulfite treated normal DNA; P) SssI and bisulfite treated DNA; and L) Ladder. The gel pictures located above the solid line are the results of COBRA analysis and the gel pictures below the solid line are the results of MSP. LRP1Bm: assay for methylated allele; LRP1Bu: assay for unmethylated allele. The results from the DLC-1 qMSP assay are not presented for the cell lines (Jurkat-positive; MN60-positive; NALM6-positive; SD 1-negative).

FIGS. 25A and B show, according to particular aspects, change in mRNA expression in Jurkat and NALM-6 cell lines post treatment with a demethylating agent and a histone deacetylase inhibitor. FIG. 25A shows genes with a 10-fold or greater increase in mRNA expression after treatment in at least one cell line. Solid columns represent the Jurkat cell line and spotted columns represent the NALM6 cell line. The symbol “//” represents a relative expression level greater than 80 with the actual level located in the text above each column.

FIG. 25B shows genes with a 2 to 10-fold increase in mRNA expression after treatment in at least one cell line. Solid columns represent the Jurkat cell line and spotted columns represent the NALM6 cell line: 1) Jurkat Control—no treatment; 2) Jurkat 5-aza treatment; 3) Jurkat TSA treatment; 4) Jurkat 5-aza and TSA treatment; 5) NALM6 Control—no treatment; 6) NALM6 5-aza treatment; 7) NALM6 TSA treatment; and 8) NALM6 5-aza and TSA treatment.

FIG. 26 shows, according to particular aspects, a novel gene selection algorithm: the final selection of differentially methylated genes (loci) is made after the tuning is performed by grouping the patients in three clusters that match the pathological diagnoses (see Example 7 herein).

FIGS. 27a-c show, according to particular aspects, the modified method “idealized methylation pattern” (IMP) method (one of two methods used in gene selection; Example 7). To determine if a gene is exclusively hypermethylated in CLL, the ideal hypermethylation profile for the CLL class (FIG. 27a; top panel) is correlated with the observed gene hypermethylation pattern (FIG. 27b; middle panel). For example, the gene from figure (FIG. 27b) is better correlated with the IMP for the CLL class (FIG. 27a) than the gene in figure (FIG. 27c; bottom panel).

FIGS. 28A and B show, according to particular aspects, a hypermethylation profile and the sample cross-correlation for a set of 160 genes selected using the inventive IHP method.

FIG. 29 shows, according to particular aspects, a representation of 46 patients in 2D using MDS and the patient correlation matrix computed using 160 genes selected using IMP (from FIG. 28B).

FIGS. 30A and B show, according to particular aspects, a hypermethylation profile and the patient cross-correlation for a set of 213 genes selected using the t-test method.

FIG. 31 shows, according to particular aspects, a representation of 46 patients in 2D using MDS and the patient correlation matrix computed using 213 genes selected using t-test (from FIG. 30B).

FIG. 32 shows additional embodiments providing for a method for simultaneous gene selection in, for example, B-cell lymphoma from methylation and expression microarrays. The approach is analogous to that described in detail in Example 7, except that rank fusion (rank averaging) is between a differentially methylated gene ranking (IMP, -test) and a differentially expressed gene ranking (IEP, t-test), resulting in a fused rank list, from which genes are optimally selected by computing patient correlation matrix, and clustering of the patient similarity matrix using C-means to select for an optimal number of genes that best match the pathologically determined lymphoma diagnoses

### DETAILED DESCRIPTION OF THE INVENTION

Particular aspects of the present invention provide novel methylation and/or expression markers that serve as biomarkers in novel methods for detection, monitoring, diagnosis, prognosis, staging, treatment response prediction/monitoring/guidance, etc., of cancer including hematopoietic malignancies, leukemia, lymphomas, etc., (e.g., non-Hodgkin's lymphomas (NHL), small B-cell lymphomas (SBCL), diffuse large B-cell lymphoma (DLBCL), follicular lymphoma (FL), mantle cell lymphoma (MCL), B-cell chronic lymphocytic leukemia/small lymphocytic lymphoma (B-CLL/SLL), chronic lymphocytic leukemia (CLL), multiple myeloma (MM), acute myelogenous leukemia (AML), acute lymphoblastic leukemia (ALL), etc.).

### Description of Preferred Methylation Profiling and Expression Profiling Embodiments:

A high-throughput array-based technique called differential methylation hybridization (DMH) was used in particular aspects of the Examples (below) to study and characterize hematopoietic malignancies, leukemia, lymphomas, etc. (and in particular instances, subtypes/stages thereof), based on establishing a set of novel methylation and/or expression biomarkers.

From the initial microarray experiments, several statistical methods were used to generate limited sets of genes for further validation by methylation specific PCR (MSP) and/or COBRA using cancer tissue and/or relevant cell lines. Hierarchical clustering of the DNA methylation data was then used to characterize a particular cancer type, or subtype, on the basis of their DNA methylation patterns/profiles, revealing, as disclosed herein, that there is diversity of characteristic DNA methylation patterns between and among the different cancers and cancer subtypes.

In EXAMPLE 1 herein, DLC-1 promoter methylation was demonstrated by quantitative analysis, to have substantial utility as a differentiation-related biomarker of non-Hodgkin's Lymphoma (NHL).

Applicants previously used an Expressed CpG Island Sequence Tags (ECIST) microarray technique (11) and identified DLC-1 as a gene whose promoter is methylated in NHLs and results in gene silencing. Example 1 discloses quantitative real-time methylation-specific PCR analysis to examine promoter methylation of DLC-1 (deleted in liver cancer 1, a putative tumor suppressor) and its relationship to gene silencing in non-Hodgkin's lymphomas (NHL). Gene promoter methylation of DLC-1 occurred in a differentiation-related manner and has substantial utility as a biomarker in non-Hodgkin's Lymphoma (NHL).

Specifically, a high frequency of DLC-1 promoter hypermethylation was found to occur across different subtypes of NHLs, but not in cases of benign follicular hyperplasia (BFH). More specifically, methylation of DLC-1 was observed in 77% (79 of 103) of NHL cases; including 62% (8 of 13) in MCL, 71% (22 of 31) in B-CLL/SLL (B-cell chronic lymphocytic leukemia/small lymphocytic lymphoma), 83% (25 of 30) in FL, and 83% (24 of 29) in DLBCL samples. When thresholded values of methylation of DLC-1 were examined, 100% specificity was obtained, with 77% sensitivity.

Expression studies demonstrated down-regulation of DLC-1 in NHL compared to normal lymph nodes, and this may be re-activated using therapies/agents that modulate methylation and acetylation.

According to additional aspects, GSTP1, CDKN1A, RASSF1A and DAPK methylation markers have substantial utility as biomarkers of cancer (e.g., non-Hodgkin's Lymphoma).

The DLC-1 gene has been mapped to chromosome 8p21.3-22, a region suspected to harbor tumor suppressor genes and deleted in several solid tumors (21-23). The DLC-1 sequence shares high homology with rat p122RhoGAP, a GTPase-activating protein for Rho family proteins, and DLC-1 protein was shown to be a RhoGAP specific for RhoA and Cdc42 (24). RhoGAPs serve as tumor suppressors by balancing the oncogenic potential of Rho proteins. Recent evidence suggests that RhoA GTPase regulates B-cell receptor (BCR) signaling and may be an important regulator of many aspects of B-cell function downstream of BCR activation (25). Consistent with this notion, the reintroduction of DLC-1 inhibits the proliferation of DLC-1-defective cancer cells (26). Applicants have herein demonstrated that DLC-1 is frequently methylated across all 4 major sub-classes of NHLs. Further, this promoter methylation is reciprocal to DLC-1 mRNA in most of the NHLs examined. Therefore, according to particular aspects of the present invention, the use of this quantitative assay has substantial utility to improve the detection rate of NHL in tissue biopsies, and from blood and/or plasma samples.

In EXAMPLE 2 herein, a CpG island microarray study of DNA methylation was performed with samples of Non-Hodgkin's Lymphomas (NHL) with different clinical behaviors. Non-Hodgkin's Lymphoma (NHL) is a group of malignancies of the immune system that encompasses subtypes with variable clinical behaviors and diverse molecular features. Small B-cell lymphomas (SBCL) are low grade NHLs including mantle cell lymphoma, B-cell chronic lymphocytic leukemia/small lymphocytic lymphoma, and grades I and II follicular lymphoma.

Differential methylation hybridization (DMH) was used to study SBCL subtypes based on a large number of potential methylation biomarkers. From these microarrays, several statistical methods were used to generate a limited set of genes for further validation by methylation specific PCR (MSP). Hierarchical clustering of the DNA methylation data was used to group each subtype on the basis of similarities in their DNA methylation patterns, revealing that there is a characteristic diversity in DNA methylation among the different subtypes. In particular, differential methylation of LHX2, POU3F3, HOX10, NRP2, PRKCE, RAMP, MLLT2, NKX6-1, LPR1B, and ARF4 markers was validated in NHL cell lines and SBCL patient samples, and demonstrated a preferential methylation pattern in germinal center-derived tumors compared to pre- and post-germinal center tumors.

According to particular aspects of the present invention, these markers define molecular portraits of distinct sub-types of SBCL that are not recognized by current classification systems and have substantial utility for detecting, distinguishing between and among, and characterizing the biology of these tumors.

Specifically, characterization of the human lymphoma epigenome was undertaken in the context of studying 3 classes of NHL. The SBCLs, a subset of NHL, exhibit a spectrum of clinical behaviors and the cell of origin of each subtype is thought to be related to a putative stage of normal B-cell differentiation. Mutational status of the variable region of immunoglobulin heavy chain (V_{H}) genes is a useful marker for identifying different developmental stages of NHLs, and relates to processes that occur in the germinal center reaction. MCL (mantle cell lymphoma) is considered to arise in cells at the pre-germinal center stage where V_{H} genes have not yet become mutated (34). In FL (follicular lymphoma), somatic hypermutation of V_{H} genes characteristic of the germinal center reaction suggests that this class of NHL derives from a germinal center stage of differentiation. Approximately half of B-CLL/SLL (B-cell chronic lymphocytic leukemia/small lymphocytic lymphoma) cases are CD38+ with unmutated V_{H} genes (poor prognosis) and the remaining half are CD38− with mutated V_{H} genes (better prognosis). Thus, B-CLL/SLL may represent two separate stages of differentiation; pre-germinal center and post-germinal center, respectively. The SBCL subtypes studied in the present Example represent a spectrum of pre-germinal center, germinal-center and post germinal-center stages of B-cell differentiation and provide a good model to study epigenetic alterations as they might relate to the various compartments of secondary lymphoid tissue cell differentiation.

High-throughput technologies have clearly advanced understanding of the gene expression repertoire of human tumors. Utilization of cDNA microarray analysis allows classification of different malignancies based on dysregulation of gene expression. In one report, hierarchical clustering analysis separated FL from MCL based on gene expression profiles (35). However, such studies do not address the underlying reason(s) for changes in gene expression. In the present Example, the CGI microarray was utilized to investigate part of the NHL epigenome of SBCL subtypes based on interrogation of promoter DNA methylation, a process that plays an role in human cancers by frequently silencing not only tumor suppressor genes, but also genes that are critical to the normal functions of cells, such as apoptosis, cell cycle regulation, cellular signaling, and gene transcription (reviewed in (29, 31)). The disruption of such cellular activities may play a role in lymphomagenesis and/or secondary events such as tumor progression or transformation.

Hierarchical clustering analysis of data from the CGI microarray identified approximately 256 named, variably methylated genes, within SBCL subtypes and recognized genes that are important to many intracellular processes. Additional CGI loci were also differentially methylated, but at this time, some are hypothetical genes and some have not yet been investigated for identity.

LHX2. The LHY2 gene belongs to a superfamily of homeobox-containing genes conserved during evolution and function as transcriptional regulatory proteins in control of lymphoid and neural cell differentiation (36).

POU. The POU family proteins also act as transcriptional factors and regulate tissue-specific gene expression at different stages of development in the nervous system (37).

NRP2. Non-kinase neuropilin 2 (NRP2) was predominantly methylated in FL (p=0.001). This gene encodes a member of the neuropilin family of receptors that binds to SEMA3C (sema domain, Ig domain, short basic domain, secreted, semaphoring 3C) protein and also interacts with vascular endothelial growth factor (VEGF) (38), an important mediator of angiogenesis, a process important in NHL as well as other tumors.

ARF4. Additionally, ADP ribosylation factor 4 (ARF4), which plays a role in vesicular trafficking and as an activator of phospholipase D, was methylated in 7/12 (58.3%) of MCL and 13/15 (87%) FL cases (p=0.001).

Phospholipase D. Phospholipase D is an enzyme involved in the CD38 signaling pathway and regulates lymphocyte activation and differentiation (39).

LRP1B. The LRP1B gene is frequently deleted in various tumor types, but in this Example shows a higher frequency of gene promoter methylation in germinal center SBCLs compared to the other subtypes (p=0.001). CGI promoter hypermethylation of this gene has also been detected in esophageal squamous cell carcinomas (40).

This Example further demonstrates the value of the high-throughput CGI microarray to rapidly interrogate 8,544 (9K) clones from a CGI library isolated by the Huang laboratory (41). In a recent study (22) comparing this 9K library to another containing 12,192 (12K) clones, only 753 were found to be common between the 2 libraries, thus suggesting that the present Example examined ˜50% of potential CGIs in the human genome. Nevertheless, this does not diminish the value of finding many new, epigenetically altered, genes that segregate with subclasses of NHL.

According to particular aspects of the present invention, the herein-disclosed validated markers have substantial utility as diagnostic tools, and for monitor treatment of NHL. The Example also illustrates a very interesting biological finding; preferential methylation of multiple gene promoters in germinal-center tumors such as FL compared to pre-germinal center tumors (MCL and some B-CLL/SLL) and post-germinal center tumors (subset of B-CLL/SLL). Without being bound by mechanism, the reasons for this may be related to the ongoing somatic hypermutations and the process of DNA strand breaks and repair (both effective and ineffective) that accompanies germinal-center biology, and may be possibly carried over into germinal-center NHLs. The findings of this Example thus provide a basis for investigations of gene promoter DNA methylation in NHLs, and provide useful insights into the functional epigenomic signatures of human lymphomas.

The epigenome becomes even more important because there has been a great deal of recent development of pharmaceutical interventions that can potentially reverse epigenetic alterations with the intent of reactivating silenced genes in cancers as a form of chemotherapy (31-33).

In EXAMPLE 3 herein, novel epigenetic Markers for non-Hodgkin's lymphoma (NHL) were discovered using a CpG island microarray analysis. Specficially, using the CpG island microarray approach, a substantial number of additional genes were identified that are, according to particular aspects of the present invention, aberrantly methylated in NHL cell lines and in primary NHLs. According to such aspects, these markers, alone or in combination, have utility detection or diagnosis. A combination of each gene can be used as a molecular marker panel for detection or diagnosis using highly sensitive quantitative methylation specific PCR technology. An advantage of such markers is that they are derived from patients' tumor DNA, which is a more stable specimen than RNA. Hypermethylation of gene loci detected in the assay could be indirect evidence for genes down-regulated in the primary tumors. Although a growing number of genes have been identified as aberrantly methylated in lymphoma (5, 6, 19), to date few studies (7-9) have studied promoter hypermethylation in the specific NHL subtypes in detail.

Applicants have not only identified genes like DLC-1 and PCDHGB7 which are methylated in the vast majority of NHLs, but also have identified some subtype-specific markers such as CCND1, CYP27B1, RARβ2 and EFNA5 which are preferentially methylated in one or two subtypes of NHLs. Using DLC-1 as an example, the ability to detect aberrant methylated DNA in 77% of tumor and 67% of plasma samples from primary NHL patients using quantitative real time MSP was demonstrated herein. Therefore, according to particular aspects, these markers have utility as biomarkers in diagnosis and classification of NHLs, especially for early detection and monitoring therapy.

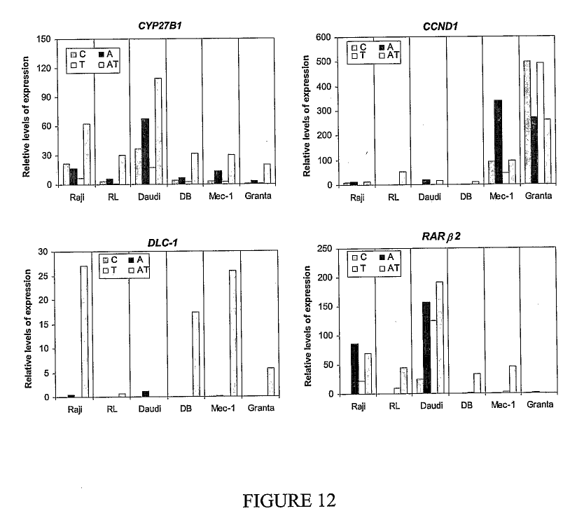

As shown herein, a candidate tumor suppressor gene DLC-1 is a frequent target of aberrant methylation in NHLs. While methylation of the gene has been previously reported in several types of non-lymphohematopoietic tumors (20-23), this is the first report of its involvement in NHL. The DLC-1 gene was mapped to 8p21.3-22, a region suspected to harbor tumor suppressor genes and recurrently deleted in several solid tumors (23-25). The DLC-1 sequence shares high homology with rat p122RhoGAP, a GTPase-activating protein for Rho family proteins and DLC-1 protein was shown to be a RhoGAP specific for RhoA and Cdc42 (26). Recent evidence suggests that RhoA GTPase regulates B-cell receptor (BCR) signaling and may be an important regulator of many aspects of B-cell function downstream of BCR activation (27). Therefore, epigenetic silencing of DLC-1 might have a profound influence on lymphomagenesis. Interestingly, DLC-1 is not expressed in peripheral blood lymphocytes but is expressed in the normal lymph node when examined by real time RT-PCR for DLC-1 mRNA and suggests tissue specific or developmental stage dependent expression. However, no methylation was found in the normal B-cells regardless of their expression status. Interestingly, reactivation of methylated DLC-1 genes in NHL cells required both DAC and TSA (FIG. 12) suggesting that DNA methylation is not the only process involved in DLC-1 gene silencing.

The chromosome translocation t(11;14)(q13;32), is seen in most MCLs (2, 28), and as a result, CCND1 is over-expressed in over 90% of MCL (2). A recent finding of complete hypomethylation at the CCND1 promoter in normal B cells suggests that although the CCND1 gene is inactive transcriptionally, the CCND1 promoter is still unmethylated in lymphoid cells that do not contain the translocation (18). It is possible that the mechanism of de novo methylation is dysregulated in NHLs, resulting in aberrant methylation of CCND1 despite its transcriptional status. This finding indicates that such DNA regions in the genome are prone to be methylated in cancer cells, which is consistent with an earlier report (29), although the factors that determine such susceptibility to methylation remain unresolved.

CYP27B1 encodes 1α-hydroxtylase (1α-OHase), an important enzyme in the vitamin D metabolic pathway. The loss of 1α-OHase and/or VDR activity could contribute to the ability of cancer cells to escape growth control mechanisms of vitamin D (30). Several studies have shown that reduced 1α-OHase activities in cancer cells decreased the susceptibility to 25(OH)D₃ induced growth inhibition (31).

Ephrin-A5, a member of the ephrin gene family is encoded by EFNA5. The EPH and EPH-related receptors comprise the largest subfamily of receptor protein-tyrosine kinases and have been implicated in mediating developmental events, particularly in the nervous system. Himanen et al. found that ephrin-A5 binds to the EphB2 receptor(32), a tumor suppressor gene (33), leading to receptor clustering, autophosphorylation, and initiation of downstream signaling.

PCDHGB7 is a member of the protocadherin gamma gene cluster, one of three related clusters tandemly linked on chromosome five. These gene clusters have an immunoglobulin-like organization (34), suggesting that a novel mechanism may be involved in their regulation and expression (35). The two cell surface molecules are known to play a role in the nervous system, but any role they may have in NHL is unclear.

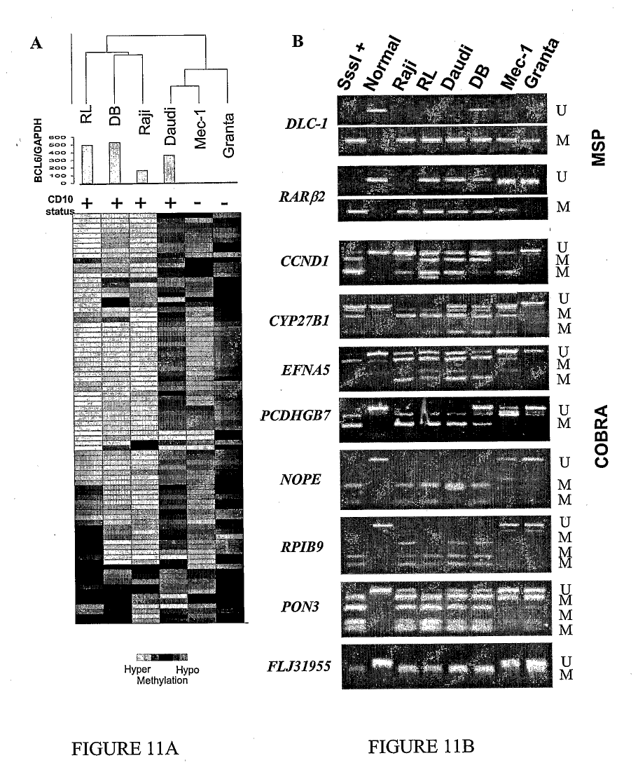

Remarkably, applicants found that there were statistically significant differences in DNA methylation between pre-germinal and germinal center derived NHLs. The mean methylation index of non-germinal center NHLs was lower than germinal center related NHLs. The mechanism and biological significance behind this experimental observation is not clear at this point. Although the effect of age on the increase in methylation cannot be excluded when comparing MCL with FL and DLBCL, age related methylation cannot explain the difference in methylation between CLL, FL and DLBCL. The increased methylation observed in germinal center derived NHL might be associated with over-expression of BCL6 (See FIG. 11). BCL6 is a Kruppel-associated box (KRAB) domain-containing zinc finger protein which is involved in the pathogenesis of NHL. A recent study showed that gene silencing induced by the KRAB-associated protein 1 (KAP-1) complex was followed by regional DNA hypermethylation at the promoter of its target genes (36) and sheds light on the potential role of DNA methylation in BCL6 mediated gene silencing.

Applicants, therefore, have performed analysis of methylation alterations at the genome level in 6 cell lines derived from a spectrum of NHL subtypes, and have identified a group of aberrantly methylated genes which have utility as epigenetic biomarkers for detection of NHL. Applicants have also demonstrated that NHL exhibits nonrandom methylation patterns in which germinal center tumors seem to be prone to de novo methylation. The mechanism behind such experimental observations is unclear, but it is unlikely that all of these methylation events were induced by global deregulation of methyltransferase activity. Instead, dysregulation of a given transcriptional regulator or signaling pathway most likely selectively leads to the aberrant methylation of a portion of downstream genes and confers a growth advantage to the tumor cells

In EXAMPLE 4 herein, multiple novel methylated genes were identified by ECISTs microarray screening, were confirmed in mulitple myeloma (MM) cell lines and primary MM samples, and were shown have substantial utility for diagnosis, prognosis and monitoring of aspects of multiple myeloma.

Expressed CpG Island Sequence Tags (ECISTs) microarray (14), is an integrated microarray system that allows assessing DNA methylation and gene expression simultaneously, and provides a powerful tool to further dissect molecular mechanisms in MMs, and to assess related pharmacologic interventions by differentiating the primary and secondary causes of pharmacological demethylation. This innovative microarray profiling of DNA methylation was used in this Example to define Epigenomic Signatures of Myelomas. Novel epigenetic biomarkers were identified that have substantial utility for diagnosis, prognosis and monitoring.

Methylation microarray profiling was conducted in the context of 4 multiple myeloma (MM) cell lines, 18 MM primary tumors and 2 normal controls. Multiple novel methylated genes were identified, and a subset of these were confirmed in MM cell lines and in primary MM samples (20 primary MM samples from our cell bank, from which DNA was isolated). Additionally, a real time methylation-specific PCR assay was developed for the tumor suppressor gene DLC-1, and was optimized in terms of sensitivity and variability. Furthermore, four MM cell lines were treated with a demethylating agent and histone deacetylase inhibitor, and RNA was isolated from the drug-treated cell lines.

To applicants' knowledge, this Example is the first genome wide methylation analysis of primary MM. The significance of the findings to the scientific field and their potential impact on health is significant in view of the insights into the underlying biology of the epigenetic process of DNA methylation in both normal and neoplastic plasma cell differentiation, and further in view of the substantial diagnostic, prognostic and monitoring utilities and for therapeutic intervention methods involving respective demethylation and/or histone acetylation agents.

In EXAMPLE 5 herein, differential methylation hybridization (DMH) was used to determine and compare the genomic DNA methylation profiles of the granulocyte subtypes of acute myelogenous leukemia (AML).

This Example determines for the first time that genomic methylation profiling can be used to distinguish between clinically recognized subtypes of acute myelogenous leukemia (AML). Aberrant DNA methylation is believed to be important in the tumorigenesis of numerous cancers by both silencing transcription of tumor suppressor genes and destabilizing chromatin. Previous studies have demonstrated that several tumor suppressor genes are hypermethylated in AML, suggesting a roll for this epigenetic process during tumorigenesis. However, it is unknown how the genomic methylation profiles differ among AML variants, or even whether AML can be distinguished on this basis from normal bone marrow or other hematologic malignancies. In this Example, the epigenomic microarray screening technique called Differential Methylation Hybridization (DMH) was applied to the analysis of 23 bone marrow samples from patients having the AML granulocytic subtypes M0 to M3 as well as normal controls.

With this method, a unique genomic methylation profile was created for each patient by screening sample DNA amplicons with an array of over 8600 CpG-rich DNA tag sequences. Cluster analysis of methylation features was then performed that demonstrated these disease subtypes could be sorted according to methylation profile similarities. From this screening, over 70 genomic loci were identified as being hypermethylated in all four examined AML subtypes relative to normal bone marrow. Three hypermethylated loci in M0 samples were found to distinguish this class from all others. Sequence analysis of these loci was performed to identify their encoded genes. Confirmation of their methylation status in AML was conducted using MS-PCR and COBRA analyses.

Results of this Example indicate that genomic methylation profiling has substantial utility not only for diagnosing AML and subtypes thereof, but also in distinguishing this disease from other hematopoietic malignancies. Moreover, analysis of the impact of methylation on the expression of the identified genes will facilitate understanding the underlying molecular pathogenesis of AML.

In EXAMPLE 6 herein, differential methylation hybridization was used to determine the Genomic DNA methylation profiles of Acute Lymphoblastic Leukemia (ALL).

To attain a global view of the methylation present within the promoters of genes in ALL patients and to identify a novel set of methylated genes associated with ALL, methylation profiles were generated for 16 patients using a CGI array consisting of clones representing more than 4 thousand unique CGI sequences spanning all human chromosomes. This is the first time, to applicants' knowledge, that a whole genome methylation scan of this magnitude has been performed in ALL. From the generated profiles, 49 candidate genes were identified that were differentially methylated in at least 25% of the patient samples. Many of these genes are novel discoveries not previously associated with aberrant methylation in ALL or in other types of cancers. Methylation in ten genes found by the CGI array to be differentially methylated in at least 50% of the patients was verified by COBRA, MSP or qMSP. The observations were concordant with the methylation arrays, and the independent verifications indicated that between 10 and 90% of these genes were methylated in every patient. The genes identified in TABLE 7 are involved in a variety of cellular processes including transcription, cell cycle, cell growth, nucleotide binding, transport and cell signaling. In conjunction with the detection of promoter methylation in the ALL samples but not in the normal controls, this indicates that these genes act as tumor suppressors in ALL.

It was determined herein that the 10 validated genes were silenced or down-regulated in NALM-6 and Jurkat ALL cell lines and that their expression could be up-regulated after treatment with a demethylating agent alone or in combination with TSA. Of the validated genes, the greatest post-treatment increase in mRNA expression was for ABCB1, RPIB9 and PCDHGA12 and these appear to be functional genes involved in the development or progression of ALL, and, according to particular aspects, have substantial utility for distinguishing development or progression of ALL. RPIB9 and ABCB1 are genes transcribed in opposite directions with overlapping CGI containing promoters. It has recently been shown that hypomethylation of the ABCB1 promoter leads to multi drug resistance (Baker et al. 2005) and that methylation of the ABCB1 promoter is linked to the down-regulation of gene expression in ALL (Garcia-Manero et al. 2002). This suggests that individuals with methylation in the ABCB1 promoter may better respond to chemotherapeutic treatment than individuals lacking methylation. Although the function of RPIB9 has yet to be confirmed, it likely functions as an activator of Rap which allows B-cells to participate in cell-cell interactions and contributes to the ability of B-lineage cells to bind to bone marrow stromal cells, a requisite process for the maturation of B-cells (McLeod 2004). Therefore, if methylation of the RPIB9 promoter suppresses its transcription, the ability of B-lineage cells to bind to bone marrow stromal cells will likely be inhibited causing the progression of B-lineage cells to halt and resulting in the proliferation of immature cells, a hallmark of ALL. Finally, PCDHGA12 is disclosed herein as an interesting functional gene for ALL in light of a recent report connecting promoter methylation and silencing of PCDHGA11 in astrocytomas and the suggestion that the inactivation of PCDHGA11 is involved in the invasive growth of astrocytoma cells into the normal brain parenchyma (Waha et al. 2005).

In summary, the methylation status of novel genes associated with ALL including NKX6-1, KCNK2, RPIB9, NOPE, PCDHGA12, SLC2A14 and DDX51 was validated Additionally, after treatment with a demethylating agent, mRNA expression was increased in vitro for all 10 genes validated, with the greatest increases occurring for ABCB1, RPIB9, and PCDHGA12. Although the precise role of these genes in ALL progression is unknown, the epigenetic profiles generated in this study, according to particular aspects of the present invention, provide insights to improve our understanding of ALL, provide both novel and noninvasive diagnostic (and/or prognostic, staging, etc.) tools, and novel therapeutic methods and targets for the treatment of ALL. The markers also have substantial utility for distinguishing B-ALL and T-ALL patients.

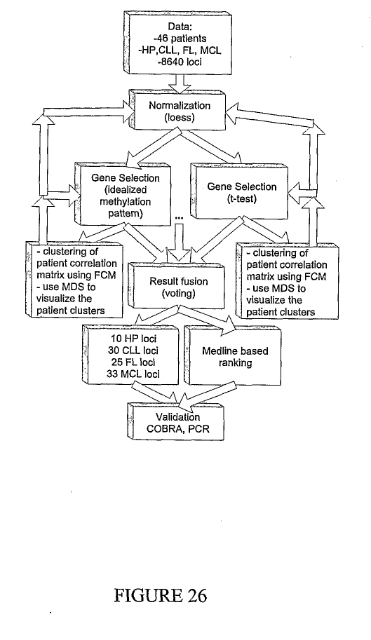

In Example 7 herein, a novel goal oriented approach for finding differentially methylated genes in, for example, small B-cell lymphoma was developed. DNA microarray data was analyzed from three types of small B-cell lymphomas that reveal the extent of CpG island methylation within the promoter and first exon regions of 8,640 loci. A gene can be represented by several loci on the array. The goal of the method is to identify loci (genes) that are uniquely hypermethylated in a specific lymphoma type and hyperplasia (HP). Hyperplasic patients are, for present purposes, considered normal. The inventive gene selection algorithm has 3 main steps (see FIG. 26): array normalization, gene selection and gene clustering. Since the sample grouping is known from the pathological analysis, the clustering step is used as a tuning tool for the first two parts of the algorithm. In addition to error analysis, multidimensional scaling (MDS) was used to visually evaluate the results of the clustering. The final gene selection was performed by fusing the results of two gene selection algorithms. To further assist (e.g., the pathologists) in assessing the selected genes, the medical literature (Medline) were ‘mined’ for associations between the selected genes and, for example, the term “lymphoma”. Initial biological evaluation indicates that the identified discriminant genes are indeed likely to be methylated and involved in essential cellular processes including apoptosis, proliferation, and transcription as well as acting as tumor suppressor genes and oncogenes. Details about each step of the algorithm are presented herein. Additional analogous fused methylation/expression embodiments are also disclosed.

Table 10 shows, according to particular preferred aspects, independently validated novel epigenetic markers for NHL and ALL.

TABLE 10
Independently validated novel epigenetic markers in NHL and ALL

| | Clone Location; | | | CpG Island Location; |
|---|---|---|---|---|
| Clone ID | (SEQ ID NO) | Gene Name | Accession # | (SEQ ID NO) |
| FJ46G1 | chr9: 123858628-123858970; | LHX2 | AF124735 | chr9: 123852801-123860507; |
| | (100) | | | (101) |
| FJ45F11 | chr3: 57557703-57558663; | ARF4 | BC016325 | chr3: 57558061-57558651; |
| | (103) | | | (104) |
| FJ25G8 | chr2: 142721862-142722346; | LRP1B | AF176832 | chr2: 142721457-142722285; |
| | (106) | | | (107) |
| FJ46A4 | chr2: 45782052-45782913; | PRKCE | NM_005400 | chr2: 45788830-45791336; |
| | (109) | | | (110) |
| FJ32F2 | chr4: 85773754-85774366; | NKX6-1 | NM_006168 | chr4: 85774839-85777978; |
| | (112) | | | (113) |
| FJ27D1 | chr12: 52675489-52676226; | HOXC10 | BC001293 | chr12: 52675381-52675787; |
| | (115) | | | (116) |
| FJ63F2 | chr2: 104927795-104928343; | POU3F3 | NM_006236 | chr2: 104927370-104932006; |
| | (118) | | | (119) |
| FJ46C3 | chr2: 206376414-206376687; | NRP2 | BC009222 | chr2: 206375106-206376822; |
| | (121) | | | (122) |
| FJ47G6 | chr1: 208596523-208597879; | RAMP | BC033297 | chr1: 208597233-208597759; |
| | (124) | | | (125) |
| FJ8F8 | chr8: 13034243-13034709; | DLC-1 | NM_006094 | chr8: 13034462-13035285; |
| | (127) | | | (128) |
| Sanger | chr3: 25444632-25445406; | RARB | NM_000965 | Chr3: 25,444,258-25,445,160 |
| 26F2 | (130) | | | |
| FR1A6 | chr12: 56446588-56447155; | CYP27B1 | BC001776 | chr12: 56445123-56446267; |
| | (132) | | | (133) |
| FJ3F12 | chr5: 140767835-140768293; | PCDHGB7 | NM_018927 | chr5: 140777347-140777885; |
| | (135) | | | (136) |
| FJ31B11 | chr5: 107036786-107037187; | EFNA5 | NM_001962 | chr5: 107033030-107036090; |
| | (138) | | | (139) |
| FJ43G12 | chr11: 69161136-69161494; | CCND1 | NM_053056 | chr11: 69160318-69167777; |
| | (141) | | | (142) |
| FJ60C11 | chr7: 94669774-94670779; | PON3 | NM_000940 | chr7: 94670211-94670773; |
| | (144) | | | (145) |
| FJ30A12 | chr5: 38293115-38293710; | FLJ39155 | NM_152403 | chr5: 38293583-38294893; |
| | (147) | | | (148) |
| FJ12A3 | chr1: 211643229-211643982; | KCNK2 | AF004711 | chr1: 211644447-211645031; |
| | (150) | | | (151) |
| FJ32F2 | chr4: 85773754-85774366; | NKX6-1 | NM_006168 | chr4: 85771177-85772053; |
| | (153) | | | (154) and chr4: 85774839-85777978; |
| | | | | (155) |
| FJ7H3 | chr5: 140790472-140790822; | PCDHGA12 | NM_003735 | chr5: 140790679-140792801; |
| | (157) | | | (158) |
| FJ30F9 | chr7: 86902729-86903236; | RP1B9 | NM_138290 | chr7: 86901610-86903095; |
| | (160) | | | (161) |
| FJj30F9 | chr7: 86902729-86903236 | ABCB1 | NM_000927 | chr7: 86901610-86903095 |
| FJ23G11 | chr12: 7915942-7916816; | SLC2A14 | NM_153449 | chr12: 7916632-7917175; |
| | (163) | | | (164) |
| FJ55C3 | chr12: 131293874-131294410; | DDX51 | NM_175066 | chr12: 131294097-131295699; |
| | (166) | | | (167) |
| FJ71F3 | chr15: 63476002-63476565; | NOPE | NM_020962 | chr15: 63476196-63476415; |
| | (169) | | | (170) and chr15: |
| | | | | 63475093-63475592; |
| | | | | (171) |
| FJ78C8 | chr18: 49205528-49206202; | DCC | NM_005215 | chr18: 48122376-48122757; |
| | (173) | | | (174) |

| | Amplicon Location; | | Diseases |
|---|---|---|---|
| Clone ID | (SEQ ID NO) | Assay | Studied |
| FJ46G1 | chr9: 123858851-123858949; | MSP | NHL |
| | (102) | | |
| FJ45F11 | chr3: 57558364-57558563; | MSP | NHL |
| | (105) | | |
| FJ25G8 | chr2: 142722049-142722154; | MSP | NHL, |
| | (108) | | ALL |
| FJ46A4 | chr2: 45782662-45782800; | MSP | NHL |
| | (111) | | |
| FJ32F2 | chr4: 85774136-85774253; | MSP | NHL |
| | (114) | | |
| FJ27D1 | chr12: 52675687-52675873; | MSP | NHL |
| | (117) | | |
| FJ63F2 | chr2: 104927960-10492983; | MSP | NHL |
| | (120) | | |
| FJ46C3 | chr2: 206376438-206376606; | MSP | NHL |
| | (123) | | |
| FJ47G6 | chr1: 208597643-208597766; | MSP | NHL |
| | 126) | | |
| FJ8F8 | chr8: 13035037-13035185; | qMSP | NHL, |
| | (129) | | ALL |
| Sanger | chr3: 25,444,859-25,444,988; | MSP | NHL |
| 26F2 | (131) | | |
| FR1A6 | chr12: 56446852-56447155; | COBRA | NHL |
| | (134) | | |
| FJ3F12 | chr5: 140,777,593-140,777,963; | COBRA | NHL |
| | (137) | | |
| FJ31B11 | chr5: 107035404-107035587; | COBRA | NHL |
| | (138) | | |
| FJ43G12 | chr11: 69,163,118-69,163,378; | COBRA | NHL |
| | (143) | | |
| FJ60C11 | chr7: 94670531-94670808; | COBRA | NHL |
| | (146) | | |
| FJ30A12 | chr5: 38294642-38294937; | COBRA | NHL |
| | (149) | | |
| FJ12A3 | chr1: 211643793-211644022; | COBRA | ALL |
| | (152) | | |
| FJ32F2 | chr4: 85773783-85773994; | COBRA | ALL |
| | (156) | | |
| FJ7H3 | chr5: 140790654-140790834; | COBRA | ALL |
| | (159) | | |
| FJ30F9 | chr7: 86902721-86903123; | COBRA | ALL |
| | (162) | | |
| FJj30F9 | chr7: 86902721-86903123 | COBRA | ALL |
| FJ23G11 | chr12: 7916511-7916783; | COBRA | ALL |
| | (165) | | |
| FJ55C3 | chr12: 131294031-131294283; | COBRA | ALL |
| | (168) | | |
| FJ71F3 | chr15: 63476161-63476562; | COBRA | ALL |
| | (172) | | |
| FJ78C8 | chr18: 48199801-48200041; | COBRA | ALL |
| | (175) | | |

TABLE 11 shows, according to particular preferred aspects, markers for FL and MCL as identified by methylation hybridization as described in the EXAMPLES herein.

| | | T7 | M13 | Chro- | | | | | | |
|---|---|---|---|---|---|---|---|---|---|---|
| | | Sequence | Sequence | mosome | | | | Distance | | Gene/Assession |
| No. | Clone ID | Length | Length | Aligned | Alignment Address | Strand | TSS | to TSS | Direction | Number |
| 1 | FJ#23D6 | 879 | 826 | 5 | 43638478-43640026 | + | 43638581 | 0 | within | NM_012343 |
| | | | | 5 | 43638478-43640026 | + | 43639063 | 0 | within | NNT/U40490 |
| | | | | 5 | 43638478-43640026 | + | 43639063 | 0 | within | NM_182977 |
| 2 | FJ#40H11 | 705 | 705 | 22 | 38039861-38040545 | − | 38035470 | 4391 | upstream | AY320405 |
| | | | | 22 | 38039861-38040545 | − | 38037997 | 1864 | upstream | RPL3/BC004323 |
| | | | | 22 | 38039861-38040545 | − | 38039014 | 847 | upstream | RPL3/BC022790 |
| | | | | 22 | 38039861-38040545 | − | 38040115 | 0 | within | RPL3/BC012786 |
| | | | | 22 | 38039861-38040545 | − | 38040128 | 0 | within | NM_000967 |
| 3 | FJ#13D12 | 420 | 803 | 19 | 58297334-58298137 | − | 58298468 | 331 | upstream | ZNF160/BC000807 |
| | | | | 19 | 58297334-58298137 | − | 58298488 | 351 | upstream | NM_033288 |
| | | | | 19 | 58297334-58298137 | − | 58298488 | 351 | upstream | NM_198893 |
| | | | | 19 | 58353278-58353332 | − | 58354096 | 764 | upstream | NM_032584 |
| | | | | 19 | 58387931-58387955 | − | 58388415 | 460 | upstream | NM_024733 |
| 4 | FJ#40F9 | 919 | 835 | 2 | 69880005-69881175 | + | 69880756 | 0 | within | BC063672 |
| | | | | 2 | 69880005-69881175 | + | 69880931 | 0 | within | NM_001153 |
| 5 | FJ#3B4 | 475 | 831 | 19 | 17391327-17391555 | + | 17391911 | 356 | downstream | LOC93343/BC011840 |
| | | | | 19 | 17391327-17391555 | + | 17391911 | 356 | downstream | NM_138401 |
| 6 | FJ#46B6 | 746 | 495 | 1 | 25339237-25339786 | + | 25344320 | 4534 | downstream | NM_016124 |
| | | | | 1 | 25339237-25339786 | + | 25344320 | 4534 | downstream | NM_016225 |
| | | | | 1 | 25339237-25339786 | + | 25344338 | 4552 | downstream | RHD/X63097 |
| | | | | 1 | 25339237-25339786 | + | 25344354 | 4568 | downstream | RHD/AY449385 |
| | | | | 1 | 25339237-25339786 | + | 25344354 | 4568 | downstream | AF037626 |
| | | | | 1 | 25339237-25339786 | + | 25344354 | 4568 | downstream | AB037270 |
| | | | | 1 | 25409579-25410115 | + | 25410126 | 11 | downstream | SMP1/AL136627 |
| | | | | 1 | 25409579-25410115 | + | 25410126 | 11 | downstream | NM_014313 |
| 7 | FJ#21B2 | 857 | 948 | 19 | 8457871-8459154 | + | 8456661 | 1210 | downstream | HNRPM/BC064588 |
| | | | | 19 | 8457871-8459154 | + | 8458765 | 0 | within | AL713781 |
| 8 | FJ#47D2 | 283 | 282 | 17 | 34562266-34562548 | − | 34561298 | 968 | upstream | PLXDC1/AF378753 |
| | | | | 17 | 34562266-34562548 | − | 34561298 | 968 | upstream | NM_020405 |
| 9 | FJ#46A2 | 788 | 666 | 16 | 23597626-23598702 | + | 23597701 | 0 | within | PLK1/BC002369 |
| | | | | 16 | 23597626-23598702 | + | 23597701 | 0 | within | NM_005030 |
| 10 | FJ#73B9 | 732 | 732 | 4 | 88285240-88285972 | + | 88285318 | 0 | within | MLLT2/L13773 |
| | | | | 4 | 88285240-88285972 | + | 88285318 | 0 | within | NM_005935 |
| 11 | FJ#27D1 | 738 | 559 | 12 | 52675489-52676226 | + | 52680143 | 3917 | downstream | NM_006897 |
| | | | | 12 | 52675489-52676226 | + | 52680169 | 3943 | downstream | HOXC9/BC053894 |
| | | | | 12 | 52675489-52676226 | + | 52680241 | 4015 | downstream | HOXC9/BC032769 |
| 12 | FJ#41D7 | 654 | 653 | 1 | 117313967-117314595 | + | 117314990 | 395 | downstream | NM_003594 |
| | | | | 1 | 117313967-117314595 | + | 117314996 | 401 | downstream | TTF2/AF080255 |
| | | | | 1 | 117313967-117314595 | + | 117315006 | 411 | downstream | TTF2/BC030058 |
| 13 | FJ#25A2 | 521 | 523 | 2 | 231551970-231552160 | + | 231555132 | 2972 | downstream | ITM2C/AF271781 |
| | | | | 2 | 231551970-231552160 | + | 231555132 | 2972 | downstream | NM_030926 |
| | | | | 2 | 231551970-231552160 | + | 231555150 | 2990 | downstream | ITM2C/AK090975 |
| | | | | 2 | 231551970-231552160 | + | 231555179 | 3019 | downstream | ITM2C/BC050668 |
| | | | | 2 | 231551970-231552160 | + | 231555187 | 3027 | downstream | ITM2C/BC002424 |
| | | | | 2 | 231551970-231552160 | + | 231555199 | 3039 | downstream | ITM2C/BC025742 |
| 14 | FJ#40D1 | 767 | 764 | 20 | 29790458-29791120 | + | 29790564 | 0 | within | NM_012112 |
| | | | | 20 | 29790458-29791120 | + | 29790798 | 0 | within | TPX2/AF287265 |
| | | | | 20 | 29790458-29791120 | + | 29790805 | 0 | within | TPX2/BC020207 |
| 15 | FJ#46G1 | 442 | 350 | 9 | 123858628-123858970 | + | 123854215 | 4413 | downstream | LHX2/AF124735 |
| 16 | FJ#46C1 | 714 | 502 | 9 | 27518208-27518960 | + | 27514311 | 3897 | downstream | IFNK/AF146759 |
| | | | | 9 | 27518208-27518960 | + | 27514311 | 3897 | downstream | NM_020124 |
| | | | | 9 | 27518208-27518960 | − | 27519744 | 784 | upstream | MOBKL2B/AL832572 |
| | | | | 9 | 27518208-27518960 | − | 27519850 | 890 | upstream | NM_024761 |
| 17 | FJ#46C3 | 321 | 321 | 2 | 206376414-206376687 | + | 206372067 | 4347 | downstream | NRP2/BC009222 |
| | | | | 2 | 206376414-206376687 | + | 206372729 | 3685 | downstream | NM_201264 |
| | | | | 2 | 206376414-206376687 | + | 206372729 | 3685 | downstream | NM_018534 |
| | | | | 2 | 206376414-206376687 | + | 206372729 | 3685 | downstream | NM_201267 |
| | | | | 2 | 206376414-206376687 | + | 206372729 | 3685 | downstream | NM_003872 |
| | | | | 2 | 206376414-206376687 | + | 206372729 | 3685 | downstream | NM_201266 |
| | | | | 2 | 206376414-206376687 | + | 206372729 | 3685 | downstream | NM_201279 |
| | | | | 2 | 206376414-206376687 | + | 206373520 | 2894 | downstream | NRP2/AF016098 |
| | | | | 2 | 206376414-206376687 | + | 206373520 | 2894 | downstream | NRP2/AF280544 |
| | | | | 2 | 206376414-206376687 | + | 206373520 | 2894 | downstream | NRP2/AF280545 |
| | | | | 2 | 206376414-206376687 | + | 206373520 | 2894 | downstream | NRP2/AF280546 |
| 18 | FJ#14H4 | 337 | 628 | 2 | 69781644-69781696 | − | 69781863 | 167 | upstream | AAK1/BC002695 |
| | | | | 2 | 69781644-69781696 | − | 69782500 | 804 | upstream | AAK1/AB028971 |
| | | | | 2 | 69781644-69781696 | − | 69782500 | 804 | upstream | NM_014911 |
| 19 | FJ#53G12 | 814 | 832 | 5 | 113724888-113725712 | + | 113725914 | 202 | downstream | KCNN2/AF239613 |
| | | | | 5 | 113724888-113725712 | + | 113725914 | 202 | downstream | NM_021614 |
| 20 | FJ#43E9 | 588 | 432 | 11 | 71317490-71318078 | + | 71317730 | 0 | within | NM_018320 |
| | | | | 11 | 71317490-71318078 | + | 71317730 | 0 | within | NM_194452 |
| | | | | 11 | 71317490-71318078 | + | 71317730 | 0 | within | NM_194453 |
| | | | | 11 | 71317490-71318078 | + | 71317749 | 0 | within | RNF121/AK023139 |
| | | | | 11 | 71317490-71318078 | + | 71317757 | 0 | within | RNF121/BC009672 |
| 21 | FJ#69B5 | 663 | 663 | 14 | 55303156-55303206 | + | 55302715 | 441 | downstream | BC067891 |
| | | | | 19 | 54685543-54685645 | + | 54682676 | 2867 | downstream | NM_012423 |
| | | | | 19 | 54685543-54685645 | + | 54682693 | 2850 | downstream | RPL13A/BC000514 |
| | | | | 19 | 54685543-54685645 | + | 54684918 | 625 | downstream | RPL13A/BC004900 |
| | | | | 19 | 54685543-54685645 | + | 54685357 | 186 | downstream | RPL13A/AB082924 |
| | | | | 19 | 54685543-54686168 | + | 54682676 | 2867 | downstream | NM_012423 |
| | | | | 19 | 54685543-54686168 | + | 54682693 | 2850 | downstream | RPL13A/BC000514 |
| | | | | 19 | 54685543-54686168 | + | 54684918 | 625 | downstream | RPL13A/BC004900 |
| | | | | 19 | 54685543-54686168 | + | 54685357 | 186 | downstream | RPL13A/AB082924 |
| | | | | 19 | 54685543-54685933 | + | 54682676 | 2867 | downstream | NM_012423 |
| | | | | 19 | 54685543-54685933 | + | 54682693 | 2850 | downstream | RPL13A/BC000514 |
| | | | | 19 | 54685543-54685933 | + | 54684918 | 625 | downstream | RPL13A/BC004900 |
| | | | | 19 | 54685543-54685933 | + | 54685357 | 186 | downstream | RPL13A/AB082924 |
| | | | | 19 | 54685543-54686616 | + | 54682676 | 2867 | downstream | NM_012423 |
| | | | | 19 | 54685543-54686616 | + | 54682693 | 2850 | downstream | RPL13A/BC000514 |
| | | | | 19 | 54685543-54686616 | + | 54684918 | 625 | downstream | RPL13A/BC004900 |
| | | | | 19 | 54685543-54686616 | + | 54685357 | 186 | downstream | RPL13A/AB082924 |
| | | | | 19 | 54685543-54686616 | + | 54691445 | 4829 | downstream | NM_001015 |
| | | | | 19 | 54685543-54686616 | + | 54691499 | 4883 | downstream | RPS11/BC007945 |
| | | | | 19 | 54685543-54686871 | + | 54682676 | 2867 | downstream | NM_012423 |
| | | | | 19 | 54685543-54686871 | + | 54682693 | 2850 | downstream | RPL13A/BC000514 |
| | | | | 19 | 54685543-54686871 | + | 54684918 | 625 | downstream | RPL13A/BC004900 |
| | | | | 19 | 54685543-54686871 | + | 54685357 | 186 | downstream | RPL13A/AB082924 |
| | | | | 19 | 54685543-54686871 | + | 54691445 | 4574 | downstream | NM_001015 |
| | | | | 19 | 54685543-54686871 | + | 54691499 | 4628 | downstream | RPS11/BC007945 |
| | | | | 19 | 54684915-54685645 | + | 54682676 | 2239 | downstream | NM_012423 |
| | | | | 19 | 54684915-54685645 | + | 54682693 | 2222 | downstream | RPL13A/BC000514 |
| | | | | 19 | 54684915-54685645 | + | 54684918 | 0 | within | RPL13A/BC004900 |
| | | | | 19 | 54684915-54685645 | + | 54685357 | 0 | within | RPL13A/AB082924 |
| | | | | 19 | 54685300-54685645 | + | 54682676 | 2624 | downstream | NM_012423 |
| | | | | 19 | 54685300-54685645 | + | 54682693 | 2607 | downstream | RPL13A/BC000514 |
| | | | | 19 | 54685300-54685645 | + | 54684918 | 382 | downstream | RPL13A/BC004900 |
| | | | | 19 | 54685300-54685645 | + | 54685357 | 0 | within | RPL13A/AB082924 |
| | | | | 19 | 54685543-54685933 | + | 54682676 | 2867 | downstream | NM_012423 |
| | | | | 19 | 54685543-54685933 | + | 54682693 | 2850 | downstream | RPL13A/BC000514 |
| | | | | 19 | 54685543-54685933 | + | 54684918 | 625 | downstream | RPL13A/BC004900 |
| | | | | 19 | 54685543-54685933 | + | 54685357 | 186 | downstream | RPL13A/AB082924 |
| | | | | 19 | 54685847-54686168 | + | 54682676 | 3171 | downstream | NM_012423 |
| | | | | 19 | 54685847-54686168 | + | 54682693 | 3154 | downstream | RPL13A/BC000514 |
| | | | | 19 | 54685847-54686168 | + | 54684918 | 929 | downstream | RPL13A/BC004900 |
| | | | | 19 | 54685847-54686168 | + | 54685357 | 490 | downstream | RPL13A/AB082924 |
| | | | | 19 | 54685847-54685933 | + | 54682676 | 3171 | downstream | NM_012423 |
| | | | | 19 | 54685847-54685933 | + | 54682693 | 3154 | downstream | RPL13A/BC000514 |
| | | | | 19 | 54685847-54685933 | + | 54684918 | 929 | downstream | RPL13A/BC004900 |
| | | | | 19 | 54685847-54685933 | + | 54685357 | 490 | downstream | RPL13A/AB082924 |
| | | | | 19 | 54685847-54686616 | + | 54682676 | 3171 | downstream | NM_012423 |
| | | | | 19 | 54685847-54686616 | + | 54682693 | 3154 | downstream | RPL13A/BC000514 |
| | | | | 19 | 54685847-54686616 | + | 54684918 | 929 | downstream | RPL13A/BC004900 |
| | | | | 19 | 54685847-54686616 | + | 54685357 | 490 | downstream | RPL13A/AB082924 |
| | | | | 19 | 54685847-54686616 | + | 54691445 | 4829 | downstream | NM_001015 |
| | | | | 19 | 54685847-54686616 | + | 54691499 | 4883 | downstream | RPS11/BC007945 |
| | | | | 19 | 54685847-54686871 | + | 54682676 | 3171 | downstream | NM_012423 |
| | | | | 19 | 54685847-54686871 | + | 54682693 | 3154 | downstream | RPL13A/BC000514 |
| | | | | 19 | 54685847-54686871 | + | 54684918 | 929 | downstream | RPL13A/BC004900 |
| | | | | 19 | 54685847-54686871 | + | 54685357 | 490 | downstream | RPL13A/AB082924 |
| | | | | 19 | 54685847-54686871 | + | 54691445 | 4574 | downstream | NM_001015 |
| | | | | 19 | 54685847-54686871 | + | 54691499 | 4628 | downstream | RPS11/BC007945 |
| | | | | 19 | 54684915-54685933 | + | 54682676 | 2239 | downstream | NM_012423 |
| | | | | 19 | 54684915-54685933 | + | 54682693 | 2222 | downstream | RPL13A/BC000514 |
| | | | | 19 | 54684915-54685933 | + | 54684918 | 0 | within | RPL13A/BC004900 |
| | | | | 19 | 54684915-54685933 | + | 54685357 | 0 | within | RPL13A/AB082924 |
| | | | | 19 | 54685300-54685933 | + | 54682676 | 2624 | downstream | NM_012423 |
| | | | | 19 | 54685300-54685933 | + | 54682693 | 2607 | downstream | RPL13A/BC000514 |
| | | | | 19 | 54685300-54685933 | + | 54684918 | 382 | downstream | RPL13A/BC004900 |
| | | | | 19 | 54685300-54685933 | + | 54685357 | 0 | within | RPL13A/AB082924 |
| | | | | 19 | 54685543-54686168 | + | 54682676 | 2867 | downstream | NM_012423 |
| | | | | 19 | 54685543-54686168 | + | 54682693 | 2850 | downstream | RPL13A/BC000514 |
| | | | | 19 | 54685543-54686168 | + | 54684918 | 625 | downstream | RPL13A/BC004900 |
| | | | | 19 | 54685543-54686168 | + | 54685357 | 186 | downstream | RPL13A/AB082924 |
| | | | | 19 | 54686108-54686168 | + | 54682676 | 3432 | downstream | NM_012423 |
| | | | | 19 | 54686108-54686168 | + | 54682693 | 3415 | downstream | RPL13A/BC000514 |
| | | | | 19 | 54686108-54686168 | + | 54684918 | 1190 | downstream | RPL13A/BC004900 |
| | | | | 19 | 54686108-54686168 | + | 54685357 | 751 | downstream | RPL13A/AB082924 |
| | | | | 19 | 54685847-54686168 | + | 54682676 | 3171 | downstream | NM_012423 |
| | | | | 19 | 54685847-54686168 | + | 54682693 | 3154 | downstream | RPL13A/BC000514 |
| | | | | 19 | 54685847-54686168 | + | 54684918 | 929 | downstream | RPL13A/BC004900 |
| | | | | 19 | 54685847-54686168 | + | 54685357 | 490 | downstream | RPL13A/AB082924 |
| | | | | 19 | 54686108-54686616 | + | 54682676 | 3432 | downstream | NM_012423 |
| | | | | 19 | 54686108-54686616 | + | 54682693 | 3415 | downstream | RPL13A/BC000514 |
| | | | | 19 | 54686108-54686616 | + | 54684918 | 1190 | downstream | RPL13A/BC004900 |
| | | | | 19 | 54686108-54686616 | + | 54685357 | 751 | downstream | RPL13A/AB082924 |
| | | | | 19 | 54686108-54686616 | + | 54691445 | 4829 | downstream | NM_001015 |
| | | | | 19 | 54686108-54686616 | + | 54691499 | 4883 | downstream | RPS11/BC007945 |
| | | | | 19 | 54686108-54686871 | + | 54682676 | 3432 | downstream | NM_012423 |
| | | | | 19 | 54686108-54686871 | + | 54682693 | 3415 | downstream | RPL13A/BC000514 |
| | | | | 19 | 54686108-54686871 | + | 54684918 | 1190 | downstream | RPL13A/BC004900 |
| | | | | 19 | 54686108-54686871 | + | 54685357 | 751 | downstream | RPL13A/AB082924 |
| | | | | 19 | 54686108-54686871 | + | 54691445 | 4574 | downstream | NM_001015 |
| | | | | 19 | 54686108-54686871 | + | 54691499 | 4628 | downstream | RPS11/BC007945 |
| | | | | 19 | 54684915-54686168 | + | 54682676 | 2239 | downstream | NM_012423 |
| | | | | 19 | 54684915-54686168 | + | 54682693 | 2222 | downstream | RPL13A/BC000514 |
| | | | | 19 | 54684915-54686168 | + | 54684918 | 0 | within | RPL13A/BC004900 |
| | | | | 19 | 54684915-54686168 | + | 54685357 | 0 | within | RPL13A/AB082924 |
| | | | | 19 | 54685300-54686168 | + | 54682676 | 2624 | downstream | NM_012423 |
| | | | | 19 | 54685300-54686168 | + | 54682693 | 2607 | downstream | RPL13A/BC000514 |
| | | | | 19 | 54685300-54686168 | + | 54684918 | 382 | downstream | RPL13A/BC004900 |
| | | | | 19 | 54685300-54686168 | + | 54685357 | 0 | within | RPL13A/AB082924 |
| | | | | 19 | 54685543-54686616 | + | 54682676 | 2867 | downstream | NM_012423 |
| | | | | 19 | 54685543-54686616 | + | 54682693 | 2850 | downstream | RPL13A/BC000514 |
| | | | | 19 | 54685543-54686616 | + | 54684918 | 625 | downstream | RPL13A/BC004900 |
| | | | | 19 | 54685543-54686616 | + | 54685357 | 186 | downstream | RPL13A/AB082924 |
| | | | | 19 | 54685543-54686616 | + | 54691445 | 4829 | downstream | NM_001015 |
| | | | | 19 | 54685543-54686616 | + | 54691499 | 4883 | downstream | RPS11/BC007945 |
| | | | | 19 | 54686108-54686616 | + | 54682676 | 3432 | downstream | NM_012423 |
| | | | | 19 | 54686108-54686616 | + | 54682693 | 3415 | downstream | RPL13A/BC000514 |
| | | | | 19 | 54686108-54686616 | + | 54684918 | 1190 | downstream | RPL13A/BC004900 |
| | | | | 19 | 54686108-54686616 | + | 54685357 | 751 | downstream | RPL13A/AB082924 |
| | | | | 19 | 54686108-54686616 | + | 54691445 | 4829 | downstream | NM_001015 |
| | | | | 19 | 54686108-54686616 | + | 54691499 | 4883 | downstream | RPS11/BC007945 |
| | | | | 19 | 54685847-54686616 | + | 54682676 | 3171 | downstream | NM_012423 |
| | | | | 19 | 54685847-54686616 | + | 54682693 | 3154 | downstream | RPL13A/BC000514 |
| | | | | 19 | 54685847-54686616 | + | 54684918 | 929 | downstream | RPL13A/BC004900 |
| | | | | 19 | 54685847-54686616 | + | 54685357 | 490 | downstream | RPL13A/AB082924 |
| | | | | 19 | 54685847-54686616 | + | 54691445 | 4829 | downstream | NM_001015 |
| | | | | 19 | 54685847-54686616 | + | 54691499 | 4883 | downstream | RPS11/BC007945 |
| | | | | 19 | 54686493-54686616 | + | 54682676 | 3817 | downstream | NM_012423 |
| | | | | 19 | 54686493-54686616 | + | 54682693 | 3800 | downstream | RPL13A/BC000514 |
| | | | | 19 | 54686493-54686616 | + | 54684918 | 1575 | downstream | RPL13A/BC004900 |
| | | | | 19 | 54686493-54686616 | + | 54685357 | 1136 | downstream | RPL13A/AB082924 |
| | | | | 19 | 54686493-54686616 | + | 54691445 | 4829 | downstream | NM_001015 |
| | | | | 19 | 54686493-54686616 | + | 54691499 | 4883 | downstream | RPS11/BC007945 |
| | | | | 19 | 54686493-54686871 | + | 54682676 | 3817 | downstream | NM_012423 |
| | | | | 19 | 54686493-54686871 | + | 54682693 | 3800 | downstream | RPL13A/BC000514 |
| | | | | 19 | 54686493-54686871 | + | 54684918 | 1575 | downstream | RPL13A/BC004900 |
| | | | | 19 | 54686493-54685871 | + | 54685357 | 1136 | downstream | RPL13A/AB082924 |
| | | | | 19 | 54686493-54686871 | + | 54691445 | 4574 | downstream | NM_001015 |
| | | | | 19 | 54686493-54686871 | + | 54691499 | 4628 | downstream | RPS11/BC007945 |
| | | | | 19 | 54684915-54686616 | + | 54682676 | 2239 | downstream | NM_012423 |
| | | | | 19 | 54684915-54686616 | + | 54682693 | 2222 | downstream | RPL13A/BC000514 |
| | | | | 19 | 54684915-54686616 | + | 54684918 | 0 | within | RPL13A/BC004900 |
| | | | | 19 | 54684915-54686616 | + | 54685357 | 0 | within | RPL13A/AB082924 |
| | | | | 19 | 54684915-54686616 | + | 54691445 | 4829 | downstream | NM_001015 |
| | | | | 19 | 54684915-54686616 | + | 54691499 | 4883 | downstream | RPS11/BC007945 |
| | | | | 19 | 54685300-54686616 | + | 54682676 | 2624 | downstream | NM_012423 |
| | | | | 19 | 54685300-54686616 | + | 54682693 | 2607 | downstream | RPL13A/BC000514 |
| | | | | 19 | 54685300-54686616 | + | 54684918 | 382 | downstream | RPL13A/BC004900 |
| | | | | 19 | 54685300-54686616 | + | 54685357 | 0 | within | RPL13A/AB082924 |
| | | | | 19 | 54685300-54686616 | + | 54691445 | 4829 | downstream | NM_001015 |
| | | | | 19 | 54685300-54686616 | + | 54691499 | 4883 | downstream | RPS11/BC007945 |
| | | | | 19 | 54685543-54686871 | + | 54682676 | 2867 | downstream | NM_012423 |
| | | | | 19 | 54685543-54686871 | + | 54682693 | 2850 | downstream | RPL13A/BC000514 |
| | | | | 19 | 54685543-54686871 | + | 54684918 | 625 | downstream | RPL13A/BC004900 |
| | | | | 19 | 54685543-54686871 | + | 54685357 | 186 | downstream | RPL13A/AB082924 |
| | | | | 19 | 54685543-54686871 | + | 54691445 | 4574 | downstream | NM_001015 |
| | | | | 19 | 54685543-54686871 | + | 54691499 | 4628 | downstream | RPS11/BC007945 |
| | | | | 19 | 54686108-54686871 | + | 54682676 | 3432 | downstream | NM_012423 |
| | | | | 19 | 54686108-54686871 | + | 54682693 | 3415 | downstream | RPL13A/BC000514 |
| | | | | 19 | 54686108-54686871 | + | 54684918 | 1190 | downstream | RPL13A/BC004900 |
| | | | | 19 | 54686108-54686871 | + | 54685357 | 751 | downstream | RPL13A/AB082924 |
| | | | | 19 | 54686108-54686871 | + | 54691445 | 4574 | downstream | NM_001015 |
| | | | | 19 | 54686108-54686871 | + | 54691499 | 4628 | downstream | RPS11/BC007945 |
| | | | | 19 | 54685847-54686871 | + | 54682676 | 3171 | downstream | NM_012423 |
| | | | | 19 | 54685847-54686871 | + | 54682693 | 3154 | downstream | RPL13A/BC000514 |
| | | | | 19 | 54685847-54686871 | + | 54684918 | 929 | downstream | RPL13A/BC004900 |
| | | | | 19 | 54685847-54686871 | + | 54685357 | 490 | downstream | RPL13A/AB082924 |
| | | | | 19 | 54685847-54686871 | + | 54691445 | 4574 | downstream | NM_001015 |
| | | | | 19 | 54685847-54686871 | + | 54691499 | 4628 | downstream | RPS11/BC007945 |
| | | | | 19 | 54686493-54686871 | + | 54682676 | 3817 | downstream | NM_012423 |
| | | | | 19 | 54686493-54686871 | + | 54682693 | 3800 | downstream | RPL13A/BC000514 |
| | | | | 19 | 54686493-54686871 | + | 54684918 | 1575 | downstream | RPL13A/BC004900 |
| | | | | 19 | 54686493-54686871 | + | 54685357 | 1136 | downstream | RPL13A/AB082924 |
| | | | | 19 | 54686493-54686871 | + | 54691445 | 4574 | downstream | NM_001015 |
| | | | | 19 | 54686493-54686871 | + | 54691499 | 4628 | downstream | RPS11/BC007945 |
| | | | | 19 | 54686797-54686871 | + | 54682676 | 4121 | downstream | NM_012423 |
| | | | | 19 | 54686797-54686871 | + | 54682693 | 4104 | downstream | RPL13A/BC000514 |
| | | | | 19 | 54686797-54686871 | + | 54684918 | 1879 | downstream | RPL13A/BC004900 |
| | | | | 19 | 54686797-54686871 | + | 54685357 | 1440 | downstream | RPL13A/AB082924 |
| | | | | 19 | 54686797-54686871 | + | 54691445 | 4574 | downstream | NM_001015 |
| | | | | 19 | 54686797-54686871 | + | 54691499 | 4628 | downstream | RPS11/BC007945 |
| | | | | 19 | 54684915-54686871 | + | 54682676 | 2239 | downstream | NM_012423 |
| | | | | 19 | 54684915-54686871 | + | 54682693 | 2222 | downstream | RPL13A/BC000514 |
| | | | | 19 | 54684915-54686871 | + | 54684918 | 0 | within | RPL13A/BC004900 |
| | | | | 19 | 54684915-54686871 | + | 54685357 | 0 | within | RPL13A/AB082924 |
| | | | | 19 | 54684915-54686871 | + | 54691445 | 4574 | downstream | NM_001015 |
| | | | | 19 | 54684915-54686871 | + | 54691499 | 4628 | downstream | RPS11/BC007945 |
| | | | | 19 | 54685300-54686871 | + | 54682676 | 2624 | downstream | NM_012423 |
| | | | | 19 | 54685300-54686871 | + | 54682693 | 2607 | downstream | RPL13A/BC000514 |
| | | | | 19 | 54685300-54686871 | + | 54684918 | 382 | downstream | RPL13A/BC004900 |
| | | | | 19 | 54685300-54686871 | + | 54685357 | 0 | within | RPL13A/AB082924 |
| | | | | 19 | 54685300-54686871 | + | 54691445 | 4574 | downstream | NM_001015 |
| | | | | 19 | 54685300-54686871 | + | 54691499 | 4628 | downstream | RPS11/BC007945 |
| | | | | 19 | 54685300-54685645 | + | 54682676 | 2624 | downstream | NM_012423 |
| | | | | 19 | 54685300-54685645 | + | 54682693 | 2607 | downstream | RPL13A/BC000514 |
| | | | | 19 | 54685300-54685645 | + | 54684918 | 382 | downstream | RPL13A/BC004900 |
| | | | | 19 | 54685300-54685645 | + | 54685357 | 0 | within | RPL13A/AB082924 |
| | | | | 19 | 54685300-54686168 | + | 54682676 | 2624 | downstream | NM_012423 |
| | | | | 19 | 54685300-54686168 | + | 54682693 | 2607 | downstream | RPL13A/BC000514 |
| | | | | 19 | 54685300-54686168 | + | 54684918 | 382 | downstream | RPL13A/BC004900 |
| | | | | 19 | 54685300-54686168 | + | 54685357 | 0 | within | RPL13A/AB082924 |
| | | | | 19 | 54685300-54685933 | + | 54682676 | 2624 | downstream | NM_012423 |
| | | | | 19 | 54685300-54685933 | + | 54682693 | 2607 | downstream | RPL13A/BC000514 |
| | | | | 19 | 54685300-54685933 | + | 54684918 | 382 | downstream | RPL13A/BC004900 |
| | | | | 19 | 54685300-54685933 | + | 54685357 | 0 | within | RPL13A/AB082924 |
| | | | | 19 | 54685300-54686616 | + | 54682676 | 2624 | downstream | NM_012423 |
| | | | | 19 | 54685300-54686616 | + | 54682693 | 2607 | downstream | RPL13A/BC000514 |
| | | | | 19 | 54685300-54686616 | + | 54684918 | 382 | downstream | RPL13A/BC004900 |
| | | | | 19 | 54685300-54686616 | + | 54685357 | 0 | within | RPL13A/AB082924 |
| | | | | 19 | 54685300-54686616 | + | 54691445 | 4829 | downstream | NM_001015 |
| | | | | 19 | 54685300-54686616 | + | 54691499 | 4883 | downstream | RPS11/BC007945 |
| | | | | 19 | 54685300-54686871 | + | 54682676 | 2624 | downstream | NM_012423 |
| | | | | 19 | 54685300-54686871 | + | 54682693 | 2607 | downstream | RPL13A/BC000514 |
| | | | | 19 | 54685300-54686871 | + | 54684918 | 382 | downstream | RPL13A/BC004900 |
| | | | | 19 | 54685300-54686871 | + | 54685357 | 0 | within | RPL13A/AB082924 |
| | | | | 19 | 54685300-54686871 | + | 54691445 | 4574 | downstream | NM_001015 |
| | | | | 19 | 54685300-54686871 | + | 54691499 | 4628 | downstream | RPS11/BC007945 |
| | | | | 19 | 54684915-54685366 | + | 54682676 | 2239 | downstream | NM_012423 |
| | | | | 19 | 54684915-54685366 | + | 54682693 | 2222 | downstream | RPL13A/BC000514 |
| | | | | 19 | 54684915-54685366 | + | 54684918 | 0 | within | RPL13A/BC004900 |
| | | | | 19 | 54684915-54685366 | + | 54685357 | 0 | within | RPL13A/AB082924 |
| | | | | 19 | 54685300-54685366 | + | 54682676 | 2624 | downstream | NM_012423 |
| | | | | 19 | 54685300-54685366 | + | 54682693 | 2607 | downstream | RPL13A/BC000514 |
| | | | | 19 | 54685300-54685366 | + | 54684918 | 382 | downstream | RPL13A/BC004900 |
| | | | | 19 | 54685300-54685366 | + | 54685357 | 0 | within | RPL13A/AB082924 |
| | | | | 19 | 54684915-54685645 | + | 54682676 | 2239 | downstream | NM_012423 |
| | | | | 19 | 54684915-54685645 | + | 54682693 | 2222 | downstream | RPL13A/BC000514 |
| | | | | 19 | 54684915-54685645 | + | 54684918 | 0 | within | RPL13A/BC004900 |
| | | | | 19 | 54684915-54685645 | + | 54685357 | 0 | within | RPL13A/AB082924 |
| | | | | 19 | 54684915-54686168 | + | 54682676 | 2239 | downstream | NM_012423 |
| | | | | 19 | 54684915-54686168 | + | 54682693 | 2222 | downstream | RPL13A/BC000514 |
| | | | | 19 | 54684915-54686168 | + | 54684918 | 0 | within | RPL13A/BC004900 |
| | | | | 19 | 54684915-54686168 | + | 54685357 | 0 | within | RPL13A/AB082924 |
| | | | | 19 | 54684915-54685933 | + | 54682676 | 2239 | downstream | NM_012423 |
| | | | | 19 | 54684915-54685933 | + | 54682693 | 2222 | downstream | RPL13A/BC000514 |
| | | | | 19 | 54684915-54685933 | + | 54684918 | 0 | within | RPL13A/BC004900 |
| | | | | 19 | 54684915-54685933 | + | 54685357 | 0 | within | RPL13A/AB082924 |
| | | | | 19 | 54684915-54686616 | + | 54682676 | 2239 | downstream | NM_012423 |
| | | | | 19 | 54684915-54686616 | + | 54682693 | 2222 | downstream | RPL13A/BC000514 |
| | | | | 19 | 54684915-54686616 | + | 54684918 | 0 | within | RPL13A/BC004900 |
| | | | | 19 | 54684915-54686616 | + | 54685357 | 0 | within | RPL13A/AB082924 |
| | | | | 19 | 54684915-54686616 | + | 54691445 | 4829 | downstream | NM_001015 |
| | | | | 19 | 54684915-54686616 | + | 54691499 | 4883 | downstream | RPS11/BC007945 |
| | | | | 19 | 54684915-54686871 | + | 54682676 | 2239 | downstream | NM_012423 |
| | | | | 19 | 54684915-54686871 | + | 54682693 | 2222 | downstream | RPL13A/BC000514 |
| | | | | 19 | 54684915-54686871 | + | 54684918 | 0 | within | RPL13A/BC004900 |
| | | | | 19 | 54684915-54686871 | + | 54685357 | 0 | within | RPL13A/AB082924 |
| | | | | 19 | 54684915-54686871 | + | 54691445 | 4574 | downstream | NM_001015 |
| | | | | 19 | 54684915-54686871 | + | 54691499 | 4628 | downstream | RPS11/BC007945 |
| | | | | 19 | 54684915-54684991 | + | 54682676 | 2239 | downstream | NM_012423 |
| | | | | 19 | 54684915-54684991 | + | 54682693 | 2222 | downstream | RPL13A/BC000514 |
| | | | | 19 | 54684915-54684991 | + | 54684918 | 0 | within | RPL13A/BC004900 |
| | | | | 19 | 54684915-54684991 | + | 54685357 | 366 | downstream | RPL13A/AB082924 |
| | | | | 19 | 54684915-54685366 | + | 54682676 | 2239 | downstream | NM_012423 |
| | | | | 19 | 54684915-54685366 | + | 54682693 | 2222 | downstream | RPL13A/BC000514 |
| | | | | 19 | 54684915-54685366 | + | 54684918 | 0 | within | RPL13A/BC004900 |
| | | | | 19 | 54684915-54685366 | + | 54685357 | 0 | within | RPL13A/AB082924 |
| 22 | FJ#33D12 | 783 | 783 | 10 | 94322787-94323571 | − | 94323813 | 242 | upstream | IDE/M21188 |
| | | | | 10 | 94322787-94323571 | − | 94323813 | 242 | upstream | NM_004969 |
| 23 | FJ#32F2 | 622 | 618 | 4 | 85773754-85774366 | − | 85776566 | 2200 | upstream | NKX6-1/NM_006168 |
| 24 | FJ#54H12 | 705 | 705 | x | 52870728-52871428 | − | 52869197 | 1531 | upstream | NM_014138 |
| | | | | x | 52808646-52809350 | + | 52810882 | 1532 | downstream | AF370413 |
| | | | | x | 52808646-52809350 | + | 52810882 | 1532 | downstream | NM_014138 |
| 25 | FJ#55F11 | 597 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 26 | FJ#26C4 | 225 | 686 | 12 | 10765801-10766442 | − | 10767171 | 729 | upstream | CSDA/BC021926 |
| | | | | 12 | 10765801-10766442 | − | 10767171 | 729 | upstream | NM_003651 |
| | | | | 12 | 10765801-10766442 | − | 10767173 | 731 | upstream | CSDA/BC009744 |
| 27 | FJ#40H5 | 328 | 329 | 1 | 222557795-222558123 | + | 222557155 | 640 | downstream | NM_002107 |
| | | | | 1 | 222557795-222558123 | + | 222558412 | 289 | downstream | H3F3A/M11353 |
| 28 | FJ#25F4 | 517 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |

TABLE 12 shows, according to particular preferred aspects, markers for ALL as identified by methylation hybridization as described in the EXAMPLES herein.

| | | T7 | M13 | | | | | Dis- | | |
|---|---|---|---|---|---|---|---|---|---|---|
| | | Se- | Se- | Chro- | | | | tance | | |
| | | quence | quence | mosome | | | | to | | |
| No. | Clone ID | Length | Length | Aligned | Alignment Address | Strand | TSS | TSS | Direction | Gene/Assession Number |
| 1 | FJ#3A9 | 787 | 787 | unknown | −1-−1 | unknown | −1 | −1 | unknown | CIDE-3 |
| | | | | 21 | 30232914-30233702 | − | 30233814 | 112 | upstream | GRIK1/AJ249208 |
| | | | | 21 | 30232914-30233702 | − | 30234101 | 399 | upstream | GRIK1/L19058 |
| | | | | 21 | 30232914-30233702 | − | 30234101 | 399 | upstream | NM_175611 |
| | | | | 21 | 30232914-30233702 | − | 30234101 | 399 | upstream | NM_000830 |
| 2 | FJ#3C1 | 258 | 393 | 16 | 21517573-21517834 | + | 21518379 | 545 | downstream | DREV1/AJ278577 |
| | | | | 16 | 21517573-21511834 | + | 21518379 | 545 | downstream | DREV1/AJ278578 |
| | | | | 16 | 21517573-21517834 | + | 21518412 | 578 | downstream | DREV1/BC000195 |
| | | | | 16 | 21517573-21517834 | + | 21518412 | 578 | downstream | NM_016025 |
| | | | | 16 | 21517573-21517834 | + | 21518579 | 745 | downstream | DREV1/AF497245 |
| | | | | 16 | 21517573-21517834 | + | 21518633 | 799 | downstream | DREV1/AF151839 |
| 3 | FJ#2E11 | 797 | 877 | 15 | 35176950-35178006 | − | 35177795 | 0 | within | NM_172315 |
| | | | | 15 | 35176950-35178006 | − | 35178889 | 883 | upstream | NM_172316 |
| | | | | 15 | 35176950-35178006 | − | 35179996 | 1990 | upstream | NM_020149 |
| | | | | 15 | 35176950-35178006 | − | 35179996 | 1990 | upstream | NM_170674 |
| | | | | 15 | 35176950-35178006 | − | 35179996 | 1990 | upstream | NM_170675 |
| | | | | 15 | 35176950-35178006 | − | 35179996 | 1990 | upstream | NM_170676 |
| | | | | 15 | 35176950-35178006 | − | 35179996 | 1990 | upstream | NM_170677 |
| | | | | 15 | 35176950-35178006 | − | 35180673 | 2667 | upstream | MEIS2/BC050431 |
| | | | | 15 | 35176950-35178006 | − | 35180792 | 2786 | upstream | NM_002399 |
| | | | | 15 | 35176950-35178006 | − | 35180796 | 2790 | upstream | MEIS2/BC001844 |
| 4 | FJ#7A5 | 474 | 474 | unknown | −1-−1 | unknown | −1 | −1 | unknown | AK123224 |
| | | | | 16 | 30349234-30349708 | − | 30348874 | 360 | upstream | XTP3TPA/BC001344 |
| | | | | 16 | 30349234-30349708 | − | 30348874 | 360 | upstream | NM_024096 |
| 5 | FJ#8A3 | 461 | 461 | unknown | −1-−1 | unknown | −1 | −1 | unknown | FLJ43403 |
| | | | | 15 | 38773851-38774312 | + | 38774660 | 348 | downstream | NM_002875 |
| | | | | 15 | 38773851-38774312 | + | 38774660 | 348 | downstream | NM_133487 |
| | | | | 15 | 38773851-38774312 | + | 38774685 | 373 | downstream | RAD51/D14134 |
| 6 | FJ#8A5 | 633 | 633 | 1 | 6597888-6598522 | − | 6596993 | 895 | upstream | AK090472 |
| | | | | 1 | 6597888-6598522 | − | 6597195 | 693 | upstream | BC034039 |
| | | | | 1 | 6597888-6598522 | − | 6597327 | 561 | upstream | AB007938 |
| 7 | FJ#7E5 | 0 | 577 | unknown | −1-−1 | unknown | −1 | −1 | unknown | IMAGE: 5262055 |
| 8 | FJ#8E11 | 474 | 474 | 20 | 25156077-25156512 | − | 25155371 | 706 | upstream | AF058296 |
| 9 | FJ#10G9 | 555 | 555 | 16 | 29845411-29845966 | − | 29845046 | 365 | upstream | KCTD13/BC036228 |
| | | | | 16 | 29845411-29845966 | − | 29845046 | 365 | upstream | NM_178863 |
| 10 | FJ#11A5 | 416 | 416 | 6 | 43705205-43705621 | + | 43700328 | 4877 | downstream | AF116627 |
| | | | | 6 | 43705205-43705621 | − | 43701279 | 3926 | upstream | GTPBP2/BC020980 |
| | | | | 6 | 43705205-43705621 | − | 43702129 | 3076 | upstream | GTPBP2/BC028347 |
| | | | | 6 | 43705205-43705621 | − | 43703025 | 2180 | upstream | GTPBP2/AK000430 |
| | | | | 6 | 43705205-43705621 | − | 43704749 | 456 | upstream | GTPBP2/AF168990 |
| | | | | 6 | 43705205-43705621 | − | 43704770 | 435 | upstream | GTPBP2/AB024574 |
| | | | | 6 | 43705205-43705621 | − | 43704914 | 291 | upstream | GTPBP2/BC064968 |
| | | | | 6 | 43705205-43705621 | − | 43704914 | 291 | upstream | NM_019096 |
| 11 | FJ#12A3 | 515 | 753 | 1 | 211643229-211643982 | + | 211644994 | 1012 | downstream | KCNK2/AF004711 |
| | | | | 1 | 211643229-211643982 | + | 211645030 | 1048 | downstream | KCNK2/AF171068 |
| | | | | 1 | 211643229-211643982 | + | 211645030 | 1048 | downstream | NM_014217 |
| 12 | FJ#15A5 | 470 | 884 | 11 | 56950485-56951578 | − | 56948108 | 2377 | upstream | PRG2/Z26248 |
| | | | | 11 | 56950485-56951578 | − | 56951099 | 0 | within | NM_014096 |
| | | | | 11 | 56950485-56951578 | − | 56951170 | 0 | within | SLC43A3/AK075552 |
| | | | | 11 | 56950485-56951578 | − | 56951170 | 0 | within | NM_199329 |
| | | | | 11 | 56950485-56951578 | − | 56951629 | 51 | upstream | NM_017611 |
| 13 | FJ#15A9 | 130 | 564 | 17 | 1679228-1679726 | + | 1680094 | 368 | downstream | RPA1/BC018126 |
| | | | | 17 | 1679228-1679726 | + | 1680094 | 368 | downstream | NM_002945 |
| | | | | 17 | 1679228-1679726 | − | 1679839 | 113 | upstream | SMYD4/BC035077 |
| | | | | 17 | 1679228-1679726 | − | 1679839 | 113 | upstream | NM_052928 |
| 14 | FJ#20G11 | 583 | 580 | 19 | 43518614-43519197 | + | 43518297 | 317 | downstream | C19orf15/AK128220 |
| | | | | 19 | 43518614-43519197 | + | 43518297 | 317 | downstream | NM_021185 |
| 15 | FJ#22C5 | 826 | 807 | unknown | −1-−1 | unknown | −1 | −1 | unknown | PSMA2 |
| | | | | 7 | 42744749-42745650 | + | 42745178 | 0 | within | NM_031903 |
| | | | | 7 | 42744749-42745650 | + | 42745219 | 0 | within | MRPL32/BC013147 |
| | | | | 7 | 42744749-42745650 | − | 42744999 | 0 | within | PSMA2/BC047697 |
| | | | | 7 | 42744749-42745650 | − | 42745011 | 0 | within | PSMA2/BX641097 |
| | | | | 7 | 42744749-42745650 | − | 42745045 | 0 | within | NM_002787 |
| 16 | FJ#25E3 | 304 | 301 | 5 | 54559229-54559500 | − | 54564476 | 4976 | upstream | UNG2/X52486 |
| | | | | 5 | 54559229-54559500 | − | 54564476 | 4976 | upstream | NM_021147 |
| 17 | FJ#23G11 | 877 | 845 | 12 | 7915942-7916816 | + | 7916800 | 0 | within | AY455283 |
| | | | | 12 | 7915942-7916816 | − | 7916749 | 0 | within | SLC2A14/BC060766 |
| | | | | 12 | 7915942-7916816 | − | 7916762 | 0 | within | SLC2A14/AF481879 |
| | | | | 12 | 7915942-7916816 | − | 7916762 | 0 | within | NM_153449 |
| 18 | FJ#25G9 | 516 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 19 | FJ#30A11 | 774 | 912 | 21 | 44955066-44956738 | − | 44955923 | 0 | within | C21orf29/AJ487962 |
| | | | | 21 | 44955066-44956738 | − | 44955923 | 0 | within | NM_144991 |
| 20 | FJ#30E9 | 689 | 690 | 10 | 17726024-17726714 | + | 17726129 | 0 | within | NM_003473 |
| | | | | 10 | 17726024-17726714 | + | 17726186 | 0 | within | STAM/BC030586 |
| | | | | 10 | 17726024-17726714 | + | 17726302 | 0 | within | STAM/U43899 |
| 21 | FJ#30E11 | 244 | 244 | 8 | 11362844-11363088 | − | 11361663 | 1181 | upstream | C8orf13/AL834122 |
| | | | | 8 | 11362844-11363088 | − | 11361663 | 1181 | upstream | NM_053279 |
| 22 | FJ#1C10 | 683 | 684 | 17 | 44155815-44156431 | − | 44154879 | 936 | upstream | PRAC/BC030950 |
| | | | | 17 | 44155815-44156431 | − | 44154881 | 934 | upstream | NM_032391 |
| | | | | 17 | 44155815-44156431 | − | 44161084 | 4653 | upstream | HOXB13/U81599 |
| | | | | 17 | 44155815-44156431 | − | 44161084 | 4653 | upstream | NM_006361 |
| 23 | FJ#2C2 | 255 | 152 | unknown | −1-−1 | unknown | −1 | −1 | unknown | SLC25A3 |
| 24 | FJ#5C12 | 0 | 528 | 18 | 48122355-48122876 | + | 48121155 | 1200 | downstream | DCC/X76132 |
| | | | | 18 | 48122355-48122876 | + | 48121155 | 1200 | downstream | NM_005215 |
| 25 | FJ#12A10 | 885 | 856 | 12 | 119587584-119588588 | + | 119587668 | 0 | within | NM_014730 |
| | | | | 12 | 119587584-119588588 | + | 119587691 | 0 | within | KIAA0152/D63486 |
| 26 | FJ#13C6 | 469 | 748 | 2 | 32175740-32176474 | − | 32176474 | 0 | within | NM_032574 |
| | | | | 2 | 32175740-32176474 | − | 32176513 | 39 | upstream | LOC84661/BC015970 |
| 27 | FJ#11E4 | 818 | 391 | 19 | 12765462-12766329 | + | 12763309 | 2153 | downstream | JUNB/BC004250 |
| | | | | 19 | 12765462-12766329 | + | 12763309 | 2153 | downstream | NM_002229 |
| 28 | FJ#23A10 | 644 | 644 | unknown | −1-−1 | unknown | −1 | −1 | unknown | BANF1 |
| | | | | 11 | 65525871-65526496 | + | 65526125 | 0 | within | BANF1/AF068235 |
| | | | | 11 | 65525871-65526496 | + | 65526125 | 0 | within | NM_003860 |
| | | | | 11 | 65525871-65526496 | − | 65526154 | 0 | within | MGC11102/AK094129 |
| | | | | 11 | 65525871-65526496 | − | 65526154 | 0 | within | NM_032325 |
| 29 | FJ#25A2 | 521 | 523 | 2 | 231551970-231552160 | + | 231555132 | 2972 | downstream | ITM2C/AF271781 |
| | | | | 2 | 231551970-231552160 | + | 231555132 | 2972 | downstream | NM_030926 |
| | | | | 2 | 231551970-231552160 | + | 231555150 | 2990 | downstream | ITM2C/AK090975 |
| | | | | 2 | 231551970-231552160 | + | 231555179 | 3019 | downstream | ITM2C/BC050668 |
| | | | | 2 | 231551970-231552160 | + | 231555187 | 3027 | downstream | ITM2C/BC002424 |
| | | | | 2 | 231551970-231552160 | + | 231555199 | 3039 | downstream | ITM2C/BC025742 |
| 30 | FJ#26C4 | 225 | 686 | 12 | 10765801-10766442 | − | 10767171 | 729 | upstream | CSDA/BC021926 |
| | | | | 12 | 10765801-10766442 | − | 10767171 | 729 | upstream | NM_003651 |
| | | | | 12 | 10765801-10766442 | − | 10767173 | 731 | upstream | CSDA/BC009744 |
| 31 | FJ#27E8 | 612 | 609 | 2 | 230612655-230613264 | + | 230612711 | 0 | within | FBXO36/BC017869 |
| | | | | 2 | 230612655-230613264 | + | 230612718 | 0 | within | FBXO36/BC033935 |
| | | | | 2 | 230612655-230613264 | + | 230612718 | 0 | within | NM_174899 |
| | | | | 2 | 230612655-230613264 | − | 230612160 | 495 | upstream | TRIP12/D28476 |
| 32 | FJ#33C10 | 271 | 271 | 3 | 2114763-2115023 | + | 2117246 | 2223 | downstream | CNTN4/AY090737 |
| | | | | 3 | 2114763-2115023 | + | 2117246 | 2223 | downstream | NM_175607 |
| 33 | FJ#32E8 | 547 | 548 | 18 | 11839826-11840374 | + | 11841425 | 1051 | downstream | CHMP1.5/BC065933 |
| | | | | 18 | 11839826-11840374 | + | 11841425 | 1051 | downstream | NM_020412 |
| | | | | 18 | 11839826-11840374 | + | 11841456 | 1082 | downstream | CHMP1.5/BC012733 |
| | | | | 18 | 11839826-11840374 | + | 11841466 | 1092 | downstream | CHMP1.5/AF281064 |
| 34 | FJ#33E12 | 283 | 283 | 15 | 94670867-94671150 | + | 94674949 | 3799 | downstream | NR2F2/BC042897 |
| | | | | 15 | 94670867-94671150 | + | 94674949 | 3799 | downstream | NM_021005 |
| 35 | FJ#1F9 | 562 | 0 | 21 | 39477469-39478047 | − | 39477181 | 288 | upstream | AY463963 |
| | | | | 21 | 39477469-39478047 | − | 39477227 | 242 | upstream | DSCR2/BC011755 |
| | | | | 21 | 39477469-39478047 | − | 39477310 | 159 | upstream | NM_003720 |
| | | | | 21 | 39477469-39478047 | − | 39477310 | 159 | upstream | NM_203433 |
| 36 | FJ#2F11 | 574 | 727 | unknown | −1-−1 | unknown | −1 | −1 | unknown | FLJ10466 |
| 37 | FJ#7F5 | 0 | 277 | 17 | 8226771-8227048 | − | 8226359 | 412 | upstream | X69392 |
| | | | | 17 | 8226771-8227048 | − | 8227234 | 186 | upstream | NM_000987 |
| | | | | 17 | 8226771-8227048 | − | 8227236 | 188 | upstream | RPL26/BC066316 |
| 38 | FJ#7H3 | 510 | 796 | 6 | 31882104-31882944 | − | 31882722 | 0 | within | LSM2/BC009192 |
| | | | | 6 | 31882104-31882944 | − | 31882722 | 0 | within | NM_021177 |
| 39 | FJ#8H1 | 1 | 441 | 1 | 52730126-52730194 | − | 52730687 | 493 | upstream | BC048301 |
| 40 | FJ#23H7 | 916 | 918 | 1 | 168481258-168482112 | + | 168482478 | 366 | downstream | CGI-01/AK027621 |
| | | | | 1 | 168481258-168482112 | + | 168482478 | 366 | downstream | NM_014955 |
| | | | | 1 | 168481258-168482112 | + | 168482492 | 380 | downstream | CGI-01/AF132936 |
| | | | | 1 | 168481258-168482112 | + | 168482492 | 380 | downstream | CGI-01/AL049669 |
| | | | | 1 | 168481258-168482112 | + | 168482492 | 380 | downstream | NM_015935 |
| | | | | 1 | 168481258-168482112 | + | 168482662 | 550 | downstream | CGI-01/AB020666 |
| | | | | 1 | 168481258-168482112 | + | 168482663 | 551 | downstream | CGI-01/BC029083 |
| | | | | 1 | 168481258-168482112 | + | 168484632 | 2520 | downstream | CGI-01/AK074552 |
| 41 | FJ#25H5 | 515 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 42 | FJ#27B5 | 423 | 707 | 2 | 228162763-228163462 | + | 228162546 | 217 | downstream | NM_004504 |
| | | | | 2 | 228162763-228163462 | + | 228162558 | 205 | downstream | HRB/BC030592 |
| 43 | FJ#30D9 | 868 | 795 | 5 | 69746502-69747354 | + | 69746971 | 0 | within | GTF2H2/AF078847 |
| | | | | 5 | 69746502-69747354 | + | 69746971 | 0 | within | NM_001515 |
| | | | | 5 | 69746502-69747354 | + | 69751864 | 4510 | downstream | BT006773 |
| | | | | 5 | 68891209-68892207 | + | 68891824 | 0 | within | GTF2H2/AF078847 |
| | | | | 5 | 68891209-68892207 | + | 68891824 | 0 | within | NM_001515 |
| | | | | 5 | 68891209-68892207 | + | 68896715 | 4508 | downstream | BT006773 |
| | | | | 5 | 70398855-70399707 | − | 70394346 | 4509 | upstream | BT006773 |
| | | | | 5 | 70398855-70399707 | − | 70399238 | 0 | within | GTF2H2/AF078847 |
| | | | | 5 | 70398855-70399707 | − | 70399238 | 0 | within | NM_001515 |
| 44 | FJ#30F9 | 508 | 507 | 7 | 86902729-86903236 | + | 86902379 | 350 | downstream | RPIB9/AK055233 |
| | | | | 7 | 86902729-86903236 | + | 86902379 | 350 | downstream | NM_138290 |
| | | | | 7 | 86902729-86903236 | + | 86902420 | 309 | downstream | RPIB9/BC022520 |
| 45 | FJ#2D2 | 639 | 639 | 1 | 201822259-201822898 | − | 201822760 | 0 | within | RBBP5/BC037284 |
| | | | | 1 | 201822259-201822898 | − | 201822765 | 0 | within | RBBP5/X85134 |
| | | | | 1 | 201822259-201822898 | − | 201822765 | 0 | within | NM_005057 |
| 46 | FJ#1F4 | 660 | 660 | 19 | 61571170-61571830 | + | 61571319 | 0 | within | ZNF542/BX640680 |
| | | | | 19 | 61571170-61571830 | + | 61571319 | 0 | within | NM_194319 |
| 47 | FJ#7B6 | 499 | 548 | 9 | 103936037-103936585 | + | 103936131 | 0 | within | BC055081 |
| | | | | 9 | 103936037-103936585 | + | 103936147 | 0 | within | BC061906 |
| | | | | 9 | 103936037-103936585 | + | 103936161 | 0 | within | SMC2L1/AF092563 |
| | | | | 9 | 103936037-103936585 | + | 103936161 | 0 | within | NM_006444 |
| 48 | FJ#8F10 | 610 | 610 | 13 | 72253949-72254560 | + | 72254237 | 0 | within | C13orf24/AF330046 |
| | | | | 13 | 72253949-72254560 | + | 72254331 | 0 | within | AY370776 |
| | | | | 13 | 72253949-72254560 | + | 72254363 | 0 | within | AY375528 |
| | | | | 13 | 72253949-72254560 | + | 72255608 | 1048 | downstream | NM_006346 |
| | | | | 13 | 72253949-72254560 | − | 72254007 | 0 | within | KIAA1008/AF330044 |
| | | | | 13 | 72253949-72254560 | − | 72254007 | 0 | within | NM_014953 |
| 49 | FJ#11B2 | 729 | 846 | 4 | 72132504-72133776 | + | 72133098 | 0 | within | NM_173468 |
| | | | | 4 | 72132504-72133776 | + | 72133143 | 0 | within | MOBKL1A/BC038112 |
| 50 | FJ#11H4 | 843 | 843 | 16 | 2322634-2322686 | − | 2319698 | 2936 | upstream | BC062779 |
| | | | | 19 | 44617886-44618729 | − | 44618426 | 0 | within | RPS16/BC004324 |
| | | | | 19 | 44617886-44618729 | − | 44618478 | 0 | within | NM_001020 |
| 51 | FJ#13H2 | 319 | 480 | 3 | 184627876-184628356 | − | 184628557 | 201 | upstream | NM_015078 |
| | | | | 3 | 184627876-184628356 | − | 184628575 | 219 | upstream | KIAA0861/BC064632 |
| | | | | 3 | 184627876-184628356 | − | 184628591 | 235 | upstream | AK124500 |
| 52 | FJ#21F10 | 822 | 889 | 16 | 66424340-66425328 | − | 66420904 | 3436 | upstream | FLJ13111/BC007864 |
| | | | | 16 | 66424340-66425328 | − | 66425255 | 0 | within | FLJ13111/BC007642 |
| | | | | 16 | 66424340-66425328 | − | 66425263 | 0 | within | FLJ13111/BC015202 |
| | | | | 16 | 66424340-66425328 | − | 66425301 | 0 | within | FLJ13111/AK023173 |
| | | | | 16 | 66424340-66425328 | − | 66425301 | 0 | within | NM_025082 |
| | | | | 16 | 66424340-66425328 | − | 66425330 | 2 | upstream | FLJ13111/AK055237 |
| | | | | 16 | 66424505-66425328 | − | 66420904 | 3601 | upstream | FLJ13111/BC007864 |
| | | | | 16 | 66424505-66425328 | − | 66425255 | 0 | within | FLJ13111/BC007642 |
| | | | | 16 | 66424505-66425328 | − | 66425263 | 0 | within | FLJ13111/BC015202 |
| | | | | 16 | 66424505-66425328 | − | 66425301 | 0 | within | FLJ13111/AK023173 |
| | | | | 16 | 66424505-66425328 | − | 66425301 | 0 | within | NM_025082 |
| | | | | 16 | 66424505-66425328 | − | 66425330 | 2 | upstream | FLJ13111/AK055237 |
| | | | | 16 | 66424340-66425231 | − | 66420904 | 3436 | upstream | FLJ13111/BC007864 |
| | | | | 16 | 66424340-66425231 | − | 66425255 | 24 | upstream | FLJ13111/BC007642 |
| | | | | 16 | 66424340-66425231 | − | 66425263 | 32 | upstream | FLJ13111/BC015202 |
| | | | | 16 | 66424340-66425231 | − | 66425301 | 70 | upstream | FLJ13111/AK023173 |
| | | | | 16 | 66424340-66425231 | − | 66425301 | 70 | upstream | NM_025082 |
| | | | | 16 | 66424340-66425231 | − | 66425330 | 99 | upstream | FLJ13111/AK055237 |
| | | | | 16 | 66424977-66425037 | − | 66420904 | 4073 | upstream | FLJ13111/BC007864 |
| | | | | 16 | 66424977-66425037 | − | 66425255 | 218 | upstream | FLJ13111/BC007642 |
| | | | | 16 | 66424977-66425037 | − | 66425263 | 226 | upstream | FLJ13111/BC015202 |
| | | | | 16 | 66424977-66425037 | − | 66425301 | 264 | upstream | FLJ13111/AK023173 |
| | | | | 16 | 66424977-66425037 | − | 66425301 | 264 | upstream | NM_025082 |
| | | | | 16 | 66424977-66425037 | − | 66425330 | 293 | upstream | FLJ13111/AK055237 |
| 53 | FJ#28B2 | 728 | 725 | 13 | 94158266-94158856 | − | 94162250 | 3394 | upstream | SOX21/X65666 |
| | | | | 13 | 94158266-94158856 | − | 94162390 | 3534 | upstream | NM_007084 |
| 54 | FJ#28F2 | 429 | 429 | 4 | 141430872-141431249 | − | 141432838 | 1589 | upstream | MAML3/AB058719 |
| | | | | 4 | 141430872-141431249 | − | 141432838 | 1589 | upstream | NM_018717 |
| 55 | FJ#30F12 | 685 | 710 | 15 | 39310571-39311477 | + | 39310728 | 0 | within | NM_007236 |
| | | | | 15 | 39310571-39311477 | + | 39310804 | 0 | within | BC051815 |
| | | | | 15 | 39310571-39311477 | + | 39310826 | 0 | within | CHP/BC031293 |
| | | | | 15 | 39310571-39311477 | − | 39310187 | 384 | upstream | MGC33637/BC030628 |
| | | | | 15 | 39310571-39311477 | − | 39310187 | 384 | upstream | NM_152596 |
| 56 | FJ#26H10 | 596 | 597 | 12 | 108801233-108801801 | + | 108800798 | 435 | downstream | MGC10854/AK092736 |
| | | | | 12 | 108801233-108801801 | + | 108801042 | 191 | downstream | NM_032300 |
| 57 | FJ#33D8 | 333 | 442 | 1 | 61260260-61260698 | + | 61260000 | 260 | downstream | NFIA/AB037860 |
| | | | | 1 | 61260260-61260698 | + | 61260315 | 0 | within | NFIA/BC022264 |
| | | | | 1 | 61260260-61260698 | + | 61260315 | 0 | within | NM_005595 |
| 58 | FJ#31F6 | 801 | 787 | 12 | 46584544-46585362 | − | 46585036 | 0 | within | VDR/J03258 |
| | | | | 12 | 46584544-46585362 | − | 46585036 | 0 | within | NM_000376 |
| 59 | FJ#32F2 | 622 | 618 | 4 | 85773754-85774366 | − | 85776566 | 2200 | upstream | NKX6-1/NM_006168 |
| 60 | FJ#36A3 | 196 | 675 | 4 | 95486163-95486347 | + | 95486375 | 28 | downstream | SMARCAD1/AY008271 |
| | | | | 4 | 95486163-95486347 | + | 95486375 | 28 | downstream | NM_020159 |
| 61 | FJ#39C3 | 589 | 588 | 14 | 56340481-56341069 | − | 56341927 | 858 | upstream | OTX2/AF093138 |
| | | | | 14 | 56340481-56341069 | − | 56342098 | 1029 | upstream | NM_172337 |
| 62 | FJ#39C7 | 540 | 273 | 18 | 48120205-48120536 | + | 48121155 | 619 | downstream | DCC/X76132 |
| | | | | 18 | 48120205-48120536 | + | 48121155 | 619 | downstream | NM_005215 |
| 63 | FJ#36E3 | 551 | 551 | 5 | 149808667-149809197 | − | 149809487 | 290 | upstream | RPS14/AF116710 |
| | | | | 5 | 149808667-149809197 | − | 149809512 | 315 | upstream | RPS14/NM_005617 |
| 64 | FJ#41A5 | 570 | 570 | 6 | 26141494-26142051 | − | 26140267 | 1227 | upstream | HIST1H3B/NM_003537 |
| | | | | 6 | 26141494-26142051 | − | 26141775 | 0 | within | HIST1H2AB/NM_003513 |
| 65 | FJ#45A3 | 710 | 658 | 5 | 168661620-168662328 | − | 168660554 | 1066 | upstream | NM_003062 |
| 66 | FJ#45A9 | 211 | 0 | 14 | 85067745-85067898 | − | 85066085 | 1660 | upstream | BX248253 |
| 67 | FJ#43E5 | 742 | 742 | 13 | 42047024-42047766 | + | 42043794 | 3230 | downstream | NM_033012 |
| | | | | 13 | 42047024-42047766 | + | 42046297 | 727 | downstream | TNFSF11/AF053712 |
| | | | | 13 | 42047024-42047766 | + | 42046297 | 727 | downstream | NM_003701 |
| | | | | 13 | 42047024-42047766 | + | 42046359 | 665 | downstream | TNFSF11/AB064268 |
| 68 | FJ#41G5 | 653 | 652 | 2 | 20172677-20173329 | − | 20173033 | 0 | within | LAPTM4A/AY359028 |
| | | | | 2 | 20172677-20173329 | − | 20173055 | 0 | within | LAPTM4A/D14696 |
| | | | | 2 | 20172677-20173329 | − | 20173057 | 0 | within | LAPTM4A/BC003158 |
| | | | | 2 | 20172677-20173329 | − | 20173073 | 0 | within | NM_014713 |
| 69 | FJ#41G7 | 315 | 315 | 12 | 55325473-55325788 | − | 55326024 | 236 | upstream | ATP5B/BC016512 |
| | | | | 12 | 55325473-55325788 | − | 55326119 | 331 | upstream | NM_001686 |
| 70 | FJ#43G1 | 597 | 1 | 19 | 54214027-54214096 | − | 54212159 | 1868 | upstream | LHB/NM_000894 |
| 71 | FJ#43G7 | 592 | 592 | 2 | 69575838-69576430 | − | 69576199 | 0 | within | HIRIP5/AJ132584 |
| | | | | 2 | 69575838-69576430 | − | 69576199 | 0 | within | NM_015700 |
| | | | | 2 | 69575838-69576430 | − | 69576258 | 0 | within | HIRIP5/AY286307 |
| | | | | 2 | 69575838-69576430 | − | 69576276 | 0 | within | HIRIP5/BX538347 |
| | | | | 2 | 69575838-69576430 | − | 69576404 | 0 | within | AY335194 |
| 72 | FJ#45G7 | 579 | 647 | 14 | 54807341-54808469 | + | 54807831 | 0 | within | NM_017943 |
| | | | | 14 | 54807341-54808469 | + | 54807921 | 0 | within | FBXO34/BX248268 |
| 73 | FJ#50E3 | 728 | 731 | 19 | 58590696-58591429 | + | 58590245 | 451 | downstream | LOC91661/BC017357 |
| | | | | 19 | 58590696-58591429 | + | 58590245 | 451 | downstream | NM_138372 |
| | | | | 19 | 58590696-58591429 | + | 58593007 | 1578 | downstream | LOC91661/BC001610 |
| | | | | 19 | 58187398-58187454 | − | 58188596 | 1142 | upstream | NM_024924 |
| | | | | 19 | 57832436-57832839 | − | 57830163 | 2273 | upstream | AK027782 |
| | | | | 19 | 57832436-57832839 | − | 57833450 | 611 | upstream | ZNF83/AK027518 |
| | | | | 19 | 57832436-57832839 | − | 57833450 | 611 | upstream | NM_018300 |
| | | | | 19 | 58136659-58136715 | − | 58137650 | 935 | upstream | MGC35402/BC046449 |
| | | | | 19 | 58136659-58136715 | − | 58137650 | 935 | upstream | NM_203307 |
| | | | | 19 | 58136659-58136715 | − | 58137659 | 944 | upstream | MGC35402/AK096828 |
| 74 | FJ#49G3 | 729 | 730 | 7 | 10752372-10753099 | − | 10752977 | 0 | within | NDUFA4/AF201077 |
| | | | | 7 | 10752372-10753099 | − | 10753053 | 0 | within | NM_002489 |
| 75 | FJ#54C5 | 404 | 582 | x | 118152819-118153392 | + | 118152144 | 675 | downstream | NM_006667 |
| | | | | x | 118152819-118153392 | + | 118152155 | 664 | downstream | PGRMC1/BC034238 |
| 76 | FJ#55C3 | 597 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 77 | FJ#60A7 | 640 | 681 | 19 | 57980812-57981089 | − | 57981828 | 739 | upstream | ZNF600/BX640933 |
| | | | | 19 | 57980812-57981089 | − | 57981828 | 739 | upstream | NM_198457 |
| | | | | 19 | 57981030-57981089 | − | 57981828 | 739 | upstream | ZNF600/BX640933 |
| | | | | 19 | 57981030-57981089 | − | 57981828 | 739 | upstream | NM_198457 |
| | | | | 19 | 57919817-57919878 | − | 57919729 | 88 | upstream | BC015370 |
| | | | | 19 | 57832419-57832481 | − | 57830163 | 2256 | upstream | AK027782 |
| | | | | 19 | 57832419-57832481 | − | 57833450 | 969 | upstream | ZNF83/AK027518 |
| | | | | 19 | 57832419-57832481 | − | 57833450 | 969 | upstream | NM_018300 |
| | | | | 19 | 58187381-58187443 | − | 58188596 | 1153 | upstream | NM_024924 |
| | | | | 19 | 58591184-58591246 | + | 58590245 | 939 | downstream | LOC91661/BC017357 |
| | | | | 19 | 58591184-58591246 | + | 58590245 | 939 | downstream | NM_138372 |
| | | | | 19 | 58591184-58591246 | + | 58593007 | 1761 | downstream | LOC91661/BC001610 |
| | | | | 19 | 57766108-57766178 | + | 57765339 | 769 | downstream | FLJ10891/AK001753 |
| | | | | 19 | 57766108-57766178 | + | 57765339 | 769 | downstream | NM_018260 |
| | | | | 19 | 57766108-57766178 | + | 57765372 | 736 | downstream | BC054884 |
| | | | | 19 | 57766108-57766178 | + | 57765728 | 380 | downstream | BC067346 |
| 78 | FJ#57E1 | 426 | 584 | 12 | 121536367-121536679 | + | 121536688 | 9 | downstream | KNTC1/D79988 |
| | | | | 12 | 121536367-121536679 | + | 121536688 | 9 | downstream | NM_014708 |
| | | | | 12 | 121536367-121536679 | − | 121536350 | 17 | upstream | FLJ11021/BC008684 |
| | | | | 12 | 121536367-121536679 | − | 121536363 | 4 | upstream | FLJ11021/BX640711 |
| | | | | 12 | 121536367-121536679 | − | 121536410 | 0 | within | FLJ11021/BC067773 |
| | | | | 12 | 121536367-121536679 | − | 121536426 | 0 | within | NM_023012 |
| | | | | 12 | 121536367-121536679 | − | 121536426 | 0 | within | NM_198261 |
| | | | | 12 | 121536367-121536679 | − | 121536427 | 0 | within | NM_198262 |
| | | | | 12 | 121536367-121536679 | − | 121536427 | 0 | within | NM_198263 |
| 79 | FJ#63C5 | 510 | 509 | 6 | 101953422-101953924 | + | 101953389 | 33 | downstream | GRIK2/BC037954 |
| | | | | 6 | 101953422-101953924 | + | 101953671 | 0 | within | BC063814 |
| | | | | 6 | 101953422-101953924 | + | 101953674 | 0 | within | NM_021956 |
| | | | | 6 | 101953422-101953924 | + | 101953674 | 0 | within | NM_175768 |
| | | | | 6 | 101953422-101953924 | + | 101953874 | 0 | within | GRIK2/U16126 |
| | | | | 6 | 101953422-101953924 | + | 101953874 | 0 | within | GRIK2/AJ252246 |
| | | | | 6 | 101953422-101953924 | + | 101953874 | 0 | within | GRIK2/AJ301610 |
| 80 | FJ#63G11 | 871 | 963 | 17 | 15527868-15528278 | − | 15526918 | 950 | upstream | TRIM16/BC053514 |
| | | | | 17 | 15527868-15528278 | − | 15526918 | 950 | upstream | NM_006470 |
| | | | | 19 | 42777516-42778141 | + | 42777570 | 0 | within | AL832100 |
| | | | | 19 | 42777516-42778141 | + | 42777626 | 0 | within | ZNF540/BX537980 |
| | | | | 19 | 42777516-42778141 | − | 42777531 | 0 | within | ZNF571/BX537401 |
| | | | | 19 | 42777516-42778141 | − | 42777531 | 0 | within | NM_016536 |
| 81 | FJ#65G5 | 946 | 916 | 5 | 70398856-70399707 | − | 70394346 | 4510 | upstream | BT006773 |
| | | | | 5 | 70398856-70399707 | − | 70399238 | 0 | within | GTF2H2/AF078847 |
| | | | | 5 | 70398856-70399707 | − | 70399238 | 0 | within | NM_001515 |
| | | | | 5 | 68891204-68892206 | + | 68891824 | 0 | within | GTF2H2/AF078847 |
| | | | | 5 | 68891204-68892206 | + | 68891824 | 0 | within | NM_001515 |
| | | | | 5 | 68891204-68892206 | + | 68896715 | 4509 | downstream | BT006773 |
| | | | | 5 | 69746502-69747353 | + | 69746971 | 0 | within | GTF2H2/AF078847 |
| | | | | 5 | 69746502-69747353 | + | 69746971 | 0 | within | NM_001515 |
| | | | | 5 | 69746502-69747353 | + | 69751864 | 4511 | downstream | BT006773 |
| 82 | FJ#70A5 | 303 | 303 | 16 | 66677340-66677643 | + | 66676875 | 465 | downstream | NFATC3/L41067 |
| | | | | 16 | 66677340-66677643 | + | 66676875 | 465 | downstream | NM_173164 |
| | | | | 16 | 66677340-66677643 | + | 66676875 | 465 | downstream | NM_004555 |
| | | | | 16 | 66677340-66677643 | + | 66676875 | 465 | downstream | NM_173163 |
| | | | | 16 | 66677340-66677643 | + | 66676875 | 465 | downstream | NM_173165 |
| 83 | FJ#68G9 | 623 | 626 | 19 | 57832402-57832481 | − | 57830163 | 2239 | upstream | AK027782 |
| | | | | 19 | 57832402-57832481 | − | 57833450 | 969 | upstream | ZNF83/AK027518 |
| | | | | 19 | 57832402-57832481 | − | 57833450 | 969 | upstream | NM_018300 |
| | | | | 19 | 58591189-58591311 | + | 58590245 | 944 | downstream | LOC91661/BC017357 |
| | | | | 19 | 58591189-58591311 | + | 58590245 | 944 | downstream | NM_138372 |
| | | | | 19 | 58591189-58591311 | + | 58593007 | 1696 | downstream | LOC91661/BC001610 |
| | | | | 19 | 58590829-58591311 | + | 58590245 | 584 | downstream | LOC91661/BC017357 |
| | | | | 19 | 58590829-58591311 | + | 58590245 | 584 | downstream | NM_138372 |
| | | | | 19 | 58590829-58591311 | + | 58593007 | 1696 | downstream | LOC91661/BC001610 |
| | | | | 19 | 58136659-58136715 | − | 58137650 | 935 | upstream | MGC35402/BC046449 |
| | | | | 19 | 58136659-58136715 | − | 58137650 | 935 | upstream | NM_203307 |
| | | | | 19 | 58136659-58136715 | − | 58137659 | 944 | upstream | MGC35402/AK096828 |
| | | | | 19 | 58590829-58591311 | + | 58590245 | 584 | downstream | LOC91661/BC017357 |
| | | | | 19 | 58590829-58591311 | + | 58590245 | 584 | downstream | NM_138372 |
| | | | | 19 | 58590829-58591311 | + | 58593007 | 1696 | downstream | LOC91661/BC001610 |
| | | | | 19 | 58590829-58590881 | + | 58590245 | 584 | downstream | LOC91661/BC017357 |
| | | | | 19 | 58590829-58590881 | + | 58590245 | 584 | downstream | NM_138372 |
| | | | | 19 | 58590829-58590881 | + | 58593007 | 2126 | downstream | LOC91661/BC001610 |
| | | | | 19 | 58187380-58187443 | − | 58188596 | 1153 | upstream | NM_024924 |
| | | | | 19 | 57766111-57766384 | + | 57765339 | 772 | downstream | FLJ10891/AK001753 |
| | | | | 19 | 57766111-57766384 | + | 57765339 | 772 | downstream | NM_018260 |
| | | | | 19 | 57766111-57766384 | + | 57765372 | 739 | downstream | BC054884 |
| | | | | 19 | 57766111-57766384 | + | 57765728 | 383 | downstream | BC067346 |
| | | | | 19 | 57980811-57980874 | − | 57981828 | 954 | upstream | ZNF600/BX640933 |
| | | | | 19 | 57980811-57980874 | − | 57981828 | 954 | upstream | NM_198457 |
| | | | | 19 | 57919817-57919878 | − | 57919729 | 88 | upstream | BC015370 |
| 84 | FJ#36A12 | 206 | 799 | 12 | 6853147-6853464 | + | 6848389 | 4758 | downstream | TPI1/BC017165 |
| | | | | 12 | 6853147-6853464 | − | 6852713 | 434 | upstream | GRCC9/BC002983 |
| | | | | 12 | 6853147-6853464 | − | 6852713 | 434 | upstream | NM_032641 |
| 85 | FJ#39A2 | 317 | 568 | 7 | 20599607-20600164 | − | 20598903 | 704 | upstream | SP8/AY167048 |
| | | | | 7 | 20599607-20600164 | − | 20599745 | 0 | within | SP8/BC038669 |
| | | | | 7 | 20599607-20600164 | − | 20599745 | 0 | within | SP8/AY167047 |
| | | | | 7 | 20599607-20600164 | − | 20599745 | 0 | within | NM_182700 |
| | | | | 7 | 20599607-20600164 | − | 20599745 | 0 | within | NM_198956 |
| 86 | FJ#41A8 | 762 | 718 | 17 | 73395410-73396172 | − | 73391764 | 3646 | upstream | AK127023 |
| | | | | 17 | 73395410-73396172 | − | 73391764 | 3646 | upstream | NM_001001685 |
| 87 | FJ#41A10 | 62 | 235 | 19 | 60808345-60808544 | + | 60803541 | 4804 | downstream | NM_153219 |
| | | | | 19 | 60808345-60808544 | + | 60803548 | 4797 | downstream | ZNF524/BC067748 |
| | | | | 19 | 60808345-60808544 | + | 60805300 | 3045 | downstream | ZNF524/BC007396 |
| 88 | FJ#41A12 | 806 | 91 | 19 | 62554224-62554913 | + | 62554486 | 0 | within | ZNF304/AJ276316 |
| | | | | 19 | 62554224-62554913 | + | 62554486 | 0 | within | NM_020657 |
| 89 | FJ#41E2 | 274 | 274 | 19 | 10387883-10388157 | + | 10392332 | 4175 | downstream | PDE4A/L20965 |
| 90 | FJ#43E4 | 927 | 777 | 8 | 136539451-136540341 | + | 136538883 | 568 | downstream | KHDRBS3/BC068536 |
| | | | | 8 | 136539451-136540341 | + | 136538897 | 554 | downstream | NM_006558 |
| | | | | 8 | 136539451-136540341 | + | 136539312 | 139 | downstream | KHDRBS3/AF051322 |
| 91 | FJ#43E8 | 386 | 385 | 19 | 52305563-52305874 | − | 52308849 | 2975 | upstream | C19orf7/AB028987 |
| 92 | FJ#43E10 | 549 | 548 | 12 | 21817405-21817908 | − | 21818882 | 974 | upstream | KCNJ8/BC000544 |
| | | | | 12 | 21817405-21817908 | − | 21819014 | 1106 | upstream | NM_004982 |
| 93 | FJ#43G2 | 641 | 641 | 16 | 81219070-81219711 | + | 81218078 | 992 | downstream | CDH13/NM_001257 |
| | | | | 16 | 81219070-81219711 | + | 81218133 | 937 | downstream | CDH3/U59289 |
| 94 | FJ#47E6 | 152 | 279 | 12 | 9283905-9284131 | − | 9284194 | 63 | upstream | AF170294 |
| 95 | FJ#48E10 | 869 | 829 | 15 | 30694776-30695758 | + | 30694982 | 0 | within | ARHGAP11A/D87717 |
| | | | | 15 | 30694776-30695758 | + | 30694982 | 0 | within | NM_199357 |
| | | | | 15 | 30694776-30695758 | + | 30694982 | 0 | within | NM_014783 |
| | | | | 15 | 30694776-30695758 | + | 30695031 | 0 | within | ARHGAP11A/BC063444 |
| | | | | 15 | 30694776-30695758 | + | 30695127 | 0 | within | ARHGAP11A/BC039563 |
| 96 | FJ#47G12 | 681 | 596 | 2 | 97720166-97720843 | + | 97721038 | 195 | downstream | COX5B/BC006229 |
| | | | | 2 | 97720166-97720843 | + | 97721038 | 195 | downstream | NM_001862 |
| | | | | 2 | 97720166-97720843 | + | 97721067 | 224 | downstream | BT006742 |
| 97 | FJ#49G8 | 409 | 752 | 7 | 115760338-115761078 | + | 115758789 | 1549 | downstream | NM_001753 |
| | | | | 7 | 115760338-115761078 | + | 115758994 | 1344 | downstream | CAV1/BC009685 |
| | | | | 7 | 115760338-115761078 | + | 115759067 | 1271 | downstream | BT007143 |
| | | | | 7 | 115760338-115761078 | + | 115760357 | 0 | within | AF172085 |
| 98 | FJ#51G2 | 420 | 421 | 7_random | 195869-196281 | − | 194591 | 1278 | upstream | AK021933 |
| 99 | FJ#66E6 | 723 | 857 | 14 | 23868826-23869660 | − | 23873704 | 4044 | upstream | ADCY4/AF497516 |
| | | | | 14 | 23868826-23869660 | − | 23873704 | 4044 | upstream | NM_139247 |
| | | | | 14 | 23868826-23869660 | − | 23874084 | 4424 | upstream | BX248285 |
| | | | | 14 | 23868826-23869660 | − | 23874117 | 4457 | upstream | ADCY4/AK126468 |
| 100 | FJ#40D9 | 789 | 788 | 19 | 57929063-57929775 | − | 57924947 | 4116 | upstream | ZNF611/AK097434 |
| | | | | 19 | 57929063-57929775 | − | 57924947 | 4116 | upstream | NM_030972 |
| 101 | FJ#40H5 | 328 | 329 | 1 | 222557795-222558123 | + | 222557155 | 640 | downstream | NM_002107 |
| | | | | 1 | 222557795-222558123 | + | 222558412 | 289 | downstream | H3F3A/M11353 |
| 102 | FJ#48D9 | 0 | 261 | 6 | 7051851-7051994 | + | 7052828 | 834 | downstream | RREB1/D49835 |
| | | | | 6 | 7051851-7051994 | − | 7053973 | 1979 | upstream | AK127740 |
| 103 | FJ#47F5 | 638 | 678 | 15 | 50869726-50870387 | − | 50869501 | 225 | upstream | ONECUT1/U96173 |
| | | | | 15 | 50869726-50870387 | − | 50869501 | 225 | upstream | NM_004498 |
| | | | | x | 108103645-108103938 | + | 108103493 | 152 | downstream | NM_173479 |
| 104 | FJ#59H3 | 679 | 680 | 19 | 45194865-45195545 | + | 45194868 | 0 | within | ZNF546/BC045649 |
| | | | | 19 | 45194865-45195545 | + | 45194868 | 0 | within | NM_178544 |
| 105 | FJ#36D2 | 816 | 676 | 1 | 86334090-86334710 | − | 86334142 | 0 | within | COL24A1/AY244357 |
| | | | | 1 | 86334090-86334710 | − | 86334142 | 0 | within | NM_152890 |
| 106 | FJ#40D8 | 788 | 788 | 19 | 57929063-57929775 | − | 57924947 | 4116 | upstream | ZNF611/AK097434 |
| | | | | 19 | 57929063-57929775 | − | 57924947 | 4116 | upstream | NM_030972 |
| 107 | FJ#36F4 | 447 | 447 | 2 | 73210959-73211339 | − | 73210620 | 339 | upstream | SFXN5/AY044437 |
| | | | | 2 | 73210959-73211339 | − | 73210620 | 339 | upstream | NM_144579 |
| 108 | FJ#38F12 | 494 | 273 | 12 | 99097125-99097359 | + | 99097041 | 84 | downstream | ACTR6/BC015107 |
| | | | | 12 | 99097125-99097359 | + | 99097041 | 84 | downstream | NM_022496 |
| | | | | 12 | 99097125-99097359 | + | 99097051 | 74 | downstream | AF161399 |
| | | | | 12 | 99097125-99097359 | + | 99097085 | 40 | downstream | AF175226 |
| 109 | FJ#41B4 | 357 | 655 | 9 | 21325108-21325775 | − | 21324073 | 1035 | upstream | KIAA1354/AK023630 |
| | | | | 9 | 21325108-21325775 | − | 21325240 | 0 | within | KIAA1354/BX538121 |
| | | | | 9 | 21325108-21325775 | − | 21325368 | 0 | within | KIAA1354/AB037775 |
| | | | | 9 | 21325108-21325775 | − | 21325371 | 0 | within | KIAA1354/AL713669 |
| | | | | 9 | 21325108-21325775 | − | 21325371 | 0 | within | NM_018847 |
| 110 | FJ#41D6 | 838 | 839 | 11 | 6236999-6238808 | + | 6237541 | 0 | within | CCKBR/D13305 |
| | | | | 11 | 6236999-6238808 | + | 6237541 | 0 | within | NM_176875 |
| | | | | 11 | 6236999-6238808 | + | 6237731 | 0 | within | AF239668 |
| | | | | 11 | 6236999-6238808 | + | 6237734 | 0 | within | BT006789 |
| 111 | FJ#41F10 | 857 | 828 | 1 | 47610137-47611260 | + | 47613708 | 2448 | downstream | FOXD2/AF042832 |
| | | | | 1 | 47610137-47611260 | + | 47613708 | 2448 | downstream | NM_004474 |
| 112 | FJ#43F4 | 197 | 713 | 9 | 99748255-99749095 | + | 99748506 | 0 | within | BC051790 |
| | | | | 9 | 99748255-99749095 | + | 99748520 | 0 | within | STX17/AK000658 |
| | | | | 9 | 99748255-99749095 | + | 99748520 | 0 | within | NM_017919 |
| 113 | FJ#41H8 | 416 | 416 | 6 | 34833073-34833489 | + | 34833289 | 0 | within | SNRPC/X12517 |
| | | | | 6 | 34833073-34833489 | + | 34833289 | 0 | within | NM_003093 |
| 114 | FJ#44H2 | 875 | 875 | 12 | 7915955-7916817 | + | 7916800 | 0 | within | AY455283 |
| | | | | 12 | 7915955-7916817 | − | 7916749 | 0 | within | SLC2A14/BC060766 |
| | | | | 12 | 7915955-7916817 | − | 7916762 | 0 | within | SLC2A14/AF481879 |
| | | | | 12 | 7915955-7916817 | − | 7916762 | 0 | within | NM_153449 |
| 115 | FJ#50B2 | 970 | 1000 | 20 | 23921502-23921561 | − | 23917416 | 4086 | upstream | GGTLA4/BC040904 |
| | | | | 20 | 23921502-23921561 | − | 23917416 | 4086 | upstream | NM_178311 |
| | | | | 22 | 22976214-22976273 | + | 22972349 | 3865 | downstream | DKFZP434P211/AL117401 |
| | | | | 22 | 22976214-22976273 | + | 22972349 | 3865 | downstream | NM_014549 |
| | | | | 22 | 21307007-21307413 | − | 21311110 | 3697 | upstream | POM121L1/NM_014348 |
| 116 | FJ#50B8 | 550 | 550 | 7 | 134312143-134312667 | − | 134310090 | 2053 | upstream | MGC5242/AK130795 |
| | | | | 7 | 134312143-134312667 | − | 134312702 | 35 | upstream | MGC5242/BC067350 |
| | | | | 7 | 134312143-134312667 | − | 134312702 | 35 | upstream | MGC5242/BC000168 |
| | | | | 7 | 134312143-134312667 | − | 134312702 | 35 | upstream | NM_024033 |
| 117 | FJ#48D6 | 747 | 757 | 1 | 85453687-85454813 | − | 85455604 | 791 | upstream | BCL10/AF082283 |
| | | | | 1 | 85453687-85454813 | − | 85455604 | 791 | upstream | NM_003921 |
| 118 | FJ#46F4 | 772 | 562 | 19 | 57832419-57832481 | − | 57830163 | 2256 | upstream | AK027782 |
| | | | | 19 | 57832419-57832481 | − | 57833450 | 969 | upstream | ZNF83/AK027518 |
| | | | | 19 | 57832419-57832481 | − | 57833450 | 969 | upstream | NM_018300 |
| | | | | 19 | 57766108-57766178 | + | 57765339 | 769 | downstream | FLJ10891/AK001753 |
| | | | | 19 | 57766108-57766178 | + | 57765339 | 769 | downstream | NM_018260 |
| | | | | 19 | 57766108-57766178 | + | 57765372 | 736 | downstream | BC054884 |
| | | | | 19 | 57766108-57766178 | + | 57765728 | 380 | downstream | BC067346 |
| | | | | 19 | 58187381-58187443 | − | 58188596 | 1153 | upstream | NM_024924 |
| | | | | 19 | 57919817-57919878 | − | 57919729 | 88 | upstream | BC015370 |
| | | | | 19 | 58591184-58591246 | + | 58590245 | 939 | downstream | LOC91661/BC017357 |
| | | | | 19 | 58591184-58591246 | + | 58590245 | 939 | downstream | NM_138372 |
| | | | | 19 | 58591184-58591246 | + | 58593007 | 1761 | downstream | LOC91661/BC001610 |
| | | | | 19 | 57980812-57981089 | − | 57981828 | 739 | upstream | ZNF600/BX640933 |
| | | | | 19 | 57980812-57981089 | − | 57981828 | 739 | upstream | NM_198457 |
| | | | | 19 | 57981030-57981089 | − | 57981828 | 739 | upstream | ZNF600/BX640933 |
| | | | | 19 | 57981030-57981089 | − | 57981828 | 739 | upstream | NM_198457 |
| | | | | 19 | 57980812-57980874 | − | 57981828 | 954 | upstream | ZNF600/BX640933 |
| | | | | 19 | 57980812-57980874 | − | 57981828 | 954 | upstream | NM_198457 |
| | | | | 19 | 57980812-57981089 | − | 57981828 | 739 | upstream | ZNF600/BX640933 |
| | | | | 19 | 57980812-57981089 | − | 57981828 | 739 | upstream | NM_198457 |
| 119 | FJ#46H4 | 561 | 558 | 2 | 104927799-104928345 | + | 104930486 | 2141 | downstream | POU3F3/NM_006236 |
| 120 | FJ#48H4 | 537 | 694 | 12 | 30798568-30799665 | − | 30797565 | 1003 | upstream | C1QDC1/AK021453 |
| | | | | 12 | 30798568-30799665 | − | 30797858 | 710 | upstream | C1QDC1/BX537569 |
| | | | | 12 | 30798568-30799665 | − | 30798715 | 0 | within | C1QDC1/AY074490 |
| | | | | 12 | 30798568-30799665 | − | 30798715 | 0 | within | C1QDC1/AY074491 |
| | | | | 12 | 30798568-30799665 | − | 30798715 | 0 | within | NM_023925 |
| | | | | 12 | 30798568-30799665 | − | 30798715 | 0 | within | NM_001002259 |
| | | | | 12 | 30798568-30799665 | − | 30798715 | 0 | within | NM_032156 |
| 121 | FJ#48H8 | 868 | 828 | 12 | 81254274-81255287 | + | 81254786 | 0 | within | FLJ22789/AK026442 |
| | | | | 12 | 81254274-81255287 | + | 81254791 | 0 | within | FLJ22789/BC029120 |
| | | | | 12 | 81254274-81255287 | + | 81254791 | 0 | within | NM_032230 |
| | | | | 12 | 81254274-81255287 | − | 81254640 | 0 | within | HSPC128/AF161477 |
| | | | | 12 | 81254274-81255287 | − | 81254640 | 0 | within | NM_014167 |
| | | | | 12 | 81254274-81255287 | − | 81254643 | 0 | within | AK001156 |
| 122 | FJ#51D2 | 705 | 703 | 12 | 2856602-2857305 | + | 2856659 | 0 | within | MGC13204/BC005106 |
| | | | | 12 | 2856602-2857305 | + | 2856659 | 0 | within | NM_031465 |
| | | | | 12 | 2856602-2857305 | − | 2853905 | 2697 | upstream | FOXM1/BT006986 |
| | | | | 12 | 2856602-2857305 | − | 2856413 | 189 | upstream | FOXM1/U74612 |
| | | | | 12 | 2856602-2857305 | − | 2856564 | 38 | upstream | NM_021953 |
| | | | | 12 | 2856602-2857305 | − | 2856564 | 38 | upstream | NM_202002 |
| | | | | 12 | 2856602-2857305 | − | 2856564 | 38 | upstream | NM_202003 |
| 123 | FJ#51D10 | 760 | 761 | 12 | 55758348-55759109 | − | 55758802 | 0 | within | AB006624 |
| 124 | FJ#54D2 | 546 | 547 | 18 | 59789147-59789687 | + | 59788242 | 905 | downstream | NM_002640 |
| | | | | 18 | 59789147-59789687 | + | 59788242 | 905 | downstream | NM_198833 |
| | | | | 18 | 59789147-59789687 | + | 59788311 | 836 | downstream | SERPINB8/L40377 |
| | | | | 18 | 59789147-59789687 | + | 59788332 | 815 | downstream | SERPINB8/BC034528 |
| 125 | FJ#51F10 | 715 | 781 | 1 | 210550852-210551624 | + | 210549680 | 1172 | downstream | PROX1/U44060 |
| | | | | 1 | 210550852-210551624 | + | 210550254 | 598 | downstream | PROX1/BC024201 |
| | | | | 1 | 210550852-210551624 | + | 210550254 | 598 | downstream | NM_002763 |
| 126 | FJ#54F2 | 483 | 484 | 4 | 163442955-163443429 | − | 163442723 | 232 | upstream | FSTL5/AB033089 |
| | | | | 4 | 163442955-163443429 | − | 163442791 | 164 | upstream | FSTL5/BC036502 |
| | | | | 4 | 163442955-163443429 | − | 163442791 | 164 | upstream | NM_020116 |
| 127 | FJ#54H12 | 705 | 705 | x | 52870728-52871428 | − | 52869197 | 1531 | upstream | NM_014138 |
| | | | | x | 52808646-52809350 | + | 52810882 | 1532 | downstream | AF370413 |
| | | | | x | 52808646-52809350 | + | 52810882 | 1532 | downstream | NM_014138 |
| 128 | FJ#55H4 | 600 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 129 | FJ#56H4 | 593 | 593 | 1 | 45718132-45718724 | + | 45718809 | 85 | downstream | NM_002482 |
| | | | | 1 | 45718132-45718724 | + | 45718809 | 85 | downstream | NM_152298 |
| | | | | 1 | 45718132-45718724 | + | 45718809 | 85 | downstream | NM_172164 |
| | | | | 1 | 45718132-45718724 | + | 45718824 | 100 | downstream | NASP/BC010105 |
| | | | | 1 | 45718132-45718724 | + | 45718827 | 103 | downstream | NASP/AF035191 |
| | | | | 1 | 45718132-45718724 | + | 45718828 | 104 | downstream | NASP/BC009933 |
| | | | | 1 | 45718132-45718724 | + | 45718911 | 187 | downstream | NASP/BT006757 |
| 130 | FJ#62D8 | 856 | 896 | 22 | 29311543-29312551 | − | 29312427 | 0 | within | PES1/BC032489 |
| | | | | 22 | 29311543-29312551 | − | 29312448 | 0 | within | NM_014303 |
| 131 | FJ#65F8 | 318 | 318 | 6 | 24883926-24884244 | + | 24883142 | 784 | downstream | NM_015895 |
| | | | | 6 | 24883926-24884244 | + | 24883162 | 764 | downstream | GMNN/BC005389 |
| 132 | FJ#65H10 | 313 | 318 | 6 | 24883926-24884244 | + | 24883142 | 784 | downstream | NM_015895 |
| | | | | 6 | 24883926-24884244 | + | 24883162 | 764 | downstream | GMNN/BC005389 |
| 133 | FJ#69B8 | 285 | 694 | 8 | 120497158-120498049 | + | 120497881 | 0 | within | NOV/AY082381 |
| | | | | 8 | 120497158-120498049 | + | 120497881 | 0 | within | NM_002514 |
| 134 | FJ#73A9 | 558 | 558 | 12 | 24947530-24948088 | + | 24946509 | 1021 | downstream | BCAT1/AK124863 |
| | | | | 12 | 24947530-24948088 | − | 24946087 | 1443 | upstream | BCAT1/U21551 |
| | | | | 12 | 24947530-24948088 | − | 24946589 | 941 | upstream | BCAT1/BC033864 |
| 135 | FJ#75A5 | 591 | 591 | 6 | 122761985-122762576 | + | 122762493 | 0 | within | HSF2/M65217 |
| | | | | 6 | 122761985-122762576 | + | 122762493 | 0 | within | NM_004506 |
| | | | | 6 | 122761985-122762576 | + | 122762505 | 0 | within | HSF2/BC005329 |
| 136 | FJ#72E5 | 705 | 734 | 10 | 92621643-92622378 | + | 92621254 | 389 | downstream | RPP30/BC006991 |
| | | | | 10 | 92621643-92622378 | + | 92621254 | 389 | downstream | NM_006413 |
| 137 | FJ#73E5 | 846 | 644 | 10 | 63478762-63479400 | + | 63479007 | 0 | within | BC066345 |
| 138 | FJ#75E11 | 526 | 526 | 5 | 107032427-107032954 | − | 107034495 | 1541 | upstream | EFNA5/U26403 |
| | | | | 5 | 107032427-107032954 | − | 107034495 | 1541 | upstream | NM_001962 |
| 139 | FJ#71G3 | 547 | 547 | 4 | 147217396-147217943 | − | 147217187 | 209 | upstream | LOC152485/AK091130 |
| | | | | 4 | 147217396-147217943 | − | 147217187 | 209 | upstream | NM_178835 |
| | | | | 4 | 147217396-147217943 | − | 147217246 | 150 | upstream | LOC152485/AF450485 |
| 140 | FJ#76A3 | 155 | 148 | 4 | 129061803-129061954 | + | 129061057 | 746 | downstream | APG-1/BC040560 |
| | | | | 4 | 129061803-129061954 | + | 129061057 | 746 | downstream | NM_014278 |
| 141 | FJ#76C5 | 671 | 671 | 6 | 80770855-80771486 | + | 80771077 | 0 | within | TTK/BC000633 |
| | | | | 6 | 80770855-80771486 | + | 80771077 | 0 | within | NM_003318 |
| | | | | 6 | 80770855-80771486 | + | 80772274 | 788 | downstream | TTK/M86699 |
| 142 | FJ#82A7 | 210 | 722 | 1 | 85885132-85885762 | − | 85885509 | 0 | within | FLJ20729/AK000736 |
| | | | | 1 | 85885132-85885762 | − | 85885784 | 22 | upstream | FLJ20729/AF308296 |
| | | | | 1 | 85885132-85885762 | − | 85886122 | 360 | upstream | FLJ20729/AL442074 |
| | | | | 1 | 85885132-85885762 | − | 85886122 | 360 | upstream | NM_017953 |
| 143 | FR#2A1 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 144 | FR#2A3 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 145 | FR#1C9 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 146 | FR#2C11 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 147 | FR#3E3 | 513 | 514 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 148 | FR#3G1 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 149 | FR#6A1 | 0 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 150 | FR#5E3 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 151 | FR#4G9 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 152 | FR#6G1 | 511 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 153 | FJ#75A2 | 557 | 557 | 15 | 38240536-38241093 | + | 38240529 | 7 | downstream | BUB1B/AF053306 |
| | | | | 15 | 38240536-38241093 | + | 38240579 | 0 | within | NM_001211 |
| 154 | FJ#75C6 | 800 | 800 | 19 | 57832386-57832462 | − | 57830163 | 2223 | upstream | AK027782 |
| | | | | 19 | 57832386-57832462 | − | 57833450 | 988 | upstream | ZNF83/AK027518 |
| | | | | 19 | 57832386-57832462 | − | 57833450 | 988 | upstream | NM_018300 |
| | | | | 19 | 57765661-57766441 | + | 57765339 | 322 | downstream | FLJ10891/AK001753 |
| | | | | 19 | 57765661-57766441 | + | 57765339 | 322 | downstream | NM_018260 |
| | | | | 19 | 57765661-57766441 | + | 57765372 | 289 | downstream | BC054884 |
| | | | | 19 | 57765661-57766441 | + | 57765728 | 0 | within | BC067346 |
| | | | | 19 | 58591203-58591263 | + | 58590245 | 958 | downstream | LOC91661/BC017357 |
| | | | | 19 | 58591203-58591263 | + | 58590245 | 958 | downstream | NM_138372 |
| | | | | 19 | 58591203-58591263 | + | 58593007 | 1744 | downstream | LOC91661/BC001610 |
| 155 | FJ#76A4 | 460 | 808 | 21 | 32166204-32167272 | + | 32167498 | 226 | downstream | HUNK/AJ271722 |
| | | | | 21 | 32166204-32167272 | + | 32167498 | 226 | downstream | NM_014586 |
| 156 | FJ#82C12 | 818 | 701 | 10 | 102016776-102017591 | − | 102017345 | 0 | within | CWF19L1/AK023984 |
| | | | | 10 | 102016776-102017591 | − | 102017369 | 0 | within | CWF19L1/BC008746 |
| | | | | 10 | 102016776-102017591 | − | 102017369 | 0 | within | NM_018294 |
| 157 | FR#3C4 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 158 | FR#3E4 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 159 | FR#3E10 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 160 | FR#2G2 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 161 | FR#2G12 | 513 | 513 | unknown | −1-−1 | unknown | −1 | −1 | unknown | BDNF |
| | | | | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 162 | FR#6C2 | 511 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 163 | FJ#71F3 | 565 | 565 | 15 | 63476002-63476565 | − | 63475820 | 182 | upstream | NOPE/AB046848 |
| 164 | FJ#75F1 | 651 | 709 | 6 | 84799819-84800485 | + | 84800138 | 0 | within | C6orf117/AK090775 |
| | | | | 6 | 84799819-84800485 | + | 84800138 | 0 | within | NM_138409 |
| 165 | FJ#76F1 | 451 | 451 | 3 | 33234357-33234808 | − | 33235711 | 903 | upstream | AB011099 |
| 166 | FJ#77F11 | 827 | 897 | 12 | 6519661-6520535 | + | 6517358 | 2303 | downstream | M28283 |
| 167 | FJ#77H1 | 0 | 809 | 8 | 103944210-103945020 | − | 103945543 | 523 | upstream | OAZIN/BC013420 |
| | | | | 8 | 103944210-103945020 | − | 103945551 | 531 | upstream | NM_015878 |
| | | | | 8 | 103944210-103945020 | − | 103945551 | 531 | upstream | NM_148174 |
| 168 | FJ#73F6 | 789 | 789 | 19 | 58326878-58327589 | − | 58327947 | 358 | upstream | ZNF415/BC063880 |
| | | | | 19 | 58326878-58327589 | − | 58327957 | 368 | upstream | ZNF415/AY283600 |
| | | | | 19 | 58326878-58327589 | − | 58327957 | 368 | upstream | NM_018355 |
| 169 | FR#3B10 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |

TABLES 13 shows, according to particular preferred aspects, markers for AML as identified by methylation hybridization as described in the EXAMPLES herein.

| No. | CloneID | T7 Sequence Length | M13 Sequence Length | Chromosome Aligned | Alignment Address | Strand | TSS | Distance to TSS | Direction | Gene/Assession Number |
|---|---|---|---|---|---|---|---|---|---|---|
| 1 | FJ#7E5 | 0 | 577 | unknown | −1-−1 | unknown | −1 | −1 | unknown | IMAGE: 5262055 |
| 2 | FJ#10G9 | 555 | 555 | 16 | 29845411-29845966 | − | 29845046 | 365 | upstream | KCTD13/BC036228 |
| | | | | 16 | 29845411-29845966 | − | 29845046 | 365 | upstream | NM_178863 |
| 3 | FJ#13G1 | 733 | 833 | 10 | 101978657-101979488 | − | 101979366 | 0 | within | CHUK/AF080157 |
| | | | | 10 | 101978657-101979488 | − | 101979366 | 0 | within | NM_001278 |
| | | | | 17 | 77735181-77735233 | − | 77736165 | 932 | upstream | LOC284001/AK074059 |
| | | | | 3 | 44642233-44642283 | + | 44641544 | 689 | downstream | ZNF197/AY074878 |
| | | | | 3 | 44642233-44642283 | + | 44641590 | 643 | downstream | BC031209 |
| | | | | 3 | 44642233-44642283 | + | 44645632 | 3349 | downstream | ZNF197/AY261677 |
| | | | | 3 | 44642233-44642283 | + | 44645646 | 3363 | downstream | ZNF197/AF011573 |
| | | | | 3 | 44642233-44642283 | + | 44645646 | 3363 | downstream | NM_006991 |
| | | | | 3 | 44642233-44642283 | + | 44645732 | 3449 | downstream | ZNF197/Z21707 |
| | | | | 3 | 158638513-158638563 | + | 158637308 | 1205 | downstream | PTX3/BC039733 |
| | | | | 3 | 158638513-158638563 | + | 158637308 | 1205 | downstream | NM_002852 |
| | | | | 4 | 103780719-103780769 | + | 103779672 | 1047 | downstream | NFKB1/BC051765 |
| | | | | 4 | 103780719-103780769 | + | 103779672 | 1047 | downstream | NM_003998 |
| | | | | 4 | 103780719-103780769 | + | 103779741 | 978 | downstream | NFKB1/M58603 |
| 4 | FJ#17E5 | 478 | 476 | unknown | −1-−1 | unknown | −1 | −1 | unknown | Meis2 |
| | | | | 15 | 35175594-35176026 | − | 35177795 | 1769 | upstream | NM_172315 |
| | | | | 15 | 35175594-35176026 | − | 35178889 | 2863 | upstream | NM_172316 |
| | | | | 15 | 35175594-35176026 | − | 35179996 | 3970 | upstream | NM_020149 |
| | | | | 15 | 35175594-35176026 | − | 35179996 | 3970 | upstream | NM_170674 |
| | | | | 15 | 35175594-35176026 | − | 35179996 | 3970 | upstream | NM_170675 |
| | | | | 15 | 35175594-35176026 | − | 35179996 | 3970 | upstream | NM_170676 |
| | | | | 15 | 35175594-35176026 | − | 35179996 | 3970 | upstream | NM_170677 |
| | | | | 15 | 35175594-35176026 | − | 35180673 | 4647 | upstream | MEIS2/BC050431 |
| | | | | 15 | 35175594-35176026 | − | 35180792 | 4766 | upstream | NM_002399 |
| | | | | 15 | 35175594-35176026 | − | 35180796 | 4770 | upstream | MEIS2/BC001844 |
| 5 | FJ#20G11 | 583 | 580 | 19 | 43518614-43519197 | + | 43518297 | 317 | downstream | C19orf15/AK128220 |
| | | | | 19 | 43518614-43519197 | + | 43518297 | 317 | downstream | NM_021185 |
| 6 | FJ#30A11 | 774 | 912 | 21 | 44955066-44956738 | − | 44955923 | 0 | within | C21orf29/AJ487962 |
| | | | | 21 | 44955066-44956738 | − | 44955923 | 0 | within | NM_144991 |
| 7 | FJ#26E1 | 849 | 848 | unknown | −1-−1 | unknown | −1 | −1 | unknown | DKFZp727G131 |
| | | | | 7 | 98800098-98801663 | + | 98800869 | 0 | within | NM_024061 |
| | | | | 7 | 98800098-98801663 | + | 98800869 | 0 | within | NM_138494 |
| | | | | 7 | 98800098-98801663 | + | 98800915 | 0 | within | VIK/AK057245 |
| | | | | 7 | 98800098-98801663 | + | 98800925 | 0 | within | VIK/BC000823 |
| | | | | 7 | 98800098-98801663 | + | 98801135 | 0 | within | VIK/BC037407 |
| | | | | 7 | 98800098-98801663 | − | 98800766 | 0 | within | DKFZp727G131/AK094113 |
| 8 | FJ#30E9 | 689 | 690 | 10 | 17726024-17726714 | + | 17726129 | 0 | within | NM_003473 |
| | | | | 10 | 17726024-17726714 | + | 17726186 | 0 | within | STAM/BC030586 |
| | | | | 10 | 17726024-17726714 | + | 17726302 | 0 | within | STAM/U43899 |
| 9 | FJ#30E11 | 244 | 244 | 8 | 11362844-11363088 | − | 11361663 | 1181 | upstream | C8orf13/AL834122 |
| | | | | 8 | 11362844-11363088 | − | 11361663 | 1181 | upstream | NM_053279 |
| 10 | FJ#35G11 | 790 | 887 | 8 | 95800731-95801800 | + | 95801326 | 0 | within | LOC286148/BX538174 |
| | | | | 8 | 95800731-95801800 | + | 95801326 | 0 | within | NM_181787 |
| 11 | FJ#4A8 | 497 | 840 | 6 | 74286319-74287167 | − | 74284923 | 1396 | upstream | EEF1A1/BC012509 |
| | | | | 6 | 74286319-74287167 | − | 74285212 | 1107 | upstream | EEF1A1/M27364 |
| | | | | 6 | 74286319-74287167 | − | 74285272 | 1047 | upstream | EEF1A1/BC014892 |
| | | | | 6 | 74286319-74287167 | − | 74285277 | 1042 | upstream | EEF1A1/BC022412 |
| | | | | 6 | 74286319-74287167 | − | 74285278 | 1041 | upstream | EEF1A1/BC065761 |
| | | | | 6 | 74286319-74287167 | − | 74285468 | 851 | upstream | EEF1A1/BC014377 |
| | | | | 6 | 74286319-74287167 | − | 74285482 | 837 | upstream | EEF1A1/BC063511 |
| | | | | 6 | 74286319-74287167 | − | 74285893 | 426 | upstream | AF322220 |
| | | | | 6 | 74286319-74287167 | − | 74285903 | 416 | upstream | EEF1A1/AY062434 |
| | | | | 6 | 74286319-74287167 | − | 74286351 | 0 | within | EEF1A1/AF174496 |
| | | | | 6 | 74286319-74287167 | − | 74286503 | 0 | within | AF267861 |
| | | | | 6 | 74286319-74287167 | − | 74287475 | 308 | upstream | NM_001402 |
| | | | | 6 | 74286319-74287167 | − | 74287476 | 309 | upstream | EEF1A1/BC066893 |
| | | | | 7 | 22324714-22324891 | − | 22324921 | 30 | upstream | AF267861 |
| | | | | 9 | 132924393-132924570 | + | 132924362 | 31 | downstream | AF267861 |
| 12 | FJ#5C12 | 0 | 528 | 18 | 48122355-48122876 | + | 48121155 | 1200 | downstream | DCC/X76132 |
| | | | | 18 | 48122355-48122876 | + | 48121155 | 1200 | downstream | NM_005215 |
| 13 | FJ#13C6 | 469 | 748 | 2 | 32175740-32176474 | − | 32176474 | 0 | within | NM_032574 |
| | | | | 2 | 32175740-32176474 | − | 32176513 | 39 | upstream | LOC84661/BC015970 |
| 14 | FJ#13G2 | 443 | 612 | 2 | 26981039-26981620 | + | 26982613 | 993 | downstream | NM_020134 |
| | | | | 10 | 101979362-101979439 | − | 101979366 | 0 | within | CHUK/AF080157 |
| | | | | 10 | 101979362-101979439 | − | 101979366 | 0 | within | NM_001278 |
| 15 | FJ#13G10 | 0 | 621 | 6 | 142509708-142510329 | + | 142510102 | 0 | within | C6orf55/AF271994 |
| | | | | 6 | 142509708-142510329 | + | 142510102 | 0 | within | NM_016485 |
| | | | | 6 | 142509708-142510329 | + | 142510115 | 0 | within | AF141341 |
| 16 | FJ#23A10 | 644 | 644 | unknown | −1-−1 | unknown | −1 | −1 | unknown | BANF1 |
| | | | | 11 | 65525871-65526496 | + | 65526125 | 0 | within | BANF1/AF068235 |
| | | | | 11 | 65525871-65526496 | + | 65526125 | 0 | within | NM_003860 |
| | | | | 11 | 65525871-65526496 | − | 65526154 | 0 | within | MGC11102/AK094129 |
| | | | | 11 | 65525871-65526496 | − | 65526154 | 0 | within | NM_032325 |
| 17 | FJ#30A10 | 492 | 492 | 20 | 29656316-29656764 | + | 29656752 | 0 | within | NM_002165 |
| | | | | 20 | 29656316-29656764 | + | 29656752 | 0 | within | NM_181353 |
| | | | | 20 | 29656316-29656764 | + | 29656765 | 1 | downstream | ID1/BC012420 |
| | | | | 20 | 29656316-29656764 | + | 29656851 | 87 | downstream | ID1/BT007443 |
| 18 | FJ#26C4 | 225 | 686 | 12 | 10765801-10766442 | − | 10767171 | 729 | upstream | CSDA/BC021926 |
| | | | | 12 | 10765801-10766442 | − | 10767171 | 729 | upstream | NM_003651 |
| | | | | 12 | 10765801-10766442 | − | 10767173 | 731 | upstream | CSDA/BC009744 |
| 19 | FJ#27E8 | 612 | 609 | 2 | 230612655-230613264 | + | 230612711 | 0 | within | FBXO36/BC017869 |
| | | | | 2 | 230612655-230613264 | + | 230612718 | 0 | within | FBXO36/BC033935 |
| | | | | 2 | 230612655-230613264 | + | 230612718 | 0 | within | NM_174899 |
| | | | | 2 | 230612655-230613264 | − | 230612160 | 495 | upstream | TRIP12/D28476 |
| 20 | FJ#26G4 | 870 | 880 | 20 | 51631285-51632258 | − | 51633043 | 785 | upstream | ZNF217/AF041259 |
| | | | | 20 | 51631285-51632258 | − | 51633043 | 785 | upstream | NM_006526 |
| 21 | FJ#32E8 | 547 | 548 | 18 | 11839826-11840374 | + | 11841425 | 1051 | downstream | CHMP1.5/BC065933 |
| | | | | 18 | 11839826-11840374 | + | 11841425 | 1051 | downstream | NM_020412 |
| | | | | 18 | 11839826-11840374 | + | 11841456 | 1082 | downstream | CHMP1.5/BC012733 |
| | | | | 18 | 11839826-11840374 | + | 11841466 | 1092 | downstream | CHMP1.5/AF281064 |
| 22 | FJ#33E8 | 762 | 763 | 2 | 27399248-27399938 | − | 27397508 | 1740 | upstream | SLC30A3/U76010 |
| | | | | 2 | 27399248-27399938 | − | 27397598 | 1650 | upstream | NM_003459 |
| 23 | FJ#32G10 | 629 | 837 | 13 | 50925308-50926526 | + | 50925480 | 0 | within | BC030118 |
| | | | | 13 | 50925308-50926526 | − | 50925135 | 173 | upstream | NM_012141 |
| | | | | 13 | 50925308-50926526 | − | 50925150 | 158 | upstream | DDX26/BC039829 |
| | | | | 13 | 50925308-50926526 | − | 50925154 | 154 | upstream | DDX26/BC013358 |
| 24 | FJ#27B5 | 423 | 707 | 2 | 228162763-228163462 | + | 228162546 | 217 | downstream | NM_004504 |
| | | | | 2 | 228162763-228163462 | + | 228162558 | 205 | downstream | HRB/BC030592 |
| 25 | FJ#7B6 | 499 | 548 | 9 | 103936037-103936585 | + | 103936131 | 0 | within | BC055081 |
| | | | | 9 | 103936037-103936585 | + | 103936147 | 0 | within | BC061906 |
| | | | | 9 | 103936037-103936585 | + | 103936161 | 0 | within | SMC2L1/AF092563 |
| | | | | 9 | 103936037-103936585 | + | 103936161 | 0 | within | NM_006444 |
| 26 | FJ#9F12 | 0 | 854 | unknown | −1-−1 | unknown | −1 | −1 | unknown | MUC4 |
| 27 | FJ#11B2 | 729 | 846 | 4 | 72132504-72133776 | + | 72133098 | 0 | within | NM_173468 |
| | | | | 4 | 72132504-72133776 | + | 72133143 | 0 | within | MOBKL1A/BC038112 |
| 28 | FJ#12F6 | 826 | 851 | 15 | 66308223-66309409 | − | 66309079 | 0 | within | CLN6/AK000568 |
| | | | | 15 | 66308223-66309409 | − | 66309079 | 0 | within | NM_017882 |
| 29 | FJ#11H4 | 843 | 843 | 16 | 2322634-2322686 | − | 2319698 | 2936 | upstream | BC062779 |
| | | | | 19 | 44617886-44618729 | − | 44618426 | 0 | within | RPS16/BC004324 |
| | | | | 19 | 44617886-44618729 | − | 44618478 | 0 | within | NM_001020 |
| 30 | FJ#13H2 | 319 | 480 | 3 | 184627876-184628356 | − | 184628557 | 201 | upstream | NM_015078 |
| | | | | 3 | 184627876-184628356 | − | 184628575 | 219 | upstream | KIAA0861/BC064632 |
| | | | | 3 | 184627876-184628356 | − | 184628591 | 235 | upstream | AK124500 |
| 31 | FJ#13H6 | 34 | 512 | unknown | −1-−1 | unknown | −1 | −1 | unknown | RGS16 |
| | | | | 1 | 179304693-179305205 | − | 179305051 | 0 | within | RGS16/BT006638 |
| | | | | 1 | 179304693-179305205 | − | 179305140 | 0 | within | RGS16/U70426 |
| | | | | 1 | 179304693-179305205 | − | 179305200 | 0 | within | NM_002928 |
| 32 | FJ#25B4 | 325 | 325 | 9 | 91264689-91265014 | − | 91265517 | 503 | upstream | NFIL3/S79880 |
| | | | | 9 | 91264689-91265014 | − | 91265517 | 503 | upstream | NM_005384 |
| 33 | FJ#23D6 | 879 | 826 | 5 | 43638478-43640026 | + | 43638581 | 0 | within | NM_012343 |
| | | | | 5 | 43638478-43640026 | + | 43639063 | 0 | within | NNT/U40490 |
| | | | | 5 | 43638478-43640026 | + | 43639063 | 0 | within | NM_182977 |
| 34 | FJ#25F4 | 517 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 35 | FJ#28F2 | 429 | 429 | 4 | 141430872-141431249 | − | 141432838 | 1589 | upstream | MAML3/AB058719 |
| | | | | 4 | 141430872-141431249 | − | 141432838 | 1589 | upstream | NM_018717 |
| 36 | FJ#32F2 | 622 | 618 | 4 | 85773754-85774366 | − | 85776566 | 2200 | upstream | NKX6-1/NM_006168 |
| 37 | FJ#36A3 | 196 | 675 | 4 | 95486163-95486347 | + | 95486375 | 28 | downstream | SMARCAD1/AY008271 |
| | | | | 4 | 95486163-95486347 | + | 95486375 | 28 | downstream | NM_020159 |
| 38 | FJ#36E3 | 551 | 551 | 5 | 149808667-149809197 | − | 149809487 | 290 | upstream | RPS14/AF116710 |
| | | | | 5 | 149808667-149809197 | − | 149809512 | 315 | upstream | RPS14/NM_005617 |
| 39 | FJ#39G7 | 632 | 626 | 13 | 36391060-36391643 | − | 36392375 | 732 | upstream | SMAD9/BC067766 |
| 40 | FJ#41A5 | 570 | 570 | 6 | 26141494-26142051 | − | 26140267 | 1227 | upstream | HIST1H3B/NM_003537 |
| | | | | 6 | 26141494-26142051 | − | 26141775 | 0 | within | HIST1H2AB/NM_003513 |
| 41 | FJ#41G7 | 315 | 315 | 12 | 55325473-55325788 | − | 55326024 | 236 | upstream | ATP5B/BC016512 |
| | | | | 12 | 55325473-55325788 | − | 55326119 | 331 | upstream | NM_001686 |
| 42 | FJ#43G7 | 592 | 592 | 2 | 69575838-69576430 | − | 69576199 | 0 | within | HIRIP5/AJ132584 |
| | | | | 2 | 69575838-69576430 | − | 69576199 | 0 | within | NM_015700 |
| | | | | 2 | 69575838-69576430 | − | 69576258 | 0 | within | HIRIP5/AY286307 |
| | | | | 2 | 69575838-69576430 | − | 69576276 | 0 | within | HIRIP5/BX538347 |
| | | | | 2 | 69575838-69576430 | − | 69576404 | 0 | within | AY335194 |
| 43 | FJ#45G7 | 579 | 647 | 14 | 54807341-54808469 | + | 54807831 | 0 | within | NM_017943 |
| | | | | 14 | 54807341-54808469 | + | 54807921 | 0 | within | FBXO34/BX248268 |
| 44 | FJ#45G9 | 496 | 496 | 1 | 146794313-146794809 | − | 146795602 | 793 | upstream | ZA20D1/AJ293573 |
| | | | | 1 | 146794313-146794809 | − | 146795602 | 793 | upstream | NM_020205 |
| | | | | 1 | 146794313-146794809 | − | 146795697 | 888 | upstream | ZA20D1/BC020622 |
| 45 | FJ#55C3 | 597 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 46 | FJ#60A7 | 640 | 681 | 19 | 57980812-57981089 | − | 57981828 | 739 | upstream | ZNF600/BX640933 |
| | | | | 19 | 57980812-57981089 | − | 57981828 | 739 | upstream | NM_198457 |
| | | | | 19 | 57981030-57981089 | − | 57981828 | 739 | upstream | ZNF600/BX640933 |
| | | | | 19 | 57981030-57981089 | − | 57981828 | 739 | upstream | NM_198457 |
| | | | | 19 | 57919817-57919878 | − | 57919729 | 88 | upstream | BC015370 |
| | | | | 19 | 57832419-57832481 | − | 57830163 | 2256 | upstream | AK027782 |
| | | | | 19 | 57832419-57832481 | − | 57833450 | 969 | upstream | ZNF83/AK027518 |
| | | | | 19 | 57832419-57832481 | − | 57833450 | 969 | upstream | NM_018300 |
| | | | | 19 | 58187381-58187443 | − | 58188596 | 1153 | upstream | NM_024924 |
| | | | | 19 | 58591184-58591246 | + | 58590245 | 939 | downstream | LOC91661/BC017357 |
| | | | | 19 | 58591184-58591246 | + | 58590245 | 939 | downstream | NM_138372 |
| | | | | 19 | 58591184-58591246 | + | 58593007 | 1761 | downstream | LOC91661/BC001610 |
| | | | | 19 | 57766108-57766178 | + | 57765339 | 769 | downstream | FLJ10891/AK001753 |
| | | | | 19 | 57766108-57766178 | + | 57765339 | 769 | downstream | NM_018260 |
| | | | | 19 | 57766108-57766178 | + | 57765372 | 736 | downstream | BC054884 |
| | | | | 19 | 57766108-57766178 | + | 57765728 | 380 | downstream | BC067346 |
| 47 | FJ#58E9 | 436 | 437 | 1 | 167370398-167370826 | + | 167368179 | 2219 | downstream | AK130711 |
| 48 | FJ#57G7 | 490 | 490 | 17 | 40923045-40923491 | − | 40923882 | 391 | upstream | PLEKHM1/AB002354 |
| | | | | 17 | 40923045-40923491 | − | 40923893 | 402 | upstream | PLEKHM1/BC064361 |
| | | | | 17 | 40923045-40923491 | − | 40923893 | 402 | upstream | NM_014798 |
| 49 | FJ#65G5 | 946 | 916 | 5 | 70398856-70399707 | − | 70394346 | 4510 | upstream | BT006773 |
| | | | | 5 | 70398856-70399707 | − | 70399238 | 0 | within | GTF2H2/AF078847 |
| | | | | 5 | 70398856-70399707 | − | 70399238 | 0 | within | NM_001515 |
| | | | | 5 | 68891204-68892206 | + | 68891824 | 0 | within | GTF2H2/AF078847 |
| | | | | 5 | 68891204-68892206 | + | 68891824 | 0 | within | NM_001515 |
| | | | | 5 | 68891204-68892206 | + | 68896715 | 4509 | downstream | BT006773 |
| | | | | 5 | 69746502-69747353 | + | 69746971 | 0 | within | GTF2H2/AF078847 |
| | | | | 5 | 69746502-69747353 | + | 69746971 | 0 | within | NM_001515 |
| | | | | 5 | 69746502-69747353 | + | 69751864 | 4511 | downstream | BT006773 |
| 50 | FJ#40A2 | 369 | 369 | 12 | 46786323-46786662 | + | 46785972 | 351 | downstream | PFKM/AK126229 |
| | | | | 12 | 46786323-46786662 | − | 46785858 | 465 | upstream | SENP1/BC045639 |
| | | | | 12 | 46786323-46786662 | − | 46785884 | 439 | upstream | SENP1/BX640784 |
| | | | | 12 | 46786323-46786662 | − | 46785908 | 415 | upstream | NM_014554 |
| | | | | 12 | 46786323-46786662 | − | 46786042 | 281 | upstream | SENP1/BX537920 |
| 51 | FJ#41A10 | 62 | 235 | 19 | 60808345-60808544 | + | 60803541 | 4804 | downstream | NM_153219 |
| | | | | 19 | 60808345-60808544 | + | 60803548 | 4797 | downstream | ZNF524/BC067748 |
| | | | | 19 | 60808345-60808544 | + | 60805300 | 3045 | downstream | ZNF524/BC007396 |
| 52 | FJ#41A12 | 806 | 91 | 19 | 62554224-62554913 | + | 62554486 | 0 | within | ZNF304/AJ276316 |
| | | | | 19 | 62554224-62554913 | + | 62554486 | 0 | within | NM_020657 |
| 53 | FJ#41C2 | 277 | 277 | 12 | 41270337-41270614 | − | 41269745 | 592 | upstream | PRICKLE1/AK056499 |
| | | | | 12 | 41270337-41270614 | − | 41269745 | 592 | upstream | NM_153026 |
| 54 | FJ#44C2 | 283 | 454 | 19 | 59105171-59106766 | + | 59107882 | 1116 | downstream | NM_031896 |
| | | | | 19 | 59105171-59106766 | + | 59107897 | 1131 | downstream | CACNG7/AF458897 |
| | | | | 19 | 59106646-59106766 | + | 59107882 | 1116 | downstream | NM_031896 |
| | | | | 19 | 59106646-59106766 | + | 59107897 | 1131 | downstream | CACNG7/AF458897 |
| | | | | 19 | 59105171-59105625 | + | 59107882 | 2257 | downstream | NM_031896 |
| | | | | 19 | 59105171-59105625 | + | 59107897 | 2272 | downstream | CACNG7/AF458897 |
| | | | | 19 | 59105171-59106766 | + | 59107882 | 1116 | downstream | NM_031896 |
| | | | | 19 | 59105171-59106766 | + | 59107897 | 1131 | downstream | CACNG7/AF458897 |
| 55 | FJ#43E8 | 386 | 385 | 19 | 52305563-52305874 | − | 52308849 | 2975 | upstream | C19orf7/AB028987 |
| 56 | FJ#62E6 | 910 | 893 | 12 | 94930849-94931896 | − | 94931813 | 0 | within | LTA4H/BC032528 |
| | | | | 12 | 94930849-94931896 | − | 94931833 | 0 | within | NM_000895 |
| 57 | FJ#50F9 | 28 | 443 | 3 | 180805213-180805641 | + | 180805276 | 0 | within | NM_002492 |
| | | | | 3 | 180805213-180805641 | + | 180805285 | 0 | within | NDUFB5/BC005271 |
| | | | | 3 | 180805213-180805641 | − | 180803293 | 1920 | upstream | MRPL47/AF285120 |
| | | | | 3 | 180805213-180805641 | − | 180805113 | 100 | upstream | MRPL47/AY212270 |
| | | | | 3 | 180805213-180805641 | − | 180805118 | 95 | upstream | MRPL47/BC032522 |
| | | | | 3 | 180805213-180805641 | − | 180805136 | 77 | upstream | NM_020409 |
| | | | | 3 | 180805213-180805641 | − | 180805136 | 77 | upstream | NM_177988 |
| 58 | FJ#55F11 | 597 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 59 | FJ#59H3 | 679 | 680 | 19 | 45194865-45195545 | + | 45194868 | 0 | within | ZNF546/BC045649 |
| | | | | 19 | 45194865-45195545 | + | 45194868 | 0 | within | NM_178544 |
| 60 | FJ#41B6 | 288 | 715 | 5 | 68498706-68499387 | + | 68498668 | 38 | downstream | NM_031966 |
| | | | | 5 | 68498706-68499387 | + | 68498750 | 0 | within | CCNB1/BC006510 |
| 61 | FJ#41D6 | 838 | 839 | 11 | 6236999-6238808 | + | 6237541 | 0 | within | CCKBR/D13305 |
| | | | | 11 | 6236999-6238808 | + | 6237541 | 0 | within | NM_176875 |
| | | | | 11 | 6236999-6238808 | + | 6237731 | 0 | within | AF239668 |
| | | | | 11 | 6236999-6238808 | + | 6237734 | 0 | within | BT006789 |
| 62 | FJ#41F10 | 857 | 828 | 1 | 47610137-47611260 | + | 47613708 | 2448 | downstream | FOXD2/AF042832 |
| | | | | 1 | 47610137-47611260 | + | 47613708 | 2448 | downstream | NM_004474 |
| 63 | FJ#41H8 | 416 | 416 | 6 | 34833073-34833489 | + | 34833289 | 0 | within | SNRPC/X12517 |
| | | | | 6 | 34833073-34833489 | + | 34833289 | 0 | within | NM_003093 |
| 64 | FJ#43H2 | 580 | 580 | 6 | 26312593-26313173 | + | 26307765 | 4828 | downstream | HIST1H2BF/NM_003522 |
| | | | | 6 | 26312593-26313173 | + | 26312851 | 0 | within | HIST1H4E/NM_003545 |
| 65 | FJ#45H4 | 455 | 455 | 22 | 45478581-45478970 | + | 45479067 | 97 | downstream | C22orf4/BC029897 |
| | | | | 22 | 45478581-45478970 | + | 45479067 | 97 | downstream | NM_014346 |
| | | | | 22 | 45478581-45478970 | + | 45479096 | 126 | downstream | C22orf4/BC002743 |
| | | | | 22 | 45478581-45478970 | + | 45480157 | 1187 | downstream | C22orf4/AK125705 |
| 66 | FJ#47B4 | 394 | 620 | 17 | 50697038-50697651 | + | 50697374 | 0 | within | NM_002126 |
| 67 | FJ#50B8 | 550 | 550 | 7 | 134312143-134312667 | − | 134310090 | 2053 | upstream | MGC5242/AK130795 |
| | | | | 7 | 134312143-134312667 | − | 134312702 | 35 | upstream | MGC5242/BC067350 |
| | | | | 7 | 134312143-134312667 | − | 134312702 | 35 | upstream | MGC5242/BC000168 |
| | | | | 7 | 134312143-134312667 | − | 134312702 | 35 | upstream | NM_024033 |
| 68 | FJ#47D6 | 565 | 565 | 15 | 63476000-63476565 | − | 63475820 | 180 | upstream | NOPE/AB046848 |
| 69 | FJ#48D6 | 747 | 757 | 1 | 85453687-85454813 | − | 85455604 | 791 | upstream | BCL10/AF082283 |
| | | | | 1 | 85453687-85454813 | − | 85455604 | 791 | upstream | NM_003921 |
| 70 | FJ#48D12 | 580 | 463 | 6 | 26312593-26313173 | + | 26307765 | 4828 | downstream | HIST1H2BF/NM_003522 |
| | | | | 6 | 26312593-26313173 | + | 26312851 | 0 | within | HIST1H4E/NM_003545 |
| 71 | FJ#46H4 | 561 | 558 | 2 | 104927799-104928345 | + | 104930486 | 2141 | downstream | POU3F3/NM_006236 |
| 72 | FJ#48H4 | 537 | 694 | 12 | 30798568-30799665 | − | 30797565 | 1003 | upstream | C1QDC1/AK021453 |
| | | | | 12 | 30798568-30799665 | − | 30797858 | 710 | upstream | C1QDC1/BX537569 |
| | | | | 12 | 30798568-30799665 | − | 30798715 | 0 | within | C1QDC1/AY074490 |
| | | | | 12 | 30798568-30799665 | − | 30798715 | 0 | within | C1QDC1/AY074491 |
| | | | | 12 | 30798568-30799665 | − | 30798715 | 0 | within | NM_023925 |
| | | | | 12 | 30798568-30799665 | − | 30798715 | 0 | within | NM_001002259 |
| | | | | 12 | 30798568-30799665 | − | 30798715 | 0 | within | NM_032156 |
| 73 | FJ#51D2 | 705 | 703 | 12 | 2856602-2857305 | + | 2856659 | 0 | within | MGC13204/BC005106 |
| | | | | 12 | 2856602-2857305 | + | 2856659 | 0 | within | NM_031465 |
| | | | | 12 | 2856602-2857305 | − | 2853905 | 2697 | upstream | FOXM1/BT006986 |
| | | | | 12 | 2856602-2857305 | − | 2856413 | 189 | upstream | FOXM1/U74612 |
| | | | | 12 | 2856602-2857305 | − | 2856564 | 38 | upstream | NM_021953 |
| | | | | 12 | 2856602-2857305 | − | 2856564 | 38 | upstream | NM_202002 |
| | | | | 12 | 2856602-2857305 | − | 2856564 | 38 | upstream | NM_202003 |
| 74 | FJ#53F4 | 580 | 581 | 4 | 66362708-66363278 | + | 66364444 | 1166 | downstream | BC017721 |
| | | | | 4 | 66362708-66363278 | − | 66363972 | 694 | upstream | EPHA5/BX537946 |
| | | | | 4 | 66362708-66363278 | − | 66364275 | 997 | upstream | NM_004439 |
| | | | | 4 | 66362708-66363278 | − | 66364275 | 997 | upstream | NM_182472 |
| | | | | 4 | 66362708-66363278 | − | 66364829 | 1551 | upstream | EPHA5/X95425 |
| 75 | FJ#54H12 | 705 | 705 | x | 52870728-52871428 | − | 52869197 | 1531 | upstream | NM_014138 |
| | | | | x | 52808646-52809350 | + | 52810882 | 1532 | downstream | AF370413 |
| | | | | x | 52808646-52809350 | + | 52810882 | 1532 | downstream | NM_014138 |
| 76 | FJ#55H4 | 600 | 597 | 12 | 131293874-131294410 | + | 131295222 | 812 | downstream | MGC3162/BC001191 |
| | | | | 12 | 131293874-131294410 | + | 131295222 | 812 | downstream | NM_024078 |
| | | | | 12 | 131293874-131294410 | + | 131295241 | 831 | downstream | AK074489 |
| | | | | 12 | 131293874-131294410 | + | 131295329 | 919 | downstream | MGC3162/BC007893 |
| | | | | 12 | 131293874-131294410 | − | 131291511 | 2363 | upstream | DDX51/BC012461 |
| | | | | 12 | 131293874-131294410 | − | 131295083 | 673 | upstream | DDX51/BC040185 |
| | | | | 12 | 131293874-131294410 | − | 131295083 | 673 | upstream | NM_175066 |
| 77 | FJ#55H8 | 237 | 239 | 2 | 112955319-112955558 | + | 112956128 | 570 | downstream | TTL/AB071393 |
| | | | | 2 | 112955319-112955558 | + | 112956128 | 570 | downstream | NM_153712 |
| 78 | FJ#56H4 | 593 | 593 | 1 | 45718132-45718724 | + | 45718809 | 85 | downstream | NM_002482 |
| | | | | 1 | 45718132-45718724 | + | 45718809 | 85 | downstream | NM_152298 |
| | | | | 1 | 45718132-45718724 | + | 45718809 | 85 | downstream | NM_172164 |
| | | | | 1 | 45718132-45718724 | + | 45718824 | 100 | downstream | NASP/BC010105 |
| | | | | 1 | 45718132-45718724 | + | 45718827 | 103 | downstream | NASP/AF035191 |
| | | | | 1 | 45718132-45718724 | + | 45718828 | 104 | downstream | NASP/BC009933 |
| | | | | 1 | 45718132-45718724 | + | 45718911 | 187 | downstream | NASP/BT006757 |
| 79 | FJ#65F8 | 318 | 318 | 6 | 24883926-24884244 | + | 24883142 | 784 | downstream | NM_015895 |
| | | | | 6 | 24883926-24884244 | + | 24883162 | 764 | downstream | GMNN/BC005389 |
| 80 | FJ#65H10 | 313 | 318 | 6 | 24883926-24884244 | + | 24883142 | 784 | downstream | NM_015895 |
| | | | | 6 | 24883926-24884244 | + | 24883162 | 764 | downstream | GMNN/BC005389 |
| 81 | FJ#66D2 | 435 | 435 | unknown | −1-−1 | unknown | −1 | −1 | unknown | AK091555 |
| 82 | FJ#74A3 | 845 | 927 | 7 | 133788518-133789560 | + | 133788809 | 0 | within | NM_001724 |
| | | | | 7 | 133788518-133789560 | + | 133788814 | 0 | within | BPGM/BC017050 |
| | | | | 7 | 133788518-133789560 | + | 133788825 | 0 | within | NM_199186 |
| 83 | FJ#74C3 | 617 | 617 | 21 | 43809556-43809937 | + | 43809547 | 9 | downstream | H2BFS/NM_017445 |
| | | | | 6 | 27914484-27914729 | + | 27914357 | 127 | downstream | HIST1H2BN/BC011372 |
| | | | | 6 | 27914484-27914729 | + | 27914418 | 66 | downstream | NM_003520 |
| | | | | 6 | 27914484-27914729 | − | 27914096 | 388 | upstream | HIST1H2AK/NM_003510 |
| | | | | 6 | 27914646-27914729 | + | 27914357 | 289 | downstream | HIST1H2BN/BC011372 |
| | | | | 6 | 27914646-27914729 | + | 27914418 | 228 | downstream | NM_003520 |
| | | | | 6 | 27914646-27914729 | − | 27914096 | 550 | upstream | HIST1H2AK/NM_003510 |
| | | | | 6 | 27222156-27222774 | + | 27222886 | 112 | downstream | HIST1H2AH/NM_080596 |
| | | | | 6 | 27222156-27222774 | − | 27222598 | 0 | within | HIST1H2BK/BC000893 |
| | | | | 6 | 27222156-27222774 | − | 27222598 | 0 | within | NM_080593 |
| | | | | 6 | 26266604-26266684 | + | 26264537 | 2067 | downstream | HIST1H1E/NM_005321 |
| | | | | 6 | 26266604-26266684 | + | 26266327 | 277 | downstream | NM_021063 |
| | | | | 6 | 26266604-26266684 | + | 26266327 | 277 | downstream | NM_138720 |
| | | | | 6 | 26266604-26266684 | + | 26266351 | 253 | downstream | HIST1H2BD/BC002842 |
| | | | | 6 | 27208197-27208283 | + | 27208799 | 516 | downstream | NM_021064 |
| | | | | 6 | 27208197-27208283 | + | 27208810 | 527 | downstream | HIST1H2AI/BC016677 |
| | | | | 6 | 27208197-27208283 | − | 27208551 | 268 | upstream | HIST1H2BJ/BC014312 |
| | | | | 6 | 27208197-27208283 | − | 27208554 | 271 | upstream | NM_021058 |
| | | | | 6 | 26231803-26231883 | + | 26232351 | 468 | downstream | NM_003512 |
| | | | | 6 | 26231803-26231883 | + | 26232396 | 513 | downstream | HIST1H2AC/BC017379 |
| | | | | 6 | 26231803-26231883 | − | 26232111 | 228 | upstream | HIST1H2BC/NM_003526 |
| | | | | 6 | 25840059-25840117 | + | 25835115 | 4944 | downstream | HIST1H2BA/BC066243 |
| | | | | 6 | 25840059-25840117 | + | 25835115 | 4944 | downstream | NM_170610 |
| | | | | 6 | 26292062-26292310 | + | 26292002 | 60 | downstream | HIST1H2BE/NM_003523 |
| | | | | 6 | 26292062-26292310 | − | 26297283 | 4973 | upstream | NM_003539 |
| | | | | 6 | 27914484-27914537 | + | 27914357 | 127 | downstream | HIST1H2BN/BC011372 |
| | | | | 6 | 27914484-27914537 | + | 27914418 | 66 | downstream | NM_003520 |
| | | | | 6 | 27914484-27914537 | − | 27914096 | 388 | upstream | HIST1H2AK/NM_003510 |
| | | | | 6 | 27914484-27914729 | + | 27914357 | 127 | downstream | HIST1H2BN/BC011372 |
| | | | | 6 | 27914484-27914729 | + | 27914418 | 66 | downstream | NM_003520 |
| | | | | 6 | 27914484-27914729 | − | 27914096 | 388 | upstream | HIST1H2AK/NM_003510 |
| | | | | 6 | 27969447-27969537 | + | 27969181 | 266 | downstream | HIST1H2BO/NM_003527 |
| | | | | 6 | 27969447-27969537 | − | 27966549 | 2898 | upstream | HIST1H3J/NM_003535 |
| | | | | 6 | 27969447-27969537 | − | 27968942 | 505 | upstream | HIST1H2AM/NM_003514 |
| 84 | FJ#75E11 | 526 | 526 | 5 | 107032427-107032954 | − | 107034495 | 1541 | upstream | EFNA5/U26403 |
| | | | | 5 | 107032427-107032954 | − | 107034495 | 1541 | upstream | NM_001962 |
| 85 | FJ#76A3 | 155 | 148 | 4 | 129061803-129061954 | + | 129061057 | 746 | downstream | APG-1/BC040560 |
| | | | | 4 | 129061803-129061954 | + | 129061057 | 746 | downstream | NM_014278 |
| 86 | FJ#80A5 | 535 | 535 | 19 | 12653111-12653647 | − | 12653663 | 16 | upstream | DHPS/BC014016 |
| | | | | 19 | 12653111-12653647 | − | 12653677 | 30 | upstream | NM_001930 |
| | | | | 19 | 12653111-12653647 | − | 12653677 | 30 | upstream | NM_013406 |
| | | | | 19 | 12653111-12653647 | − | 12653677 | 30 | upstream | NM_013407 |
| 87 | FJ#76C5 | 671 | 671 | 6 | 80770855-80771486 | + | 80771077 | 0 | within | TTK/BC000633 |
| | | | | 6 | 80770855-80771486 | + | 80771077 | 0 | within | NM_003318 |
| | | | | 6 | 80770855-80771486 | + | 80772274 | 788 | downstream | TTK/M86699 |
| 88 | FJ#82A7 | 210 | 722 | 1 | 85885132-85885762 | − | 85885509 | 0 | within | FLJ20729/AK000736 |
| | | | | 1 | 85885132-85885762 | − | 85885784 | 22 | upstream | FLJ20729/AF308296 |
| | | | | 1 | 85885132-85885762 | − | 85886122 | 360 | upstream | FLJ20729/AL442074 |
| | | | | 1 | 85885132-85885762 | − | 85886122 | 360 | upstream | NM_017953 |
| 89 | FR#4G9 | 513 | 513 | 2 | 142721862-142722346 | − | 142722306 | 0 | within | LRP1B/AK054663 |
| | | | | 2 | 142721862-142722346 | − | 142723002 | 656 | upstream | LRP1B/AF176832 |
| | | | | 2 | 142721862-142722346 | − | 142723002 | 656 | upstream | NM_018557 |
| 90 | FJ#76A4 | 460 | 808 | 21 | 32166204-32167272 | + | 32167498 | 226 | downstream | HUNK/AJ271722 |
| | | | | 21 | 32166204-32167272 | + | 32167498 | 226 | downstream | NM_014586 |
| 91 | FJ#82C12 | 818 | 701 | 10 | 102016776-102017591 | − | 102017345 | 0 | within | CWF19L1/AK023984 |
| | | | | 10 | 102016776-102017591 | − | 102017369 | 0 | within | CWF19L1/BC008746 |
| | | | | 10 | 102016776-102017591 | − | 102017369 | 0 | within | NM_018294 |
| 92 | FJ#76F7 | 576 | 576 | 1 | 223813382-223813958 | − | 223811618 | 1764 | upstream | CDC42BPA/AJ518975 |
| | | | | 1 | 223813382-223813958 | − | 223811618 | 1764 | upstream | CDC42BPA/AJ518976 |
| | | | | 1 | 223813382-223813958 | − | 223812561 | 821 | upstream | NM_003607 |
| | | | | 1 | 223813382-223813958 | − | 223812561 | 821 | upstream | NM_014826 |
| | | | | 1 | 223813382-223813958 | − | 223812910 | 472 | upstream | CDC42BPA/U59305 |

TABLE 14 shows, according to particular preferred aspects, markers for CLL CD38as identified by methylation hybridization as described in the EXAMPLES herein.

| No. | CloneID | T7 Sequence Length | M13 Sequence Length | Chromosome Aligned | Alignment Address | Strand | TSS | Distance to TSS | Direction | Gene/Assession Number |
|---|---|---|---|---|---|---|---|---|---|---|
| 1 | 12:H07 | 457 | 331 | unknown | −1-−1 | unknown | −1 | −1 | unknown | NM_130851 |
| | | | | 14 | 53490390-53490812 | − | 53491020 | 208 | upstream | NM_130851 |
| | | | | 14 | 53490390-53490812 | − | 53493279 | 2467 | upstream | BMP4/M22490 |
| | | | | 14 | 53490390-53490812 | − | 53493362 | 2550 | upstream | NM_001202 |
| | | | | 14 | 53490390-53490812 | − | 53493362 | 2550 | upstream | NM_130850 |
| 2 | 15:H11 | 905 | 941 | unknown | −1-−1 | unknown | −1 | −1 | unknown | AK000013 |
| | | | | 16 | 65143775-65144565 | + | 65140683 | 3092 | downstream | AK000013 |
| | | | | 16 | 65143775-65144565 | − | 65141581 | 2194 | upstream | TK2/Y10498 |
| | | | | 16 | 65143775-65144565 | − | 65141816 | 1959 | upstream | TK2/AF521891 |
| | | | | 16 | 65143775-65144565 | − | 65141816 | 1959 | upstream | NM_004614 |
| | | | | 16 | 65143775-65144565 | + | 65143972 | 0 | within | NM_016326 |
| | | | | 16 | 65143775-65144565 | + | 65143972 | 0 | within | NM_016951 |
| | | | | 16 | 65143775-65144565 | + | 65143972 | 0 | within | NM_181640 |
| | | | | 16 | 65143775-65144565 | + | 65143972 | 0 | within | NM_181641 |
| | | | | 16 | 65143775-65144565 | + | 65143995 | 0 | within | CKLF/BC004380 |
| 3 | 82:C09 | 917 | 247 | unknown | −1-−1 | unknown | −1 | −1 | unknown | MC5R/NM_005913 |
| | | | | 18 | 13814136-13814387 | + | 13815764 | 1377 | downstream | MC5R/NM_005913 |
| 4 | 32:B01 | 530 | 531 | unknown | −1-−1 | unknown | −1 | −1 | unknown | KIR2DL4/BC041611 |
| | | | | unknown | −1-−1 | unknown | −1 | −1 | unknown | NM_002255 |
| | | | | 19 | 60006535-60007066 | + | 60008058 | 992 | downstream | KIR2DL4/AY223513 |
| | | | | 19 | 60006535-60007066 | + | 60008058 | 992 | downstream | KIR2DL4/AY223515 |
| | | | | 19 | 60006535-60007066 | + | 60009285 | 2219 | downstream | AY052496 |
| | | | | 19 | 60006535-60007066 | + | 60009285 | 2219 | downstream | AY052497 |
| | | | | 19 | 60006535-60007066 | + | 60009285 | 2219 | downstream | AY052498 |
| | | | | 19_random | 199553-200085 | + | 201077 | 992 | downstream | KIR2DL4/AY223513 |
| | | | | 19_random | 199553-200085 | + | 201077 | 992 | downstream | KIR2DL4/AY223515 |
| | | | | 19_random | 199553-200085 | + | 201077 | 992 | downstream | KIR2DL4/AY250088 |
| | | | | 19_random | 199553-200085 | + | 202310 | 2225 | downstream | AY052496 |
| | | | | 19_random | 199553-200085 | + | 202310 | 2225 | downstream | AY052497 |
| | | | | 19_random | 199553-200085 | + | 202310 | 2225 | downstream | AY052498 |
| | | | | 19 | 60006535-60007066 | + | 60006877 | 0 | within | KIR2DL4/BC041611 |
| | | | | 19 | 60006535-60007066 | + | 60006877 | 0 | within | NM_002255 |
| | | | | 19 | 60006535-60007066 | + | 60006906 | 0 | within | KIR2DL4/U71199 |
| | | | | 19 | 60006535-60007066 | + | 60006918 | 0 | within | KIR2DL4/Y13054 |
| | | | | 19 | 60006535-60007066 | + | 60006918 | 0 | within | KIR2DL4/AF276292 |
| | | | | 19 | 60006535-60007066 | + | 60006922 | 0 | within | KIR2DL4/AF002256 |
| | | | | 19_random | 199553-200085 | + | 199896 | 0 | within | KIR2DL4/BC041611 |
| | | | | 19_random | 199553-200085 | + | 199896 | 0 | within | NM_002255 |
| | | | | 19_random | 199553-200085 | + | 199925 | 0 | within | KIR2DL4/U71199 |
| | | | | 19_random | 199553-200085 | + | 199937 | 0 | within | KIR2DL4/Y13054 |
| | | | | 19_random | 199553-200085 | + | 199937 | 0 | within | KIR2DL4/AF276292 |
| | | | | 19_random | 199553-200085 | + | 199941 | 0 | within | KIR2DL4/AF002256 |
| 5 | 32:B07 | 863 | 855 | unknown | −1-−1 | unknown | −1 | −1 | unknown | NM_144732 |
| | | | | 19 | 46459827-46460635 | + | 46462075 | 1440 | downstream | HNRPUL1/AJ007509 |
| | | | | 19 | 46459827-46460635 | + | 46462075 | 1440 | downstream | NM_007040 |
| | | | | 19 | 46459827-46460635 | + | 46462075 | 1440 | downstream | NM_144733 |
| | | | | 19 | 46459827-46460635 | + | 46462075 | 1440 | downstream | NM_144734 |
| | | | | 19 | 46459827-46460635 | + | 46462116 | 1481 | downstream | HNRPUL1/AK127057 |
| | | | | 19 | 46459827-46460635 | + | 46462166 | 1531 | downstream | HNRPUL1/BC009988 |
| | | | | 19 | 46459827-46460635 | + | 46462195 | 1560 | downstream | HNRPUL1/BC027713 |
| | | | | 19 | 46459827-46460635 | + | 46462202 | 1567 | downstream | HNRPUL1/BC004242 |
| | | | | 19 | 46459827-46460635 | + | 46460263 | 0 | within | NM_144732 |
| 6 | 6:E08 | 781 | 781 | unknown | −1-−1 | unknown | −1 | −1 | unknown | SIP/AL035305 |
| | | | | unknown | −1-−1 | unknown | −1 | −1 | unknown | NM_014412 |
| | | | | 1 | 171699520-171700300 | + | 171700856 | 556 | downstream | SIP/AL035305 |
| | | | | 1 | 171699520-171700300 | + | 171700856 | 556 | downstream | NM_014412 |
| 7 | 74:E11 | 465 | 465 | unknown | −1-−1 | unknown | −1 | −1 | unknown | PVRL2/BC003091 |
| | | | | 19 | 50042014-50042479 | + | 50041424 | 590 | downstream | PVRL2/BC003091 |
| | | | | 19 | 50042014-50042479 | + | 50041473 | 541 | downstream | NM_002856 |
| 8 | 43:C09 | 146 | 146 | unknown | −1-−1 | unknown | −1 | −1 | unknown | FLJ32447/AK057009 |
| | | | | unknown | −1-−1 | unknown | −1 | −1 | unknown | NM_153038 |
| | | | | 2 | 222985738-222985884 | + | 222988370 | 2486 | downstream | FLJ32447/AK057009 |
| | | | | 2 | 222985738-222985884 | + | 222988370 | 2486 | downstream | NM_153038 |
| | | | | 2 | 222985738-222985884 | − | 222984528 | 1210 | upstream | PAX3/U02309 |
| | | | | 2 | 222985738-222985884 | − | 222988883 | 2999 | upstream | PAX3/AY251280 |
| | | | | 2 | 222985738-222985884 | − | 222988883 | 2999 | upstream | PAX3/AY251279 |
| | | | | 2 | 222985738-222985884 | − | 222988970 | 3086 | upstream | PAX3/S69369 |
| | | | | 2 | 222985738-222985884 | − | 222989089 | 3205 | upstream | PAX3/BC063547 |
| | | | | 2 | 222985738-222985884 | − | 222989205 | 3321 | upstream | NM_181457 |
| | | | | 2 | 222985738-222985884 | − | 222989205 | 3321 | upstream | NM_181458 |
| | | | | 2 | 222985738-222985884 | − | 222989205 | 3321 | upstream | NM_181459 |
| | | | | 2 | 222985738-222985884 | − | 222989205 | 3321 | upstream | NM_181460 |
| | | | | 2 | 222985738-222985884 | − | 222989205 | 3321 | upstream | NM_181461 |
| | | | | 2 | 222985738-222985884 | − | 222989205 | 3321 | upstream | NM_000438 |
| | | | | 2 | 222985738-222985884 | − | 222989205 | 3321 | upstream | NM_013942 |
| 9 | 58:G12 | 626 | 536 | unknown | −1-−1 | unknown | −1 | −1 | unknown | AK055401 |
| | | | | 1 | 62865204-62866443 | − | 62865990 | 0 | within | AK055401 |
| 10 | 35:D07 | 163 | 163 | unknown | −1-−1 | unknown | −1 | −1 | unknown | X76978 |
| | | | | 4 | 1388950-1389113 | − | 1386987 | 1963 | upstream | X76978 |
| 11 | 29:A12 | 939 | 353 | unknown | −1-−1 | unknown | −1 | −1 | unknown | NM_033505 |
| | | | | 2 | 26481132-26481443 | + | 26480632 | 500 | downstream | NM_033505 |
| | | | | 2 | 26481132-26481443 | + | 26480642 | 490 | downstream | AB051511 |
| | | | | 2 | 26481132-26481443 | + | 26480687 | 445 | downstream | BC021229 |
| | | | | 2 | 26481132-26481443 | − | 26481336 | 0 | within | GPR113/AY358172 |
| 12 | 46:C04 | 199 | 199 | unknown | −1-−1 | unknown | −1 | −1 | unknown | SLC6A5/AF085412 |
| | | | | unknown | −1-−1 | unknown | −1 | −1 | unknown | NM_004211 |
| | | | | 11 | 20574746-20574933 | + | 20577526 | 2593 | downstream | SLC6A5/AF085412 |
| | | | | 11 | 20574746-20574933 | + | 20577526 | 2593 | downstream | NM_004211 |
| 13 | 11:F03 | 363 | 363 | unknown | −1-−1 | unknown | −1 | −1 | unknown | NM_013374 |
| | | | | 3 | 33814676-33815039 | + | 33814560 | 116 | downstream | NM_013374 |
| | | | | 3 | 33814676-33815039 | + | 33815088 | 49 | downstream | PDCD6IP/BC068454 |
| 14 | 42:A06 | 203 | 200 | unknown | −1-−1 | unknown | −1 | −1 | unknown | NEIL2/AK097389 |
| | | | | 8 | 11665248-11665451 | + | 11664626 | 622 | downstream | NEIL2/AK097389 |
| | | | | 8 | 11665248-11665451 | + | 11664665 | 583 | downstream | NEIL2/BX537529 |
| | | | | 8 | 11665248-11665451 | + | 11664665 | 583 | downstream | NEIL2/AK056206 |
| | | | | 8 | 11665248-11665451 | + | 11664665 | 583 | downstream | NM_145043 |
| 15 | 15:B11 | 788 | 332 | unknown | −1-−1 | unknown | −1 | −1 | unknown | KIAA1811/AF479827 |
| | | | | unknown | −1-−1 | unknown | −1 | −1 | unknown | NM_032430 |
| | | | | 19 | 60482958-60483290 | + | 60487345 | 4055 | downstream | KIAA1811/AF479827 |
| | | | | 19 | 60482958-60483290 | + | 60487345 | 4055 | downstream | NM_032430 |
| | | | | 19 | 60482958-60483290 | − | 60483307 | 17 | upstream | HSPBP1/AK130636 |
| | | | | 19 | 60482958-60483290 | − | 60483540 | 250 | upstream | HSPBP1/AF217996 |
| | | | | 19 | 60482958-60483290 | − | 60483540 | 250 | upstream | NM_012267 |
| 16 | 58:C10 | 787 | 644 | unknown | −1-−1 | unknown | −1 | −1 | unknown | ADM/BC015961 |
| | | | | 11 | 10283378-10285020 | + | 10283207 | 171 | downstream | ADM/BC015961 |
| | | | | 11 | 10283378-10285020 | + | 10283217 | 161 | downstream | NM_001124 |
| 17 | 8:A10 | 635 | 459 | unknown | −1-−1 | unknown | −1 | −1 | unknown | NM_199324 |
| | | | | unknown | −1-−1 | unknown | −1 | −1 | unknown | NM_017493 |
| | | | | 4 | 146458381-146458992 | − | 146453501 | 4880 | upstream | NM_199324 |
| | | | | 4 | 146458381-146458992 | − | 146453501 | 4880 | upstream | NM_017493 |
| | | | | 4 | 146458381-146458992 | − | 146456915 | 1466 | upstream | X68242 |
| 18 | 50:D11 | 919 | 708 | unknown | −1-−1 | unknown | −1 | −1 | unknown | LRRC8/AB037858 |
| | | | | 9 | 128723803-128725144 | + | 128724028 | 0 | within | LRRG8/AB037858 |
| | | | | 9 | 128723803-128725144 | − | 128723869 | 0 | within | CCBL1/BC021262 |
| | | | | 9 | 128723803-128725144 | − | 128723886 | 0 | within | NM_004059 |
| 19 | 58:G01 | 552 | 647 | unknown | −1-−1 | unknown | −1 | −1 | unknown | BX161384 |
| | | | | 14 | 23981039-23982147 | − | 23981499 | 0 | within | BX161384 |
| | | | | 14 | 23981039-23982147 | − | 23981822 | 0 | within | C14orf124/AF226050 |
| | | | | 14 | 23981039-23982147 | − | 23981822 | 0 | within | C14orf124/AK123513 |
| | | | | 14 | 23981039-23982147 | − | 23981822 | 0 | within | NM_020195 |
| | | | | 14 | 23981039-23982147 | − | 23981847 | 0 | within | C14orf124/BC000989 |
| 20 | 41:E09 | 408 | 230 | unknown | −1-−1 | unknown | −1 | −1 | unknown | GJB2/BC017048 |
| | | | | unknown | −1-−1 | unknown | −1 | −1 | unknown | NM_004004 |
| | | | | 13 | 19665105-19665497 | − | 19665037 | 68 | upstream | GJB2/BC017048 |
| | | | | 13 | 19665105-19665497 | − | 19665037 | 68 | upstream | NM_004004 |
| 21 | 55:H05 | 563 | 780 | unknown | −1-−1 | unknown | −1 | −1 | unknown | ADAM12/AY358878 |
| | | | | 10 | 128065899-128066876 | − | 128067002 | 126 | upstream | ADAM12/AY358878 |
| | | | | 10 | 128065899-128066876 | − | 128067014 | 138 | upstream | ADAM12/AF023476 |
| | | | | 10 | 128065899-128066876 | − | 128067014 | 138 | upstream | NM_003474 |
| | | | | 10 | 128065899-128066876 | − | 128067014 | 138 | upstream | NM_021641 |
| | | | | 10 | 128065899-128066876 | − | 128067055 | 179 | upstream | ADAM12/BC060804 |
| 22 | 38:A08 | 630 | 738 | unknown | −1-−1 | unknown | −1 | −1 | unknown | AK095105 |
| | | | | 1 | 145644111-145644824 | + | 145644047 | 64 | downstream | AK095105 |
| | | | | 2 | 91332865-91333578 | − | 91333642 | 64 | upstream | AK095105 |
| 23 | 68:E03 | 362 | 359 | unknown | −1-−1 | unknown | −1 | −1 | unknown | SAV1/AK023071 |
| | | | | 14 | 50203675-50204034 | − | 50204650 | 616 | upstream | SAV1/AK023071 |
| | | | | 14 | 50203675-50204034 | − | 50204773 | 739 | upstream | NM_021818 |
| 24 | 68:H06 | 820 | 874 | unknown | −1-−1 | unknown | −1 | −1 | unknown | AB014515 |
| | | | | unknown | −1-−1 | unknown | −1 | −1 | unknown | NM_153029 |
| | | | | 16 | 47200696-47201846 | − | 47201621 | 0 | within | AB014515 |
| | | | | 16 | 47200696-47201846 | − | 47201621 | 0 | within | NM_153029 |
| 25 | 50:E08 | 761 | 386 | unknown | −1-−1 | unknown | −1 | −1 | unknown | BT007007 |
| | | | | 8 | 100974582-100975343 | − | 100973425 | 1157 | upstream | BT007007 |
| | | | | 8 | 100974582-100975343 | − | 100973457 | 1125 | upstream | COX6C/BC000187 |
| | | | | 8 | 100974582-100975343 | − | 100975071 | 0 | within | NM_004374 |
| 26 | 64:F01 | 773 | 868 | unknown | −1-−1 | unknown | −1 | −1 | unknown | C9orf12/BC026154 |
| | | | | unknown | −1-−1 | unknown | −1 | −1 | unknown | NM_022755 |
| | | | | 9 | 92511236-92512698 | − | 92512102 | 0 | within | C9orf12/BC026154 |
| | | | | 9 | 92511236-92512698 | − | 92512102 | 0 | within | NM_022755 |
| 27 | 56:E01 | 765 | 823 | unknown | −1-−1 | unknown | −1 | −1 | unknown | AJ583819 |
| | | | | 6 | 100069427-100070290 | − | 100064817 | 4610 | upstream | AJ583819 |
| | | | | 6 | 100069427-100070290 | − | 100070029 | 0 | within | USP45/BC005991 |
| 28 | 48:C04 | 381 | 381 | unknown | −1-−1 | unknown | −1 | −1 | unknown | NM_178523 |
| | | | | 19 | 57334936-57335317 | − | 57335003 | 0 | within | NM_178523 |
| 29 | 43:E02 | 715 | 712 | unknown | −1-−1 | unknown | −1 | −1 | unknown | ASCL2/BC057801 |
| | | | | 11 | 2248423-2249135 | − | 2248394 | 29 | upstream | ASCL2/BC057801 |
| | | | | 11 | 2248423-2249135 | − | 2248758 | 0 | within | NM_005170 |
| 30 | 29:C08 | 652 | 652 | unknown | −1-−1 | unknown | −1 | −1 | unknown | DMRT2/AF284223 |
| | | | | unknown | −1-−1 | unknown | −1 | −1 | unknown | DMRT2/AF284225 |
| | | | | unknown | −1-−1 | unknown | −1 | −1 | unknown | NM_006557 |
| | | | | unknown | −1-−1 | unknown | −1 | −1 | unknown | NM_181872 |
| | | | | 9 | 1037508-1038155 | + | 1041613 | 3458 | downstream | DMRT2/AF284223 |
| | | | | 9 | 1037508-1038155 | + | 1041613 | 3458 | downstream | DMRT2/AF284225 |
| | | | | 9 | 1037508-1038155 | + | 1041613 | 3458 | downstream | NM_006557 |
| | | | | 9 | 1037508-1038155 | + | 1041613 | 3458 | downstream | NM_181872 |
| 31 | 15:E11 | 931 | 622 | unknown | −1-−1 | unknown | −1 | −1 | unknown | RAB3C/BC013033 |
| | | | | unknown | −1-−1 | unknown | −1 | −1 | unknown | NM_138453 |
| | | | | 5 | 57913524-57914151 | + | 57914695 | 544 | downstream | RAB3C/BC013033 |
| | | | | 5 | 57913524-57914151 | + | 57914695 | 544 | downstream | NM_138453 |
| 32 | 40:A10 | 255 | 235 | unknown | −1-−1 | unknown | −1 | −1 | unknown | NM_003081 |
| | | | | unknown | −1-−1 | unknown | −1 | −1 | unknown | NM_130811 |
| | | | | 20 | 10146129-10146378 | + | 10147476 | 1098 | downstream | NM_003081 |
| | | | | 20 | 10146129-10146378 | + | 10147476 | 1098 | downstream | NM_130811 |
| | | | | 20 | 10146129-10146378 | + | 10147489 | 1111 | downstream | SNAP25/D21267 |
| 33 | 106:H01 | 770 | 338 | unknown | −1-−1 | unknown | −1 | −1 | unknown | SALL4/AY170621 |
| | | | | unknown | −1-−1 | unknown | −1 | −1 | unknown | SALL4/AY170622 |
| | | | | unknown | −1-−1 | unknown | −1 | −1 | unknown | SALL4/AY172738 |
| | | | | 20 | 49850824-49851494 | − | 49852354 | 860 | upstream | SALL4/AY170621 |
| | | | | 20 | 49850824-49851494 | − | 49852354 | 860 | upstream | SALL4/AY170622 |
| | | | | 20 | 49850824-49851494 | − | 49852354 | 860 | upstream | SALL4/AY172738 |
| | | | | 20 | 49850824-49851494 | − | 49852421 | 927 | upstream | SALL4/AK001666 |
| | | | | 20 | 49850824-49851494 | − | 49852421 | 927 | upstream | NM_020436 |
| 34 | 121:F11 | 165 | 165 | unknown | −1-−1 | unknown | −1 | −1 | unknown | RANBP3/Y08697 |
| | | | | 19 | 5904724-5904889 | − | 5908984 | 4095 | upstream | RANBP3/Y08697 |
| 35 | 22:F06 | 776 | 776 | unknown | −1-−1 | unknown | −1 | −1 | unknown | GSH-2/AB028838 |
| | | | | unknown | −1-−1 | unknown | −1 | −1 | unknown | NM_133267 |
| | | | | 4 | 54808457-54809233 | + | 54807125 | 1332 | downstream | GSH-2/AB028838 |
| | | | | 4 | 54808457-54809233 | + | 54807125 | 1332 | downstream | NM_133267 |
| 36 | 24:B04 | 611 | 610 | unknown | −1-−1 | unknown | −1 | −1 | unknown | FLJ14936/AK092038 |
| | | | | unknown | −1-−1 | unknown | −1 | −1 | unknown | NM_032864 |
| | | | | 1 | 52581210-52581784 | + | 52582256 | 472 | downstream | FLJ14936/AK092038 |
| | | | | 1 | 52581210-52581784 | + | 52582256 | 472 | downstream | NM_032864 |
| | | | | 1 | 52581210-52581784 | + | 52582263 | 479 | downstream | FLJ14936/BC063655 |
| | | | | 1 | 52581210-52581784 | + | 52582320 | 536 | downstream | NM_032284 |
| | | | | 1 | 52581210-52581784 | + | 52583466 | 1682 | downstream | FLJ14936/AK027842 |
| | | | | 1 | 52581210-52581784 | − | 52582152 | 368 | upstream | ORC1L/U40152 |
| | | | | 1 | 52581210-52581784 | − | 52582152 | 368 | upstream | NM_004153 |
| 37 | 89:F03 | 626 | 626 | unknown | −1-−1 | unknown | −1 | −1 | unknown | AB011123 |
| | | | | 3 | 172660215-172660841 | − | 172660820 | 0 | within | AB011123 |
| 38 | 92:B12 | 295 | 3 | unknown | −1-−1 | unknown | −1 | −1 | unknown | CA2/BC011949 |
| | | | | unknown | −1-−1 | unknown | −1 | −1 | unknown | NM_000067 |
| | | | | 8 | 86564025-86564271 | + | 86563497 | 528 | downstream | CA2/BC011949 |
| | | | | 8 | 86564025-86564271 | + | 86563497 | 528 | downstream | NM_000067 |
| 39 | 45:H09 | 354 | 351 | unknown | −1-−1 | unknown | −1 | −1 | unknown | ING4/BG013038 |
| | | | | 12 | 6642667-6643018 | − | 6642514 | 153 | upstream | ING4/BC013038 |
| | | | | 12 | 6642667-6643018 | − | 6642531 | 136 | upstream | ING4/BC007781 |
| | | | | 12 | 6642667-6643018 | − | 6642546 | 121 | upstream | ING4/AF063594 |
| | | | | 12 | 6642667-6643018 | − | 6642565 | 102 | upstream | NM_016162 |
| | | | | 12 | 6642667-6643018 | − | 6642565 | 102 | upstream | NM_198287 |
| 40 | 53:B01 | 317 | 317 | unknown | −1-−1 | unknown | −1 | −1 | unknown | NM_002154 |
| | | | | unknown | −1-−1 | unknown | −1 | −1 | unknown | NM_198431 |
| | | | | 5 | 132414949-132415266 | + | 132415560 | 294 | downstream | NM_002154 |
| | | | | 5 | 132414949-132415266 | + | 132415560 | 294 | downstream | NM_198431 |
| | | | | 5 | 132414949-132415266 | + | 132415568 | 302 | downstream | HSPA4/AB023420 |
| | | | | 5 | 132414949-132415266 | + | 132415641 | 375 | downstream | HSPA4/BC002526 |
| | | | | 5 | 132414949-132415266 | + | 132415831 | 565 | downstream | HSPA4/X67643 |
| 41 | 7:H10 | 526 | 526 | unknown | −1-−1 | unknown | −1 | −1 | unknown | MOBP/D28113 |
| | | | | unknown | −1-−1 | unknown | −1 | −1 | unknown | MOBP/D28114 |
| | | | | unknown | −1-−1 | unknown | −1 | −1 | unknown | NM_006501 |
| | | | | unknown | −1-−1 | unknown | −1 | −1 | unknown | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39519109-39519206 | + | 39518557 | 552 | downstream | MOBP/D28113 |
| | | | | 3 | 39519109-39519206 | + | 39518557 | 552 | downstream | MOBP/D28114 |
| | | | | 3 | 39519109-39519206 | + | 39518557 | 552 | downstream | NM_006501 |
| | | | | 3 | 39519109-39519206 | + | 39518557 | 552 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519109-39519176 | + | 39518557 | 552 | downstream | MOBP/D28113 |
| | | | | 3 | 39519109-39519176 | + | 39518557 | 552 | downstream | MOBP/D28114 |
| | | | | 3 | 39519109-39519176 | + | 39518557 | 552 | downstream | NM_006501 |
| | | | | 3 | 39519109-39519176 | + | 39518557 | 552 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519139-39519236 | + | 39518557 | 582 | downstream | MOBP/D28113 |
| | | | | 3 | 39519139-39519236 | + | 39518557 | 582 | downstream | MOBP/D28114 |
| | | | | 3 | 39519139-39519236 | + | 39518557 | 582 | downstream | NM_006501 |
| | | | | 3 | 39519139-39519236 | + | 39518557 | 582 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519169-39519236 | + | 39518557 | 612 | downstream | MOBP/D28113 |
| | | | | 3 | 39519169-39519236 | + | 39518557 | 612 | downstream | MOBP/D28114 |
| | | | | 3 | 39519169-39519236 | + | 39518557 | 612 | downstream | NM_006501 |
| | | | | 3 | 39519169-39519236 | + | 39518557 | 612 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39519109-39519206 | + | 39518557 | 552 | downstream | MOBP/D28113 |
| | | | | 3 | 39519109-39519206 | + | 39518557 | 552 | downstream | MOBP/D28114 |
| | | | | 3 | 39519109-39519206 | + | 39518557 | 552 | downstream | NM_006501 |
| | | | | 3 | 39519109-39519206 | + | 39518557 | 552 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519109-39519206 | + | 39518557 | 552 | downstream | MOBP/D28113 |
| | | | | 3 | 39519109-39519206 | + | 39518557 | 552 | downstream | MOBP/D28114 |
| | | | | 3 | 39519109-39519206 | + | 39518557 | 552 | downstream | NM_006501 |
| | | | | 3 | 39519109-39519206 | + | 39518557 | 552 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39518976-39519335 | + | 39518557 | 419 | downstream | MOBP/D28113 |
| | | | | 3 | 39518976-39519335 | + | 39518557 | 419 | downstream | MOBP/D28114 |
| | | | | 3 | 39518976-39519335 | + | 39518557 | 419 | downstream | NM_006501 |
| | | | | 3 | 39518976-39519335 | + | 39518557 | 419 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519139-39519236 | + | 39518557 | 582 | downstream | MOBP/D28113 |
| | | | | 3 | 39519139-39519236 | + | 39518557 | 582 | downstream | MOBP/D28114 |
| | | | | 3 | 39519139-39519236 | + | 39518557 | 582 | downstream | NM_006501 |
| | | | | 3 | 39519139-39519236 | + | 39518557 | 582 | downstream | NM_182934 |
| | | | | 3 | 39519109-39519236 | + | 39518557 | 552 | downstream | MOBP/D28113 |
| | | | | 3 | 39519109-39519236 | + | 39518557 | 552 | downstream | MOBP/D28114 |
| | | | | 3 | 39519109-39519236 | + | 39518557 | 552 | downstream | NM_006501 |
| | | | | 3 | 39519109-39519236 | + | 39518557 | 552 | downstream | NM_182934 |
| | | | | 3 | 39519139-39519236 | + | 39518557 | 582 | downstream | MOBP/D28113 |
| | | | | 3 | 39519139-39519236 | + | 39518557 | 582 | downstream | MOBP/D28114 |
| | | | | 3 | 39519139-39519236 | + | 39518557 | 582 | downstream | NM_006501 |
| | | | | 3 | 39519139-39519236 | + | 39518557 | 582 | downstream | NM_182934 |
| 42 | 119:D03 | 661 | 661 | unknown | −1-−1 | unknown | −1 | −1 | unknown | KIAA0247/BC064697 |
| | | | | unknown | −1-−1 | unknown | −1 | −1 | unknown | NM_014734 |
| | | | | 14 | 69147865-69148839 | + | 69148065 | 0 | within | KIAA0247/BC064697 |
| | | | | 14 | 69147865-69148839 | + | 69148065 | 0 | within | NM_014734 |
| | | | | 14 | 69147865-69148839 | + | 69148110 | 0 | within | KIAA0247/D87434 |
| 43 | 56:G03 | 220 | 220 | unknown | −1-−1 | unknown | −1 | −1 | unknown | EIF4A2/BC015842 |
| | | | | 3 | 187983586-187983806 | + | 187984067 | 261 | downstream | EIF4A2/BC015842 |
| | | | | 3 | 187983586-187983806 | + | 187984086 | 280 | downstream | NM_001967 |
| | | | | 3 | 187983586-187983806 | + | 187984087 | 281 | downstream | EIF4A2/AF208852 |
| | | | | 3 | 187983586-187983806 | + | 187984101 | 295 | downstream | EIF4A2/BT009860 |
| | | | | 3 | 187983586-187983806 | + | 187984935 | 1129 | downstream | AY207392 |
| | | | | 3 | 187983586-187983806 | + | 187985649 | 1843 | downstream | EIF4A2/AL137681 |
| | | | | 3 | 187983586-187983806 | + | 187987048 | 3242 | downstream | EIF4A2/BC016295 |
| 44 | 53:E10 | 530 | 524 | unknown | −1-−1 | unknown | −1 | −1 | unknown | SYK/BC002962 |
| | | | | unknown | −1-−1 | unknown | −1 | −1 | unknown | NM_003177 |
| | | | | 9 | 90643120-90643604 | + | 90643624 | 20 | downstream | SYK/BC002962 |
| | | | | 9 | 90643120-90643604 | + | 90643624 | 20 | downstream | NM_003177 |
| 45 | 128:C08 | 919 | 888 | unknown | −1-−1 | unknown | −1 | −1 | unknown | C14orf31/BC020521 |
| | | | | unknown | −1-−1 | unknown | −1 | −1 | unknown | NM_152330 |
| | | | | 14 | 51186574-51187171 | + | 51188377 | 1206 | downstream | C14orf31/BC020521 |
| | | | | 14 | 51186574-51187171 | + | 51188377 | 1206 | downstream | NM_152330 |
| 46 | 68:H09 | 661 | 661 | unknown | −1-−1 | unknown | −1 | −1 | unknown | PAI-RBP1/AY032853 |
| | | | | 1 | 67607702-67608363 | − | 67607753 | 0 | within | PAI-RBP1/AY032853 |
| | | | | 1 | 67607702-67608363 | − | 67608076 | 0 | within | PAI-RBP1/BC020555 |
| | | | | 1 | 67607702-67608363 | − | 67608085 | 0 | within | PAI-RBP1/AL080119 |
| | | | | 1 | 67607702-67608363 | − | 67608085 | 0 | within | NM_015640 |
| | | | | 1 | 67607702-67608363 | − | 67608087 | 0 | within | PAI-RBP1/AF151813 |
| | | | | 1 | 67607702-67608363 | − | 67608090 | 0 | within | AK074970 |
| 47 | 41:H02 | 143 | 479 | unknown | −1-−1 | unknown | −1 | −1 | unknown | GPX1/X13710 |
| | | | | 3 | 49370864-49371108 | − | 49370720 | 144 | upstream | GPX1/X13710 |
| | | | | 3 | 49370864-49371108 | − | 49370738 | 126 | upstream | GPX1/BC000742 |
| | | | | 3 | 49370864-49371108 | − | 49370795 | 69 | upstream | NM_000581 |
| | | | | 3 | 49370864-49371108 | − | 49370795 | 69 | upstream | NM_201397 |
| | | | | 3 | 49370864-49371108 | − | 49375064 | 3956 | upstream | RHOA/BC000946 |
| 48 | 29:B11 | 501 | 447 | unknown | −1-−1 | unknown | −1 | −1 | unknown | RLN1/BC005956 |
| | | | | unknown | −1-−1 | unknown | −1 | −1 | unknown | NM_006911 |
| | | | | 9 | 5329030-5329538 | − | 5329873 | 335 | upstream | RLN1/BC005956 |
| | | | | 9 | 5329030-5329538 | − | 5329873 | 335 | upstream | NM_006911 |
| | | | | 9 | 5293864-5294372 | − | 5294580 | 208 | upstream | RLN2/X00948 |
| | | | | 9 | 5293864-5294372 | − | 5294580 | 208 | upstream | NM_005059 |
| | | | | 9 | 5293864-5294372 | − | 5294580 | 208 | upstream | NM_134441 |
| 49 | 64:A06 | 877 | 785 | unknown | −1-−1 | unknown | −1 | −1 | unknown | AK000471 |
| | | | | 22 | 30065033-30066293 | + | 30067428 | 1135 | downstream | AK000471 |
| | | | | 22 | 30065033-30066293 | − | 30066772 | 479 | upstream | ZNF278/AF254085 |
| | | | | 22 | 30065033-30066293 | − | 30066803 | 510 | upstream | NM_014323 |
| | | | | 22 | 30065033-30066293 | − | 30066803 | 510 | upstream | NM_032050 |
| | | | | 22 | 30065033-30066293 | − | 30066803 | 510 | upstream | NM_032052 |
| | | | | 22 | 30065033-30066293 | − | 30066803 | 510 | upstream | NM_032051 |
| | | | | 22 | 30065033-30066293 | − | 30066159 | 0 | within | ZNF278/AF242522 |
| 50 | 85:B02 | 627 | 664 | unknown | −1-−1 | unknown | −1 | −1 | unknown | BC011002 |
| | | | | 19 | 14046138-14046802 | + | 14044820 | 1318 | downstream | BC011002 |
| 51 | 125:B02 | 184 | 184 | unknown | −1-−1 | unknown | −1 | −1 | unknown | AF058389 |
| | | | | 15 | 86483494-86483678 | − | 86479498 | 3996 | upstream | AF058389 |
| 52 | 93:B08 | 288 | 287 | unknown | −1-−1 | unknown | −1 | −1 | unknown | NM_022337 |
| | | | | 11 | 87548364-87548640 | − | 87548247 | 117 | upstream | NM_022337 |
| | | | | 11 | 87548364-87548640 | − | 87548249 | 115 | upstream | RAB38/BC015808 |
| 53 | 80:G08 | 732 | 725 | unknown | −1-−1 | unknown | −1 | −1 | unknown | NM_000138 |
| | | | | 15 | 46724556-46725282 | − | 46724391 | 165 | upstream | NM_000138 |
| | | | | 15 | 46724556-46725282 | − | 46725277 | 0 | within | FBN1/X63556 |
| 54 | 73:H04 | 269 | 269 | unknown | −1-−1 | unknown | −1 | −1 | unknown | DJ122O8.2/BC009782 |
| | | | | unknown | −1-−1 | unknown | −1 | −1 | unknown | NM_020466 |
| | | | | 6 | 90405119-90405388 | − | 90405185 | 0 | within | DJ122O8.2/BC009782 |
| | | | | 6 | 90405119-90405388 | − | 90405185 | 0 | within | NM_020466 |
| 55 | 70:E03 | 288 | 288 | unknown | −1-−1 | unknown | −1 | −1 | unknown | NM_007308 |
| | | | | 4 | 91115726-91116014 | − | 91114009 | 1717 | upstream | NM_007308 |
| | | | | 4 | 91115726-91116014 | − | 91115311 | 415 | upstream | SNCA/L08850 |
| | | | | 4 | 91115726-91116014 | − | 91115311 | 415 | upstream | NM_000345 |
| 56 | 46:D09 | 497 | 496 | unknown | −1-−1 | unknown | −1 | −1 | unknown | DDX25/BC050360 |
| | | | | 11 | 125278145-125278481 | + | 125279600 | 1119 | downstream | DDX25/BC050360 |
| | | | | 11 | 125278145-125278481 | + | 125279612 | 1131 | downstream | NM_013264 |
| | | | | 11 | 125278145-125278481 | − | 125278315 | 0 | within | FKSG32/BC004822 |
| | | | | 11 | 125278145-125278481 | − | 125278315 | 0 | within | NM_031307 |
| 57 | 68:H08 | 807 | 822 | unknown | −1-−1 | unknown | −1 | −1 | unknown | NM_014171 |
| | | | | 2 | 46755111-46756089 | + | 46755958 | 0 | within | NM_014171 |
| | | | | 2 | 46755111-46756089 | + | 46756001 | 0 | within | CRIPT/BC018653 |
| | | | | 2 | 46755111-46756089 | − | 46755841 | 0 | within | PIGF/BC021725 |
| | | | | 2 | 46755111-46756089 | − | 46755855 | 0 | within | NM_002643 |
| | | | | 2 | 46755111-46756089 | − | 46755855 | 0 | within | NM_173074 |
| 58 | 54:A04 | 229 | 226 | unknown | −1-−1 | unknown | −1 | −1 | unknown | PCNT2/AK024009 |
| | | | | unknown | −1-−1 | unknown | −1 | −1 | unknown | NM_006031 |
| | | | | 21 | 46567168-46567396 | + | 46568482 | 1086 | downstream | PCNT2/AK024009 |
| | | | | 21 | 46567168-46567396 | + | 46568482 | 1086 | downstream | NM_006031 |
| | | | | 21 | 46567168-46567396 | + | 46568492 | 1096 | downstream | PCNT2/BC035913 |
| | | | | 21 | 46567168-46567396 | + | 46568570 | 1174 | downstream | PCNT2/AF515282 |
| | | | | 21 | 46567168-46567396 | + | 46569229 | 1833 | downstream | PCNT2/AB007862 |
| | | | | 21 | 46567168-46567396 | − | 46562499 | 4669 | upstream | C21orf58/AK098098 |
| | | | | 21 | 46567168-46567396 | − | 46562548 | 4620 | upstream | C21orf58/AY039243 |
| | | | | 21 | 46567168-46567396 | − | 46568213 | 817 | upstream | NM_058180 |
| | | | | 21 | 46567168-46567396 | − | 46568213 | 817 | upstream | NM_199071 |
| 59 | 27:B04 | 471 | 471 | unknown | −1-−1 | unknown | −1 | −1 | unknown | HIST1H2BO/NM_003527 |
| | | | | 6 | 27970131-27970387 | + | 27969181 | 950 | downstream | HIST1H2BO/NM_003527 |
| | | | | 6 | 27970131-27970387 | − | 27966549 | 3582 | upstream | HIST1H3J/NM_003535 |
| | | | | 6 | 27970131-27970387 | − | 27968942 | 1189 | upstream | HIST1H2AM/NM_003514 |
| 60 | 48:A11 | 601 | 14 | unknown | −1-−1 | unknown | −1 | −1 | unknown | AK128497 |
| | | | | 20 | 62058844-62059322 | + | 62055180 | 3664 | downstream | AK128497 |
| | | | | 20 | 62058844-62059322 | − | 62058182 | 662 | upstream | URKL1/BC033078 |
| | | | | 20 | 62058844-62059322 | − | 62058212 | 632 | upstream | URKL1/AJ605558 |
| | | | | 20 | 62058844-62059322 | − | 62058212 | 632 | upstream | URKL1/AK000524 |
| | | | | 20 | 62058844-62059322 | − | 62058212 | 632 | upstream | NM_017859 |
| 61 | 63:E04 | 827 | 838 | unknown | −1-−1 | unknown | −1 | −1 | unknown | LOC283514/AK096522 |
| | | | | unknown | −1-−1 | unknown | −1 | −1 | unknown | NM_198849 |
| | | | | 13 | 45322805-45324127 | − | 45323758 | 0 | within | LOC283514/AK096522 |
| | | | | 13 | 45322805-45324127 | − | 45323758 | 0 | within | NM_198849 |
| | | | | 13 | 45322805-45324127 | − | 45323844 | 0 | within | LOC283514/BC041372 |
| 62 | 65:E12 | 224 | 224 | unknown | −1-−1 | unknown | −1 | −1 | unknown | ABI2/X95632 |
| | | | | unknown | −1-−1 | unknown | −1 | −1 | unknown | NM_005759 |
| | | | | 2 | 204019056-204019280 | + | 204018667 | 389 | downstream | ABI2/X95632 |
| | | | | 2 | 204019056-204019280 | + | 204018667 | 389 | downstream | NM_005759 |
| | | | | 2 | 204019056-204019280 | + | 204018743 | 313 | downstream | ABI2/AF260261 |
| | | | | 2 | 204019056-204019280 | − | 204019437 | 157 | upstream | AK125205 |
| 63 | 120:B12 | 126 | 126 | unknown | −1-−1 | unknown | −1 | −1 | unknown | NM_173479 |
| | | | | x | 108103520-108103581 | + | 108103493 | 27 | downstream | NM_173479 |
| 64 | 117:B09 | 244 | 244 | unknown | −1-−1 | unknown | −1 | −1 | unknown | FLJ25952/BC050367 |
| | | | | unknown | −1-−1 | unknown | −1 | −1 | unknown | NM_153251 |
| | | | | 13 | 20930559-20930803 | − | 20931423 | 620 | upstream | FLJ25952/BC050367 |
| | | | | 13 | 20930559-20930803 | − | 20931423 | 620 | upstream | NM_153251 |
| | | | | 13 | 20930559-20930803 | − | 20931507 | 704 | upstream | BC067898 |
| 65 | 79:H01 | 301 | 301 | unknown | −1-−1 | unknown | −1 | −1 | unknown | SMURF2/AY014180 |
| | | | | unknown | −1-−1 | unknown | −1 | −1 | unknown | NM_022739 |
| | | | | 17 | 60089165-60089466 | − | 60088648 | 517 | upstream | SMURF2/AY014180 |
| | | | | 17 | 60089165-60089466 | − | 60088648 | 517 | upstream | NM_022739 |
| 66 | 59:C06 | 291 | 514 | unknown | −1-−1 | unknown | −1 | −1 | unknown | ZBTB10/AJ319673 |
| | | | | unknown | −1-−1 | unknown | −1 | −1 | unknown | NM_023929 |
| | | | | 8 | 81560357-81561215 | + | 81561002 | 0 | within | ZBTB10/AJ319673 |
| | | | | 8 | 81560357-81561215 | + | 81561002 | 0 | within | NM_023929 |
| 67 | 56:H04 | 135 | 135 | unknown | −1-−1 | unknown | −1 | −1 | unknown | PTPN9/BT007405 |
| | | | | 15 | 73658346-73658481 | − | 73658172 | 174 | upstream | PTPN9/BT007405 |
| | | | | 15 | 73658346-73658481 | − | 73658680 | 199 | upstream | PTPN9/M83738 |
| | | | | 15 | 73658346-73658481 | − | 73658680 | 199 | upstream | NM_002833 |
| 68 | 22:H08 | 186 | 111 | unknown | −1-−1 | unknown | −1 | −1 | unknown | SPRY2/BC004205 |
| | | | | 13 | 79814722-79814780 | − | 79810973 | 3749 | upstream | SPRY2/BC004205 |
| | | | | 13 | 79814722-79814780 | − | 79813087 | 1635 | upstream | SPRY2/AF039843 |
| | | | | 13 | 79814722-79814780 | − | 79813087 | 1635 | upstream | NM_005842 |
| 69 | 17:E03 | 644 | 643 | unknown | −1-−1 | unknown | −1 | −1 | unknown | NM_207581 |
| | | | | 15 | 43192511-43193155 | + | 43193963 | 808 | downstream | NM_207581 |
| | | | | 15 | 43192511-43193155 | − | 43189943 | 2568 | upstream | DUOX2/AF181972 |
| | | | | 15 | 43192511-43193155 | − | 43193651 | 496 | upstream | DUOX2/AF267981 |
| | | | | 15 | 43192511-43193155 | − | 43193651 | 496 | upstream | NM_014080 |
| 70 | 31:B07 | 651 | 651 | unknown | −1-−1 | unknown | −1 | −1 | unknown | U44425 |
| | | | | 9 | 124256889-124257540 | − | 124257244 | 0 | within | U44425 |
| | | | | 9 | 124256889-124257540 | − | 124257258 | 0 | within | BT007218 |
| | | | | 9 | 124256889-124257540 | − | 124257262 | 0 | within | PSMB7/BC000509 |
| | | | | 9 | 124256889-124257540 | − | 124257275 | 0 | within | NM_002799 |
| 71 | 41:B06 | 586 | 586 | unknown | −1-−1 | unknown | −1 | −1 | unknown | NM_173479 |
| | | | | x | 108103645-108103938 | + | 108103493 | 152 | downstream | NM_173479 |
| 72 | 11:E08 | 199 | 199 | unknown | −1-−1 | unknown | −1 | −1 | unknown | AB002324 |
| | | | | 16 | 30705034-30705233 | − | 30705990 | 757 | upstream | AB002324 |
| 73 | 27:E05 | 801 | 863 | unknown | −1-−1 | unknown | −1 | −1 | unknown | NM_001182 |
| | | | | 5 | 125958441-125959305 | − | 125958756 | 0 | within | NM_001182 |
| | | | | 5 | 125958441-125959305 | − | 125958798 | 0 | within | ALDH7A1/BC002515 |
| 74 | 60:H04 | 172 | 172 | unknown | −1-−1 | unknown | −1 | −1 | unknown | LOX/AF039291 |
| | | | | 5 | 121441880-121442052 | − | 121441828 | 52 | upstream | LOX/AF039291 |
| | | | | 5 | 121441880-121442052 | − | 121441853 | 27 | upstream | NM_002317 |
| 75 | 11:D05 | 567 | 511 | unknown | −1-−1 | unknown | −1 | −1 | unknown | BX161388 |
| | | | | 14 | 103457340-103457783 | − | 103457591 | 0 | within | BX161388 |
| | | | | 14 | 103457340-103457783 | − | 103457608 | 0 | within | C14orf2/BC001944 |
| | | | | 14 | 103457340-103457783 | − | 103457619 | 0 | within | NM_004894 |
| 76 | 80:C04 | 732 | 745 | unknown | −1-−1 | unknown | −1 | −1 | unknown | MAN1A1/X74837 |
| | | | | 6 | 119712296-119713468 | − | 119711788 | 508 | upstream | MAN1A1/X74837 |
| | | | | 6 | 119712296-119713468 | − | 119712600 | 0 | within | BC065827 |
| | | | | 6 | 119712296-119713468 | − | 119712625 | 0 | within | NM_005907 |
| 77 | 93:C02 | 670 | 670 | unknown | −1-−1 | unknown | −1 | −1 | unknown | AP4M1/AF020796 |
| | | | | unknown | −1-−1 | unknown | −1 | −1 | unknown | NM_004722 |
| | | | | 7 | 99343223-99343893 | + | 99344139 | 246 | downstream | AP4M1/BX640759 |
| | | | | 7 | 99343223-99343893 | − | 99339947 | 3276 | upstream | MCM7/AY007130 |
| | | | | 7 | 99343223-99343893 | − | 99342011 | 1212 | upstream | MCM7/AF279900 |
| | | | | 7 | 99343223-99343893 | − | 99343031 | 192 | upstream | NM_182776 |
| | | | | 7 | 99343223-99343893 | − | 99344078 | 185 | upstream | NM_005916 |
| | | | | 7 | 99343223-99343893 | + | 99343830 | 0 | within | AP4M1/AF020796 |
| | | | | 7 | 99343223-99343893 | + | 99343830 | 0 | within | NM_004722 |
| | | | | 7 | 99343223-99343893 | − | 99343650 | 0 | within | MCM7/BC013375 |
| 78 | 32:H11 | 306 | 305 | unknown | −1-−1 | unknown | −1 | −1 | unknown | AK075241 |
| | | | | 19 | 1314009-1314314 | + | 1315552 | 1238 | downstream | AK075241 |
| | | | | 19 | 1314009-1314314 | + | 1318200 | 3886 | downstream | BC008098 |
| 79 | 114:D11 | 615 | 614 | unknown | −1-−1 | unknown | −1 | −1 | unknown | FOXB1/AF071554 |
| | | | | unknown | −1-−1 | unknown | −1 | −1 | unknown | NM_012182 |
| | | | | 15 | 58079608-58080181 | + | 58084426 | 4245 | downstream | FOXB1/AF071554 |
| | | | | 15 | 58079608-58080181 | + | 58084426 | 4245 | downstream | NM_012182 |
| 80 | 27:C10 | 839 | 850 | unknown | −1-−1 | unknown | −1 | −1 | unknown | NM_002894 |
| | | | | 18 | 18767383-18768291 | + | 18767292 | 91 | downstream | NM_002894 |
| | | | | 18 | 18767383-18768291 | + | 18767318 | 65 | downstream | RBBP8/U72066 |
| | | | | 18 | 18767383-18768291 | + | 18768710 | 419 | downstream | RBBP8/BC030590 |
| | | | | 18 | 18767383-18768291 | + | 18767836 | 0 | within | NM_203291 |
| | | | | 18 | 18767383-18768291 | + | 18767836 | 0 | within | NM_203292 |
| 81 | 26:E04 | 929 | 887 | unknown | −1-−1 | unknown | −1 | −1 | unknown | PTAFR/BC063000 |
| | | | | unknown | −1-−1 | unknown | −1 | −1 | unknown | NM_000952 |
| | | | | 1 | 28185436-28185558 | − | 28187333 | 1775 | upstream | PTAFR/BC063000 |
| | | | | 1 | 28185436-28185558 | − | 28187333 | 1775 | upstream | NM_000952 |
| 82 | 35:C10 | 194 | 195 | unknown | −1-−1 | unknown | −1 | −1 | unknown | NM_005413 |
| | | | | 2 | 45078887-45079073 | + | 45080511 | 1438 | downstream | NM_005413 |
| | | | | 2 | 45078887-45079073 | + | 45080687 | 1614 | downstream | SIX3/AJ012611 |
| | | | | 2 | 45078887-45079073 | + | 45080708 | 1635 | downstream | AL162671 |
| 83 | 80:G07 | 509 | 509 | unknown | −1-−1 | unknown | −1 | −1 | unknown | NM_194278 |
| | | | | 14 | 73296330-73296839 | − | 73296745 | 0 | within | NM_194278 |
| 84 | 110:B03 | 411 | 411 | unknown | −1-−1 | unknown | −1 | −1 | unknown | LOC112885/BC012187 |
| | | | | unknown | −1-−1 | unknown | −1 | −1 | unknown | NM_138415 |
| | | | | 13 | 97594343-97594725 | + | 97593434 | 909 | downstream | NM_001001715 |
| | | | | 13 | 97594343-97594725 | + | 97593434 | 909 | downstream | NM_005766 |
| | | | | 13 | 97594343-97594725 | + | 97593722 | 621 | downstream | FARP1/AB008430 |
| | | | | 22 | 43722859-43723005 | − | 43726118 | 3113 | upstream | LOC112885/BC012187 |
| | | | | 22 | 43722859-43723005 | − | 43726118 | 3113 | upstream | NM_138415 |
| 85 | 100:B04 | 707 | 807 | unknown | −1-−1 | unknown | −1 | −1 | unknown | NM_006859 |
| | | | | unknown | −1-−1 | unknown | −1 | −1 | unknown | NM_194451 |
| | | | | 4 | 39282467-39283464 | + | 39283230 | 0 | within | NM_006859 |
| | | | | 4 | 39282467-39283464 | + | 39283230 | 0 | within | NM_194451 |
| | | | | 4 | 39282467-39283464 | + | 39283243 | 0 | within | BC062751 |
| | | | | 4 | 39282467-39283464 | + | 39283264 | 0 | within | LIAS/BC023635 |
| | | | | 4 | 39282467-39283464 | − | 39283103 | 0 | within | NM_000661 |
| 86 | 1:A07 | 627 | 627 | unknown | −1-−1 | unknown | −1 | −1 | unknown | SOCS3/BC060858 |
| | | | | unknown | −1-−1 | unknown | −1 | −1 | unknown | NM_003955 |
| | | | | 17 | 73872256-73872883 | − | 73867753 | 4503 | upstream | SOCS3/BC060858 |
| | | | | 17 | 73872256-73872883 | − | 73867753 | 4503 | upstream | NM_003955 |
| 87 | 122:E08 | 202 | 203 | unknown | −1-−1 | unknown | −1 | −1 | unknown | NM_172316 |
| | | | | 15 | 35183098-35183301 | − | 35178889 | 4209 | upstream | NM_172316 |
| | | | | 15 | 35183098-35183301 | − | 35179996 | 3102 | upstream | NM_020149 |
| | | | | 15 | 35183098-35183301 | − | 35179996 | 3102 | upstream | NM_170674 |
| | | | | 15 | 35183098-35183301 | − | 35179996 | 3102 | upstream | NM_170675 |
| | | | | 15 | 35183098-35183301 | − | 35179996 | 3102 | upstream | NM_170676 |
| | | | | 15 | 35183098-35183301 | − | 35179996 | 3102 | upstream | NM_170677 |
| | | | | 15 | 35183098-35183301 | − | 35180673 | 2425 | upstream | MEIS2/BC050431 |
| | | | | 15 | 35183098-35183301 | − | 35180792 | 2306 | upstream | NM_002399 |
| | | | | 15 | 35183098-35183301 | − | 35180796 | 2302 | upstream | MEIS2/BC001844 |
| 88 | 10:D01 | 266 | 266 | unknown | −1-−1 | unknown | −1 | −1 | unknown | AK126015 |
| | | | | 5 | 92957219-92957485 | + | 92961818 | 4333 | downstream | AK126015 |
| 89 | 113:C06 | 223 | 223 | unknown | −1-−1 | unknown | −1 | −1 | unknown | MMP25/AJ272137 |
| | | | | unknown | −1-−1 | unknown | −1 | −1 | unknown | NM_022718 |
| | | | | unknown | −1-−1 | unknown | −1 | −1 | unknown | NM_022468 |
| | | | | 16 | 3035713-3035936 | + | 3036682 | 746 | downstream | MMP25/AJ272137 |
| | | | | 16 | 3035713-3035936 | + | 3036682 | 746 | downstream | NM_022718 |
| | | | | 16 | 3035713-3035936 | + | 3036682 | 746 | downstream | NM_022468 |
| | | | | 16 | 3035713-3035936 | + | 3037532 | 1596 | downstream | MMPL1/AJ003144 |
| | | | | 16 | 3035713-3035936 | + | 3037532 | 1596 | downstream | NM_004142 |
| 90 | 113:D09 | 346 | 346 | unknown | −1-−1 | unknown | −1 | −1 | unknown | RNF34/AF306709 |
| | | | | 12 | 120299581-120299935 | + | 120300563 | 628 | downstream | RNF34/AF306709 |
| | | | | 12 | 120299581-120299935 | + | 120300621 | 686 | downstream | NM_025126 |
| | | | | 12 | 120299581-120299935 | + | 120300621 | 686 | downstream | NM_194271 |
| | | | | 12 | 120299581-120299935 | + | 120300805 | 870 | downstream | BC029038 |
| 91 | 65:G08 | 606 | 689 | unknown | −1-−1 | unknown | −1 | −1 | unknown | SHMT2/BC032584 |
| | | | | unknown | −1-−1 | unknown | −1 | −1 | unknown | SHMT2/AK055053 |
| | | | | 12 | 55910079-55910995 | + | 55909760 | 319 | downstream | SHMT2/BC032584 |
| | | | | 12 | 55910079-55910995 | + | 55909760 | 319 | downstream | SHMT2/AK055053 |
| | | | | 12 | 55910079-55910995 | + | 55909818 | 261 | downstream | SHMT2/BC011911 |
| | | | | 12 | 55910079-55910995 | + | 55909818 | 261 | downstream | NM_005412 |
| | | | | 12 | 55910079-55910995 | + | 55909828 | 251 | downstream | SHMT2/BT006866 |
| 92 | 25:F01 | 505 | 505 | unknown | −1-−1 | unknown | −1 | −1 | unknown | MGC4504/BC001683 |
| | | | | unknown | −1-−1 | unknown | −1 | −1 | unknown | NM_024111 |
| | | | | 15 | 39032942-39033440 | + | 39032982 | 0 | within | MGC4504/BC001683 |
| | | | | 15 | 39032942-39033440 | + | 39032982 | 0 | within | NM_024111 |
| 93 | 18:E08 | 594 | 594 | unknown | −1-−1 | unknown | −1 | −1 | unknown | ONECUT1/U96173 |
| | | | | unknown | −1-−1 | unknown | −1 | −1 | unknown | NM_004498 |
| | | | | 15 | 50873810-50874397 | − | 50869501 | 4309 | upstream | ONECUT1/U96173 |
| | | | | 15 | 50873810-50874397 | − | 50869501 | 4309 | upstream | NM_004498 |
| 94 | 1:G11 | 961 | 571 | unknown | −1-−1 | unknown | −1 | −1 | unknown | GPR14/NM_018949 |
| | | | | 17 | 77921639-77922214 | + | 77925489 | 3275 | downstream | GPR14/NM_018949 |
| 95 | 58:E12 | 199 | 201 | unknown | −1-−1 | unknown | −1 | −1 | unknown | URG4/AB040940 |
| | | | | 7 | 43718765-43718966 | − | 43719150 | 184 | upstream | URG4/AB040940 |
| | | | | 7 | 43718765-43718966 | − | 43719427 | 461 | upstream | URG4/AY078404 |
| | | | | 7 | 43718765-43718966 | − | 43719427 | 461 | upstream | NM_017920 |
| | | | | 7 | 43718765-43718966 | − | 43719429 | 463 | upstream | URG4/BC018426 |
| | | | | 7 | 43718765-43718966 | − | 43719443 | 477 | upstream | URG4/BX640797 |
| 96 | 10:A09 | 721 | 721 | unknown | −1-−1 | unknown | −1 | −1 | unknown | GGN/AF538037 |
| | | | | 19 | 43569963-43570684 | − | 43570504 | 0 | within | GGN/AF538037 |
| | | | | 19 | 43569963-43570684 | − | 43570508 | 0 | within | GGN/AF538035 |
| | | | | 19 | 43569963-43570684 | − | 43570508 | 0 | within | GGN/AF538036 |
| | | | | 19 | 43569963-43570684 | − | 43570508 | 0 | within | NM_152657 |
| | | | | 19 | 43569963-43570684 | − | 43570508 | 0 | within | NM_182477 |
| | | | | 19 | 43569963-43570684 | − | 43570514 | 0 | within | GGN/AK057356 |
| 97 | 25:E01 | 362 | 362 | unknown | −1-−1 | unknown | −1 | −1 | unknown | TBX3/BC025258 |
| | | | | 12 | 113586072-113586380 | − | 113584115 | 1957 | upstream | TBX3/BC025258 |
| | | | | 12 | 113586072-113586380 | − | 113584689 | 1383 | upstream | NM_005996 |
| | | | | 12 | 113586072-113586380 | − | 113584689 | 1383 | upstream | NM_016569 |
| 98 | 60:C10 | 727 | 689 | unknown | −1-−1 | unknown | −1 | −1 | unknown | CD226/U56102 |
| | | | | unknown | −1-−1 | unknown | −1 | −1 | unknown | NM_006566 |
| | | | | 18 | 65773765-65774157 | − | 65775140 | 983 | upstream | CD226/U56102 |
| | | | | 18 | 65773765-65774157 | − | 65775140 | 983 | upstream | NM_006566 |
| 99 | 5:F02 | 405 | 405 | unknown | −1-−1 | unknown | −1 | −1 | unknown | BC028123 |
| | | | | 11 | 10518479-10518884 | + | 10519394 | 510 | downstream | BC028123 |
| | | | | 11 | 10518479-10518884 | − | 10519340 | 456 | upstream | RNF141/BC018104 |
| | | | | 11 | 10518479-10518884 | − | 10519350 | 466 | upstream | NM_016422 |
| 100 | 57:F09 | 629 | 604 | unknown | −1-−1 | unknown | −1 | −1 | unknown | NM_013433 |
| | | | | 19 | 12695457-12696086 | − | 12694078 | 1379 | upstream | NM_013433 |
| | | | | 19 | 12695457-12696086 | − | 12694369 | 1088 | upstream | TNPO2/AF019039 |
| 101 | 50:D05 | 965 | 511 | unknown | −1-−1 | unknown | −1 | −1 | unknown | AMOTL2/BC025981 |
| | | | | 3 | 135564917-135565826 | − | 135567869 | 2043 | upstream | AMOTL2/BC025981 |
| | | | | 3 | 135564917-135565826 | − | 135569346 | 3520 | upstream | AMOTL2/BC011454 |
| 102 | 15:G05 | 832 | 703 | unknown | −1-−1 | unknown | −1 | −1 | unknown | DDX1/X70649 |
| | | | | unknown | −1-−1 | unknown | −1 | −1 | unknown | NM_004939 |
| | | | | 2 | 15682226-15683012 | + | 15682367 | 0 | within | DDX1/X70649 |
| | | | | 2 | 15682226-15683012 | + | 15682367 | 0 | within | NM_004939 |
| | | | | 2 | 15682226-15683012 | + | 15682620 | 0 | within | DDX1/BC012739 |
| 103 | 21:H05 | 716 | 709 | unknown | −1-−1 | unknown | −1 | −1 | unknown | ASMT/U11090 |
| | | | | unknown | −1-−1 | unknown | −1 | −1 | unknown | NM_004043 |
| | | | | x | 1760934-1761794 | + | 1758174 | 2760 | downstream | ASMT/U11090 |
| | | | | x | 1760934-1761794 | + | 1758174 | 2760 | downstream | NM_004043 |
| | | | | y | 1760934-1761794 | + | 1758174 | 2760 | downstream | ASMT/U11090 |
| | | | | y | 1760934-1761794 | + | 1758174 | 2760 | downstream | NM_004043 |
| 104 | 22:B12 | 466 | 434 | unknown | −1-−1 | unknown | −1 | −1 | unknown | LGALS1/BC020675 |
| | | | | unknown | −1-−1 | unknown | −1 | −1 | unknown | NM_002305 |
| | | | | 22 | 36395660-36396091 | + | 36396142 | 51 | downstream | LGALS1/BC020675 |
| | | | | 22 | 36395660-36396091 | + | 36396142 | 51 | downstream | NM_002305 |
| | | | | 22 | 36395660-36396091 | + | 36397492 | 1401 | downstream | LGALS1/BT006775 |
| | | | | 22 | 36395660-36396091 | + | 36400163 | 4072 | downstream | LGALS1/S44881 |
| 105 | 62:A05 | 126 | 126 | unknown | −1-−1 | unknown | −1 | −1 | unknown | NM_173479 |
| | | | | x | 108103520-108103589 | + | 108103493 | 27 | downstream | NM_173479 |
| 106 | 19:F04 | 508 | 508 | unknown | −1-−1 | unknown | −1 | −1 | unknown | LYRIC/BC045642 |
| | | | | unknown | −1-−1 | unknown | −1 | −1 | unknown | NM_178812 |
| | | | | 8 | 98724880-98725388 | + | 98725582 | 194 | downstream | LYRIC/BC045642 |
| | | | | 8 | 98724880-98725388 | + | 98725582 | 194 | downstream | NM_178812 |
| | | | | 8 | 98724880-98725388 | + | 98725683 | 295 | downstream | LYRIC/BC009324 |
| 107 | 94:A12 | 855 | 855 | unknown | −1-−1 | unknown | −1 | −1 | unknown | MGC4549/BC007516 |
| | | | | unknown | −1-−1 | unknown | −1 | −1 | unknown | NM_032377 |
| | | | | 19 | 11530687-11531542 | − | 11526181 | 4506 | upstream | MGC4549/BC007516 |
| | | | | 19 | 11530687-11531542 | − | 11526181 | 4506 | upstream | NM_032377 |
| 108 | 7:G09 | 813 | 851 | unknown | −1-−1 | unknown | −1 | −1 | unknown | SPTLC2/AB011098 |
| | | | | unknown | −1-−1 | unknown | −1 | −1 | unknown | NM_004863 |
| | | | | 14 | 77152192-77153089 | − | 77152863 | 0 | within | SPTLC2/AB011098 |
| | | | | 14 | 77152192-77153089 | − | 77152863 | 0 | within | NM_004863 |
| 109 | 124:C09 | 556 | 556 | unknown | −1-−1 | unknown | −1 | −1 | unknown | MOV10/AK023297 |
| | | | | 1 | 112928785-112929341 | + | 112929357 | 16 | downstream | MOV10/AK023297 |
| | | | | 1 | 112928785-112929341 | + | 112929361 | 20 | downstream | MOV10/AB046851 |
| | | | | 1 | 112928785-112929341 | + | 112929383 | 42 | downstream | MOV10/AK074174 |
| | | | | 1 | 112928785-112929341 | + | 112929508 | 167 | downstream | NM_020963 |
| 110 | 65:C06 | 456 | 538 | unknown | −1-−1 | unknown | −1 | −1 | unknown | BC028721 |
| | | | | 19 | 14950801-14951928 | − | 14951469 | 0 | within | BC028721 |
| 111 | 33:B08 | 198 | 198 | unknown | −1-−1 | unknown | −1 | −1 | unknown | SIAT8A/AY569975 |
| | | | | 12 | 22379750-22379948 | − | 22378439 | 1311 | upstream | SIAT8A/AY569975 |
| | | | | 12 | 22379750-22379948 | − | 22378872 | 878 | upstream | BC046158 |
| | | | | 12 | 22379750-22379948 | − | 22378915 | 835 | upstream | SIAT8A/X77922 |
| | | | | 12 | 22379750-22379948 | − | 22378915 | 835 | upstream | NM_003034 |
| 112 | 33:B10 | 296 | 295 | unknown | −1-−1 | unknown | −1 | −1 | unknown | CDK6/BC052264 |
| | | | | unknown | −1-−1 | unknown | −1 | −1 | unknown | NM_001259 |
| | | | | 7 | 92110935-92111230 | − | 92107863 | 3072 | upstream | CDK6/BC052264 |
| | | | | 7 | 92110935-92111230 | − | 92107863 | 3072 | upstream | NM_001259 |
| 113 | 109:B01 | 138 | 8 | unknown | −1-−1 | unknown | −1 | −1 | unknown | ZNF34/AL833814 |
| | | | | 8 | 145987898-145988036 | − | 145983514 | 4384 | upstream | ZNF34/AL833814 |
| | | | | 8 | 145987898-145988036 | − | 145988105 | 69 | upstream | RPL8/BC000047 |
| | | | | 8 | 145987898-145988036 | − | 145988533 | 497 | upstream | NM_033301 |
| | | | | 8 | 145987898-145988036 | − | 145988570 | 534 | upstream | RPL8/BC000077 |
| | | | | 8 | 145987898-145988036 | − | 145988572 | 536 | upstream | NM_000973 |
| 114 | 15:F07 | 547 | 547 | unknown | −1-−1 | unknown | −1 | −1 | unknown | C19orf7/AB028987 |
| | | | | 19 | 52308085-52308571 | − | 52308849 | 278 | upstream | C19orf7/AB028987 |
| 115 | 10:A08 | 547 | 547 | unknown | −1-−1 | unknown | −1 | −1 | unknown | C19orf7/AB028987 |
| | | | | 19 | 52308123-52308632 | − | 52308849 | 217 | upstream | C19orf7/AB028987 |
| 116 | 121:E08 | 203 | 203 | unknown | −1-−1 | unknown | −1 | −1 | unknown | NM_172316 |
| | | | | 15 | 35183098-35183301 | − | 35178889 | 4209 | upstream | NM_172316 |
| | | | | 15 | 35183098-35183301 | − | 35179996 | 3102 | upstream | NM_020149 |
| | | | | 15 | 35183098-35183301 | − | 35179996 | 3102 | upstream | NM_170674 |
| | | | | 15 | 35183098-35183301 | − | 35179996 | 3102 | upstream | NM_170675 |
| | | | | 15 | 35183098-35183301 | − | 35179996 | 3102 | upstream | NM_170676 |
| | | | | 15 | 35183098-35183301 | − | 35179996 | 3102 | upstream | NM_170677 |
| | | | | 15 | 35183098-35183301 | − | 35180673 | 2425 | upstream | MEIS2/BC050431 |
| | | | | 15 | 35183098-35183301 | − | 35180792 | 2306 | upstream | NM_002399 |
| | | | | 15 | 35183098-35183301 | − | 35180796 | 2302 | upstream | MEIS2/BC001844 |
| 117 | 23:B07 | 601 | 600 | unknown | −1-−1 | unknown | −1 | −1 | unknown | NM_002624 |
| | | | | unknown | −1-−1 | unknown | −1 | −1 | unknown | NM_145896 |
| | | | | unknown | −1-−1 | unknown | −1 | −1 | unknown | NM_145897 |
| | | | | 12 | 51975636-51976234 | + | 51975583 | 53 | downstream | NM_002624 |
| | | | | 12 | 51975636-51976234 | + | 51975583 | 53 | downstream | NM_145896 |
| | | | | 12 | 51975636-51976234 | + | 51975583 | 53 | downstream | NM_145897 |
| | | | | 12 | 51975636-51976234 | + | 51975592 | 44 | downstream | PFDN5/AB055803 |
| | | | | 12 | 51975636-51976234 | + | 51975592 | 44 | downstream | PFDN5/AB055804 |
| | | | | 12 | 51975636-51976234 | + | 51975592 | 44 | downstream | PFDN5/AB055805 |
| | | | | 12 | 51975636-51976234 | + | 51975602 | 34 | downstream | PFDN5/D89667 |
| | | | | 12 | 51975636-51976234 | + | 51975618 | 18 | downstream | BT007195 |
| | | | | 12 | 51975636-51976234 | + | 51979768 | 3534 | downstream | C12orf10/AF289485 |
| | | | | 12 | 51975636-51976234 | + | 51979768 | 3534 | downstream | NM_021640 |
| | | | | 12 | 51975636-51976234 | + | 51979800 | 3566 | downstream | C12orf10/BC013956 |
| | | | | 12 | 51975636-51976234 | + | 51980028 | 3794 | downstream | C12orf10/BC028904 |
| 118 | 9:G03 | 910 | 879 | unknown | −1-−1 | unknown | −1 | −1 | unknown | PRKY/Y15801 |
| | | | | y | 7183785-7184487 | + | 7185373 | 886 | downstream | PRKY/Y15801 |
| | | | | y | 7183785-7184487 | + | 7185374 | 887 | downstream | NM_002760 |
| | | | | x | 3626413-3626807 | − | 3625010 | 1403 | upstream | PRKX/X85545 |
| | | | | x | 3626413-3626807 | − | 3625010 | 1403 | upstream | NM_005044 |
| 119 | 9:H03 | 656 | 656 | unknown | −1-−1 | unknown | −1 | −1 | unknown | DKFZp667B1218/BC034978 |
| | | | | unknown | −1-−1 | unknown | −1 | −1 | unknown | NM_177966 |
| | | | | 3 | 57516974-57517570 | + | 57517042 | 0 | within | DKFZp667B1218/BC034978 |
| | | | | 3 | 57516974-57517570 | + | 57517042 | 0 | within | NM_177966 |
| | | | | 3 | 57516974-57517570 | + | 57517064 | 0 | within | DKFZp667B1218/AK074423 |
| | | | | 3 | 57516974-57517570 | + | 57517509 | 0 | within | DKFZp667B1218/AL831824 |
| 120 | 15:H08 | 545 | 545 | unknown | −1-−1 | unknown | −1 | −1 | unknown | FCMD/AB008226 |
| | | | | unknown | −1-−1 | unknown | −1 | −1 | unknown | NM_006731 |
| | | | | 9 | 105399739-105400256 | + | 105399978 | 0 | within | FCMD/AB008226 |
| | | | | 9 | 105399739-105400256 | + | 105399978 | 0 | within | NM_006731 |
| 121 | 14:E11 | 618 | 612 | unknown | −1-−1 | unknown | −1 | −1 | unknown | NM_020311 |
| | | | | 2 | 237258709-237259324 | + | 237260442 | 1118 | downstream | NM_020311 |
| 122 | 16:B10 | 419 | 419 | unknown | −1-−1 | unknown | −1 | −1 | unknown | C3F/BC065194 |
| | | | | 12 | 6995485-6995904 | − | 6996034 | 130 | upstream | C3F/BC065194 |
| | | | | 12 | 6995485-6995904 | − | 6996103 | 199 | upstream | NM_005768 |
| 123 | 16:F10 | 322 | 322 | unknown | −1-−1 | unknown | −1 | −1 | unknown | AF091072 |
| | | | | 19 | 5742063-5742385 | − | 5738675 | 3388 | upstream | AF091072 |
| | | | | 19 | 5742063-5742385 | − | 5741335 | 728 | upstream | LOC56931/BC008362 |
| | | | | 19 | 5742063-5742385 | − | 5742102 | 0 | within | LOC56931/AL365411 |
| | | | | 19 | 5742063-5742385 | − | 5742190 | 0 | within | LOC56931/BC009973 |
| | | | | 19 | 5742063-5742385 | − | 5742190 | 0 | within | NM_020175 |
| | | | | 19 | 5742063-5742385 | − | 5742217 | 0 | within | LOC56931/BC004549 |

TABLE 15 shows, according to particular preferred aspects, markers for CLL, FL and MCL as identified by methylation hybridization as described in the EXAMPLES herein.

| | | T7 | M13 | | | |
|---|---|---|---|---|---|---|
| | | Sequence | Sequence | Chromosome | | |
| No. | Clone ID | Length | Length | Aligned | Alignment Address | Strand |
| 1 | FJ#69B12 | 663 | 72 | x | 67501692-67502259 | + |
| | | | | x | 67501692-67502259 | + |
| 2 | FJ#72A12 | 738 | 874 | 20 | 33793376-33794301 | − |
| | | | | 20 | 33793376-33794301 | − |
| | | | | 20 | 33793376-33794301 | − |
| | | | | 20 | 33793376-33794301 | − |
| | | | | 20 | 33793376-33794301 | − |
| | | | | 20 | 33793376-33794301 | − |
| | | | | 20 | 33793376-33794301 | − |
| | | | | 20 | 33793376-33794301 | − |
| 3 | FJ#26C4 | 225 | 686 | 12 | 10765801-10766442 | − |
| | | | | 12 | 10765801-10766442 | − |
| | | | | 12 | 10765801-10766442 | − |
| 4 | FJ#25G8 | 513 | 521 | 2 | 142722167-142722313 | − |
| | | | | 2 | 142722167-142722313 | − |
| | | | | 2 | 142722167-142722313 | − |
| 5 | FJ#9E10 | 501 | 501 | 3 | 174785313-174785814 | + |
| 6 | FJ#45F11 | 906 | 543 | 3 | 57557703-57558663 | − |
| | | | | 3 | 57557703-57558663 | − |
| 7 | FJ#63F2 | 471 | 550 | 2 | 104927795-104928343 | + |
| 8 | FJ#40H11 | 705 | 705 | 22 | 38039861-38040545 | − |
| | | | | 22 | 38039861-38040545 | − |
| | | | | 22 | 38039861-38040545 | − |
| | | | | 22 | 38039861-38040545 | − |
| | | | | 22 | 38039861-38040545 | − |
| 9 | FJ#40D1 | 767 | 764 | 20 | 29790458-29791120 | + |
| | | | | 20 | 29790458-29791120 | + |
| | | | | 20 | 29790458-29791120 | + |
| 10 | FJ#3B4 | 475 | 831 | 19 | 17391327-17391555 | + |
| | | | | 19 | 17391327-17391555 | + |
| 11 | FJ#27D1 | 738 | 559 | 12 | 52675489-52676226 | + |
| | | | | 12 | 52675489-52676226 | + |
| | | | | 12 | 52675489-52676226 | + |
| 12 | FJ#54E1 | 301 | 971 | 17 | 631917-632747 | + |
| | | | | 17 | 631917-632747 | + |
| | | | | 17 | 631917-632747 | − |
| | | | | 17 | 631917-632747 | − |
| | | | | 17 | 631917-632747 | − |
| | | | | 17 | 631917-632747 | − |
| 13 | FJ#46B3 | 516 | 514 | 6 | 26307668-26308182 | + |
| | | | | 6 | 26307668-26308182 | + |
| | | | | 6 | 26307668-26308182 | − |
| | | | | 6 | 26307668-26308182 | − |
| | | | | 6 | 26307668-26308182 | − |
| | | | | 6 | 27969432-27969482 | + |
| | | | | 6 | 27969432-27969482 | − |
| | | | | 6 | 27969432-27969482 | − |
| 14 | FJ#46G1 | 442 | 350 | 9 | 123858628-123858970 | + |
| 15 | FJ#27B4 | 855 | 823 | 6 | 28327249-28328106 | − |
| | | | | 6 | 28327249-28328106 | − |
| | | | | 6 | 28327249-28328106 | − |
| | | | | 6 | 28435561-28435779 | + |
| | | | | 7 | 98748573-98748626 | + |
| | | | | 7 | 98748573-98748626 | + |
| | | | | 7 | 98748573-98748626 | + |
| | | | | 7 | 98748573-98748626 | + |
| 16 | FJ#39H10 | 788 | 765 | 22 | 30474203-30474989 | + |
| | | | | 22 | 30474203-30474989 | + |
| 17 | FJ#41D7 | 654 | 653 | 1 | 117313967-117314595 | + |
| | | | | 1 | 17313967-117314595 | + |
| | | | | 1 | 17313967-117314595 | + |
| 18 | FJ#40F9 | 919 | 835 | 2 | 69880005-69881175 | + |
| | | | | 2 | 69880005-69881175 | + |
| 19 | FJ#9F12 | 0 | 854 | unknown | −1-−1 | unknown |
| 20 | FJ#73B9 | 732 | 732 | 4 | 88285240-88285972 | + |
| | | | | 4 | 88285240-88285972 | + |
| 21 | FJ#46A2 | 788 | 666 | 16 | 23597626-23598702 | + |
| | | | | 16 | 23597626-23598702 | + |
| 22 | FJ#11H11 | 764 | 765 | unknown | −1-−1 | unknown |
| | | | | 7 | 142600276-142601041 | + |
| 23 | FJ#25A2 | 521 | 523 | 2 | 231551970-231552160 | + |
| | | | | 2 | 231551970-231552160 | + |
| | | | | 2 | 231551970-231552160 | + |
| | | | | 2 | 231551970-231552160 | + |
| | | | | 2 | 231551970-231552160 | + |
| | | | | 2 | 231551970-231552160 | + |
| 24 | FJ#47D2 | 283 | 282 | 17 | 34562266-34562548 | − |
| | | | | 17 | 34562266-34562548 | − |
| 25 | FJ#3B12 | 523 | 849 | unknown | −1-−1 | unknown |
| | | | | 2 | 201502252-201503188 | + |
| | | | | 2 | 201502252-201503188 | + |
| | | | | 2 | 201502252-201503188 | + |
| | | | | 2 | 201502252-201503188 | + |
| 26 | FJ#40F1 | 729 | 729 | unknown | −1-−1 | unknown |
| 27 | FJ#21B2 | 857 | 948 | 19 | 8457871-8459154 | + |
| | | | | 19 | 8457871-8459154 | + |
| 28 | FJ#43E9 | 588 | 432 | 11 | 71317490-71318078 | + |
| | | | | 11 | 71317490-71318078 | + |
| | | | | 11 | 71317490-71318078 | + |
| | | | | 11 | 71317490-71318078 | + |
| | | | | 11 | 71317490-71318078 | + |
| 29 | FJ#33D12 | 783 | 783 | 10 | 94322787-94323571 | − |
| | | | | 10 | 94322787-94323571 | − |
| 30 | FJ#46C1 | 714 | 502 | 9 | 27518208-27518960 | + |
| | | | | 9 | 27518208-27518960 | + |
| | | | | 9 | 27518208-27518960 | − |
| | | | | 9 | 27518208-27518960 | − |
| 31 | FJ#46C3 | 321 | 321 | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| 32 | FJ#53G12 | 814 | 832 | 5 | 113724888-113725712 | + |
| | | | | 5 | 113724888-113725712 | + |
| 33 | FJ#46C2 | 882 | 556 | 12 | 56452255-56453288 | + |
| | | | | 12 | 56452255-56453288 | + |
| | | | | 12 | 56452255-56453288 | + |
| | | | | 12 | 56452255-56453288 | + |
| | | | | 12 | 56452255-56453288 | + |
| | | | | 12 | 56452255-56453288 | − |
| | | | | 12 | 56452255-56453288 | − |
| | | | | 12 | 56452255-56453288 | − |
| | | | | 12 | 56452255-56453288 | − |
| 34 | FJ#25G3 | 513 | 513 | 2 | 142721862-142722346 | − |
| | | | | 2 | 142721862-142722346 | − |
| | | | | 2 | 142721862-142722346 | − |
| 35 | FJ#23F12 | 844 | 843 | 16 | 27468205-27469402 | + |
| | | | | 16 | 27468205-27469402 | − |
| | | | | 16 | 27468205-27469402 | − |
| | | | | 16 | 27468205-27469402 | − |
| 36 | FJ#21B1 | 304 | 306 | 15 | 67531330-67531636 | + |
| | | | | 15 | 67531330-67531636 | + |
| | | | | 15 | 67531330-67531636 | + |
| | | | | 15 | 67531330-67531636 | + |
| 37 | FJ#32F2 | 622 | 618 | 4 | 85773754-85774366 | − |
| 38 | FJ#23A10 | 644 | 644 | unknown | −1-−1 | unknown |
| | | | | 11 | 65525871-65526496 | + |
| | | | | 11 | 65525871-65526496 | + |
| | | | | 11 | 65525871-65526496 | − |
| | | | | 11 | 65525871-65526496 | − |
| 39 | FJ#41H8 | 416 | 416 | 6 | 34833073-34833489 | + |
| | | | | 6 | 34833073-34833489 | + |
| 40 | FJ#54F9 | 0 | 775 | 8 | 104102136-104102863 | + |
| | | | | 8 | 104102136-104102863 | + |

| | | Distance | | |
|---|---|---|---|---|
| No. | TSS | to TSS | Direction | Gene/Assession Number |
| 1 | 67501906 | 0 | within | MGC21416/BC012469 |
| | 67501906 | 0 | within | NM_173834 |
| 2 | 33793287 | 89 | upstream | RNPC2/L10911 |
| | 33793584 | 0 | within | RNPC2/BX640714 |
| | 33793587 | 0 | within | RNPC2/BX640812 |
| | 33793607 | 0 | within | NM_004902 |
| | 33793607 | 0 | within | NM_184234 |
| | 33793607 | 0 | within | NM_184237 |
| | 33793607 | 0 | within | NM_184241 |
| | 33793607 | 0 | within | NM_184244 |
| 3 | 10767171 | 729 | upstream | CSDA/BC021926 |
| | 10767171 | 729 | upstream | NM_003651 |
| | 10767173 | 731 | upstream | CSDA/BC009744 |
| 4 | 142722306 | 0 | within | LRP1B/AK054663 |
| | 142723002 | 689 | upstream | LRP1B/AF176832 |
| | 142723002 | 689 | upstream | NM_018557 |
| 5 | 174785178 | 135 | downstream | NLGN1/AB028993 |
| 6 | 57558118 | 0 | within | NM_001660 |
| | 57558151 | 0 | within | ARF4/BC016325 |
| 7 | 104930486 | 2143 | downstream | POU3F3/NM_006236 |
| 8 | 38035470 | 4391 | upstream | AY320405 |
| | 38037997 | 1864 | upstream | RPL3/BC004323 |
| | 38039014 | 847 | upstream | RPL3/BC022790 |
| | 38040115 | 0 | within | RPL3/BC012786 |
| | 38040128 | 0 | within | NM_000967 |
| 9 | 29790564 | 0 | within | NM_012112 |
| | 29790798 | 0 | within | TPX2/AF287265 |
| | 29790805 | 0 | within | TPX2/BC020207 |
| 10 | 17391911 | 356 | downstream | LOC93343/BC011840 |
| | 17391911 | 356 | downstream | NM_138401 |
| 11 | 52680143 | 3917 | downstream | NM_006897 |
| | 52680169 | 3943 | downstream | HOXC9/BC053894 |
| | 52680241 | 4015 | downstream | HOXC9/BC032769 |
| 12 | 632262 | 0 | within | FLJ10581/AF177344 |
| | 632262 | 0 | within | NM_018146 |
| | 632269 | 0 | within | CGI-150/AF177342 |
| | 632284 | 0 | within | CGI-150/AK001488 |
| | 632297 | 0 | within | CGI-150/AF177343 |
| | 632297 | 0 | within | NM_016080 |
| 13 | 26307765 | 0 | within | HIST1H2BF/NM_003522 |
| | 26312851 | 4669 | downstream | HIST1H4E/NM_003545 |
| | 26307419 | 249 | upstream | HIST1H3D/BC031333 |
| | 26307443 | 225 | upstream | NM_003530 |
| | 26307450 | 218 | upstream | HIST1H2AD/NM_021065 |
| | 27969181 | 251 | downstream | HIST1H2BO/NM_003527 |
| | 27966549 | 2883 | upstream | HIST1H3J/NM_003535 |
| | 27968942 | 490 | upstream | HIST1H2AM/NM_003514 |
| 14 | 123854215 | 4413 | downstream | LHX2/AF124735 |
| 15 | 28327981 | 0 | within | ZNF307/BC014031 |
| | 28327981 | 0 | within | NM_019110 |
| | 28328021 | 0 | within | ZNF307/AK056698 |
| | 28435342 | 219 | downstream | ZNF306/BT007427 |
| | 98746946 | 1627 | downstream | NM_145102 |
| | 98746948 | 1625 | downstream | ZFP95/BC030790 |
| | 98747254 | 1319 | downstream | NM_014569 |
| | 98747282 | 1291 | downstream | ZFP95/AB023232 |
| 16 | 30474622 | 0 | within | BC057797 |
| | 30475402 | 413 | downstream | AB014545 |
| 17 | 117314990 | 395 | downstream | NM_003594 |
| | 117314996 | 401 | downstream | TTF2/AF080255 |
| | 117315006 | 411 | downstream | TTF2/BC030058 |
| 18 | 69880756 | 0 | within | BC063672 |
| | 69880931 | 0 | within | NM_001153 |
| 19 | −1 | −1 | unknown | MUC4 |
| 20 | 88285318 | 0 | within | MLLT2/L13773 |
| | 88285318 | 0 | within | NM_005935 |
| 21 | 23597701 | 0 | within | PLK1/BC002369 |
| | 23597701 | 0 | within | NM_005030 |
| 22 | −1 | −1 | unknown | ZYX |
| | 142596206 | 4070 | downstream | ZYX/U15158 |
| 23 | 231555132 | 2972 | downstream | ITM2C/AF271781 |
| | 231555132 | 2972 | downstream | NM_030926 |
| | 231555150 | 2990 | downstream | ITM2C/AK090975 |
| | 231555179 | 3019 | downstream | ITM2C/BC050668 |
| | 231555187 | 3027 | downstream | ITM2C/BC002424 |
| | 231555199 | 3039 | downstream | ITM2C/BC025742 |
| 24 | 34561298 | 968 | upstream | PLXDC1/AF378753 |
| | 34561298 | 968 | upstream | NM_020405 |
| 25 | −1 | −1 | unknown | CAV1 |
| | 201502117 | 135 | downstream | Z70221 |
| | 201502150 | 102 | downstream | BZW1/D13630 |
| | 201502152 | 100 | downstream | BZW1/BC001804 |
| | 201502152 | 100 | downstream | NM_014670 |
| 26 | −1 | −1 | unknown | GPC3 |
| 27 | 8456661 | 1210 | downstream | HNRPM/BC064588 |
| | 8458765 | 0 | within | AL713781 |
| 28 | 71317730 | 0 | within | NM_018320 |
| | 71317730 | 0 | within | NM_194452 |
| | 71317730 | 0 | within | NM_194453 |
| | 71317749 | 0 | within | RNF121/AK023139 |
| | 71317757 | 0 | within | RNF121/BC009672 |
| 29 | 94323813 | 242 | upstream | IDE/M21188 |
| | 94323813 | 242 | upstream | NM_004969 |
| 30 | 27514311 | 3897 | downstream | IFNK/AF146759 |
| | 27514311 | 3897 | downstream | NM_020124 |
| | 27519744 | 784 | upstream | MOBKL2B/AL832572 |
| | 27519850 | 890 | upstream | NM_024761 |
| 31 | 206372067 | 4347 | downstream | NRP2/BC009222 |
| | 206372729 | 3685 | downstream | NM_201264 |
| | 206372729 | 3685 | downstream | NM_018534 |
| | 206372729 | 3685 | downstream | NM_201267 |
| | 206372729 | 3685 | downstream | NM_003872 |
| | 206372729 | 3685 | downstream | NM_201266 |
| | 206372729 | 3685 | downstream | NM_201279 |
| | 206373520 | 2894 | downstream | NRP2/AF016098 |
| | 206373520 | 2894 | downstream | NRP2/AF280544 |
| | 206373520 | 2894 | downstream | NRP2/AF280545 |
| | 206373520 | 2894 | downstream | NRP2/AF280546 |
| 32 | 113725914 | 202 | downstream | KCNN2/AF239613 |
| | 113725914 | 202 | downstream | NM_021614 |
| 33 | 56452649 | 0 | within | DKFZP586D0919/BC016395 |
| | 56452649 | 0 | within | NM_015433 |
| | 56452649 | 0 | within | NM_206914 |
| | 56452705 | 0 | within | DKFZP586D0919/AK024983 |
| | 56452727 | 0 | within | DKFZP586D0919/AL050100 |
| | 56452152 | 103 | upstream | METTL1/BC000550 |
| | 56452181 | 74 | upstream | NM_023032 |
| | 56452181 | 74 | upstream | NM_023033 |
| | 56452522 | 0 | within | NM_005371 |
| 34 | 142722306 | 0 | within | LRP1B/AK054663 |
| | 142723002 | 656 | upstream | LRP1B/AF176832 |
| | 142723002 | 656 | upstream | NM_018557 |
| 35 | 27468970 | 0 | within | AB011128 |
| | 27464346 | 3859 | upstream | GTF3C1/U06485 |
| | 27468775 | 0 | within | GTF3C1/U02619 |
| | 27468775 | 0 | within | NM_001520 |
| 36 | 67532212 | 576 | downstream | NM_001003 |
| | 67532212 | 576 | downstream | NM_213725 |
| | 67532225 | 589 | downstream | RPLP1/AY303789 |
| | 67532229 | 593 | downstream | RPLP1/BC003369 |
| 37 | 85776566 | 2200 | upstream | NKX6-1/NM_006168 |
| 38 | −1 | −1 | unknown | BANF1 |
| | 65526125 | 0 | within | BANF1/AF068235 |
| | 65526125 | 0 | within | NM_003860 |
| | 65526154 | 0 | within | MGC11102/AK094129 |
| | 65526154 | 0 | within | NM_032325 |
| 39 | 34833289 | 0 | within | SNRPC/X12517 |
| | 34833289 | 0 | within | NM_003093 |
| 40 | 104102486 | 0 | within | NM_001695 |
| | 104102501 | 0 | within | ATP6V1C1/BC010960 |

TABLE 16 shows, according to particular preferred aspects, markers for CLL, FL and MCL as identified by methylation hybridization as described in the EXAMPLES herein.

| | | T7 | M13 | | | |
|---|---|---|---|---|---|---|
| | | Sequence | Sequence | Chromosome | | |
| No. | Clone ID | Length | Length | Aligned | Alignment Address | Strand |
| 1 | FJ#38F12 | 494 | 273 | 12 | 99097125-99097359 | + |
| | | | | 12 | 99097125-99097359 | + |
| | | | | 12 | 99097125-99097359 | + |
| | | | | 12 | 99097125-99097359 | + |
| 2 | FJ#3A2 | 761 | 613 | 1 | 40392373-40393477 | + |
| | | | | 1 | 40392373-40393477 | + |
| 3 | FJ#21B1 | 304 | 306 | 15 | 67531330-67531636 | + |
| | | | | 15 | 67531330-67531636 | + |
| | | | | 15 | 67531330-67531636 | + |
| | | | | 15 | 67531330-67531636 | + |
| 4 | FJ#32F2 | 622 | 618 | 4 | 85773754-85774366 | − |
| 5 | FJ#26C4 | 225 | 686 | 12 | 10765801-10766442 | − |
| | | | | 12 | 10765801-10766442 | − |
| | | | | 12 | 10765801-10766442 | − |
| 6 | FJ#25G8 | 513 | 521 | 2 | 142722167-142722313 | − |
| | | | | 2 | 142722167-142722313 | − |
| | | | | 2 | 142722167-142722313 | − |
| 7 | FJ#10G5 | 485 | 485 | 21 | 18539685-18540170 | + |
| | | | | 21 | 18539685-18540170 | + |
| 8 | FJ#25F4 | 517 | 513 | 2 | 142721862-142722346 | − |
| | | | | 2 | 142721862-142722346 | − |
| | | | | 2 | 142721862-142722346 | − |
| 9 | FJ#9E10 | 501 | 501 | 3 | 174785313-174785814 | + |
| 10 | FJ#46C3 | 321 | 321 | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| | | | | 2 | 206376414-206376687 | + |
| 11 | FJ#46C1 | 714 | 502 | 9 | 27518208-27518960 | + |
| | | | | 9 | 27518208-27518960 | + |
| | | | | 9 | 27518208-27518960 | − |
| | | | | 9 | 27518208-27518960 | − |
| 12 | FJ#46G1 | 442 | 350 | 9 | 123858628-123858970 | + |
| 13 | FJ#51G7 | 220 | 216 | 12 | 29424773-29424851 | − |
| | | | | 12 | 29424773-29424851 | − |
| | | | | 12 | 29424773-29424851 | − |
| | | | | 12 | 29424773-29424851 | − |
| | | | | 12 | 29424773-29424851 | − |
| 14 | FJ#46A2 | 788 | 666 | 16 | 23597626-23598702 | + |
| | | | | 16 | 23597626-23598702 | + |
| 15 | FJ#46E6 | 767 | 567 | 6 | 13436298-13437047 | − |
| | | | | 6 | 13436298-13437047 | − |
| | | | | 6 | 13436298-13437047 | − |
| | | | | 6 | 13436298-13437047 | − |
| 16 | FJ#54E1 | 301 | 971 | 17 | 631917-632747 | + |
| | | | | 17 | 631917-632747 | + |
| | | | | 17 | 631917-632747 | − |
| | | | | 17 | 631917-632747 | − |
| | | | | 17 | 631917-632747 | − |
| | | | | 17 | 631917-632747 | − |
| 17 | FJ#46C2 | 882 | 556 | 12 | 56452255-56453288 | + |
| | | | | 12 | 56452255-56453288 | + |
| | | | | 12 | 56452255-56453288 | + |
| | | | | 12 | 56452255-56453288 | + |
| | | | | 12 | 56452255-56453288 | + |
| | | | | 12 | 56452255-56453288 | − |
| | | | | 12 | 56452255-56453288 | − |
| | | | | 12 | 56452255-56453288 | − |
| | | | | 12 | 56452255-56453288 | − |
| 18 | FJ#53G12 | 814 | 832 | 5 | 113724888-113725712 | + |
| | | | | 5 | 113724888-113725712 | + |
| 19 | FJ#41D7 | 654 | 653 | 1 | 117313967-117314595 | + |
| | | | | 1 | 117313967-117314595 | + |
| | | | | 1 | 117313967-117314595 | + |
| 20 | FJ#40D1 | 767 | 764 | 20 | 29790458-29791120 | + |
| | | | | 20 | 29790458-29791120 | + |
| | | | | 20 | 29790458-29791120 | + |
| 21 | FJ#27B4 | 855 | 823 | 6 | 28327249-28328106 | − |
| | | | | 6 | 28327249-28328106 | − |
| | | | | 6 | 28327249-28328106 | − |
| | | | | 6 | 28435561-28435779 | + |
| | | | | 7 | 98748573-98748626 | + |
| | | | | 7 | 98748573-98748626 | + |
| | | | | 7 | 98748573-98748626 | + |
| | | | | 7 | 98748573-98748626 | + |
| 22 | FJ#25A2 | 521 | 523 | 2 | 231551970-231552160 | + |
| | | | | 2 | 231551970-231552160 | + |
| | | | | 2 | 231551970-231552160 | + |
| | | | | 2 | 231551970-231552160 | + |
| | | | | 2 | 231551970-231552160 | + |
| | | | | 2 | 231551970-231552160 | + |
| 23 | FJ#54H6 | 575 | 576 | 6 | 26357798-26358374 | + |
| | | | | 6 | 26357798-26358374 | − |
| | | | | 6 | 26357798-26358374 | − |
| | | | | 6 | 26357798-26358374 | − |
| 24 | FJ#54C4 | 779 | 795 | 19 | 57223235-57223943 | − |
| | | | | 19 | 57223235-57223943 | − |
| | | | | 19 | 57223235-57223943 | − |
| 25 | FJ#47G6 | 855 | 627 | 1 | 208596523-208597879 | + |
| | | | | 1 | 208596523-208597879 | + |
| | | | | 1 | 208596523-208597879 | + |
| | | | | 1 | 208596523-208597879 | − |
| | | | | 1 | 208596523-208597879 | − |
| | | | | 1 | 208596523-208597879 | − |
| | | | | 1 | 208596523-208597879 | − |
| 26 | FJ#73B9 | 732 | 732 | 4 | 88285240-88285972 | + |
| | | | | 4 | 88285240-88285972 | + |

| | | Distance | | |
|---|---|---|---|---|
| No. | TSS | to TSS | Direction | Gene/Assession Number |
| 1 | 99097041 | 84 | downstream | ACTR6/BC015107 |
| | 99097041 | 84 | downstream | NM_022496 |
| | 99097051 | 74 | downstream | AF161399 |
| | 99097085 | 40 | downstream | AF175226 |
| 2 | 40392871 | 0 | within | NM_005857 |
| | 40393003 | 0 | within | ZMPSTE24/Y13834 |
| 3 | 67532212 | 576 | downstream | NM_001003 |
| | 67532212 | 576 | downstream | NM_213725 |
| | 67532225 | 589 | downstream | RPLP1/AY303789 |
| | 67532229 | 593 | downstream | RPLP1/BC003369 |
| 4 | 85776566 | 2200 | upstream | NKX6-1/NM_006168 |
| 5 | 10767171 | 729 | upstream | CSDA/BC021926 |
| | 10767171 | 729 | upstream | NM_003651 |
| | 10767173 | 731 | upstream | CSDA/BC009744 |
| 6 | 142722306 | 0 | within | LRP1B/AK054663 |
| | 142723002 | 689 | upstream | LRP1B/AF176832 |
| | 142723002 | 689 | upstream | NM_018557 |
| 7 | 18539020 | 665 | downstream | CHODL/AF257472 |
| | 18539020 | 665 | downstream | NM_024944 |
| 8 | 142722306 | 0 | within | LRP1B/AK054663 |
| | 142723002 | 656 | upstream | LRP1B/AF176832 |
| | 142723002 | 656 | upstream | NM_018557 |
| 9 | 174785178 | 135 | downstream | NLGN1/AB028993 |
| 10 | 206372067 | 4347 | downstream | NRP2/BC009222 |
| | 206372729 | 3685 | downstream | NM_201264 |
| | 206372729 | 3685 | downstream | NM_018534 |
| | 206372729 | 3685 | downstream | NM_201267 |
| | 206372729 | 3685 | downstream | NM_003872 |
| | 206372729 | 3685 | downstream | NM_201266 |
| | 206372729 | 3685 | downstream | NM_201279 |
| | 206373520 | 2894 | downstream | NRP2/AF016098 |
| | 206373520 | 2894 | downstream | NRP2/AF280544 |
| | 206373520 | 2894 | downstream | NRP2/AF280545 |
| | 206373520 | 2894 | downstream | NRP2/AF280546 |
| 11 | 27514311 | 3897 | downstream | IFNK/AF146759 |
| | 27514311 | 3897 | downstream | NM_020124 |
| | 27519744 | 784 | upstream | MOBKL2B/A3L832572 |
| | 27519850 | 890 | upstream | NM_024761 |
| 12 | 123854215 | 4413 | downstream | LHX2/AF124735 |
| 13 | 29425344 | 493 | upstream | PTX1/BC064522 |
| | 29425350 | 499 | upstream | PTX1/AK074520 |
| | 29425353 | 502 | upstream | PTX1/AL834128 |
| | 29425362 | 511 | upstream | PTX1/AF183410 |
| | 29425363 | 512 | upstream | NM_016570 |
| 14 | 23597701 | 0 | within | PLK1/BC002369 |
| | 23597701 | 0 | within | NM_005030 |
| 15 | 13436593 | 0 | within | NM_016495 |
| | 13436736 | 0 | within | TBC1D7/BC050465 |
| | 13436755 | 0 | within | TBC1D7/AK057228 |
| | 13436755 | 0 | within | TBC1D7/BC007054 |
| 16 | 632262 | 0 | within | FLJ10581/AF177344 |
| | 632262 | 0 | within | NM_018146 |
| | 632269 | 0 | within | CGI-150/AF177342 |
| | 632284 | 0 | within | CGI-150/AK001488 |
| | 632297 | 0 | within | CGI-150/AF177343 |
| | 632297 | 0 | within | NM_016080 |
| 17 | 56452649 | 0 | within | DKFZP586D0919/BC016395 |
| | 56452649 | 0 | within | NM_015433 |
| | 56452649 | 0 | within | NM_206914 |
| | 56452705 | 0 | within | DKFZP586D0919/AK024983 |
| | 56452727 | 0 | within | DKFZP586D0919/AL050100 |
| | 56452152 | 103 | upstream | METTL1/BC000550 |
| | 56452181 | 74 | upstream | NM_023032 |
| | 56452181 | 74 | upstream | NM_023033 |
| | 56452522 | 0 | within | NM_005371 |
| 18 | 113725914 | 202 | downstream | KCNN2/AF239613 |
| | 113725914 | 202 | downstream | NM_021614 |
| 19 | 117314990 | 395 | downstream | NM_003594 |
| | 117314996 | 401 | downstream | TTF2/AF080255 |
| | 117315006 | 411 | downstream | TTF2/BC030058 |
| 20 | 29790564 | 0 | within | NM_012112 |
| | 29790798 | 0 | within | TPX2/AF287265 |
| | 29790805 | 0 | within | TPX2/BC020207 |
| 21 | 28327981 | 0 | within | ZNF307/BC014031 |
| | 28327981 | 0 | within | NM_019110 |
| | 28328021 | 0 | within | ZNF307/AK056698 |
| | 28435342 | 219 | downstream | ZNF306/BT007427 |
| | 98746946 | 1627 | downstream | NM_145102 |
| | 98746948 | 1625 | downstream | ZFP95/BC030790 |
| | 98747254 | 1319 | downstream | NM_014569 |
| | 98747282 | 1291 | downstream | ZFP95/AB023232 |
| 22 | 231555132 | 2972 | downstream | ITM2C/AF271781 |
| | 231555132 | 2972 | downstream | NM_030926 |
| | 231555150 | 2990 | downstream | ITM2C/AK090975 |
| | 231555179 | 3019 | downstream | ITM2C/BC050668 |
| | 231555187 | 3027 | downstream | ITM2C/BC002424 |
| | 231555199 | 3039 | downstream | ITM2C/BC025742 |
| 23 | 26359857 | 1483 | downstream | HIST1H2BH/NM_003524 |
| | 26355184 | 2614 | upstream | HIST1H4G/NM_003547 |
| | 26358812 | 438 | upstream | HIST1H3H/BC067492 |
| | 26358814 | 440 | upstream | HIST1H3F/NM_021018 |
| 24 | 57223414 | 0 | within | ZNF614/BC004930 |
| | 57223429 | 0 | within | NM_025040 |
| | 57223476 | 0 | within | ZNF614/AK097156 |
| 25 | 208597525 | 0 | within | RAMP/AF195765 |
| | 208597525 | 0 | within | NM_016448 |
| | 208597582 | 0 | within | RAMP/AK027651 |
| | 208597257 | 0 | within | DKFZP434B168/AK001363 |
| | 208597273 | 0 | within | DKFZP434B168/BC020523 |
| | 208597279 | 0 | within | DKFZP434B168/AK001598 |
| | 208597279 | 0 | within | NM_015434 |
| 26 | 88285318 | 0 | within | MLLT2/L13773 |
| | 88285318 | 0 | within | NM_005935 |

TABLE 17 shows, according to particular preferred aspects, markers for FL and CLL as identified by methylation hybridization as described in the EXAMPLES herein.

| | | T7 | M13 | | | |
|---|---|---|---|---|---|---|
| | | Sequence | Sequence | Chromosome | | |
| No. | Clone ID | Length | Length | Aligned | Alignment Address | Strand |
| 1 | FJ#14H4 | 337 | 628 | 2 | 69781644-69781696 | − |
| | | | | 2 | 69781644-69781696 | − |
| | | | | 2 | 69781644-69781696 | − |
| 2 | FJ#47D2 | 283 | 282 | 17 | 34562266-34562548 | − |
| | | | | 17 | 34562266-34562548 | − |
| 3 | FJ#21B2 | 857 | 948 | 19 | 8457871-8459154 | + |
| | | | | 19 | 8457871-8459154 | + |
| 4 | FJ#3B12 | 523 | 849 | unknown | −1-−1 | unknown |
| | | | | 2 | 201502252-201503188 | + |
| | | | | 2 | 201502252-201503188 | + |
| | | | | 2 | 201502252-201503188 | + |
| | | | | 2 | 201502252-201503188 | + |
| 5 | FJ#23D6 | 879 | 826 | 5 | 43638478-43640026 | + |
| | | | | 5 | 43638478-43640026 | + |
| | | | | 5 | 43638478-43640026 | + |
| 6 | FJ#47A12 | 134 | 437 | 17 | 7322134-7323200 | − |
| 7 | FJ#23H7 | 916 | 918 | 1 | 168481258-168482112 | + |
| | | | | 1 | 168481258-168482112 | + |
| | | | | 1 | 168481258-168482112 | + |
| | | | | 1 | 168481258-168482112 | + |
| | | | | 1 | 168481258-168482112 | + |
| | | | | 1 | 168481258-168482112 | + |
| | | | | 1 | 168481258-168482112 | + |
| | | | | 1 | 168481258-168482112 | + |
| 8 | FJ#11H11 | 764 | 765 | unknown | −1-−1 | unknown |
| | | | | 7 | 142600276-142601041 | + |
| 9 | FJ#5D5 | 0 | 840 | 11 | 72680888-72681650 | + |
| 10 | FJ#29H4 | 419 | 421 | unknown | −1-−1 | unknown |
| 11 | FJ#15D4 | 282 | 772 | 1 | 198348160-198349144 | + |
| | | | | 1 | 198348160-198349144 | + |
| | | | | 1 | 198348160-198349144 | + |
| 12 | FJ#41H8 | 416 | 416 | 6 | 34833073-34833489 | + |
| | | | | 6 | 34833073-34833489 | + |
| 13 | FJ#63F2 | 471 | 550 | 2 | 104927795-104928343 | + |

| | | Distance | | |
|---|---|---|---|---|
| No. | TSS | to TSS | Direction | Gene/Assession Number |
| 1 | 69781863 | 167 | upstream | AAK1/BC002695 |
| | 69782500 | 804 | upstream | AAK1/AB028971 |
| | 69782500 | 804 | upstream | NM_014911 |
| 2 | 34561298 | 968 | upstream | PLXDC1/AF378753 |
| | 34561298 | 968 | upstream | NM_020405 |
| 3 | 8456661 | 1210 | downstream | HNRPM/BC064588 |
| | 8458765 | 0 | within | AL713781 |
| 4 | −1 | −1 | unknown | CAV1 |
| | 201502117 | 135 | downstream | Z70221 |
| | 201502150 | 102 | downstream | BZW1/D13630 |
| | 201502152 | 100 | downstream | BZW1/BC001804 |
| | 201502152 | 100 | downstream | NM_014670 |
| 5 | 43638581 | 0 | within | NM_012343 |
| | 43639063 | 0 | within | NNT/U40490 |
| | 43639063 | 0 | within | NM_182977 |
| 6 | 7323668 | 468 | upstream | ZBTB4/AB040971 |
| 7 | 168482478 | 366 | downstream | CGI-01/AK027621 |
| | 168482478 | 366 | downstream | NM_014955 |
| | 168482492 | 380 | downstream | CGI-01/AF132936 |
| | 168482492 | 380 | downstream | CGI-01/AL049669 |
| | 168482492 | 380 | downstream | NM_015935 |
| | 168482662 | 550 | downstream | CGI-01/AB020666 |
| | 168482663 | 551 | downstream | CGI-01/BC029083 |
| | 168484632 | 2520 | downstream | CGI-01/AK074552 |
| 8 | −1 | −1 | unknown | ZYX |
| | 142596206 | 4070 | downstream | ZYX/U15158 |
| 9 | 72685211 | 3561 | downstream | P2RY6/BT006771 |
| 10 | −1 | −1 | unknown | BLK |
| 11 | 198349106 | 0 | within | NAV1/AY043013 |
| | 198349106 | 0 | within | NM_020443 |
| | 198349575 | 431 | downstream | NAV1/AJ488101 |
| 12 | 34833289 | 0 | within | SNRPC/X12517 |
| | 34833289 | 0 | within | NM_003093 |
| 13 | 104930486 | 2143 | downstream | POU3F3/NM_006236 |

### Example 1

### DLC-1 Promoter Methylation was Demonstrated Herein, by Quantitative Analysis, to have Substantial Utility as a Differentiation-Related Biomarker of Non-Hodgkin's Lymphoma

### Example Overveiw

DNA methylation is an epigenetic modification that may lead to gene silencing of genes. This Example discloses real-time methylation-specific PCR analysis to examine promoter methylation of DLC-1 (deleted in liver cancer 1, a putative tumor suppressor) and its relationship to gene silencing in non-Hodgkin's lymphomas (NHL). Applicants previously used an Expressed CpG Island Sequence Tags (ECIST) microarray technique (11) and identified DLC-1 as a gene whose promoter is methylated in NHLs and results in gene silencing. As demonstrated herein, gene promoter methylation of DLC-1 occurred in a differentiation-related manner and has substantial utility as a biomarker in non-Hodgkin's Lymphoma (NHL).

Experimental Design. A quantitative real-time methylation specific PCR ASP) assay was developed for examining DLC-1 promoter methylation. DNA was examined from 13 non-neoplastic samples including 6 cases of benign follicular hyperplasia, 29 diffuse large. B cell lymphoma, 30 follicular lymphoma, 31 B-cell chronic lymphocytic leukemia/small lymphocytic lymphoma, and 13 mantle cell lymphoma patient samples. RNA was extracted from 5 normal controls, 9 DLBCL (diffuse large B-cell lymphoma), 10 FL (, follicular lymphoma), 11 CLL (chronic lymphocytic leukemia), and 9 MCL (mantle cell lymphoma) patient samples to determine expression of DLC-1.

Results. A high frequency of DLC-1 promoter hypermethylation was found to occur across different subtypes of NHLs, but not in cases of benign follicular hyperplasia (BFH). The expression of the DLC-1 mRNA was also shown to be down-regulated in NHLs compared to normal lymphoid cells, and this may be re-activated using therapies that modulate methylation and acetylation. More specifically, methylation of DLC-1 was observed in 77% (79 of 103) of NHL cases; including 62% (8 of 13) in MCL, 71% (22 of 31) in B-CLL/SLL (B-cell chronic lymphocytic leukemia/small lymphocytic lymphoma), 83% (25 of 30) in FL, and 83% (24 of 29) in DLBCL samples. Expression studies demonstrate down-regulation of DLC-1 in NHL compared to normal lymph nodes. When thresholded values of methylation of DLC-1 were examined, 100% specificity was obtained, with 77% sensitivity.

### Materials and Methods:

Clinical Samples. Tissue and blood samples were obtained from patients after diagnostic evaluation for suspected lymphoma at the Ellis Fischel Cancer Center (Columbia, Mo.) and the Holden Comprehensive Cancer Center (Iowa City, Iowa) in compliance with local Institutional Review Boards. DNA was isolated from a total of 126 specimens consisting of the following: 31 from patients with B-CLL/SLL, 30 from FL, 13 MCL, and 29 from DLBCL. In addition, 13 non-neoplastic samples were included. All cases of B-CLL/SLL had peripheral blood and bone marrow involvement, and thus were technically categorized as CLL. These are all referred to in this Example as B-CLL/SLL. Total RNA was extracted from 5 normal controls, 9 DLBCL, 10 FL, 11 CLL, and 9 MCL patient samples using the RNeasy kit (Qiagen, Valencia, Calif.).

Bisulfite treatment. Genomic DNA (0.2 to 1 μg) was treated with sodium bisulfite using the EZ DNA methylation kit according to the manufacturer's recommendations (Zymo Research, Orange, Calif.). This treatment converts unmethylated, but not methylated, cytosine to uracil in the genome. For the preparation of 100% methylated DNA, a blood DNA sample was treated with SssI methyltransferase that methylates all cytosine residues of CpG dinucleotides in the genome. Sodium bisulfite modification of the test and SssI-treated DNA samples were then performed as described above.

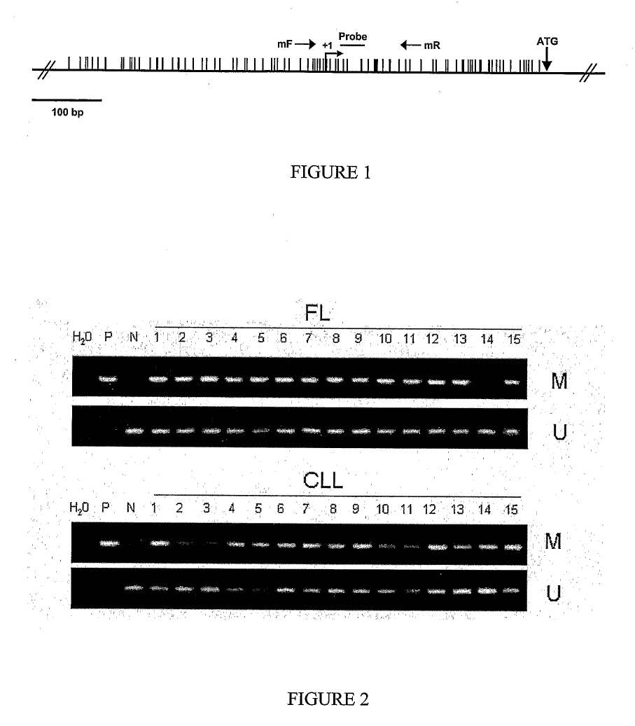

Standard and Quantitative Real Time MSP assay. FIG. 1 illustrates a portion of the DLC-1 promoter region of interest, the relative positions of CG dinucleotides, and the interrogation sites of the primers and probes used in this study. Aliquots of 100 ng of bisulfite treated DNA were used for each standard MSP assay. The published primers (M(+): 5′-CCC AAC GAA AAA ACC CGA CTA ACG-3′ (SEQ ID NO:1); M(−): 5′-TTT AAA GAT CGA AAC GAG GGA GCG-3′ (SEQ ID NO:2); U(+): 5′-AAA CCC AAC AAA AAA ACC CAA CTA ACA-3′ (SEQ ID NO:3); U(−): 5′-TTT TTT AAA GAT TGA AAT GAG CGA GTG-3′ (SEQ ID NO:4)) were used for the PCR amplification of methylated and unmethylated alleles in two separate reactions (12). Real-time MSP uses the same two amplification primers specific for methylated sequences and an additional, amplicon-specific, and fluorogenic hybridization probe (Probe: FAM/AAG TTC GTG AGT CGG CGT TTT TGA/BHQ_{—}1 (SEQ ID NO:5)) whose target sequence is located within the amplicon (FIG. 1). The probe was labeled with two fluorescent dyes, with FAM at the 5′-end and BHQ1 at the 3′-end. The primers/probe set for real-time MSP were synthesized by Integrated DNA Technologies (IDT; Coralville, Iowa). The bisulfite treated DNA was used for PCR amplification with appropriate reagents in QPCR mix (ABgene) as recommended by the manufacturer. The reaction was carried out in 40-45 cycles using a SmartCycler™ real-time PCR instrument (Cepheid).

Quantitative Real-Time RT PCR assay. Total RNA (2 μg) was pre-treated with DNase I to remove potential DNA contaminants and reverse-transcribed in the presence of SuperScript III™ reverse transcriptase (Invitrogen). The CDNA generated was used for PCR amplification with appropriate reagents in QPCR mix (ABgene) as recommended by the manufacturer. The Taqman™ probe and primer sets for real-time PCR were purchased from Applied Biosystem's Assay-on-Demand™ services. The reaction was carried out in 40-45 cycles using a SmartCycler™ real-time PCR instrument (Cepheid). All cDNA samples were synthesized in parallel. Separate parallel reactions were run for GAPDH CDNA using a series of diluted cDNA samples as templates to generate standardization curves. The mRNA levels were derived from the standardization curves and expressed as relative changes after normalization to those of GAPDH.

### Results:

Methylation status of DLC-1 CpG island in NHLs. A conventional MSP assay for DLC-1 was performed initially in 30 FL and B-CLL/SLL samples, primarily to confirm applicants' observations from ECISTs experiments. Representative MSP assay examples are illustrated in FIG. 2. In primary NHL samples, frequently consisting of a mixture of NHL cells and normal T- and B-cells, both methylated and unmethylated bands were present. The presence of unmethylated bands in all of the samples analyzed reflected the presence of residual nonmalignant cells and confirmed the integrity of the DNA in these samples.

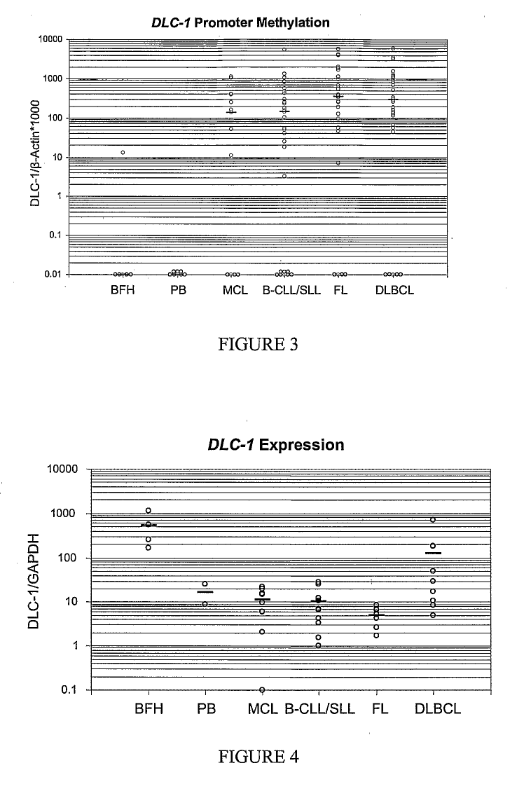

To quantify the methylation level in each sample, a probe was designed to include the CGI (CpG island) in the DLC-1 promoter (FIG. 1), in which hypermethylation is known to be correlated with a lack of gene expression in other tumors (13). The methylation analysis was expanded from all the samples described above to now include additional samples from patients with MCL, CLL, FL and DLBCL. The DLC-1 methylation frequencies were 71%, 62%, 83%, and 83%, respectively (FIG. 3). When this quantitative MSP method was compared to standard MSP, the consistency between the two methods was 100%. The relative methylation level of each sample, as measured by the ratio of DLC-1: β-actin×1000, varies among the 4 sub-classes of NHL studied. The median methylation level was 135 (range from 0 to 1099) for MCL, 141 (range from 0 to 5378) for B-CLL/SLL, 348 (range from 0 to 5683) for FL and 295 (range from 0 to 5912) for DLBCL (FIG. 3). Significantly, according to particular aspects of the present disclosure, both the frequency and relative level of methylation of DLC-1 seems to correlate with the putative stages of differentiation. The methylation level is relatively higher in germinal center-related NHLs such as FL and DLBCL (some cases are post-germinal center), as compared to MCL and B-CLL/SLL which are usually derived from pre- or post-germinal center cells. The increased methylation level was not attributable to the variability in tumor cell percentage. The proportion of tumor in all samples was >80% (range 74-97%) as determined by flow cytometry analysis, with no statistical difference between classes (p>0.05).

Loss of Expression of DLC-1 mRNA in NHLs. The mRNA expression level of DLC-1 was normalized against GAPDH as a housekeeping gene. As shown in FIG. 4, DLC-1 mRNA could be detected in lymph node samples of BFH and weakly in peripheral blood lymphocytes, suggesting a tissue or developmental stage-specific expression or possibly indicating other silencing mechanisms might exist in normal leukocytes other than methylation. DLC-1 mRNA was also weakly expressed in some cases of MCL, B-CLL/SLL, and FL, and somewhat stronger in DLBCL cases. When overall DLC-1 mRNA expression was compared between tumor and normal lymph node, its expression was lower in tumors. The reciprocal relationship between DLC-1 promoter methylation and its expression indicates, according to particular aspects of the present disclosure, that promoter methylation is a major mechanism for DLC-1 silencing in germinal center related NHLs.

Clinical Sensitivity and Specificity of Quantitative Methylation Specific PCR. The ideal disease biomarker test should exhibit high (100%) sensitivity and high (100%) specificity. These are quantifiable features of a defined, standardized biomarker/measurement system. In probabilistic terms, the ideal test should always detect the presence of NHL when present in the patient. This means the true positive rate (TPR) should be 100%. Few if any biomarker testing systems achieve 100% TPR, although this can be approached by refinement of technology and testing interpretation. TPR is synonymous with the widely used term clinical sensitivity. Furthermore, the ideal test should never signal the presence of NHL when it is absent. Thus, the false positive rate (FPR) should be 0%. Among clinical investigators, a more widely used test statistic, specificity, is formally identical to the quantity [1-FPR], thus with 0% FPR, the test would have 100% specificity.

The candidate biomarker methylated DLC-1 was measured on a binary scale positive or negative), and the TPR (the proportion of tumors that are biomarker positive) and the FPR (the proportion of BFH (benign follicular hyperplasia) samples that are biomarker positive), were used to summarize our ability to discriminate between NHL and BFH. Sensitivity (TPR) was calculated as (TP/(TP+FN)). In some cases, it has been found beneficial to set quantitative thresholds in analysis of methylation data (14). When we set an empirical threshold for positivity at 13 in FIG. 3, this resulted in a sensitivity of 61.5% (MCL), 71% (B-CLL/SLL), 83.9% (FL), and 82.8% (DLBCL), with overall NHL sensitivity 76.9%. Specificity (1-FPR) was 100%, since there were no FP results. If we did not set a threshold at 13, but included all cases with a level >0.1, then this resulted in a sensitivity of 69.2% (MCL), 74.2% (B-CLL/SLL), 86.7% (FL), and 82.8% (DLBCL), with overall NHL sensitivity 79.6%. Specificity (1-FPR) was now decreased to 92.3%, since there was 1 FP result in the control samples.

Intra- and Inter-Assay Variability. To reliably determine a quantitative cut-off for positivity, it is important to understand the limits of the variability of the assay system. In a first example, the intra-assay variability was examined. Three NHL cell lines, Daudi, Raji, and Granta 519, were used in this experiment. Five aliquots of each cell line (15 total samples) were bisulfite-treated and examined for quantitative levels of DLC-1 methylation within the same analytical run on the same day to represent the variation that might be expected within a single analytical run. The intra-assay co-efficient of variation (CV) ranged from 0.42%-0.64% when the variable was the qMSP cycle number (Cₜ). For the P-actin internal control, the range of the CV was 0.34%-0.74%. When the ratio of DLC-1 methylation: P-actin was plotted on the standard curve, the CV increased to a range of 9.92%-16.6%, dependent on the cell line. To test the inter-assay variability, 5 aliquots of each cell line were independently treated and assayed on 5 separate days to represent the variation that might occur between different analytical runs. The inter-assay CV for DLC-1 ranged from 0.82%-2.31% when the variable was the Cₜ. For the β-actin internal control, the range of CV was 0.70%-1.92%. When the ratio of DLC-1 methylation: β-actin was plotted on the standard curve, the CV increased to a range of 5.71%-17.5%, dependent on the cell line. Preferably, the intra- and inter-assay variability should be known when selecting thresholds and determining the level that can reliably considered positive versus negative, and particularly, according to particular aspects, where the assay is to be used for monitoring treatments where the upward or downward trend is important. The present CVs are consistent with those reported by others for RT-PCR or PCR assays (15, 16).

Plasma DLC-1 DNA Methylation. For a subset of 15 patients with B-CLL/SLL, FL, or DLBCL, paired tumor and plasma samples were available. Of these, 12/15 samples demonstrated concordant results, with 10/12 samples showing methylation in both the tumor and in plasma and 2/12 did not show methylation in either the tumor or in plasma. The 3 discordant samples all demonstrated tumor methylation, but none was detected in the plasma samples. Two of the 3 were from patients with localized stage I FL. Plasma was selected as the sample based on preliminary observations that serum may be less reliable for this purpose. Although both serum and plasma have been examined for total DNA levels, and generally higher levels are reported in serum (17, 18), Boddy, et al (19) (incorporated by reference herein) demonstrated that a 2-spin method of separating plasma from cellular elements provided the most consistency and reliability. This 2-spin method was also used in our study. For all these samples, we examined DLC-1 methylation not only in the tumor and in plasma, but also from buffy coat preparation of peripheral blood cells. In all cases of B-CLL/SLL and FL where methylation was present in the tumor, it was also present in buffy coat cells. However, in the case of DLBCL, methylation was present in the tumor and plasma, but not in buffy coat cells, which is consistent with the fact that most patients with DLBCL (other than those with advanced disease) do not have detectable circulating tumor cells in blood.

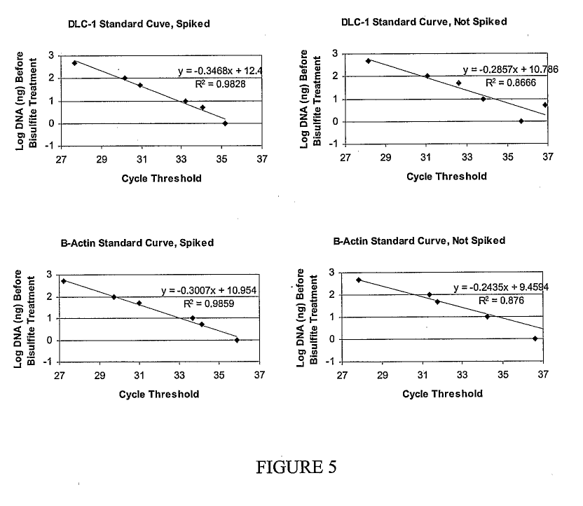

Assay Sensitivity of Detecting Low Levels of DNA Methylation. The assay sensitivity was determined by using various amounts of input DNA and, following treatment with sodium bisulfite, determining the least amount of methylated DLC-1 that could be detected in the assay. A standard curve was produced at multiple levels of input DNA from the lymphoma cell line RL ranging from 1 ng to 500 ng (FIG. 5). In these experiments, it was possible to reliably detect DLC-1 methylation from as little of 5 ng of DNA. Since >50 ng are typically obtained from 2 mL of plasma, the assay should not be limited by sensitivity.

Treatment of DNA with sodium bisulfite in known to result in destruction of as much as 90% of DNA (20). Thus, at very low levels of DNA, such as that found in plasma, it is quite possible to destroy enough that the assay becomes insensitive and quite variable. One potential way to improve this situation is to add carrier DNA to the extracted DNA prior to bisulfite treatment. The standard curve was compared at multiple levels of input DNA (ranging from 1 ng to 500 ng) in the presence and absence of 1 μg of salmon sperm DNA added prior to treatment. As shown in FIG. 5, at higher levels of input DNA (100 ng, 500 ng), there was no difference in the PCR Cₜ to detect a positive result. However, at the 10 ng level, the Cₜ value without added sperm DNA was 36.17, while in the presence of sperm DNA the Cₜ was lowered to 34.7, and at the 50 ng level, there was also a difference (Cₜ 34 versus 32.5). Overall, the slope regression was 0.9919 with, and 0.9734 without added DNA. There were no observable differences in Cₜ or slope of the regression line with the β-actin control.

Additional markers. According to addition aspects of the present invention, GSTP1, CDKN1A, RASSF1A and DAPK methylation markers have substantial utility as biomarkers of cancer (e.g., non-Hodgkin's Lymphoma).

### Example 2

### A CpG Island Microarray Study of DNA Methylation was Performed with Samples of Non-Hodgkin's Lymphomas (NHLs) with Different Clinical Behaviors

### Example Overveiw

Non-Hodgkin's Lymphoma (NHL) is a group of malignancies of the immune system that encompasses subtypes with variable clinical behaviors and diverse molecular features. Small B-cell lymphomas (SBCL) are low grade NHLs including mantle cell lymphoma, B-cell chronic lymphocytic leukemia/small lymphocytic lymphoma, and grades I and II follicular lymphoma. Despite the progress made in classification of NHLs based on histological features, cell surface markers and cytogenetics, and despite identification of DNA hypermethylation of some genes such as p57(KIP2), p15(INK4B) (6, 7), DAPK (8) and p73 (9) as being frequent in lymphoid malignancies, there is a substantial need in the art for novel compositions and methods for molecular classification.

Experimental design. Expression profiling is known to be useful for precise classification of different tumor types and subtypes, and expression microarray studies can provide information to assess clinical aggressiveness and to guide the choice of treatment in FL (12). Alizadeh et al (13) used a lymphochip to monitor gene expression signatures of diffuse large B cell lymphoma subgroups derived from distinct stages of B cell differentiation, and several groups have demonstrated that tumor classification can also be achieved by microarray based DNA methylation profiling (14, 15). By contrast, few published reports have focused on the identification of genes whose methylation profiles differ between currently recognized SBCLs.

Results. A high-throughput array-based technique called differential methylation hybridization was used in this Example to study SBCL subtypes based on a large number of potential methylation biomarkers. A total of 43 genomic DNA microarray experiments were analyzed. From these microarrays, several statistical methods were used to generate a limited set of genes for further validation by methylation specific PCR (MSP). Hierarchical clustering of the DNA methylation data was used to group each subtype on the basis of similarities in their DNA methylation patterns, revealing, as disclosed herein, that there is diversity in DNA methylation among the different subtypes.

In particular, differential methylation of LHY2, POU3F3, HOX10, NRP2, PRKCE, RAMP, MLLT2, NKX6-1, LPR1B, and ARF4 markers was validated in NHL cell lines and SBCL patient samples, and demonstrated a preferential methylation pattern in germinal center-derived tumors compared to pre- and post-germinal center tumors.

According to particular aspects of the present invention, these markers define molecular portraits of distinct sub-types of SBCL that are not recognized by current classification systems and have substantial utility for detecting and characterizing the biology of these tumors.

### Materials and Methods:

Lymphoma Cell Lines. Six common NHL cell lines were used to study methylation patterns across different subtypes of lymphoma; RL, Daudi, DB, Raji, Granta 519 and Mec-1. RL is a germinal center cell line of FL derivation from a male patient with the t (14; 18) gene rearrangement (16). The Daudi cell line is a derived from CD77+ Burkitt's lymphoma and is often used as a model of germinal center function (17). DB is a DLBCL cell line that has undergone isotype switching (17) and Raji cells are of germinal cell derivation (18). The cell surface marker CD10 is expressed on RL, Raji, DB, and DLBCL, therefore suggesting a germinal center relationship among theses cell lines. Granta 519 is a pre-germinal center cell line derived from a MCL patient (19). The Mec-1 cell line is derived from the peripheral blood of a patient with transformed B-CLL/SLL (20). Granta 519 and Mec-1 do not express CD10. These cells were acquired through the American Type Culture Collection (ATCC) or the Deutsche Sammlung von Mikroorganismen und Zellkulturen (DSMZ), and all were maintained in RPM1 1640 medium supplemented with 10% fetal bovine serum.

Patient Samples. Tissue and blood samples were obtained from patients following diagnostic evaluation at Ellis Fischel Cancer Center in Columbia, Mo., in compliance with the local Institutional Review Board. DNA was isolated from a total of 43 patient samples and control DNA was isolated from peripheral blood collected from volunteers whose mean age was <30 years using the QIAamp™ DNA Blood Minikit (Qiagen, Valencia, Calif.). Samples from 16 patients with FL, 12 with MCL, and 15 with B-CLL/SLL were used in this study. All cases of B-CLL/SLL had peripheral blood and bone marrow involvement, therefore were technically categorized as chronic lymphocytic leukemia, and are referred to herein as B-CLL/SLL. All specimens contained >80% neoplastic cells as determined by flow cytometry. Flow cytometry reports were available for 11 of 15 B-CLL/SLL patients used in this study; 5 patient samples were CD38+ and 6 CD38−. Cells from 3 patients with benign follicular hyperplasia (BFH) were also obtained.

Preparation of CGI Island Microarray. PCR products (on average 500 bp) of a microarray panel containing 8,544 sequenced CGI clones were prepared as previously described (21, 22). A pin-and-ring microarrayer GMS 417 (Genetic MicroSystems, Boston, Mass.) was used to spot unpurified PCR products as microdots on Corning UltraGAP II™ (Corning Life Science, Acton, Mass.) slides coated with amino-silane. The slides were then processed using the Corning Pronto Microarray™ (Corning Life Science, Acton, Mass.) reagents according to the manufacturer's recommendations.

Amplicon Preparation and Microarray Hybridization. DNA samples were prepared for hybridization via the DMH protocol (12). Succinctly, 2 μg of genomic DNA was restricted with MseI, a 4-base TTAA endonuclease that restricts bulk DNA down to less than 200 base pairs while preserving the GC-rich CGIs. The resulting sticky ends of the restriction digest are ligated using 0.5 nmol of the PCR linkers H24/H12 (H24: 5′-AGG CAA CTG TGC TAT CCG AGG GAT-3′ (SEQ ID NO: 6) and H12: 5′-TAA TCC CTC GGA-3′) (SEQ ID NO: 7). After a test PCR for successful ligation, DNA was directly digested with the methylation-sensitive endonucleases BstUI and HpaII, respectively (New England Biolabs, Beverly, Mass.). The amplicons were purified after a 20-cycle PCR reaction with QIAquick™ (Qiagen) columns and used for aa-dUTP (amino-allyl dUTP) incorporation using the BioPrime™ labeling kit (Invitrogen, Carlsbad, Calif.). Fluorescence amplicons representing pools of methylated NHL DNA (Cy5) relative to normal DNA (Cy3) were combined in a sex-matched manner and each mixture was co-hybridized to the CGI microarray chip as described (23-25). In females, 1 copy of the X chromosome is largely inactivated by DNA methylation. Therefore, women are expected to exhibit methylation of 1 allele of certain genes, such as the androgen receptor (AR) gene, whereas this occurs only in malignancy in males (26).

Microarray Data Analysis. Each locus on the slide appears as a colored dot comprised of red (from Cy5) and green (Cy3). The intensity levels of red and green in each spot signify the amount of methylation found in cancer (red) and normal (green) cells. Both were background-corrected and a global normalization applied with the assumption that the methylation level of both cancer and normal cells is similar in most loci (red/green≈1). Those loci with (red+green)≧T (where T=700) were flagged as good quality spots and sorted based on their log ratio of fluorescence. The normalization ratio was defined between the 20^{th} and 80^{th} percentile of that sorted list in an effort to minimize extreme ratio values caused by extremely small red or green values. Spots that were too low in intensity or disturbed by artifacts (along with all known housekeeping genes and repeat sequences) were assigned a normalized ratio of 1. After array normalization, an across-array analysis was performed for each locus. Only those loci with at least 25% of their between-array samples having a true normalized ratio (not artificially assigned to 1) were selected for analysis. These filtered loci were then subjected to further statistical testing to determine those loci that were differentially methylated across subtypes of NHL. The Kruskal-Wallis test, because of its ability to compare more than two data distributions and is a nonparametric method that does not assume normalcy in the data, was performed on the group of samples at each locus. The p-value threshold was calculated using the Benjamini and Hochberg method (27). The p-values of all loci were sorted in ascending order, p₍₁₎≦p₍₂₎≦ . . . ≦p_{(G)}, where G is the number of across-array filtered loci. Let J be the largest index j for which:

$${p}_{( j )} \leq \frac{j}{G} {φ}_{F} .$$

Then, the loci corresponding to the P-values p₍₁₎≦p₍₂₎≦ . . . ≦p_{(J)} were classified as differentially methylated. Nucleotide sequencing results came from the Der Laboratory, Toronto, Canada (http://derlab.med.utoronto.ca/CpGIslandsMain.php). Sequence identification information was obtained by the BLAST™ method.

Methylation Confirmation Analysis by MSP. The DNA methylation status of selected candidate genes from specific regions of the microarray clusters was confirmed using MSP. Each selected gene was first analyzed on cell line DNA and secondly on patient DNA. The following ten selected genes were examined; MLLT2, LHX2, LRP1B, HOX10, NKX6-1, ARF4, NRP2, RAMP, NRP2, and POUF3. One μg of genomic DNA was treated with sodium bisulfite to induce a chemical conversion of unmethylated (but not methylated) cytosine to uracil according to the manufacturer's instructions (EZ DNA Methylation Kit; Zymo Research, Orange, Calif.). For positive controls, normal lymphocyte DNA was treated with SssI methyltransferase (New England Biolabs), which methylates all the cytosines in the genome. The primer sequences used to confirm selected genes are listed in TABLE 1 and the MSP protocol was as described (25, 26). Methylated and unmethylated primers were designed using MethPrimer™ (wwW.urogene.org/methprimer/index.html). Products (5-9 μl) were directly loaded on a 2.5-3% agarose gel stained with SYBR Green (Cambrex Bio Science Rockland, Me.) visualized under UV light and quantified using Kodak gel documentation system.

Statistical analysis. For comparisons of gene promoter methylation between classes of NHLs, the chi-square statistic, as implemented in SAS (Cary, N.C.) software, was employed.

TABLE 1

| Primer sequences for 10 CGI loci, MSP conditions and expected product sizes. | | | | | | | |
|---|---|---|---|---|---|---|---|
| | CpG | | | | | | |
| Gene Name | Island | Methylated Primer | Length | Anneling Tm | Unmethylated Primer | Length | Anneling Tm |
| HOX10 | Yes | Antisense: 5′-TTTTAAAGTTACGGTTTGTCGG-3′ | 186 | 60 | Antisense: 5′-TTAAAGTTATGGTTTGTTGG-3′ | 181 | 60 |
| | | Sense: 5′-CTCAAAACCACTAAAACTCCGAA-3′ | | | Sense: 5′-AAAACCACTAAAACTCCAAA-3′ | | |
| ARF4 | Yes | Antisense: 5′-TCGGAACTAACCTTTATTATTTCGA-3′ | 210 | 62 | Antisense: 5′-TGGAAGTAAGGTTTATTATTTTGA-3′ | 209 | 60 |
| | | Sense: 5′-AAAATTAACCAATTTCGCTAACGTA-3′ | | | Sense: 5′-AAAATTAACCAATTTCACTAACATA-3′ | | |
| BLK | Yes | Antisense: 5′-GTTTATTTTAGCGGAAAAAGGC-3′ | 174 | 58 | Antisense: 5′-GTTTATTTTAGTGGAAAAAGGTGT-3′ | 175 | 61 |
| | | Sense: 5′-AACCTATAAAACACACACGTACGTA-3′ | | | Sense: 5′-CAACCTATAAAACACACACATATCATA-3′ | | |
| LHX2 | Yes | Antisense: TTTAGTTTATTTCGTTGGGGTAAAC-3′ | 199 | 62 | Antisense: 5′-TAGTTTATTTTGTTGGGGTAAATGG-3′ | 198 | 68 |
| | | Sense: 5′-CAAATAATTCAACTTCCACTCGAA-3′ | | | Sense: 5′-TCAAATAATTCAACTTCCACTCAAA-3′ | | |
| LRP1B | Yes | Antisense: 5′-AGTTTGCGTTGGAGATTGTTC-3′ | 105 | 57 | Antisense: 5′-AAGTTTGTGTTGGAGATTGTTTG-3′ | 108 | 57 |
| | | Sense: 5′-AATAACATTTATAAATACCGCCGTT-3′ | | | Sense: 5′-CCAATAACATTTATAAATACCACCATT | | |
| MLLT2 | Yes | Antisense: 5′-AGAGTAGGTAGTTTCGTAATATCGG-3′ | 124 | 58 | Antisense: 5′-GAGAGTAGGTAGTTTTGTAATATTGG-3′ | 127 | 66 |
| | | Sense: 5′-AATCTTCCGTCCATAAACGC-3′ | | | Sense: 5′-AAAATCTTCCATCCATAAACACC-3′ | | |
| NKX6-1 | Yes | Antisense: 5′-TTTTAGAGTGGTCGTTTGTAGTCG-3′ | 117 | 60 | Antisense: 5′-TTTTAGAGTGGTTGTTTGTAGTTGA-3′ | 116 | 60 |
| | | Sense: 5′-AAATCTCGTATATTTTCTCTTTCCGT-3′ | | | Sense: AATCTCATATATTTTCTCTTTCCATC-3′ | | |
| RAMP | Yes | Antisense: 5′-ATGAATTTCGTTAGTTTCGAGTAGC-3′ | 123 | 60 | Antisense: 5′-GAATTTTGTTAGTTTTGAGTAGTGG-3′ | 122 | 60 |
| | | Sense: 5′-CTCAACTAAAACTTTTCCTCCGAC-3′ | | | Senss: 5′-TCTCAACTAAAACTTTTCCTCCAAC-3′ | | |
| POU3F3 | Yes | Antisense: 5′-TGTATATATATATATACGAGGAAGCGG-3′ | 187 | 60 | Antisense: 5′-TGTATATATATATATATGAGGAAGTGG-3′ | 195 | 60 |
| | | Sense: 5′-GATCAACGAAACCGTACGAT-3′ | | | Sense: 5′-AAAATACCAATCAACAAAACCATACA-3′ | | |
| NRP2 | Yes | Antisense: 5′-TTTTAGAGATTAGCGTTGTAGTCGA-3′ | 168 | 60 | Antisense: 5′-TTTTAGAGATTAGTGTTGTAGTTGA-3′ | 169 | 60 |
| | | Sense: 5′- AAACCGAAACTAAAACCTCCG-3′ | | | Sense: 5′-AAAACCAAAACTAAAACCTCCAC-3′ | | |
| PRKCE | Yes | Antisense: 5′-TCGGTAAGTTTGTAGTGATAAAGTC-3′ | 136 | 60 | Antisense: 5′-TTGGTAAGTTTGTAGTGATAAAGTTGT-3′ | 142 | 60 |
| | | Sense: 5′-CTCGAAAACCACTAAAACGAA-3′ | | | Sense: 5′-AAACCTCAAAAACCACTAAAACAAA-3′ | | |

SEQ ID NOS, pairwise--from left to right, and from top to bottom are: SEQ ID NOS:8-51.

### Results:

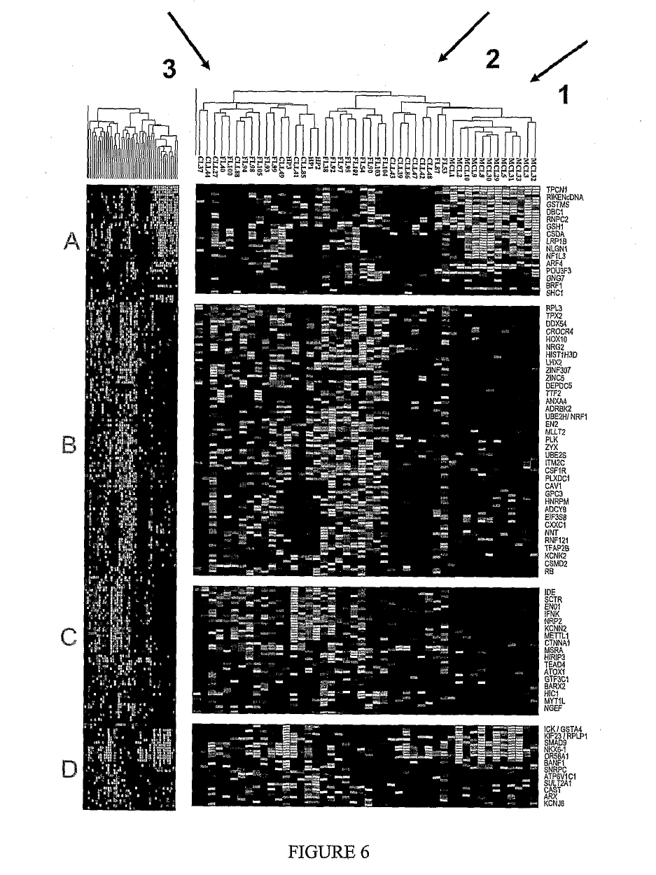

Segregation of SBCL subtypes by hierarchical clustering. Genomic DNA methylation microarray technology was used to characterize the three SBCL subtypes; MCL, B-CLL/SLL and FL. The cell of origin in each of these lymphomas is related to progressive stages of normal lymphoid cell differentiation activated in association with, or without, antigen in peripheral lymphoid tissues. This investigation included a total of 16 de novo patient samples from those with FL, 15 B-CLL/SLL, 12 MCL and 3 samples of BFH that were all probed for the presence of methylated DNA, mainly in the promoter and 1^{st} exon regions of genes and initially analyzed by hierarchical clustering. The relationship between the experimental results and patient samples of each type of SBCL is shown in FIG. 6. The upper dendrogram illustrates the relationships of patient samples to each other on the basis of DNA methylation patterns; those most alike cluster under a single branch of the dendrogram. As depicted, the hierarchical clustering algorithm grouped SBCLs according to the similarity in their DNA methylation patterns. In all, 256 CGI loci were classified as differentially methylated in at least 1 subtype of SBCL. It should be pointed out that there is not a 1-to-1 relationship between the very large number of loci from the main dataset in the panel to the left of FIG. 6, the expanded areas from the regions of interest (A-D), and the list of named genes on the right side of the figure. For each specific CGI locus of interest, the related gene was identified by searching the associated database of CGI sequences found at the Der laboratory web site (http://derlab.med.utoronto.ca/CpGlslands/CpGIslandsMain.php). Moving from left to right represents a “drilling down” into the microarray data to ultimately discover named genes that are differentially methylated. For example, the branch indicated by the arrow labeled “1” includes all the MCL samples, but no others. This separation appears to involve mainly clusters of gene loci from within regions A and D of the overall hierarchical cluster, as well as the paucity of methylated loci from within regions B and C where considerable methylation is indicated for FL and a subset of B-CLL/SLL samples. Thus, the observed patterns of DNA methylation in MCL patients were distinct from FL and a subset of B-CLL/SLL patients, but associated with another subset indicated by arrow “2” in FIG. 6. Further analysis of the profiles separated the B-CLL/SLL patients into 2 distinct groups. Six of 15 (40%) B-CLL/SLL samples (indicated by arrow “2”) clustered adjacent to MCL, an aggressive pre-germinal center subtype of NHL (1). Flow cytometry revealed that 2/6 (33%) of these were CD38+, 2/6 (33%) were CD38−, and flow cytometry results were not available for the remaining 2 samples. Conversely, 9/15 (60%) B-CLL/SLL samples clustered adjacent to FL (indicated by arrow “3”). Of these, 4/9 (44.4%) were CD38+, 3/9 (33%) were CD38−, and flow cytometry results were not available for the remaining 2 samples. While there is no clear association of methylation with CD38 expression, an observation that may be secondary to the small number of samples of each type, this observation still suggests that DNA methylation patterns in B-CLL/SLL may not be homogeneous and perhaps methylation patterns relate to unrecognized subsets of B-CLL/SLL. A larger study of gene methylation specifically in B-CLL/SLL is currently under way and should address this issue. Those B-CLL/SLL samples that clustered near MCL (arrow “2”) were characterized in the overall cluster as having few loci illustrated as methylated in regions A, B, and C, but a small block within region D that was conspicuously indicated as hypermethylated, similar to block D in MCL cases.

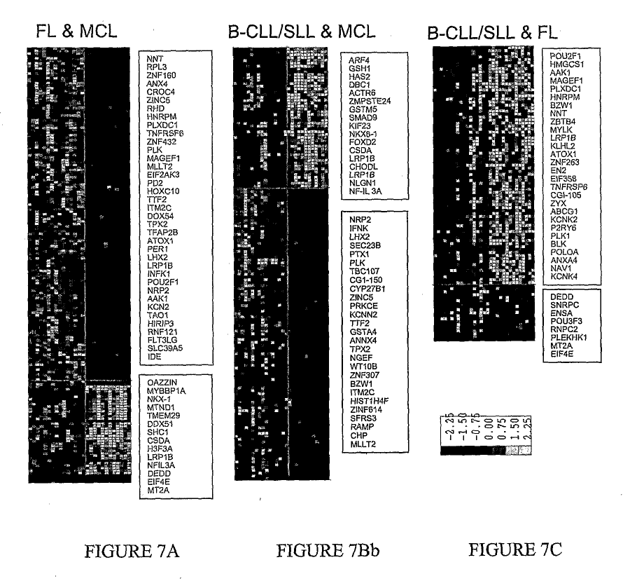

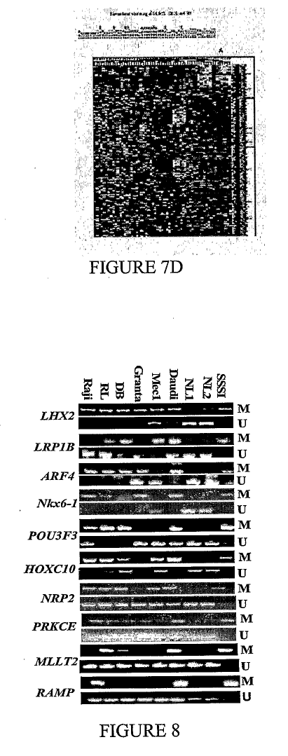

Cells from FL are similar in their biological characteristics to cells found in reactive secondary follicles or germinal centers of lymph nodes. From a quantitative standpoint there appear to be more CGI loci hypermethylated in FL patients than the MCL and a subset of B-CLL/SLL samples (FIG. 6). Nevertheless, according to particular aspects of the present invention, prominent blocks of methylated gene loci were revealed in this hierarchical clustering process that indicated the ability to separate the 3 classes of SBCLs, and perhaps subclasses within B-CLL/SLL. Therefore, to further examine relationships between classes, data from the middle region of FIG. 6 including cases of FL, MCL, and B-CLL/SLL was re-clustered in a pair-wise manner as indicated (FL versus MCL, FIG. 7A; B-CLL/SLL versus MCL, FIG. 7B; B-CLL/SLL versus FL, FIG. 7C). In the case of FL versus MCL (FIG. 7A), a large number of hypermethylated loci distinguished each class; 38 named genes were hypermethylated in FL compared to MCL and 14 named genes were hypermethylated in MCL compared to FL. The remaining loci were either hypothetical genes or regions of DNA that did not fall within or near a gene promoter or 1^{st} exon region. Similarly, 17 named genes were hypermethylated in MCL compared to B-CLL/SLL, and 35 named genes were hypermethylated in B-CLL/SLL compared to MCL (FIG. 7B). Finally, 29 named genes were hypermethylated in FL compared to B-CLL/SLL and only 8 were hypermethylated in B-CLL/SLL compared to FL (FIG. 7C). Interestingly, reciprocal subsets of B-CLL/SLL cases still cluster with MCL (FIG. 7B) and another subset clusters with FL (FIG. 7C). Sequence characterization and chromosomal location of differentially methylated CGI loci are shown in TABLE 2. Most of these loci are located in the promoter or the first exon regions of known genes with a known function, but in some cases are found in introns.

TABLE 2: Information on genes selected from various regions of all differentially methylated clusters from FIGS. 6 and 7. Shown are the gene name, accession number, chromosomal location, whether each contains a CpG island, and the purported main function of each. Our sequenced clones were viewed through the BLAT SEARCH WEBSITE./

TABLE 2

| Gene Name | Assession no. | Chromosome | CpG Island | Gene Function |
|---|---|---|---|---|
| AAK1 | NM_014911 | 2p13.3 | YES | AP2 associated Kinase1 |
| ABCG1 | NM_207630 | 21q22.3 | NO | ATP binding cassette transporter G1 |
| ACTR6 | NM_022496 | 12q23.1 | YES | Activated protein 6 |
| ALX4 | AB058691 | 11p11.2 | YES | Aristaless-like homoebox 4 |
| ANX4 | NM_001153 | 2p13.3 | YES | Annexin A4 |
| ARF4 | BC016325 | 3p21.2-p21.1 | YES | ADP-ribosylation factor 4 |
| ARX | AY038071 | Xp22.1-p21.3 | YES | Aristaless related homeobox |
| ATOX2 | NM_004045 | 5q33.1 | YES | Antioxidant protein 1 |
| BLK | NM_001715 | 8p23.1 | NO | B lymphoid tyrosine kinase |
| BZW1 | NM_014670 | 2q33.1 | YES | Basic leucine zipper and W2 domains 1 |
| CG1-150 | AF177342 | 17p13.3 | NO | Hypothetical protein |
| CHODL | AF257472 | 12q12.1 | YES | Chondrolectin |
| CHP | NM_007236 | 15q15.1 | YES | Calcium binding protein |
| CROC4 | NM_006365 | 1q22 | YES | Transcriptional activator |
| CSDA | BC021926 | 12q13.2 | YES | Cold Shock Domain Protein A |
| CYP27B1 | NM_000785 | 12q14.1 | YES | Cytochrome P450 |
| DBC1 | AF027734 | 9q32-q33 | NO | Deleted in Bladder Cancer I |
| DEDD | BC046149 | 1q23.3 | YES | Death factor domain containing |
| DKFZP586D0919 | BC016395 | 12q14.1 | YES | Hepatocellular carcinoma-associated antigen HCA557a, isoform a |
| DOX54 | BC005848 | 12q24.13 | NO | Dead box polypeptide 54 |
| EIF2AK3 | NM_004836 | 2p11.2 | YES | Eukaryotic translation initiation factor 2-alphakinase 3 |
| EIF3S8 | BC001571 | 16p11.2 | YES | Eukaryotic translation initiation factor 3 |
| EIF4E | NM_001968 | 4q23 | YES | Eukaryotic translation initiation factor |
| EN2 | NM_001427 | 7q36.3 | YES | engralled homolog 2 |
| ENSA | NM_207042 | 1q21.2 | YES | Endosulfine alpha isoform 3 |
| FOXD2 | NM_004474 | 1p33 | YES | Forkhead box D2 |
| GSH1 | AB044157 | 13q12.2 | YES | GS homeobox 1 |
| GSTA4 | NM_001512 | 6p12.1 | NO | Glutatione S-transferaseA4 |
| GSTM5 | LO2321 | 1p13.3 | NO | Glutothione s-transferase M5 |
| GTF3C1 | U02619 | 16p12 | YES | General transcription factor IIIC |
| H3F3A | NM_002107 | 1q41 | YES | H 3 histon family 3A |
| HAS2 | NM_005328 | 8q24.13 | YES | Hyaluronan synthase 2 |
| HIRIP3 | BC000588 | 16p11.2 | YES | HIRA Interacting protein 3 |
| HIST1H4F | NM_003540 | 6p22.2 | NO | Histon 1, H2ad |
| HMGCS1 | NM_002130 | 5p12 | YES | 3 hydroxy 3-methylglutaryl-coenzymeA synthase |
| HNRPM | NM_005968 | 19p13.1 | NO | M4 protein deletion mutant |
| HOXC10 | BC001293 | 12q13.3 | YES | Homeo box C10 |
| IDE | M21188 | 10q23-q25 | YES | Insulin-degrading enzyme |
| INFK | NM_020124 | 9p21.2 | YES | Interferon like protein precursor |
| ITM2C | AF271781 | 2q37.1 | YES | Integral membrane Protein 2C |
| | | | | Potassium intermediate/small conductance calcium activated channel |
| KCN2 | NM_021614 | 5q22.3 | YES | superfamily N, member 2 |
| KCNK2 | NM_00101742 | 1q41 | NO | Potassium channel superfamily K membrane 2 isoform |
| KCNK4 | NM_016611 | 11q13.1 | YES | Potassium channel superfamily K member 4 isoform |
| KIAA0152 | D63486 | 12q24.31 | YES | Hypothetical protein KIAA0152 |
| KIF23 | NM_004856 | 15q23 | YES | Kinesin family member 23 |
| KLHL2 | NM_007246 | 4q32.3 | YES | Kelch-like 2 |
| LHX2 | AF124735 | 9q33-q34.1 | YES | LIM homeobox 2 |
| LRP1B | AF176832 | 2q21.2 | YES | Low Density lipoprotein receptor related protein (deleted in tumors) |
| LRP1B | AF176832 | 2q21.2 | YES | Low density lipoprotein-related protein 1B (deleted in tumors) |
| MAGEF1 | BC010056 | 3q13 | YES | Melanoma-associated antigen F1 |
| MGC21416 | BC012469 | Xq13.1 | YES | Hypothetical protein LOC286451 |
| MLLT2 | L13773 | 4q21 | YES | Myeloid/lymphoid or mixed-lineage leukemia |
| MT2A | NM_005953 | 16q12.2 | YES | Metallothionein 2A |
| MTND1 | NM_173708 | chr. M | NO | NADH dehydrogenase 1 |
| MYBBP1A | NM_014520 | 17q13.2 | YES | MYB binding protein (P160) 1 A |
| MYLk | NM_053030 | 3q21.1 | NO | Myosin light chain kinase Isoform 5 |
| NAV1 | NM_020443 | 1q32.1 | YES | Neuron navigator |
| NF-IL 3A | NM_005384 | 9q22.31 | NO | Nuclear factor interleukin 3 regulated |
| NGEF | BC031573 | 2q37 | NO | Neuronal guanine nucleotide exchange factor |
| NKX6-1 | NM_006168 | 4q21.2-q22 | NO | NK6 transcription factor related, locus 1 |
| NLGN1 | AB028993 | 3q26.31 | NO | Neuroligin 1 |
| NNT | AL831822 | 5p13.1-5cen | YES | Nicotinamide nucleotide transhydrogenase |
| NRP2 | BC009222 | 2q33.3 | YES | NRP2 protein |
| OAZZIN | BC013420 | 8q22.3 | YES | Ornithine decarboxylase antizyme inhibitor |
| P2RY6 | NM_1767981 | 11q13.4 | NO | Pyrimidinergic receptor P2Y |
| PD2 | NM_019088 | 19q13.2 | NO | PD2 protein |
| PER1 | NM_002616 | 17p13.1 | YES | Period 1 |
| PES1 | BC032489 | 22q12.1 | YES | Pescadillo homolog 1 |
| PLEKHK1 | NM_145307 | 10q21.2 | YES | Rhotekin 2 |
| PLK | BC002369 | 16p12.1 | YES | Polo-like kinase 1 |
| PLXDC1 | NM_020405 | 17q12 | YES | Tumor endothelial marker 3 precursor |
| POLA | NM_016937 | Xp21.3 | YES | Polymerase DNA directed |
| POU2F1 | BC052274 | 1q24.2 | YES | POU domain class 2 transcriptional factor 1 |
| POU3F3 | NM_006236 | 2q12.1 | YES | POU domain, class 3, transcription factor 3 |
| PRKCE | NM_005400 | 2p21 | YES | Protein kinase C, epsilon |
| PTX1 | BC064522 | 12p11.22 | YES | Hypothetical protein |
| RAMP | BC033297 | 1 | YES | L2DTL protein (RA-regulated nuclear matrix-associated protein) |
| RHD | NM_016124 | 1p36.11 | Yes | Blood group D antigen DBA |
| RNF121 | AK023139 | 11q13.4 | NO | Ring finger protein 121 |
| RNPC2 | L10911 | 20q11.22 | YES | Hypothetical protein DKFZp686A11192 |
| RPL3 | BC004323 | 12q13.1 | YES | Hypothetical protein L3 |
| SEC23B | NM_032986 | 20p11.23 | YES | Sec23homologB |
| SFRS3 | NM_003017 | 6p21.31 | NO | Splicing factor arginine/serine rich 3 |
| SHC1 | NM_003029 | 1q22 | YES | Src Homology 2 domain containing transforming protein 1 |
| SLC39A5 | BC027884 | 12q13.3 | NO | Solute Carrier family 39 (metal ion transporter) |
| SMAD9 | BC067766 | 13q12-q14 | NO | MADH9 protein |
| SNRPC | X12517 | 6p21.31 | YES | Small nuclear ribonucleoprotein polypeptide C |
| TAO1 | AF061943 | 16p11.2 | YES | Prostate derived STE20 like kinase PSK |
| TBC107 | BC050465 | 6p24.1 | YES | Hypothetical protein |
| TFAP2B | NM_003221 | 6p12.3 | YES | Transcriptional factor AP-2 beta |
| TMEM29 | NM_014138 | chr. X | YES | Transmembrane protein 29 |
| TNFRSF6 | NM_000043 | 10q23.31 | NO | Tumor necrosis factor receptor superfamily member 6 |
| TPX2 | AF287265 | 20q11.2 | YES | Hepatocellular carcinoma-associated antigen 90 |
| TTF2 | BC030058 | 1p13.1 | YES | Similar to transcription termination factor, RNA polymerase II |
| WT10B | NM-005430 | 12q13.12 | NO | Wingless type MMTU integration site family |
| ZBTB4 | NM_020899 | 17p13.1 | YES | Zinc finger and BT3 domain containing protein 4 |
| ZINC1 | D76435 | 3q24 | YES | Zic family member 1 |
| ZINC5 | NM_0331321 | 13q32.3 | YES | Zinc family member 5 |
| ZMPSTE24 | NM_005857 | 1p34.2 | YES | Zinc metallo proteinase |
| ZNF160 | NM_198893 | 19q13.41 | YES | Zinc finger protein 160 |
| ZNF263 | BC008805 | 16p13.3 | YES | Zinc finger protein 263 |
| ZNF307 | NM_019110 | 6p22.1 | YES | Zinc finger protein 307 |
| ZNF432 | NM_014650 | 19q13.41 | YES | Zinc finger protein 432 |
| ZNF614 | NM_025040 | 19q.41 | YES | Zinc finger protein 432 |
| ZYX | NM_003461 | 7q34 | NO | ZYX protein |

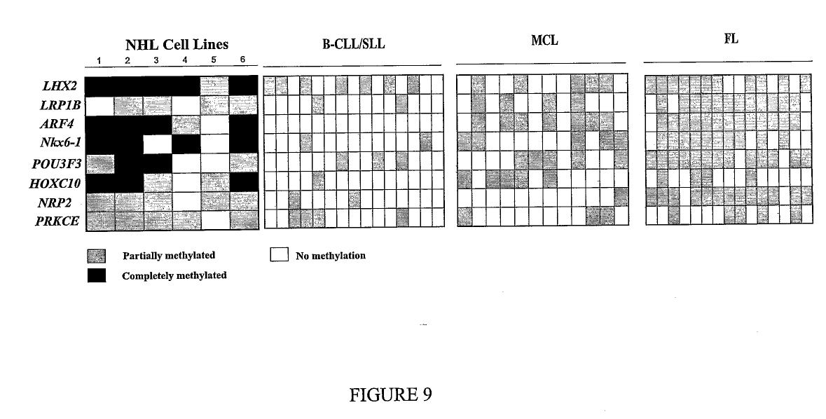

Confirmation of Microarray findings by MSP. Microarrays are excellent discovery tools, but additional confirmation of selected results is prudent to have full confidence in the findings. In order to independently confirm the DNA methylation status of 10 known genes (NKX6-1, LRP1B, MLLT2, LHX2, ARF4, HOX10, RAMP, NRP2, POU3F3, PRKCE) selected to represent each region of the hierarchical clusters, MSP primers were produced and used to test a series of NHL cell lines (FIG. 8) and SBCL patients (FIG. 9). Nine of these 10 genes were methylated in both cell lines and in de novo NHL tumors. The MLLT2 gene was examined, but was not methylated in any patient samples despite the methylation shown in the RL cell line (FIGS. 8 and 9). Thus, this gene was not included in any further analyses. Hypermethylation of only 1 gene, LIM homeobox protein 2 (LHX2), was present in all NHL cell lines and a high proportion of patient samples, whereas the remaining genes were differentially methylated in the various cell lines, an observation that would be expected given the relationships of the cell lines to various stages of differentiation. Interestingly, the remaining genes were predominantly methylated in the germinal center derived cell lines (Raji, RL, DB, and Daudi) but less so in Granta 519 and Mec-1 cell lines derived from MCL and B-CLL/SLL, respectively.

Analysis of CGI Methylation patterns in de novo SBCL samples. The methylation patterns of cancer cell lines do not always reflect the presence of methylation in primary tumors. There is evidence that CGI methylation in several tissue-specific genes is secondary to intrinsic properties of cell lines (28). However, in this study consistency was found between promoter methylation of the selected genes in NHL cell lines and primary NHLs. The nine genes confirmed as above were examined in 42 NHL and 3 BFH samples using MSP (FIG. 9). Methylation of POU3F3 was observed in 3/15 (20%) B-CLL/SLL cases, 5/12 (41.6%) MCL cases and 13/15 (87%) FL cases (p=0.01). For each of the genes confirmed in patient samples, there was a higher incidence of DNA methylation in germinal center-related FL than in pre-germinal center-related NHLs (MCL and B-CLL/SLL) (FIG. 9). Due to the nature of the disease, patient samples were not purely tumor DNA (>80% neoplastic cells), therefore the unmethylated allele amplified in each patient sample, representing either normal tissue found within the tumor or the heterogeneity of methylation within the tumor sample itself. It is important to point out that MSP is more sensitive in identifying one locus at a time; however, the technique (DMH) we used to generate a hierarchical clustering algorithm is for large scale interrogation of highly methylated CGI loci. Therefore, the frequencies of methylation shown in MSP might not strictly correlate with DMH results.

Relationships between SBCL classes, the percentage of patient samples methylated in each gene promoter, and the statistical significance of these observations using the chi-square test are presented in TABLE 3.

TABLE 3
Statistical evaluation of comparative DNA methylation. For each gene
validated in patient samples, the proportion of samples from each class
of NHL that were methylated, and the pair-wise chi-square analysis are shown.

| | | | | | | | B-CLL/SLL/ | | |
|---|---|---|---|---|---|---|---|---|---|
| N = 42 | B-CLL/SLL | | MCL | | FLI | | MCL | B-CLL/SLL/FL | FL/MCL |
| Genes | M | % | M | % | M | % | P-Value results are all ≦ the number shown | | |
| LHX2 | 7/15 | 46.6 | 5/12 | 41.6 | 11/15 | 73 | 1.0 | 0.2 | 0.1 |
| LRP1B | 2/15 | 13.3 | 4/12 | 33.3 | 13/15 | 86.6 | 1.0 | 0.001 | 0.01 |
| ARF4 | 0/15 | 0 | 7/12 | 58.3 | 13/15 | 86.6 | 0.001 | 0.001 | 0.1 |
| NKX6-1 | 2/15 | 13.3 | 5/12 | 41.6 | 10/15 | 66.6 | 0.1 | 0.01 | 0.2 |
| POU3F3 | 3/15 | 20 | 5/12 | 41.6 | 13/15 | 86.6 | 1.0 | 0.001 | 0.025 |
| HOX10 | 1/15 | 6.6 | 5/12 | 41.6 | 4/15 | 26.6 | 0.05 | 0.2 | 1.0 |
| NRP2 | 2/15 | 15.3 | 1/12 | 8.3 | 13/15 | 86.6 | 1.0 | 0.001 | 0.001 |
| PRKCE | 4/15 | 26.6 | 3/12 | 25 | 5/15 | 33.3 | 1.0 | 1.0 | 1.0 |

For instance, in the comparison of B-CLL/SLL (n=15) with MCL (n=12), of the 9 gene promoters examined, only ARF4 (p=0.001) and HOX10 (p=0.05) revealed differences at p=/<0.05. The others were not statistically different between the 2 classes. The greatest differences were seen when comparing FL (n=15) to either B-CLL/SLL or MCL. For the comparison of FL to B-CLL/SLL, only 3 gene promoters were not significantly different at p=/<0.05; LHX2, HOX10, and PRKCE. In comparison of FL to MCL, only 4 gene promoters, LRP1B, BLK, POU3F3, and NRP2 were statistically different. In the case of POU3F3, while all 3 classes revealed DNA methylation, they were all similar in proportion. Therefore, we were able to confirm that promoter DNA methylation, as discovered in the microarray experiments, was present in 9 of the 10 genes tested in de novo NHL samples, while all 10 were methylated in NHL cell lines.

### Example 3

### Novel Epigenetic Markers for Non-Hodgkin's Lymphoma (NHL) were Discovered Using a CpG Island Microarray

### Example Overview

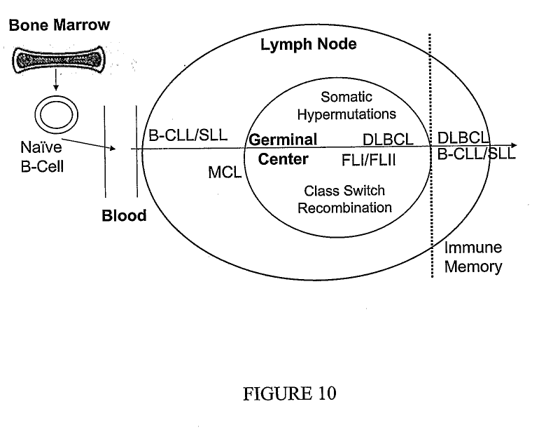

Non-Hodgkin's Lymphoma (NHL) is the 5^{th} most common malignancy in the U.S., accounting for approximately 56,390 new cases in 2005 (1). Mature B-cell NHLs including B-cell chronic lymphocytic leukemia/small lymphocytic lymphoma (B-CLL/SLL), mantle cell lymphoma (MCL), follicular lymphoma (FL), and diffuse large B-cell lymphoma (DLBCL) comprise the majority of all NHL cases (2) and each of these diseases is closely related to a normal counterpart in B-cell differentiation (3) (FIG. 10)

A CpG island microarray-based technique was previsouly developed for genome-wide methylation analysis in breast and ovarian cancer (10, 11). In this Example, applicants used this approach to identify a group of genes silenced by DNA methylation in 6 NHL cell lines that are derived from different subtypes of NHL. A sub panel of the novel methylated genes was further examined in primary NHL samples and stage-related methylation in NHLs was discovered.

More specifically, 30 novel methylated genes were identified in these cell lines and ten of them were independently confirmed. Methylation of six of these genes was then further examined in 75 primary NHL specimens comprised of four subtypes representing different stages of maturation. Each gene (DLC-1, PCDHGB7, CYP27B1, EFNA5, CCND1 and RARβ2) was frequently hypermethylated in these NHLs (87%, 78%, 61%, 53%, 40%, and 38% respectively), but not in benign follicular hyperplasia. While some genes were methylated in almost all cases, others were differentially methylated in specific subtypes. Particularly, tumor suppressor candidate gene DLC-1 methylation was detected in a large portion of primary tumor and plasma DNA samples by using quantitative methylation specific PCR analysis. This promoter hypermethylation inversely correlated with DLC-1 gene expression in primary NHL samples. Thus, according to aspects of the present invention, CpG island microarray was used to identify novel methylated gene markers relevant to molecular pathways in NHLs, and having substantial utility as biomarkers of disease, and subtypes thereof.

### Materials and Methods:

Cell Lines and Drug Treatments. Human NHL lines RL, Daudi, DB, Raji, Granta 519 and Mec-1 were maintained in RPMI 1640 media. The germinal center related cell line RL is derived from a male patient with FL and the t(14,18) gene rearrangement (12), and Daudi and Raji cells are of germinal center derivation. The postgerminal center cell line DB is a DLBCL cell line that has undergone isotype switching (12). All four of these cell lines expressed surface CD10, thus suggesting a germinal center relationship (9). Granta 519 is an MCL cell line over-expressing cyclin D1 (13) and Mec-1 is a transformed B-CLL/SLL cell line (14). For gene reactivation experiments, cells were cultured in the presence of vehicle (PBS) or DAC (1.0 μM; medium changed every 24 h). After 4 days, cells were either harvested or further treated with TSA (1.0 μM) for 12 h and then harvested. Some cells were also treated with TSA alone for 12 h before harvest. Genomic DNA or total RNA was isolated using Qiagen™ kits (Qiagen, Valencia Calif.) and used for methylation and gene expression analysis, respectively.

Tissue Samples. Tissue and blood samples were obtained from patients after diagnostic evaluation for suspected lymphoma at the Ellis Fischel Cancer Center (Columbia, Mo.) and the Holden Comprehensive Cancer Center (Iowa City, Iowa) in compliance with local Institutional Review Boards. DNA was isolated from a total of 126 specimens; 8 from peripheral blood of healthy volunteers, 5 from patients with benign follicular hyperplasia (BFH¹), 13 MCL (mean age, 52.7 years; range, 39-87 years), 30 with B-CLL/SLL (mean age, 66.9 years; range, 56-84 years), 30 from FL (mean age, 62.0 years; range, 50-75 years), and 29 DLBCL (mean age, 57.0 years; range, 45-75 years). All cases of B-CLL/SLL had peripheral blood and bone marrow involvement, and thus were technically categorized as CLL. These are all referred to herein as B-CLL/SLL. Retrospective analysis of flow cytometric data collected at the time of diagnosis for a subset of cases revealed that FL specimens comprised 75% neoplastic B-cells (n=9, range 36-90%), MCL specimens comprise 88% neoplastic cells (n=4, range 85-91%), CLL specimens comprise 80% neoplastic cells (n=12, range 39-94%), and DLBCL specimens comprise 75% neoplastic cells (n=7, range 38-99%). Total RNA was extracted from 2 samples of normal peripheral blood lymphocytes, 3 normal lymph nodes, 9 DLBCL, 10 FL, 11 CLL, and 9 MCL patient samples using the RNeasy™ kit (Qiagen, Valencia, Calif.). A 2-spin method of separating plasma from cellular elements (15) was used in our study. Plasma DNA was isolated from peripheral blood of 15 NHL patients using the QiaAmp™ Blood kit.

Preparation of CpG Island Microarray. The production of microarray panel containing 8,640 CpG island clones was prepared as described (11). Amplified PCR products were spotted, in the presence of 20% DMSO, on UltraGap™ slides (Corning Life Science, Acton, Mass.). The slides were post-processed immediately before the hybridization using Pronto Universal Microarray Reagents (Corning Life Science, Acton, Mass.). In addition, sequences from CpG islands of 42 known tumor suppressor genes were PCR amplified and printed on the same slides. The whole CGI library was recently sequenced by the Microarray Centre of University Health Network, Toronto, Canada and the sequences can be viewed at htt://s-der10 med.utoronto.ca/CpGIslands.htm. Out of the 8640 CpG island fragments, 4564 unique genomic loci were identified.

Preparation of Amplicons for Methylation Analysis. Amplicon preparation for methylation analysis was performed as previously described (16, 17). Briefly, 2 μg genomic DNA was digested with MseI and then ligated to a PCR-linker. The ligated DNA was then directly digested with methylation-sensitive endonucleases, HpaII and BstUI, and amplified with a linker primer by PCR (11). The amplified products (or amplicons) were purified for fluorescence labeling. Incorporation of aa-dUTP¹ into amplicons (5 μg) was conducted using the Bioprime DNA Labeling System (Invitrogen, Carlsbad, Calif.). Cy5 and Cy3 fluorescence dyes were coupled to aa-dUTP-labeled test and reference amplicons, respectively, and co-hybridized to the CpG island microarray panel. Hybridization and the post-hybridization washing were done according to the manufacturer's procedures (Corning Life Sciences, Acton, Mass.). Hybridized slides were scanned with the GenePix™ 4200A scanner (Axon, Union City, Calif.) and the acquired images were analyzed with the software GenePix™ Pro 5.1

Microarray data analysis. The Cy3 and Cy5 fluorescence intensities were obtained for each hybridized spot. Array spots with fluorescence signals close to the background signal, reflecting PCR or printing failures, were excluded from the data analysis. Because Cy5 and Cy3 labeling efficiencies varied among samples, the Cy5/Cy3 ratios from each image were normalized according to a global mean method in Genepix™ Pro 5.1. This internal control panel included 20 Mse I fragments that have no internal Bst UI and Hpa II restriction sites spotted at several concentrations on each array. The adjusted Cy5/Cy3 ratio for each CGI locus was then calculated and data were exported in a spreadsheet format for analysis. The hybridization experiments were repeated and only those reproducible spots were chosen for analysis.

Methylation Specific PCR (MSP) and Combined Bisulfite and Restriction Analysis (COBRA). 2 μg of genomic DNA was treated with sodium bisulfite according to the manufacturer's recommendations (Ez™ DNA methylation kit; Zymo Research, Orange, Calif.). For the preparation of 100% methylated DNA, a blood DNA sample was treated with M. SssI methyltransferase (New England Biolabs, Beverly, Mass.) that methylated all cytosine residues of CpG dinucleotides in the genomic DNA. Sodium bisulfite modification of the test and SssI-treated DNA samples was then performed as described above. Bisulfite-treated genomic DNA was used as a template for PCR with specific primers located in the CpG island regions of each selected gene. For MSP, allele specific primers which cover 2-3 CpG dinucleotides were designed to differentiate methylated and unmethylated sequences. Amplification was performed using AmpliTaq™ Gold polymerase (Applied Biosystems, Foster City, Calif.). For COBRA, after amplification, PCR products were digested with the restriction enzyme BstUI (New England Biolabs, Beverly, Mass.), which recognizes sequences unique to the methylated and bisulfite-unconverted alleles. The digested DNA samples were separated in parallel on 3% agarose gels, stained with SYBR green and quantified using a Kodak gel documentation system. The additional COBRA primers used are: CCND1, 5′GGTTTGGGTAATAA GTTGTAGGGA (sense strand) (SEQ ID NO:52) and 5′-CAACCATAAAACA CCAACTCCTATAC (antisense strand) (SEQ ID NO:53); EFNA5, 5′-TTTAAGGAGGGAAAGAGGAGTAGTT (sense strand) (SEQ ID NO:54) and 5′-AAATC CCTCCAACTCCTAAAT AAAC (antisense strand) (SEQ ID NO:55); PCDHGB7, 5′-TGGGGTAGAATAAA GGTAGTAGTAAAGGAA (sense strand) (SEQ ID NO:56) and 5′-ACAATCCCACACAAAACCTCTAAAC (antisense strand) (SEQ ID NO:57); NOPE, 5′-TTTTTTGTTTTATTTATTTTAGTTTTAGTT (sense strand) (SEQ ID NO:58) and 5′-AAAACCCATCTCCACAAATATCAT (antisense strand) (SEQ ID NO:59); RPIB9, 5′-ATTGGAATTGATATA AAG TTT AGG GTT (sense strand) (SEQ ID NO:60) and 5′-ACCCCCTTAAACAAATATAAAAAAC (antisense strand) (SEQ ID NO:61); PON3, 5′-TTTTTGGGTAGAGGTTAAGGTTTAA (sense strand) (SEQ ID NO:62) and 5′-CCCCAAATCCTAAAAAAAATAAATTA (antisense strand) (SEQ ID NO:63); FLJ39155, 5′-GGTTTTTGTTTTTGGTTTTTAGTTT (sense strand) (SEQ ID NO:64) and 5′-ATCTAAAAAATTAATCATTCTTTTAATAAA (antisense strand) (SEQ ID NO:65).

DLC-1 Quantitative Real Time MSP Assay. The real-time MSP uses two amplification primers specific for methylated sequences and an additional, amplicon-specific, and fluorogenic hybridization probe (Probe: FAM/AAG TTC GTG AGT CGG CGT TTT TGA/BHQ1 (SEQ ID NO:5) whose target sequence is located within the amplicon. The probe was labeled with two fluorescent dyes, with FAM at the 5′-end and BHQ1 at the 3′-end, and synthesized by IDT (Coralville, Iowa). The bisulfite treated DNA was used for PCR amplification with appropriate reagents in QPCR mix (ABgene, Rochester, N.Y.) as recommended by the manufacturer. The reaction was carried out in 40-45 cycles using a SmartCycler™ real-time PCR instrument (Cepheid, Kingwood Tex.).

Real-time RT-PCR. Total RNA (2 μg) was pre-treated with DNase I to remove potential DNA contaminants and reverse-transcribed in the presence of SuperScript III™ reverse transcriptase (Invitrogen, Carlsbad, Calif.). The generated cDNA was used for PCR amplification with the system described above. The Taqman™ probe and primer sets for real time PCR were purchased from Applied Biosystems (Foster City, Calif.). Separate parallel reactions were run for GAPDH cDNA using a series of diluted cDNA samples as templates to generate standardization curves. The mRNA levels were derived from the standardization curves and expressed as relative changes after normalization to those of GAPDH.

### Results:

Methylation profiling in NHL cell lines. The microarray (16) was used to identify hypermethylated CpG island loci in the 6 NHL cell lines. Cy5- and Cy3-labeled amplicons, representing differential pools of methylated DNA in NHL cell lines relative to normal lymphocyte samples in a sex matched manner, were used as targets for microarray hybridization. Genomic DNA fragments containing methylated restriction sites were protected from the digestion and could be amplified by linker-PCR, whereas the equivalent allele fragments containing the unmethylated restriction sites were digested and thus could not be amplified in the normal lymphocytes. As similar to cDNA microarray experiments, the significance of methylation changes is determined by the comparison of the ratio of two reporters, Cy5 and Cy3. These hypermethylated CpG island loci appeared as “red” spots after microarray hybridization because greater signal intensities were obtained from the Cy5-labeled (red) NHL amplicons, than from those of the Cy3-labeled (green) control amplicons. When a cut-off value of the normalized Cy5/Cy3 ratio was set at >2 for the positive loci, a total of 86 methylated CpG loci (1.88% of 4564 CpG island fragments) were identified in Raji, 74 (1.62%) in Daudi, 68 (1.49%) in RL, 71 (1.55%) in DB, 51 (0.87%) in Mec-1 and 26 (0.56%) in Granta 519. Fifty two loci (1.14%) were found commonly methylated in at least 4 of the 6 NHL cell lines. This same cut-off ratio was effective in identifying hypermethylated CpG islands in breast tumors in applcants' previous study (11). Using the methylation microarray data of 83 named genes that are methylated in at least two cell lines, cluster analysis was conducted. Clustering of the pattern of methylation yielded a profile that allowed discrimination between germinal center derived lymphomas DB and RL, and non-germinal center lymphoma Granta 519 and Mec-1 (FIG. 11A). Interestingly the Burkitt's lymphoma cell lines possess different patterns of methylation in which Raji is grouped with DB and RL and Daudi is grouped with Granta and Mec-1. The cluster is somewhat related with the BCL6 and CD10 expression pattern as measured by real time PCR, and flow cytometry. BCL6 and CD10 positive cell lines seem to have acquired more methylation during transformation than BCL6 and CD10 negative cell lines.

Independent Verification of Methylation. Among the 30 most interesting genes based on review of literature (TABLE 4), the microarray findings of 10 known genes (PCDHGB7, EFNA5, CYP27B1, CCND1, DLC-1, NOPE, RPIB9, FLJ39155, PON3 and RARβ2) whose function might relate to cancer were selected for independent confirmation by COBRA and MSPCR analyses. Hypermethylation of these genes was found in the 6 NHL cell lines (FIG. 11B). The most frequently methylated, DLC-1, was methylated in all 6 cell lines. The remaining 9 genes were predominantly methylated in the germinal center derived cell lines, but to a less extent in the Mec-1 and Granta 519 cell lines which corresponds to the microarray findings in general. Particularly, by semiquantitative COBRA assays, NOPE and RPIB9 were found to be partially methylated in Mec-1 and Granta 519 cell lines, but completely methylated in the other four germinal center related lymphoma cell lines. Furthermore, the methylation status of CCND1 in the Granta 519 cell line is consistent with the findings of a recent report (18).

TABLE 4
List of genes most frequently methylated in NHL cell lines

| | GenBank | | Chromosome location | | | Cell line |
|---|---|---|---|---|---|---|
| Gene name | accession No. | Description | of CGI clones | Context | CpG island | methylated |
| DLC1^{a} | NM_006094 | Deleted in liver cancer 1 | chr8: 13034245-13034706 | 1st intron | Yes | 6 |
| PCDHGB7 | BC051788 | Protocadherin gamma subfamily B 7 | chr5: 140777313-140777950 | 1st exon | Yes | 5 |
| C21orf29 | AJ487962 | Chromsome 21 open frame 29 | chr21: 44955066-44956738 | 1st exon | Yes | 5 |
| STAM | BC030586 | Signal transducing adaptor molecule | chr10: 17726024-17726714 | 1st exon | Yes | 5 |
| C8orf13 | AL834122 | Chromosome 8 open reading frame 13 | chr8: 11362844-11363088 | Promoter | Yes | 5 |
| NASP | BC010105 | Nuclear autoantigenic sperm protein | chr1: 45718132-45718724 | 1st exon | Yes | 5 |
| RPIB9 | AK055233 | Rap2-binding protein 9 | chr7: 86902729-86903236 | 1st exon | Yes | 5 |
| NXPH1 | AB047362 | Neurexophilin 1 | chr7: 8255425-8255932 | 2nd intron | Yes | 5 |
| DDX51 | BC040185 | Homo sapiens DEAD box polypeptide 51 | Chr12: 131293874-131294410 | 2nd exon | Yes | 5 |
| DYRK4 | BC031244 | Dual-specificity tyrosine-(Y)- | chr12: 4583747-4584711 | Exon 6 | No | 5 |
| | | phosphorylation regulated kinase 4 | | | | |
| ZNF304 | AJ276316 | Zinc finger protein 304 | chr19: 62554224-62554913 | 1st exon | Yes | 5 |
| BCAT2 | BC004243 | BCAT2 protein | chr19: 53990469-53990898 | 1st exon | Yes | 5 |
| CCND1 | BC023620 | Cyclic D1 | chr11: 69165114-69165484 | 1st exon | Yes | 4 |
| MAD2L1BP | NM_001003690 | MAD2L1 binding protein isoform 1 | chr6: 43705205-43705621 | 1st exon | Yes | 4 |
| KCNK2 | AF004711 | TREK-1 potassium channel mRNA | chr1: 211643229-211643982 | Promoter | Yes | 4 |
| HMGCS1 | BC000297 | 3-hydroxy-3-methylglutaryl coenzyme A | chr5: 43348822-43349805 | 1st exon | Yes | 4 |
| RYL26 | BC066316 | Ribosomal protein L26 | chr17: 8226771-8221048 | 1st exon | Yes | 4 |
| NKX6 1 | NM_006168 | NK6 transcription factor related, locus 1 | chr4: 85773754-85774366 | 2nd exon | Yes | |
| ZCCHC11 | BC048301 | Zinc finger CCHC domain containing 11 | chr1: 52729841-52730282 | 1st intron | Yes | 4 |
| LRP1B | AF176832 | Low density lipoprotein-related protein 1B | chr2: 142721862-142722346 | 1st exon | Yes | 4 |
| EFNA5 | U26403 | ephrin-A5 | chr5: 107035237-107035819 | Promoter | Yes | 4 |
| SMC2L1 | AF092563 | SMC2 structural maintenance of | chr9: 103936037-103936585 | 1st exon | Yes | 4 |
| | | chromosomes 2-like 1 | | | | |
| PLOD2 | BC037169 | Procollagen-lysine, 2-oxoglutarate 5- | chr3: 147362180-147362504 | Promoter | Yes | 4 |
| | | dioxygenase (Lysine hydroxylase) | | | | |
| TMEM29 | AF370413 | DKPZp667C0711e | chrX: 52808646-52809350 | 1st exon | Yes | 4 |
| NOPE | AB046848 | KIAA1628 protein | chr15: 63476002-63476565 | 1st exon | Yes | 4 |
| CYP27B1 | BC001776 | Cytochrome P450, family 27, subfamily B, | chr12: 56,446,589-56,447,155 | 1st exon | Yes | 4 |
| | | polypeptide 1 | | | | |
| FLJ39155 | AK096474 | hypothetical protein FLJ39155 | chr5: 38293115-38293710 | Promoter | Yes | 3 |
| RPS16 | BC004324 | Ribosomal protein S16 | chr16: 28893019-28893612 | 1st exon | Yes | 3 |
| PON3 | L48516 | Paraoxonase 3 | chr7: 94669774-94670779 | 1st exon | Yes | 3 |
| RARB2 | NM_000965 | Retinoic acid receptor | chr3: 25,444,258-25,445,160 | 1st exon | Yes | 3 |

^{a}Sequences of the clones can be obtained from http://s-der10.med.utoronto.ca/CpGIslands.htm.

Reactivation of methylated genes by a demethylating agent and HDAC inhibitor. Real time RT-PCR was performed on 4 of these 10 genes in the cell lines treated with DAC and TSA (FIG. 12). CYP27B1 and RARβ2 were observed to be weakly to moderately up-regulated after DAC treatment, but there was a synergistic effect after combined DAC and TSA treatment in most of the cell lines. There was a synergistic effect for CCND1 in Raji, RL, Daudi, and DB cell lines in which CCND1 was significantly methylated, but not in Mec-1 and Granta 519 cells in which CCND1 is not methylated. Interestingly, the treatment with DAC down regulated CCND1 expression in the Granta 519 cell line. DLC-1 was induced only under combination drug treatment indicating involvement of both methylation and histone deacetylation in its epigenetic control. However in Daudi cell lines, combined epigenetic drug treatments failed to reactivate DLC-1 expression and a similar result was obtained for RARβ2 in the Granta 519 cell line.

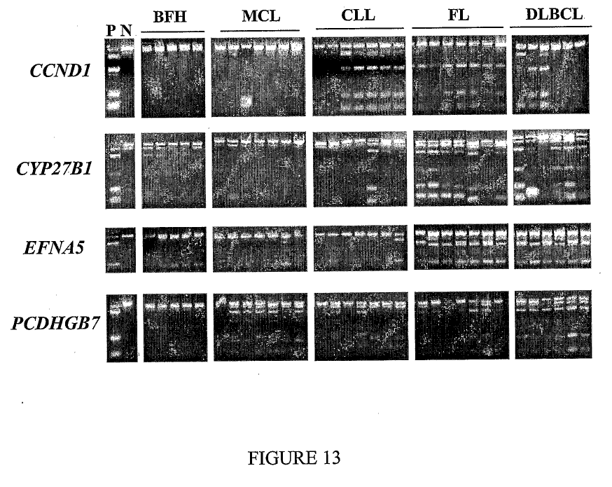

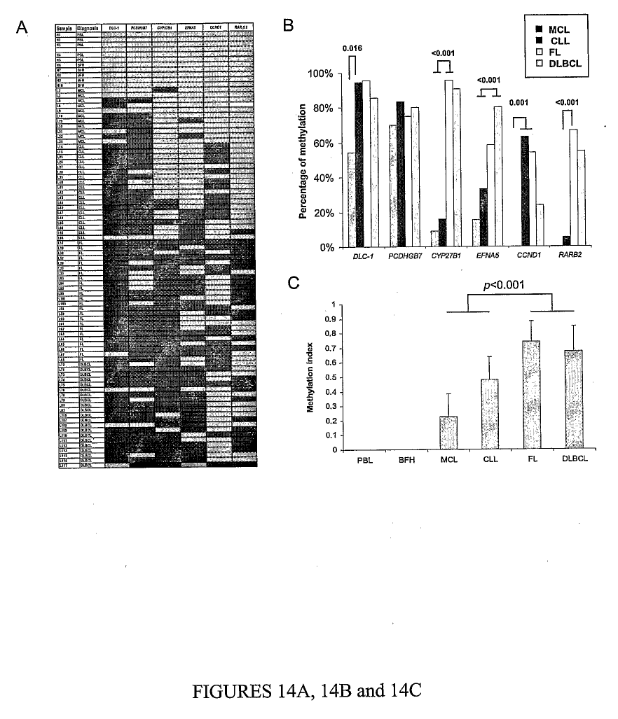

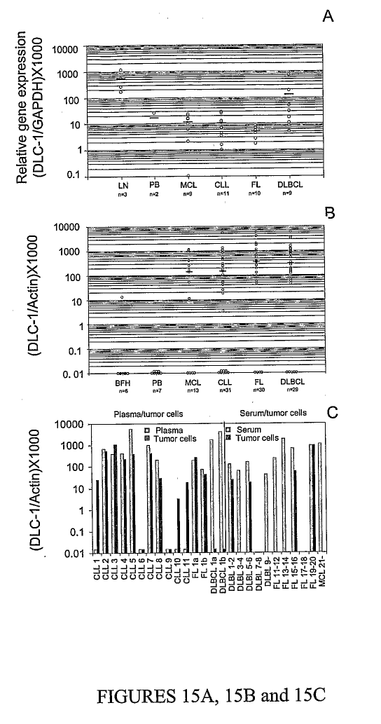

Hypermethylation in primary NHLs. The methylation profile of cancer cell lines does not always reflect the pattern of methylation in primary tumors. Therefore, the promoter methylation of 6 gene subset was selected and confirmed in a larger panel of NHLs (75 cases) including B-CLL/SLL, MCL, FL and DLBCL by COBRA and MSP analysis. Representative COBRA results of four of the genes are illustrated in FIG. 13. All six of the identified methylation-silenced genes in the cell line models were methylated in a significant proportion of NHL across the spectrum of subtypes (FIG. 14A). CpG island promoter hypermethylation of DLC-1 was the most common, being present in 87% of primary NHL, where PCDGHB7 was second most commonly methylated in 78% of NHL cases studied. Aberrant methylation was also detected in 61% of primary NHL for CYP27B1, 52% for EFNA5, and 40% for CCND1. Overall, RARβ2 methylation was found in 38% which is consistent with previous findings (19). Furthermore, a lymphoma subtype-related profile was observed (See FIG. 14B). For example CCND1 was methylated in FL and CLL, but not in MCL (p=0.001). This corresponding relationship is consistent with high levels of expression of cyclin D1 in MCL but not in FL and B-CLL/SLL (2). CYP27B1 and RARβ2 were mainly methylated in FL and DLBCL as compared to MCL and B-CLL/SLL (p<0.001). All 6 genes were not methylated in normal lymphocytes and BFH, confirming that the aberrant methylation is associated with malignancy.

Overall, simultaneous promoter methylation in ≧3 genes occurred in 9/14 (64%) of B-CLL/SLL, 2/10 (20%) of MCL, 15/15 (100%) of FL and 12/13 (92%) of DLBCL. As shown in FIG. 14A, only two cases of MCL are completely unmethylated for all 6 genes studied. Therefore, using the 6 epigenetic markers it is possible to detect 96% of NHL cases, indicating that gene methylation has substantantial utility as diagnostic test. To determine whether different types of NHLs displayed evidence of coordination of methylation at multiple loci, the Mann-Whitney U test was used to compare the mean methylation indices. This index is defined as the ratio of the number of methylated genes divided by the total number of genes analyzed between two variables. Significant differences were found in between the subtypes of NHLs, for instance, MCL vs CLL, FL or DLBCL (p<0.001), CLL vs FL or DLBCL (p<0.01). There is no statistical difference between FL and DLBCL (p>0.05). In general, germinal center related lymphomas (FL, DLBCL) have more methylation than non-germinal center lymphoma (MCL, CLL) (p<0.001, FIG. 14C). Although MCL patients are relative younger on average, there is no statistical difference in age between CLL, FL and DLBCL (p>0.05).

Down-regulation of DLC-1 gene expression in primary NHLs. The mRNA expression level of DLC-1 was quantified by real time RT-PCR in 5 normal controls and 39 primary NHL samples. As shown in FIG. 15B, DLC-1 mRNA could be detected in normal lymph node samples and weakly in peripheral blood lymphocytes suggesting a tissue or developmental stage-specific expression or possibly indicating other silencing mechanisms might exist in normal leukocytes other than methylation. DLC-1 mRNA was also weakly expressed in some cases of MCL, B-CLL/SLL, and FL, and somewhat stronger in DLBCL cases. When overall DLC-1 mRNA expression was compared between tumor and normal lymph node, its expression was lower in tumors. The reciprocal relationship between DLC-1 promoter methylation and its expression suggests that promoter methylation is a major mechanism for DLC-1 silencing in germinal center related NHLs.

Quantitative analysis of DLC-1 methylation in tumor and plasma samples of NHL patients. To test the idea of utilizing DLC-1 as a biomarker, a real time quantitative MSP assay was designed and expanded the methylation analysis from all the samples described above to now include additional samples from patients with MCL, CLL, FL and DLBCL. When a cut off ratio of DLC-1: β-actin×1000 was set as 15, the DLC-1 methylation frequencies were 71%, 62%, 83%, and 83%, respectively (FIG. 15A). When this quantitative MSP method was compared to standard MSP, the consistency between the two methods was 93%. The relative methylation level of each sample, as measured by the ratio of DLC-1: β-actin×1000, varies among the 4 sub-classes of NHL studied. The median methylation level was 135 (range from 0 to 1099) for MCL, 141 (range from 0 to 5378) for B-CLL/SLL, 348 (range from 0 to 5683) for FL and 295 (range from 0 to 5912) for DLBCL (FIG. 12). Interestingly, both the frequency and relative level of methylation of DLC-1 seems to correlate with the putative stages of differentiation. The methylation level is relatively higher in germinal center-related NHLs such as FL and DLBCL (some cases are post-germinal center), as compared to MCL and B-CLL/SLL which are usually derived from pre- or post-germinal center cells. The increased methylation level was not attributable to the variability in tumor cell percentage or age (p>0.05).

For a subset of 15 patients with B-CLL/SLL, FL, or DLBCL, paired tumor and plasma samples were available. Of these, 12/15 samples demonstrated concordant results, with 10/12 samples showing methylation in both the tumor and in plasma and 2/12 did not show methylation in either the tumor or in plasma. The 3 discordant samples all demonstrated tumor methylation, but none was detected in the plasma samples. Two of the 3 were from patients with localized stage I FL. For all these samples, we examined DLC-1 methylation not only in the tumor and in plasma, but also from buffy coat preparations of peripheral blood cells. In all cases of B-CLL/SLL and FL where methylation was present in the tumor, it was also present in buffy coat cells. However, in the case of DLBCL, methylation was present in the tumor and plasma, but not in buffy coat cells, which is consistent with the fact that most patients with DLBCL (other than those with advanced disease) do not have detectable circulating tumor cells in blood.

### Example 4

### Multiple Novel Methylated Genes were Identified by ECISTs Microarray Screening, Confirmed in Multiple Myeloma (MM) Cell Lines and Primary MM Samples, and have Substantial Utility for Diagnosis, Prognosis and Monitoring of Aspects of MM

### Example Overview

Experimental design. Expressed CpG Island Sequence Tags (ECISTs) microarray (14), is an integrated microarray system that allows assessing DNA methylation and gene expression simultaneously, and provides a powerful tool to further dissect molecular mechanisms in MMs, and to assess related pharmacologic interventions by differentiating the primary and secondary causes of pharmacological demethylation. This innovative microarray profiling of DNA methylation was used in this Example to define Epigenomic Signatures of Myelomas. Novel epigenetic biomarkers were identified that have substantial utility for diagnosis and prognosis.

Results. In this Example, methylation microarray profiling was conducted in the context of 4 multiple myeloma (MM) cell lines, 18 MM primary tumors and 2 normal controls. Multiple novel methylated genes were identified, and a subset of these were confirmed in MM cell lines and in primary MM samples (20 primary MM samples from our cell bank, from which DNA was isolated). Additionally, a real time methylation-specific PCR assay was developed for the tumor suppressor gene DLC-1, and was optimized in terms of sensitivity and variability. Furthermore, four MM cell lines were treated with a demethylating agent and histone deacetylase inhibitor, and RNA was isolated from the drug-treated cell lines.

### Materials and Methods:

Cultured B-cell lines and drug treatment. Myeloma lines U266, NCI-H929, RPMI 8226 and KAS 6/1 were maintained in RPMI 1640 media supplemented with 10% fetal bovine serum (FBS). KAS 6/1 cells were supplemented with IL-6 at a concentration of 10 ng/mL. For ‘gene reactivatio’ experiments, cells were cultured in the presence of vehicle (PBS) or 5-aza-2′-deoxycytidine (1.0 μM; medium changed every 24 h). After 4 days, cells were either harvested or further treated with TSA (1.0 μM) for 12 h and then harvested. Some cells were also treated with TSA alone for 12 h before harvest. Genomic DNA or total RNA was isolated using Qiagen™ kits (Qiagen, Valencia Calif.) and used for methylation and gene expression analysis, respectively.

Tissue sample preparation. Plasma cells were enriched by immunomagnetic separation. Cell suspensions were incubated with an anti-CD138 (Beckman Coulter, Fla.) respectively at 4° C. for 30 min, washed twice in PBS containing FCS (0.5%), and incubated in the cold for 15 min with magnetic beads coated with α-mouse IgG (Dynal, N.Y.). CD138 is known as Syndecan-1 and is expressed on normal and malignant plasma cells but not on circulating B-cells, T-cells and monocytes. B-cell subsets were examined by flow cytometry analysis.

Methylation microarray analysis. The approach was adapted from a previously described protocol (15). Briefly, 2 μg genomic DNA was restricted with MseI, a 4-base TTAA endonuclease that restricts bulk DNA into small fragments (<2000-bp), but retains GC-rich CpG islands. The ‘sticky ends’ of the digests were ligated with 0.5 nmol PCR linkers H-24/H-12 (H-24: 5′-AGG CAA CTG TGC TAT CCG AGG GAT (SEQ ID NO:6), and H-12: 5′-TAA TCC CTC GGA (SEQ ID NO:7)). Linker-ligated DNA was digested by McrBC, a restriction enzyme that only cuts methylated DNA sequences (16). About 20 ng of the linker-ligated-uncut samples and 20 ng linker-ligated-McrBC-cut DNA were amplified by PCR. The amplified products (or amplicons) were purified for fluorescence labeling. Incorporation of aa-dUTP into amplicons was conducted using the Bioprime™ DNA Labeling System (Invitrogen, Carlsbad, Calif.). Cy5 and Cy3 fluorescence dyes were coupled to aa-dUTP-labeled McrBC-cut and uncut amplicons respectively, and co-hybridized to the 12K CpG island microarray panel. Hybridization and the post-hybridization washing were done according to the manufacturer's procedures (Corning Life Sciences, Acton, Mass.). Hybridized slides were scanned with the GenePix™ 4200A scanner (Axon, Union City, Calif.) and the acquired images were analyzed with the software GenePix™ Pro 5.1.

Microarray data analysis. The hybridization output is the measured intensities of the two fluorescent reporters, Cy3 and Cy5, false-colored with green or red and overlaid one on the other. The fluorescence ratios calculated for each CpG island (digested/undigested) reflect the degree of DNA methylation for each CpG island locus. Mitochondrial DNA is unmethylated (17), therefore signals intensities of both channels coming from mitochondrial clones are expected to be equal. Data from arrays analyzing methylation were normalized based on signals of 60 spots containing mitochondrial clones. These spots were spotted in each of 48 blocks. Their pixel intensities covered the whole signal range of the microarray. After normalization, a ratio that approaches 0 indicates a methylated CpG island—no production of labeled PCR product following McrBC digestion, while the undigested reference will yield labeled PCR product. A ratio approaching 1 indicates an unmethylated CpG island—fluorescently labeled PCR product will be obtained in both the McrBC digested test sample and the undigested reference. The average Cy5/Cy3 ratio of two experiments (dye-swapped) was used for comparison.

Confirmation of methylation analysis by MSP and COBRA. Methods for bisulfite modification of DNA and subsequent PCR techniques used in this study are as described earlier (14). 1 μg of genomic DNA was treated with sodium bisulfite according to the manufacture's recommendations (Ez™ DNA methylatin kit; Zymo Research, Organe, Calif.). This treatment converts unmethylated, but not methylated, cytosine to uracil in the genome. For the preparation of 100% methylated DNA, a blood DNA sample was treated with M. SssI methyltransferase that methylates all cytosine residues of CpG dinucleotides in the genome. Sodium bisulfite modification of the test and SssI-treated DNA samples were then performed as described above. Bisulfite-treated genomic DNA (100-200 ng) was used as a template for PCR with specific primers located in the CpG island regions of multiple genes. For MSP, allele specific primers were designed to differentiate methylated and unmethylated sequences. Amplification was performed using AmpliTaq Gold™ polymerase. For COBRA, after amplification, PCR products were digested with the restriction enzyme BstUI (New England Biolabs), which recognizes sequences unique to the methylated and bisulfite-unconverted alleles. The digested and undigested control DNA samples were separated in parallel on 3% agarose gels, stained with SYBR green and quantified using Kodak gel documentation system.

Development of real time methylation specific PCR. Bisulfite treatment of the DNA was performed as described above. The real time methylation specific PCR uses two amplification primers and an additional, amplicon-specific, and fluorogenic hybridization probe whose target sequence is located within the amplicon. The published primers (M(+): 5′-CCC AAC GAA AAA ACC CGA CTA ACG-3′ (SEQ ID NO:1); M(−): 5′-TTT AAA GAT CGA AAC GAG GGA GCG-3′ (SEQ ID NO:2); U(+): 5′-AAA CCC AAC AAA AAA ACC CAA CTA ACA-3′ (SEQ ID NO:3); U(−): 5′-TTT TTT AAA GAT TGA AAT GAG GGA GTG-3′ (SEQ ID NO:4)) for DLC-1 were used for the PCR amplification of methylated and unmethylated alleles in two separate reactions. The real-time methylation specific PCR uses the same two amplification primers specific for methylated sequences and an additional, amplicon-specific, and fluorogenic hybridization probe (Probe: FAM/AAG TTC GTG AGT CGG CGT TTT TGA/BHQ_{—}1 (SEQ ID NO:5)) whose target sequence is located within the amplicon. The probe was labeled with two fluorescent dyes, with FAM at the 5′-end and with BHQ1 at the 3′-end. The primers/probe set for real-time methylation specific PCR were synthesized by IDT. The bisulfite treated DNA was used for PCR amplification with appropriate reagents in QPCR mix (ABgene) as recommended by the manufacturer. The reaction was carried out in 40-45 cycles using a SmartCycler™ real-time PCR instrument (Cepheid).

### Results:

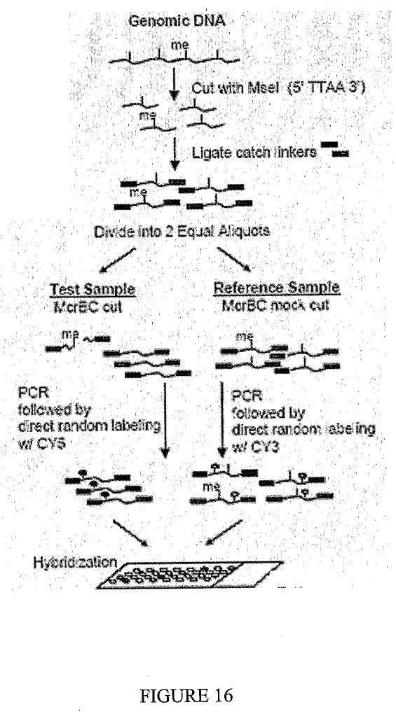

Methylation profiling of four myeloma cell lines. The microarray was first used to identify hypermethylated CpG island loci in four MM cell lines. Cy3- and Cy5-labeled amplicons, representing differentially methylated pools of genomic DNA were co-hybridized on the 12K CpG island microarray. Genomic DNA fragments containing methylated CpG sites in the McrBC-cut sample were digested by McrBC and can not be amplified by linker-PCR, whereas the equivalent allele can be amplified in the uncut sample (FIG. 16). Spots hybridized predominantly with the uncut amplicon but not with the McrBC-cut amplicon, indicative of methylated CpG sites in the DNA sample, are expected to show up green. The presence of “yellow” spots indicates a roughly equal amount of bound DNA from McrBC-cut and uncut amplicons, indicative of unmethylated CpG sites in the DNA sample. Therefore, the fluorescence ratio calculated for each CpG island (digested/undigested) reflects the degree of DNA methylation for each CpG island locus. Mitochondrial DNA is unmethylated (17), therefore signals intensities of both channels coming from mitochondrial clones are expected to be equal. Data from arrays analyzing methylation were normalized based on signals of spots containing mitochondrial clones. After normalization, a ratio that approaches 0 indicates a methylated CpG island—no production of labeled PCR product following McrBC digestion while the undigested reference will yield labeled PCR product. A ratio approaching 1 indicates an unmethylated CpG island—fluorescently labeled PCR product will be obtained in both the McrBC digested test sample and the undigested reference. The hybridization experiments were repeated using “dye-swap” method, and only those reproducible spots were chosen for analysis. DNA samples from normal male and female lymphocytes are processed in the same way as indicated above.

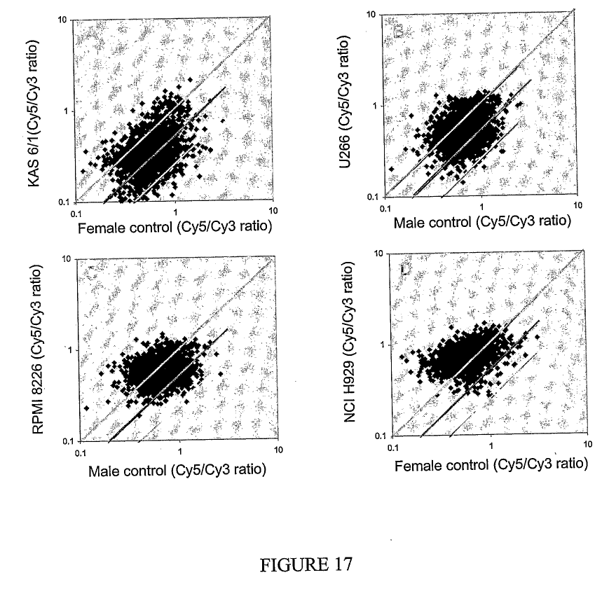

FIG. 17 shows the scatter plots of Cy5/cy3 ratio of four MM cells as compared with normal lymphocyte control in a sex matched manner. A lower Cy5/cy3 ratio in the cancer cell line as compared to the normal control indicates hypermethylation and a higher Cy5/Cy3 ratio in the cancer cell line indicates hypomethylation. The methylation index for each CpG island was defined as the Cy5/Cy3 ratio from tumor sample divided by the Cy5/Cy3 ratio from a normal control sample. A z-statistic test was conducted using the methylation index ratios and the z-score for each CpG locus was calculated. When a cut-off value of the z-score was set at <−1.96 (95% confidence) for the positive loci, a total of 81 methylated CpG loci (2.0% of 3962 CpG island fragments) were identified in KAS 6/1, 62 (1.56%) in U266, 44 (1.11%) in RPMI 8226, and 56 (1.41%) in NCI H929. KAS 6/1, an IL-6-dependent MM cell line, shows a great number of genes methylated as compare to normal control. Recent report shows that IL-6 could induce promoter hypermethylation through up-regulation of DNMT1 or STAT3, which is consistent with the instant findings (18).

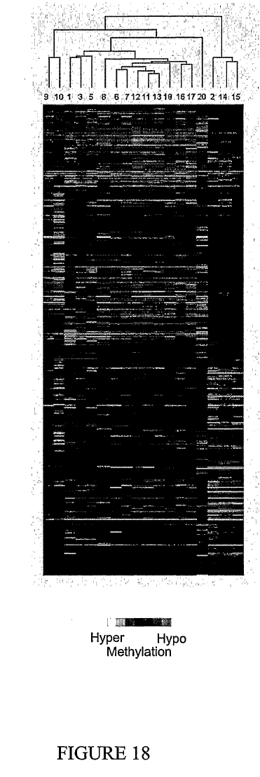

Methylation profiling of 18 cases of primary myelomas. Primary myeloma samples from 18 cases were then studied using the microarray strategy described above. The Cy5/Cy3 ratios ratios, which represent the level of methylation of each CpG island locus from 3,962 annotated genes were used for initial analyses. The methylation index ratio for each CpG island locus in each tumor samples was calculated as described above. The ratios were then used for cluster analyses (FIG. 18). Although the sample size in this analysis is relative small, it seems that a non-random methylation pattern was observed in the 18 cases of primary myeloma. The association of the clusters with any clinicopathological data is currently under investigation.

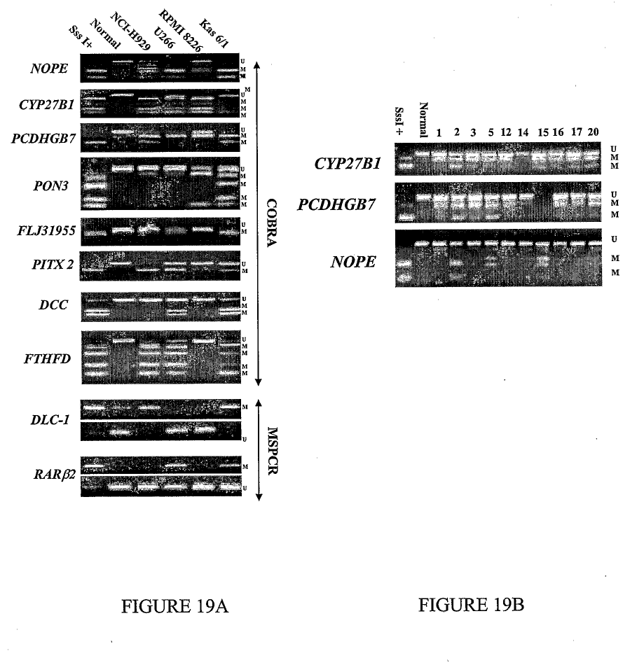

Confirmation Study in Cell Lines. As an initial test, the microarray findings of 10 known genes (PCDHGB7, CYP27B1, DLC-1, NOPE, FLJ39155, PON3, PITX2, DCC, FTHFD and RARβ2) whose function might relate to cancer were independently confirmed by COBRA and MSP analyses. Hypermethylation of these genes was confirmed in the 4 MM cell lines (FIG. 19A). The most frequently methylated, PCDGHB7, CYP27B1, and NOPE were methylated in all 4 cell lines. The remaining 7 genes are methylated in 1 to 3 cell lines. Consistent with the microarray findings, all 10 genes were found to be methylated in Kas 6/1, the IL-6 dependent cell line.

Confirmation Studies in Primary Myelomas. A subset of 3 of the above-identified genes was selected and the promoter methylation was confirmed in 10 cases of primary MMs. Representative COBRA results of the three genes are illustrated in FIG. 19B. All the three most frequently methylated genes in the cell line models were methylated in a significant proportion of primary MMs. Aberrant methylation can be detected in 80% of primary MM for CYP27B1, 80% for PCDHGB7, and 30% for NOPE. Most of the methylated genes discovered in this Example have not been reported in MMs before. Although the function of some of these genes in MM biology may be uncertain, some of them (e.g., DLC-1, DCC, and PITX2) have been demonstrated as tumor suppressor genes in other type of tumors.

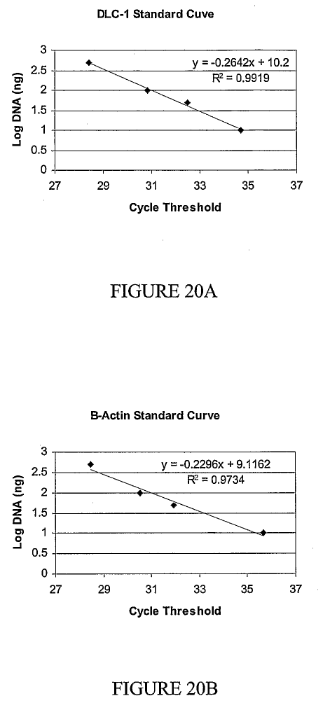

A real-time methylation-specific PCR assay with high sensitivity and reproducibility was developed. As disclosed herein, DLC-1, a candidate tumor suppressor gene (19), was methylated in a large portion of leukemia and lymphoma. A real time quantitative methylation specific PCR (qMSP) assay was therefore developed for DLC-1 gene. To quantify the methylation level of DLC-1 in each sample analyzed, a probe was designed to include the CpG island in the DLC-1 promoter, the hypermethylation of which is known to be correlated with a lack of DLC-1 gene expression. The relative methylation levels in a particular sample are measured by the ratio of DLC-1 ACTIN×1000. To reliably determine a quantitative cut-off for positivity, the intra-assay and inter-assay variability was examined. Three lymphoma cell lines were used, and each was divided into 5 separate aliquots and treated with sodium bisulfite in preparation for qMSP analysis. All 5 samples were analyzed in the same group on the same day to represent the variation that might be expected within a single analytical run. The intra-assay co-efficient of variation (CV) ranged from 0.422-0.644 when the variable was the qMSP cycle number (Cₜ). For the β-actin internal control, the range of CV was 0.346-0.746. When the ratio of DLC-1 methylation: β-actin was plotted on the standard curve, the CV increased to a range of 9.92-16.6, dependent on the cell line. To test the inter-assay variability, 5 aliquots of each cell line were independently treated and assayed on 5 separate days. The inter-assay CV for DLC-1 ranged from 0.820-2.31 when the variable was the Cₜ. For the β-actin internal control, the range of CV was 0.709-1.92. When the ratio of DLC-1 methylation: β-actin was plotted on the standard curve, the CV increased to a range of 5.71-17.5, dependent on the cell line. The assay sensitivity was determined by using serial dilutions of Raji cell DNA before bisulfite treatment and determining the least amount of methylated DLC-1 that could be detected in the assay. In this case, tumor DNA could be detected at a dilution of 1:10,000. As show in FIG. 20A, the methylated DLC-1 DNA can be detected from as low as 10 ng of bisulfite treated Raji DNA, and the Cₜ value was 36.17. Overall, the slope regression was 0.9919 for the DLC-1 standard curve, and 0.9734 for the β-actin standard curve.

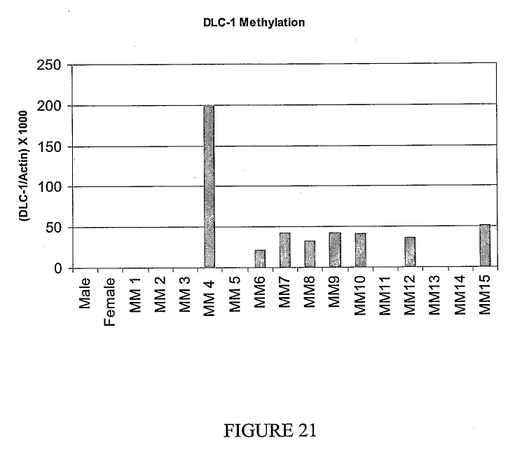

Quantitative analysis of DLC-1 methylation in primary MMs. 15 primary MM samples were analyzed using the qMSP assay developed above (FIG. 21). DLC-1 promoter hypermethylation was positively detected in 8 out of 15 MM samples (53%). The quantitative value of the methylation in MM is relatively smaller than lymphoma, particularly follicular lymphoma and large B-cell lymphoma. Although the effect of low amount of methylation on DLC-1 gene expression is unknown at this point, DLC-1 has substantial utility as a MM biomarker and the instant qMSP assay demonstrated great sensitivity and specificity.

### Example 5

### Differential Methylation Hybridization was Used to Determine and Compare the Genomic DNA Methylation Profiles of the Granulocyte Subtypes of Acute Myelogenous Leukemia (AML), and Also to Distinguish AML and ALL

### Example Overview

Rationale and experimental design. The intent of this Example was to determine whether genomic methylation profiling could be used to distinguish between clinically recognized subtypes of acute myelogenous leukemia (AML). Aberrant DNA methylation is believed to be important in the tumorigenesis of numerous cancers by both silencing transcription of tumor suppressor genes and destabilizing chromatin. Previous studies have demonstrated that several tumor suppressor genes are hypermethylated in AML, suggesting a roll for this epigenetic process during tumorigenesis. However, it is unknown how the genomic methylation profiles differ among AML variants, or even whether AML can be distinguished on this basis from normal bone marrow or other hematologic malignancies. In this Example, the epigenomic microarray screening technique called Differential Methylation Hybridization (DMH) was applied to the analysis of 23 bone marrow samples from patients having the AML granulocytic subtypes M0 to M3 as well as normal controls.

Results. With this method, a unique genomic methylation profile was created for each patient by screening sample DNA amplicons with an array of over 8600 CpG-rich DNA tag sequences. Cluster analysis of methylation features was then performed that demonstrated these disease subtypes could be sorted according to methylation profile similarities. From this screening, over 70 genomic loci were identified as being hypermethylated in all four examined AML subtypes relative to normal bone marrow. Three hypermethylated loci in M0 samples were found to distinguish this class from all others. Sequence analysis of these loci was performed to identify their encoded genes. Confirmation of their methylation status in AML was conducted using MS-PCR and COBRA analyses.

Results of this Example indicate that genomic methylation profiling has substantial utility not only for diagnosing AML and subtypes thereof, but also in distinguishing this disease from other hematopoietic malignancies. Moreover, analysis of the impact of methylation on the expression of the identified genes will facilitate understanding the underlying molecular pathogenesis of AML.

### Materials and Methods:

Differential Methylation Hybridization (DMH). Differential Methylation Hybridization screening was applied, essentially as described elsewhere herein above, to the analysis of 23 bone marrow samples from patients having the AML granulocytic FAB subtypes M0 to M3 as well as disease-free bone marrow samples. MS-PCR, COBRA and Cluster analysis was performed essentially as described herein above.

### Results:

DMH screening of 23 bone marrow samples identified over 70 genomic loci as being hypermethylated in all four examined AML subtypes relative to normal bone marrow, and particular loci are listed in TABLE 5.

TABLE 5
Hypermethylated Genes in AML Identified Using CGI Array.

| | Accession number | Hypermethylated |
|---|---|---|
| Gene | (SEQ ID NOS) | % |
| LRP1B | See Table 10 above | 74 |
| CSDA | | 65 |
| BX161496 | | 65 |
| FBXO36 | | 65 |
| DDX51 | See Table 10 above | 57 |
| ZNF304 | | 57 |
| NKX6-1 | See Table 10 above | 57 |
| DDX51 | See Table 10 above | 52 |
| ATP5B | | 52 |
| MYBBP1A | | 52 |
| SMC2L1 | | 52 |
| H3F3A | | 48 |
| MGC13204/FOXM1 | | 48 |
| MCF2L2 | | 48 |
| NASP | | 43 |
| FOXD2 | | 43 |
| DYRK4 | | 43 |
| DPYSL5 | | 43 |
| TAB3 | | 43 |
| ZA20D1 | | 39 |
| MGC13102 | | 39 |
| KCNK2 | | 39 |
| ALX4 | | 39 |
| GPR68 | | 39 |
| GNAL | | 39 |
| C3orf4 | | 39 |
| GTPBP2/MAD2L1BP | | 39 |
| STAM | | 35 |
| EXOSC8 | NM_181503 | 35 |
| | Clone SEQ ID NO: 176 | |
| | (chr13: 36472745-36473016) | |
| | CGI SEQ ID NO: 177 | |
| | (chr13: 36472793-36473223) | |
| | Amplicon SEQ ID NO: 178 | |
| | (chr13: 36472749-36473030) | |
| NOPE | See Table 10 above | 35 |
| SEN2L | | 35 |
| HMGCS1 | | 35 |
| MGC5242 | | 35 |
| OAZIN | | 35 |
| C8orf13 | | 35 |
| BCL10 | | 30 |
| GCLM | | 30 |
| RPL26 | | 30 |
| ID1 | | 30 |
| C21orf29 | | 30 |
| HIST1H4E | | 30 |
| c6orf55 | | 30 |
| DDX51 | | 26 |
| TUBGCP3 | | 26 |
| SMAD9 | NM_005905 | 26 |
| | (Clone SEQ ID NO: 179) | |
| | chr13: 36391067-36391675 | |
| | (CGI SEQ ID NO: 180) | |
| | chr13: 36391897-36392752 | |
| | (Amplicon SEQ ID NO: 181) | |
| | chr13: 36391451-36391632 | |
| PLEKHG2 | | 26 |
| HIST1H2AB | | 26 |
| RP1B9 | See Table 10 above | 26 |

Sequence analysis of these loci (DNA tags) was performed to identify their encoded genes, revealing several genes not previously associated with abnormal methylation in AML, including the dual-specificity tyrosine phosphorylation regulated kinase 4, structural maintenance of chromosome 2-like-1, and the exportin 5 genes. In particular aspects, three hypermethylated loci in M0 samples were found to distinguish this class from all others.

Confirmation of their methylation status in AML was conducted using MS-PCR and COBRA analyses (FIGS. 22A-O).

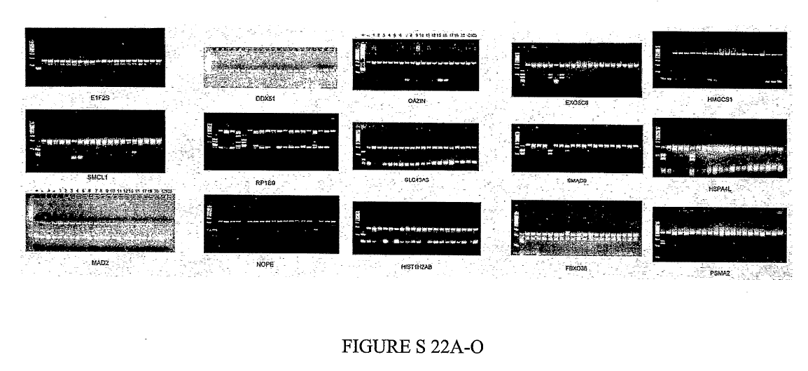

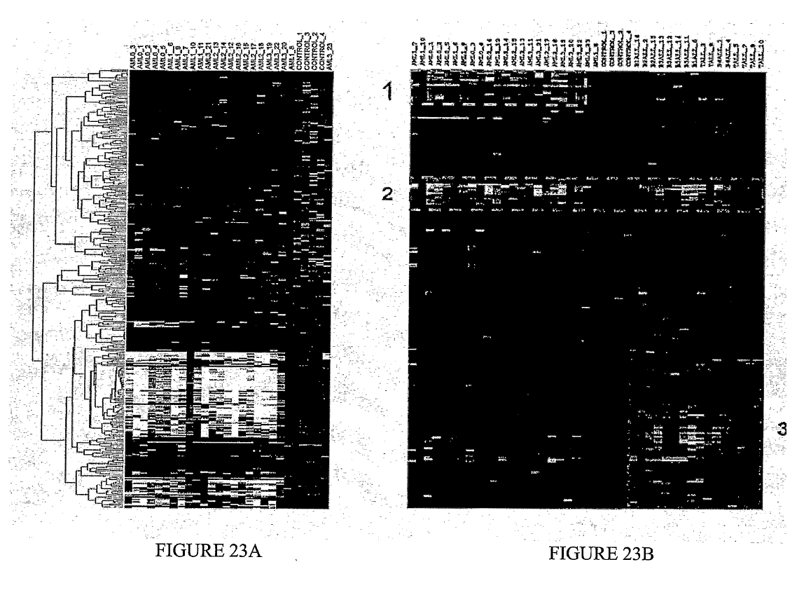

Cluster analysis of methylation features from each sample was then performed, demonstrating that the FAB M0-M3 subtypes could be discriminated on the basis of their methylation profile patterns (FIG. 23A). FIG. 23A shows, according to particular aspects, cluster analysis of sample methylation features, demonstrating that the FAB M0-M3 subtypes could be discriminated on the basis of their methylation profile patterns.

Distinguishing between AML and ALL. FIG. 23B shows, according to additional aspects, hierarchical clustering of DNA methylation in AML and ALL. Methylation microarray analysis revealed distinctive methylation patterns in AML and ALL patients from different subtypes: Region “1” illustrates loci hypermethylated in AML; Region “2” shows loci hypermethylated in both AML and ALL; and Region “3” shows loci hypermethylated in ALL patients.

In additional experiments, differential methylation of 508 chromosomal loci in ALL and AML was evaluated and used to differentiate these two diseases. The cluster image created from the DMH experiments demonstrated a clear delineation between ALL and AML samples of various subtypes. Furthermore, the cluster illustrated numerous hypermethylated and hypomethylated loci. For example, a prominent cluster of hypermethylated loci in AML is seen in one region of an array and a similar cluster is seen including hypomethylated loci in ALL samples. The following genes were found to be hypermethylated in AML and may be possible tumor suppressor genes: DPYSL5, ARL61P2, SLIT2, HSPA4L, HOXB13, and CKS2.

Therefore, the present compositions and methods enable discrimination between ALL and AML using differential methylation patterns, and methylation patterns in ALL and AML provide a blueprint for the behavior of this heterogeneous disease. The methylation patterns identified in ALL and AML have substantial diagnostice prognostic utility.

### Example 6

### Differential Methylation Hybridization was Used to Determine the Genomic DNA Methylation Profiles of Acute Lymphoblastic Leukemia (ALL)

### Example Overview

Rationale and experimental design. Previous studies investigating the aberrant methylation of gene promoters in ALL have associated hypermethylated promoters with prognosis (Roman-Gomez et al. 2004), cytogenetic alterations (Shteper et al. 2001; Maloney,et al. 1998), subtype (Zheng et al. 2004) and relapse (Matsushita et al. 2004). However, elucidaticdation of the aberrant methylation profiles in ALL is limited by the small number of CGIs analyzed to date, The intent of this Example was to determine whether genomic methylation profiling could be used to identify and distinguish Acute Lymphoblastic Leukemia (ALL). Aberrant DNA methylation is believed to be important in the tumorigenesis of numerous cancers by both silencing transcription of tumor suppressor genes and destabilizing chromatin. Until the present work, it was unknown whether ALL could be distinguished from normal bone marrow on this basis. In this Example, the epigenomic microarray screening technique called Differential Methylation Hybridization (DMH) was applied to the analysis of bone marrow samples from patients having ALL, as well as from normal controls.

Results. In this Example, to attain a global view of methylation within the promoters of genes in ALL patients and to identify a novel set of hypermethylated genes associated with ALL, methylation profiles for 16 patients were generated using DMH and a CpG island array that contains clones representing more than 4 thousand unique genes spanning all human chromosomes. From the generated profiles, 49 candidate genes were identified to be differentially methylated in at least 25% of patient samples. The presence of methylation in DCC, DLC-1, DDX51, KCNK2, LRP1B, NKX6-1, NOPE, PCDHGA12, RPIB9/ABCB1(MDR1) and SLC2A14 was verified by COBRA, MSP or qMSP. We examined the expression of these genes in 2 ALL cell lines (Jurkat, NALM-6) pre- and post-treatment with 5-aza and TSA by semi-quantitative real-time RT-PCR. In all cases, methylation corresponded to the down-regulation or silencing of the gene and up-regulation of gene expression was achieved after treatment.

Therefore, particular aspects of the present invention provide ALL-specific epigenetic profiles having substantial utility for subtype classification, prognosis and treatment response in ALL patients.

### Materials and Methods:

Tissue specimens. Bone marrow samples of patients diagnosed with leukemia at the Ellis Fischel Cancer Center (Columbia, Mo.) were obtained with the Institutional Review Board approval. DNA was isolated using the QIAamp™ DNA Mini Kit (Qiagen, Valencia, Calif.) according to the manufacturer's specifications from 16 specimens: 6 from patients diagnosed with T-ALL and 10 from patients diagnosed with pre B-ALL (TABLE 6).

TABLE 6
Patient characteristics.

| Patient | Age | Sex | Blast Lineage | Immunophenotype | Cytogenetics |
|---|---|---|---|---|---|
| 1 | 21 | M | B-ALL | 19; −10; 20 | Del19(p13) |
| 2 | 35 | F | B-ALL | 19; 10 | Phil t(9; 22) BCR-ABL |
| 3 | 16 | F | T-ALL | Unknown | Normal |
| 4 | 8 | M | B-ALL | 19; −10; 20 | Unknown |
| 5 | 5 | M | T-ALL | Unknown | Unknown |
| 6 | 14 mo | F | B-ALL | 19; −10 | t(4; 11; 13)(q21; q23; q12) MLL |
| 7 | 16 | M | T-ALL | Unknown | Normal |
| 8 | 17 | M | T-ALL | Unknown | Var(21) |
| 9 | 2 | F | T-ALL | Unknown | Unknown |
| 10 | 17 | M | T-ALL | Unknown | Unknown |
| 11 | 4 | F | B-ALL | 19; 10; 20 | 44-47, X-X |
| 12 | 3 | M | B-ALL | 19; 10; 20 | Normal |
| 13 | 55 | F | B-ALL | 19; 10; 20 | Normal |
| 14 | 51 | M | B-ALL | 19; 10; 20 | Phil t(9:22) BCR-ABL |
| 15 | 2 | M | B-ALL | 19; 10 | Hyperdiploid |
| 16 | 18 mo | M | B-ALL | 19; −10 | t(11; 19)(q23; p13) MLL |

Amplicon development and differential methylation hybridization (DMH). Amplicons were generated and DMH was performed as previously described (Huang et al 1999; incorporated by reference herein). Briefly, 2 μg of genomic DNA from malignant and non-malignant cells were digested with MseI followed by ligation of PCR linkers and digestion with methylation sensitive endonucleases (HpaII and BstUI). PCR was then performed amplifying only methylated fragments or fragments containing no internal HpaII or BstUI sites. The amplicons from the malignant and normal sample were labeled with Cy5 or Cy3 fluorescence dye respectively and cohybridized to a panel of 8,640 short CpG island tags arrayed on a glass slide. The slides were scanned with GenePix™ 4200a scanner and signal intensities of hybridized spots were analyzed with the GenePix™ 4.0 software program (Molecular Devices Corporation, Sunnyvale, Calif.).

To determine which clones were differentially methylated in the tumor versus the normal samples, we used global normalization for each array then performed across-array analysis for each spot. The Kruskal-Wallis non-parametric test was then used to identify clones that were differentially methylated in ALL and non-malignant samples.

Clone sequences. Sequences from differentially methylated CpG clones were extracted from the Der laboratory website (http://derlab.med.utoronto.ca/CpGIslands/). BLAST searches were performed to determine if these clone sequences were associated with the promoter region of known genes and if these regions contained CpG islands. Finally, we used these sequences were used to develop primers for RT-PCR and PCR using MethPrimer™ and Primer3™ respectively.

Methylation specific PCR (MSP) and combined bisulfite and restriction analysis (COBRA). Two μg of DNA was treated with sodium bisulfite according to the manufacturer's recommendations (Ez™ DNA methylation kit; Zymo Research, Orange, Calif.). Bisulfite treated DNA was used as a template for PCR with specific primers designed using Primer3™ and that were located in the CpG island regions of each tested gene (TABLE 7).

TABLE 7

Primers used for COBRA and Real-time SYBR Green analyses.

| | | | | | |
|---|---|---|---|---|---|
| | SEQ | | SEQ | Annealing | Product |
| Sense Primer (5′ to 3′) | ID NO | Antisense Primer (5′ to 3′) | ID NO | Temp (° C.) | size² |

| | | | | | | |
|---|---|---|---|---|---|---|
| COBRA¹ | | | | | | |
| DCC | GGATATTTTAGAAAAGTGAGAG | 66 | CAAATCATCAATAAACCACATCCAAA | 67 | 55 | 300 |
| DDX51 | TTTTTTATTTGTTTTATTTAAGGTGTT | 68 | TCTACTAAACTTACCCCTATCCTCC | 69 | 56 | 250 |
| KCNK2 | TTTAGTAAAGGGGTTTTGTTTTGAG | 70 | AACCCTAACTTCTTCCAATCTACAC | 71 | 56 | 230 |
| NKX6-1 | TTTTGTATATTTGGAGGGATAGGTAT | 72 | CCTTTTATTCATCAAAAATTTACCC | 73 | 54 | 210 |
| NOPE | TTTTTTGTTTTATTTATTTTAGTTTTAGTT | 58 | AAAACCCATCTCCACAAATATCAT | 59 | 56 | 210 |
| PCDHGA12 | AATGTTTAGATTTAATGTATATTTGATGGT | 74 | CTCCAAAAACCTAAAACTAAAACCC | 75 | 56 | 180 |
| RP1B9 | ATTGGAATTGATATAAAGTTTAGGGTT | 60 | ACCCCCTTAAACAAATATAAAAAAC | 61 | 56 | 400 |
| SLC2A14 | GGTTTTAAGGTTAGTTTTTTAGAGT | 76 | AAACAATTAATAAATCCCAAC | 77 | 54 | 270 |
| Real-time | | | | | | |
| ABCB1 | TGTATGCTCAGAGTTTGCAGGT | 78 | TTCCAAAGATGTGTGCTTTCC | 79 | 58 | 60 |
| DCC | CCGAAAGTCCCTTACACACC | 80 | CATGGGTCTTAGGAAGAGTGG | 81 | 58 | 60 |
| DDX51 | CACACTGCTCCTGAAAGTGC | 82 | TTCAGTTAGCATTCGGAGGAA | 83 | 58 | 50 |
| HPRT1² | TGACACTGGCAAAACAATGCA | 84 | GGTCCTTTTCACCAGCAAGCT | 85 | 58 | 90 |
| KCNK2 | TAACAACTATTGGATTTGGTGACTAC | 86 | GCCCTACAAGGATCCAGAAC | 87 | 58 | 100 |
| LRP1B | CATGATCACAACGATGGAGGT | 88 | CTTGAAAGCACTGGGTCCTC | 89 | 58 | 90 |
| NKX6-1 | CTTCTGGCCCGGAGTGAT | 90 | TCTTCCCGTCTTTGTCCAAC | 91 | 58 | 100 |
| NOPE | ACAGGGCTGAAGTGCACAG | 92 | CTTGGTTGAGCCCAGGAGA | 93 | 58 | 90 |
| PCDHGA12 | TGCTGTCAGGTGATTCGGTA | 94 | AGAAACGCCAGTCCGTGTT | 95 | 58 | 80 |
| RPIB9 | GGCCAGTCACAAGAAGGAGA | 96 | GAGATCCACAGAGGCCAAGT | 97 | 58 | 100 |
| SLC2A14 | TCCACGCTCATGACTGTTTC | 98 | CAGGCCACAAAGACCAAGAT | 99 | 58 | 90 |

¹All COBRA amplicons were digested with BstUI except for DDX51 (TaqaI) and KCNK2 (HpyCH4IV).
²Product sizes are approximate.
³HPRT1 primer sequence from Vandesompele et al. (2002).

The purified PCR products were restricted with BstU1, TaqaI or HpyCH4IV according to manufacturer's recommendations (New England Biolabs). The MSP primers (M(+): 5′-AAT AAC ATT TAT AAA TAC CGC CGT T-3′ (SEQ ID NO:25); M(−): 5′-AGT TTG CGT TGG AGA TTG TTC-3′ (SEQ ID NO:24); U(+): 5′-CCA ATA ACA TTT ATA AAT ACC ACC ATT-3′ (SEQ ID NO:27); U(−): 5′-AAG TTT GTG TTG GAG ATT GTT TG-3′) (SEQ ID NO:26) were used in PCR to differentiate methylated and unmethylated sequences in LRP1B. Electrophoresis was performed using a 3% agarose gel stained with SYBR green or a 1.5% agarose gel stained with ethidium bromide to visualize COBRA and MSP products respectively.

Quantitative real time methylation specific PCR (qMSP). qMSP was performed as described previously (Lehmann et al 2002). Briefly, 100 ng of bisulfite treated DNA and the DLC-1 primers (M(+): 5′-CCC AAC GAA AAA ACC CGA CTA ACG-3′(SEQ ID NO:1); M(−): 5′-TTT AAA GAT CGA AAC GAG GGA GCG-3′ (SEQ ID NO:2); U(+): 5′-AAA CCC AAC AAA AAA ACC CAA CTA ACA-3′ (SEQ ID NO:3); U(−): 5′-TTT TTT AAA GAT TGA AAT GAG GGA GTG-3′ (SEQ ID NO:4)) and probe (FAM/AAG TTC GTG AGT CGG CGT TTT TGA/BHQ_{—}1 (SEQ ID NO:5)) were used for the PCR amplification of methylated and unmethylated alleles in two separate reactions. ABgene QPCR mix was used, and the reaction was performed for 40-45 cycles using a SmartCycler™ real-time PCR instrument (Cepheid).

Cell line treatment. ALL cell lines, Jurkat and NALM-6 were purchased from DSMZ (Braunschweig, Germany) and were grown in flasks with RPMI 1640 medium supplemented with 10% fetal bovine serum (FBS), L-glutamine and gentamicin. Treatment was conducted during the log phase of growth with 5-aza-2-deoxycytidine (5-aza) and trichostatin A (TSA) and the control cells were not treated. Jurkat cells were seeded at 8×10⁶ cells/mL and NALM-6 cells were seeded at 5×10⁶ cells/mL. In culture, TSA was added at a 1 μM concentration and incubated for 6 hr, while 5-aza was added at a 1 μM concentration and incubated for 54 and 78 hr in Jurkat and NALM-6 respectively with a media change every 24 hr. The cell culture that received both TSA and 5-aza treatment was first incubated with 5-aza as previously described, followed by an additional 6 hr of incubation with TSA. RNA and DNA from the cultured cells were extracted for use in RT-PCR and COBRA respectively using the previously mentioned kits.

Semiquantitative real time PCR. Total RNA (2 μg) from cell line treatments was pre-treated with DNase I to remove potential DNA contaminants and was then reverse-transcribed in the presence of SuperScript™ II reverse transcriptase (Invitrogen). The generated cDNA was used for PCR amplification with appropriate reagents in the reaction mix with SYBR Green and fluorescein (ABgene) as recommended by the manufacturer. GAPDH and HPRT1 were used as the housekeeping genes in the Taqman™ and SYBR Green real time assays, respectively. The DLC-1 and GAPDH Taqman™ probe and primer set for real-time PCR were purchased from Applied Biosystem's Assay-on-Demand services. The reaction was carried out using a SmartCycler™ real-time PCR instrument (Cepheid). The cycling conditions included an initial 15 min hot start at 95° C. followed by 45 cycles at 95° C. for 15 sec and 60° C. for 1 min. Primers were developed for SYBR Green assays using Primer3 (TABLE 7). The reactions were carried out using the iCycler™ (Biorad). The cycling conditions included an initial 15 min hot start at 95° C. followed by 50 cycles at 95° C. for 15 sec, 58° C. for 30 sec and 72° C. for 30 sec. All samples were run in triplicate and fold changes were determined using the 2^{-ΔΔCT} method (Livak & Schmittgen 2001).

### Results:

To generate epigenetic profiles of selected ALL patients, DNA was extracted from bone marrow aspirate from patients collected at the time of diagnosis and from 4 healthy donors and the samples were compared to a pooled sample of DNA from peripheral blood leukocytes by dual hybridization to a CpG island array. After global normalization, the Kruskal-Wallis non-parametric statistical test was used in an across-array analysis to identify those genes differentially methylated in the patient samples but not in the normal bone marrow controls when compared to the pooled normal DNA. From this analysis, we identified a set of candidate diagnostic genes which were hypermethylated in at least 25% of the patient samples and in none of the normal control bone marrow samples, and which had at least a 1.8-fold difference in methylation between patient and pooled normal DNA (TABLE 8, below). This set of candidate genes includes the ATP-binding cassette, subfamily B member 1 (ABCB1/MDR1), which has previously been shown to be aberrantly methylated in ALL patients (Garcia-Manero et al. 2003) and genes associated with aberrant methylation in other malignancies including deleted in liver cancer 1 (DLC-1), deleted in colorectal cancer (DCC) and the low density lipoprotein receptor-related protein 1B (LRP1B).

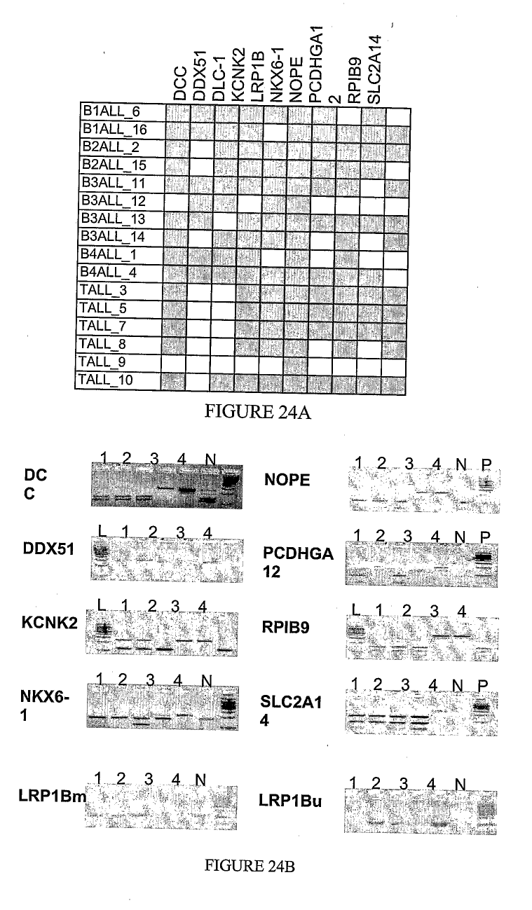

We validated the results from the CpG island array experiment in the patient samples and 4 ALL cell lines using COBRA, MSP or qMSP for 10 of the genes found to be methylated in at least 50% of the studied patients (FIG. 24).

FIGS. 24A and B show, according to particular aspects, validation of promoter methylation in 10 genes identified in CpG island array analysis. FIG. 24A shows validation in 16 ALL patients. DLC-1 was validated by real-time qMSP assay, LRP1B was validated by MSP and the remaining genes were validated by COBRA. Shaded blocks indicate methylation detected and white blocks indicate no methylation detected. Each column represents an individual gene and each row represents an individual patient.

FIG. 24B shows validation in 4 ALL cell lines: 1) Jurkat; 2) MN-60; 3) NALM-6; 4) SD-1; N) bisulfite treated normal DNA; P) SssI and bisulfite treated DNA; and L) Ladder. The gel pictures located above the solid line are the results of COBRA analysis and the gel pictures below the solid line are the results of MSP. LRP1Bm: assay for methylated allele; LRP1Bu: assay for unmethylated allele. The results from the DLC-1 qMSP assay are not presented for the cell lines (Jurkat-positive; MN60-positive; NALM6-positive; SD1-negative).

Despite the small sample size, we detected some interesting methylation patterns. For example, the NK6 transcription factor related locus 1 (NKX6-1) gene was methylated in 100% of the examined patients and cell lines and the DEAD box polypeptide 51 (DDX51) gene was methylated in 70% of the B-ALL and in none of the T-ALL patients which indicates the utility of these genes as a biomarkers for ALL and for distinguishing between B-ALL and T-ALL cases.

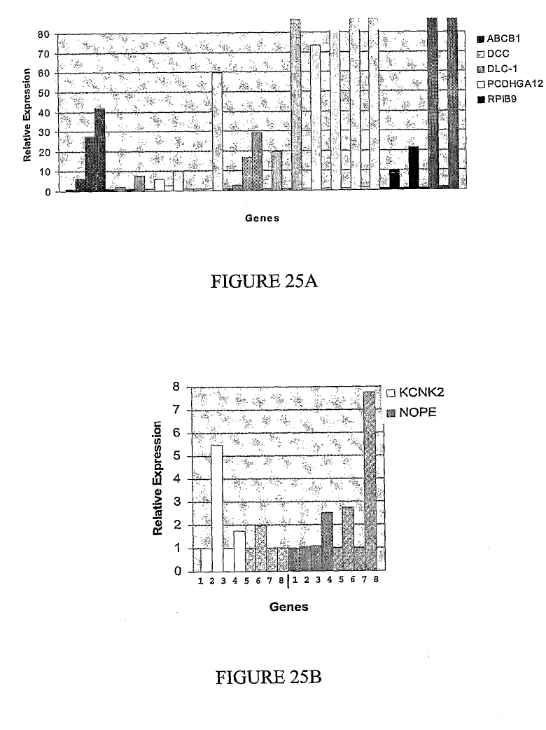

Examination of the effects of gene promoter methylation in vitro by real-time reverse transcription-PCR. To determine whether the promoter methylation detected in the validated gene set was responsible for the down-regulation of these genes in ALL, the in vitro effects of treatment with a demethylating agent, 5-aza-2-deoxycytidine (5-aza), and a histone deacetylase inhibitor, trichostatin A (TSA), was examined both individually and in combination using a B-ALL cell line (NALM-6) and a T-ALL cell line (Jurkat) by real-time reverse transcription PCR. At the baseline, detection of mRNA for 8 of the 10 genes was negative or weak in the untreated (control) cell lines. However, the mRNA expression patterns of ABCB1, DCC, DLC-1, PCDHGA12 and RPIB9 were all increased by at least 10-fold post-treatment (FIG. 25A) and the expression of KCNK2 and NOPE increased by at least 2 fold post-treatment (FIG. 25B).

FIGS. 25A and B show, according to particular aspects, change in mRNA expression in Jurkat and NALM-6 cell lines post treatment with a demethylating agent and a histone deacetylase inhibitor. FIG. 25A shows genes with a 10-fold or greater increase in mRNA expression after treatment in at least one cell line. Solid columns represent the Jurkat cell line and spotted columns represent the NALM6 cell line. The symbol “//” represents a relative expression level greater than 80 with the actual level located in the text above each column.

FIG. 25B shows genes with a 2 to 10-fold increase in mRNA expression after treatment in at least one cell line. Solid columns represent the Jurkat cell line and spotted columns represent the NALM6 cell line: 1) Jurkat Control—no treatment; 2) Jurkat 5-aza treatment; 3) Jurkat TSA treatment; 4) Jurkat 5-aza and TSA treatment; 5) NALM6 Control-no treatment; 6) NALM6 5-aza treatment; 7) NALM6 TSA treatment; and 8) NALM6 5-aza and TSA treatment.

Additionally, while DDX51 and SLC2A14 were moderately expressed in the control cell lines, approximately a 2-fold increase in mRNA expression post-treatment was observed. Finally, only a slight increase (<2-fold) in the transcript levels of LRP1B and NKX6-1 was observed after one or more treatments. These data indicate that the expression of these genes is controlled at some level by methylation and/or deacetylation.

Example Summary. To attain a global view of the methylation present within the promoters of genes in ALL patients and to identify a novel set of methylated genes associated with ALL, methylation profiles were generated for 16 patients using a CGI array consisting of clones representing more than 4 thousand unique CGI sequences spanning all human chromosomes. This is the first time, to applicants' knowledge, that a whole genome methylation scan of this magnitude has been performed in ALL. From the generated profiles, 49 candidate genes were identified that were differentially methylated in at least 25% of the patient samples. Many of these genes are novel discoveries not previously associated with aberrant methylation in ALL or in other types of cancers. Methylation in ten genes found by the CGI array to be differentially methylated in at least 50% of the patients was verified by COBRA, MSP or qMSP. The observations were concordant with the methylation arrays, and the independent verifications indicated that between 10 and 90% of these genes were methylated in every patient. The genes identified in TABLE 7 are involved in a variety of cellular processes including transcription, cell cycle, cell growth, nucleotide binding, transport and cell signaling. In conjunction with the detection of promoter methylation in the ALL samples but not in the normal controls, this indicates that these genes act as tumor suppressors in ALL.

TABLE 8
Hypermethylated genes identified using CGI array.

| Gene | Accession number | Gene Function | Methylation %¹ |
|---|---|---|---|
| NKX6-1 | NM_006168 | Regulation of transcription | 100 |
| KCNK2 | NM_001017424 | Potassium ion transport | 87.5 |
| DCC | NM_005215 | Induction of apotptosis | 81.25 |
| LRP1B | NM_018557 | Protein transport | 75 |
| RP1B9/ABCB1 | NM_138290/NM_000927 | Unknown/Multidrug resistance | 75 |
| DLC-1 | NM_182643 | Negative regulation cell growth | 68.75 |
| NOPE | NM_020962 | Cell adhesion | 68.75 |
| PCDHGA12 | NM_003735 | Cell adhesion | 62.5 |
| SLC2A14 | NM_153449 | Carbohydrate transport | 62.5 |
| DDX51 | NM_175066 | Nucleic acid binding | 50 |
| H3F3A | NM_002107 | DNA binding | 50 |
| TUBGCP3² | NM_006322 | Microtubule nucleation | 50 |
| ZNF304 | NM_020657 | Regulation of transcription | 50 |
| GPR68² | NM_003485 | G-protein coupled receptor protein signaling pathway | 50 |
| ATP5B | NM_001686 | Protein transport | 43.75 |
| BANF1 | NM_003860 | DNA binding | 43.75 |
| FOXD2 | NM_004474 | Regulation of transcription | 43.75 |
| HMGCS1 | NM_002130 | Lipid metabolism | 43.75 |
| MAD2L1BP | NM_001003690 | Regulation of mitosis | 43.75 |
| MCF2L2 | NM_015078 | Guanine nucleotide exchange factor | 43.75 |
| NFATC2² | NM_173091 | Regulation of transcription | 43.75 |
| PRICKLE1 | NM_153026 | Zinc ion binding | 43.75 |
| SMAD9 | NM_005905 | Regulation of transcription | 43.75 |
| TAB3 | NM_152787 | Catalyzes transcription of DNA into RNA | 43.75 |
| ZC3H6 | NM_198581 | Nucleic acid binding | 43.75 |
| GCLM | NM_002061 | Ligase activity | 37.5 |
| HLF | NM_002126 | Regulation of transcription | 37.5 |
| ID1 | NM_002165 | Regulation of transcription | 37.5 |
| NASP | NM_172164 | DNA packaging | 37.5 |
| ZA20D1 | NM_020205 | Ubiquitin cycle | 37.5 |
| DYRK4² | NM_003845 | Protein aa phosphorylation | 37.5 |
| OAZIN | NM_015878 | Polyamine biosynthesis | 37.5 |
| BCL10 | NM_003921 | Negative regulation cell cycle | 31.25 |
| BRMS1 | NM_015399 | Negative regulation cell cycle | 31.25 |
| MYBBP1A | NM_014520 | Regulation of transcription | 31.25 |
| RPLP1 | NM_001003 | Protein biosynthesis | 31.25 |
| SEN2L | NM_025265 | mRNA processing | 31.25 |
| SLC9A3 | NM_004174 | Ion transport | 31.25 |
| TFAP2D² | NM_172238 | Regulation of transcription | 31.25 |
| ZCCHC11 | NM_001009881 | Nucleic acid binding | 31.25 |
| PCSK6² | NM_002570 | Cell-cell signalling | 31.25 |
| RPS16 | NM_001020 | Protein biosynthesis | 31.25 |
| BCAT2 | NM_001190 | Metabolism | 25 |
| CDCA7 | NM_031942 | Cytokinesis | 25 |
| DOK5 | NM_018431 | Insulin receptor binding | 25 |
| ENTPD6² | NM_001247 | Hydrolase activity | 25 |
| EXOSC8 | NM_181503 | RNA processing | 25 |
| OTX2² | NM_021728 | Regulation of transcription | 25 |
| ZNF77 | NM_021217 | Regulation of transcription | 25 |

¹Methylation % is the percentage of ALL patients with methylation at a particular locus.
²No CpG island present in clone. These clones do contain CG dinucleotides. Bolded entries were chosen for validation studies and percentage methylation refers to results from validation studies.

It was determined herein that the 10 validated genes were silenced or down-regulated in NALM-6 and Jurkat ALL cell lines and that their expression could be up-regulated after treatment with a demethylating agent alone or in combination with TSA. Of the validated genes, the greatest post-treatment increase in mRNA expression was for ABCB1, RPIB9 and PCDHGA12 and these appear to be functional genes involved in the development or progression of ALL, and, according to particular aspects, have substantial utility for distinguishing development or progression of ALL. RPIB9 and ABCB1 are genes transcribed in opposite directions with overlapping CGI containing promoters. It has recently been shown that hypomethylation of the ABCB1 promoter leads to multi drug resistance (Baker et al. 2005) and that methylation of the ABCB1 promoter is linked to the down-regulation of gene expression in ALL (Garcia-Manero et al. 2002). This suggests that individuals with methylation in the ABCB1 promoter may better respond to chemotherapeutic treatment than individuals lacking methylation. Although the function of RPIB9 has yet to be confirmed, it likely functions as an activator of Rap which allows B-cells to participate in cell-cell interactions and contributes to the ability of B-lineage cells to bind to bone marrow stromal cells, a requisite process for the maturation of B-cells (McLeod 2004). Therefore, if methylation of the RPIB9 promoter suppresses its transcription, the ability of B-lineage cells to bind to bone marrow stromal cells will likely be inhibited causing the progression of B-lineage cells to halt and resulting in the proliferation of immature cells, a hallmark of ALL. Finally, PCDHGA12 is disclosed herein as an interesting functional gene for ALL in light of a recent report connecting promoter methylation and silencing of PCDHGA11 in astrocytomas and the suggestion that the inactivation of PCDHGA11 is involved in the invasive growth of astrocytoma cells into the normal brain parenchyma (Waha et al. 2005).

In summary, the methylation status of novel genes associated with ALL including NKX6-1, KCNK2, RPIB9, NOPE, PCDHGA12, SLC2A14 and DDX51 was validated Additionally, after treatment with a demethylating agent, mRNA expression was increased in vitro for all 10 genes validated, with the greatest increases occurring for ABCB1, RPIB9, and PCDHGA12. Although the precise role of these genes in ALL progression is unknown, the epigenetic profiles generated in this study, according to particular aspects of the present invention, provide insights to improve our understanding of ALL, provide both novel and noninvasive diagnostic (and/or prognostic, staging, etc.) tools, and novel therapeutic methods and targets for the treatment of ALL.

### Example 7

### A Novel Goal Oriented Approach for Finding Differentially Methylated Genes in, e.g., Small B-Cell Lymphoma was Developed

### Overview

This Example illustrates a novel ‘goal driven’ approach and methods for the identification of differentially methylated genes in DNA microarray data. The goal driven method is applied in this exemplary embodiment to small B-cell lymphoma (SBCL), and permits an accurate discrimination between three types of SBCL and normal patients. Various steps of the algorithm (e.g., data normalization and gene finding) are ‘tuned’ such that final sample clustering optimally matches corresponding pathologically-determined lymphoma diagnoses. More specifically, the gene-finding step comprises two methods, the results of which are fused to reduce the frequency/amount of ‘false positives.’ The output of the fusion step consists in three lists of differential methylated genes (marker candidates). At least one methylation assay (e.g., a combination of bisulfite restriction analysis (COBRA), and methylation-specific PCR (MSP)) is then used (e.g., by pathologists) to validate the differential methylation of these genes (i.e., to validate the candidate differentially methylated markers). Optionally, to further assist in validation, the candidate genes obtained in the gene-finding step are ranked, based on their frequency of appearance in a suitable literature database (e.g., Medline abstracts). For example, in the instant Example, some of the identified genes (e.g., validated differentially methylated genes) are known to be involved in critical pathways such as apoptosis and proliferation while others function as tumor suppressor genes or oncogenes.

### Methodolgy Background:

There are many papers devoted to two-color cDNA microarray processing algorithms. In general, the cDNA microarray processing has four steps: preprocessing, normalization, expression analysis (or feature extraction) and data classification (or pattern discovery).

In spotted cDNA arrays, probes from a cDNA library are deposited as a solution on the surface of the support (plastic or glass) using a set of pins. The RNAs from the test and the reference samples are labeled with different fluorescent dyes (Cy5-red and Cy3-green, respectively) and then hybridized on the array. The expression (methylation) level of individual genes corresponds to the intensity levels of each dye measured at each spot.

The preprocessing consists in the extraction of the intensity values for the two channels, Cy3 (green) and Cy5 (red), and the background at each spot on the microarray. This involves various image processing techniques that we do not detail here. In the present work describe below, these values were provided by a GenePix™ 4000 microarray scanner (Axon Instruments, Union City, Calif.).

Next, one has to normalize the data to account for variability factors such as dye (green and red), pin number, spot location on the array, and array (sample). Among the most used normalization methods we mention: the loess method [Yang 2002], the ANOVA method, the quantile method [Bolstad 2003] and the variance stabilization method [Huber 2002].

The feature (gene) selection step consists in finding the subset of genes that can best discriminate between the different types of leukemia. Various methods can be used for this purpose such as “idealized expression pattern” [Golub 1999], chi-square, T-test, correlation based feature selection [Yeoh 2002], principal component analysis [Khan 2001], and permutation tests [Lee 2004].

Methods such as support vector machines [Furrey 2000, Yeoh 2002], K-nearest neighbor [Golub 1999], neural networks [Khan 2001], decision trees [Yeoh 2002], and fuzzy c-means [Asyali 2005] were used for classifying the samples based on the gene expression. For clustering the sample correlation matrix hierarchical clustering was used. An alternative approach was suggested by Claverie [Claverie 1999] that employs fuzzy c-means for the same task. Applicants have found that this method performs better that the hierarchical clustering for grouping the sample correlation matrix and, therefore, it was used in the method of this Example.

Finally, a group of methods are noteworthy that combine the feature selection with classification denoted as co-clustering (bi-clustering, two-way clustering) algorithms: CTWC [Getz 2000], Residue minimization [Cheng 2001], spectral graph [Cho 2004], marker propagation [Oyanagi 2001], fuzzy co-clustering [Oh 2001, Kummamuru 2003].

### Materials and Methods:

A diagram of the gene selection method used in this paper is presented in FIG. 26. The detailed explanation of each step is as follows:

1. Normalization: The normalization was performed using the loess method [Yang 2002]. [Ozy: xxx, the best came out to be: back-corrected, pin-based, order 1, span 0.2]. A normalization across samples was performed for each gene (locus) by subtracting the mean and dividing by the standard deviation.

2. and 3. Idealized Methylation Pattern. For the gene selection step we used two methods in order to reduce the number of genes that were not relevant to our search (to reduce false positives):

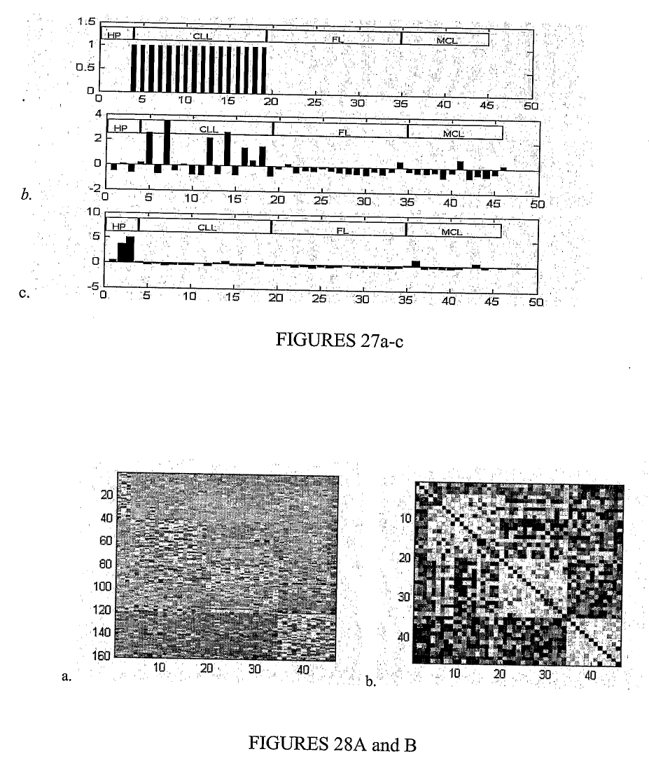

The first method employed was a modified version of the “idealized expression pattern” [Golub 1999]. The modified method is referred to herein as “idealized methylation pattern” (IMP), because methylation and not expression is detected in the present experiments. The IMP method is briefly explained in FIG. 27. For each gene gᵢ, the cross-correlation Cᵢⱼ of its methylation pattern was computed with the ideal profile for class j, IMPⱼ, as:

$${C}_{ij} = \frac{1}{3} {\sum }_{k = 1}^{3} {g}_{ik} {IMP}_{jk} \frac{1}{16} {\sum }_{k = 4}^{19} {g}_{ik} {IMP}_{jk} + \frac{1}{15} {\sum }_{k = 20}^{34} {g}_{ik} {IMP}_{jk} + \frac{1}{12} {\sum }_{k = 35}^{46} {g}_{ik} {IMP}_{jk} . \qquad ( 1 )$$

In computing the correlation, the samples in each class are weighted by the cardinality of each class. Then the genes were ranked (from high to low) by their correlation value. For each class we selected the first 40 genes in the list.

The second gene selection method was based on a pair-wise t-test. The right tailed t-test was used to determine if the mean of the methylation values in one class is higher than the mean of the values in the other classes. For example, to determine if a gene gᵢ was exclusively hypemethylated in HP (FIG. 27c), we employed pair-wise t-tests together with the following rule: “The mean of methylation of gᵢ in HP> the mean of methylation of gᵢ in CLL AND The mean of methylation of gᵢ in HP> the mean of methylation of gᵢ in FL AND The mean of methylation of gᵢ in HP> the mean of methylation of gᵢ in MCL”. The t-tests were performed with a p-value p=0.05.

4. Clustering of the sample (patients) correlation matrix. Each patient Pⱼ, j=1 . . . 46, is characterized by a set of 8,640 methylation values {gⱼₖ}, k=1 . . . 8,640. The patient correlation matrix (“PCM”) is computed as:

$$P C {M}_{ij} = \frac{{\sum }_{k = 1}^{8640} {g}_{ik} {g}_{jk}}{\sqrt{{\sum }_{k = 1}^{8640} {g}_{ik}^{2}} \sqrt{{\sum }_{k = 1}^{8640} {g}_{jk}^{2}}} . \qquad ( 2 )$$

The correlation matrix is a similarity matrix, that is, PCMᵢⱼ is 1 for very similar patients and is 0 for very dissimilar patients. If we consider the row i in PCM as a feature vector that describes how similar patient i is to the other patients [Clayerie 1999], then we can use fuzzy c-means [Bezdek 1981] for clustering. In applicants' experience, fuzzy c-means proved to produce better results than the hierarchical clustering on similarity matrices, and is thus preferred.

5. Multidimensional scaling (MDS) for cluster visualization. One of the most important goals in visualizing clustered data is to get a sense of how near or far points are from each other. Often, one can do this with a scatter plot. However, for some analyses, the data at hand might not be in the form of points (objectual) at all, but rather in the form of pair-wise similarities or dissimilarities between samples (relational). Moreover, even if one has the data in objectual form, if the feature dimensionality is higher than 3, the points cannot be represented in an easily understandable form (2D or 3D scatter plot). For this latter case, one could use some form of projection such as principal component analysis (PCA). However, for the case of the microarray experiments, PCA provides a very poor approximation because the number of sample (patients) is 2-3 orders of magnitude smaller than the number of features (genes). In our experience, one eigenvalue (one dimension) explains about 1/NP (NP, number of patients, 43 in our case) of the data, hence considering the first 3 highest eigenvalues results in an approximation error of about 100(NP−3)/NP % (93% in the present case).

Multidimensional scaling (MDS) [Cox 2001] is a set of methods that address the above problems. MDS allows the visualization of the sample distribution for many kinds of distance or dissimilarity measures and can produce a representation of the data in a small number of dimensions. MDS does not require raw data, but only a matrix of pair-wise distances or dissimilarities. MDS methods are grouped in Euclidean (considers that the sample space is Euclidean) and non-Euclidean (the sample space is non-Euclidean, for example the space of all the country capitals in the world). In our experiments, we used the Euclidean (Classical) MDS implemented in Matlab® (cmdscale from the Statistics package) and the patient correlation matrix, PCM. The approximation error obtained using the MDS dimensionality reduction is less than 1%.

MDS was employed to assess the clustering produced by the FCM (PCM). In addition, the obtained clusters were inspected for possible sub-clusters that will signal possible lymphoma sub-types.

6. Selected Gene Filtering by Result Fusion. The genes selected by the IMP and t-test methods were filtered using a two-out-of-two voting scheme (result fusion; voting). Only genes selected by both methods as being uniquely methylated in a given class were chosen for further validation with COBRA and methylation specific PCR. This particular fusion approach ignores the rank of a gene and the performance of each selection method. Alternatively, more selection algorithms could be used along with a rank and performance based fusion.

7. Literature Look-up of the Selected Genes. Both COBRA and methylation specific PCR are time consuming. For this reason, in particular embodiments by investigating another dimension of the selected genes was invested (the publishing dimension) to further assist (e.g., the pathologists) in choosing which genes to analyze first. To accomplish, the number of papers where each gene co-occurred with the term “lymphoma” were counted. The premise of this approach is that if a selected gene has been mentioned many times as being linked to lymphoma, then it has a higher chance to be differentially hypermethylated in one type of lymphoma than a gene that was not investigated yet. The search was conducted by matching the MeSH terms present in the article abstracts with our selected genes and the MeSH term “lymphoma”.

### Results.

The follow results were obtained on a 46 patient dataset. The dataset consists in methylation microarrays from 3 patients diagnosed with hyperplasia (HP), but considered normals, 16 patients diagnosed with CLL, 15 patients diagnosed with FL and 12 patients diagnosed with MCL. Each array contains 8,640 loci that represent CpG islands (DNA regions rich in the Cytosine-Guanine pair) from the promoter and first exon regions of a number of genes. For a specific locus, one can find the related gene by searching the database provided by the Der Laboratory at the University of Toronto (http://s-der10. med.utoronto.ca/CpGIslands.htm).

The results of the IMP selection method are presented in FIG. 28. For each gene we computed the cross-correlation with the desired class profile. Then the genes were ranked (from high to low) by their cross-correlation value. For each class we selected the first 40 genes in the list. FIG. 28A shows the methylation profile of the 160 selected genes (vertical) for all 46 samples (horizontal). One can easily observe the blocky appearance (red denotes hypermethylation). To assess the discrimination power of this set of 160 genes we computed the sample cross-correlation matrix (FIG. 28B).

To cluster the samples, fuzzy C-means was used (instead of hierarchical clustering) on the cross-correlation matrix. By clustering the rows of the matrix (FIG. 28B) a perfect separation of the leukemia types was obtained, that is, the first 3 samples are HP, the next 16 are CLL, the next 15 are FL and the last 12 are MCL. In this instance, the same result was obtained by considering only the top 20 correlated genes for each class, but not when considering only the top 10 genes for each class.

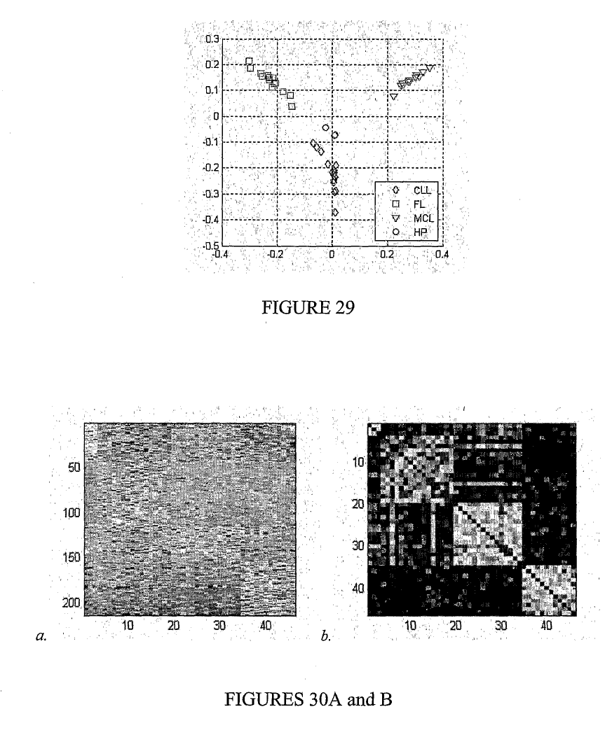

Using MDS with the patient correlation matrix (FIG. 28B), the relative position of the 46 patients was analyzed (FIG. 29). Several observation were made, based on FIG. 28. First, the 3 lymphoma types appear well separated, confirming the result obtained using fuzzy C-means. Hence, the methylation array has substantial utility to differentiate between CLL, MCL and FL. Second, the normals (HP) are somewhat closer to CLL but they are well separated from MCL and FL. It is somewhat surprising that fuzzy C-means managed to separate the HP from the CLL patients.

The result obtained using the t-test selection method is next presented. The number of genes selected this way was 213, respectively, 43, 73, 37 and 60. The methylation profile of the genes selected for each class are shown in FIG. 30A, and the patient correlation matrix in FIG. 30B. The sample clustering performed using fuzzy C-means and the matrix from FIG. 30B resulted in 1 clustering error (1 CLL was called FL).

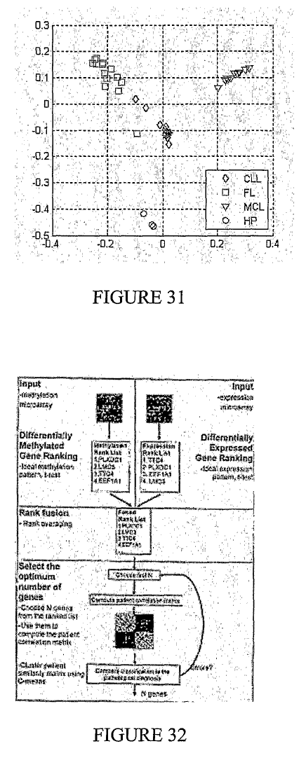

The patient correlation matrix from FIG. 30B was then used with MDS to visualize the relations between patients as defined by the genes selected using t-test (FIG. 31).

It is obvious in FIG. 31 which CLL patient was clustered as FL (the one surrounded by a square). However, it is less obvious why the circled FL patient was not classified as a CLL. However, it is clear the t-test method does not separate the CLL from FL as well as the IMP method. However, by looking at FIG. 31, one can conclude that the separation of the normal (HP) patients from the ill patients (CLL+HP+MCL) is better in this case than in the IMP case. In addition, the fact that the HP seems closer to CLL than to FL and MCL agrees with pathologist's intuition. This fact can be also observed in FIG. 29.

Fusion. To refine (remove false positives) we fused the selected gene sets obtained using the IMP method and the t-test method. Out of the 40 exclusively hypermethylated loci found for each class using the IMP selection method, only respectively 10, 30, 25 and 33 were confirmed as such by the t-test method. From the above 98 loci, only 49 were associated with genes (see TABLE 9).

To further assist (e.g., the pathologist) in the validation of the computational results presented in TABLE 9, Medline® was searched for abstracts that mention the genes in TABLE 2 in a lymphoma context. For example, the search for the abstracts that mentioned MEIS1 was performed using the strategy: “(lymphoma OR leukemia) AND MEIS1”. For the HP genes, the searched used only the gene name. The number of the abstracts retrieved for each lymphoma type is shown in TABLE 9 adjacent to the gene name.

TABLE 9
Genes associated with the differentially hypermethylated loci in hyperplasia (HP), chronic
lymphocytic leukemia (CLL), follicular lymphoma (FL) and mantle cell lymphoma (MCL).

| HP | | CLL | | FL | | MCL | |
|---|---|---|---|---|---|---|---|
| genes | #abstacts | genes | #abstacts | genes | #abstacts | genes | #abstacts |
| DPYSL2 | 10 | MEIS1 | 69 | SCD | 24 | MAP4 | 7 |
| SUPV3L1 | 6 | EIF4EBP1 | 15 | HCN3 | 0 | RPS16 | 6 |
| EFNA5 | 1 | BCL11B | 13 | HNRPA2B1 | 0 | GMNN | 3 |
| MRPL44 | 1 | CHN2 | 5 | TEPP | 0 | EIF4A2 | 2 |
| LRCH2 | 0 | FAF1 | 2 | KCNJ10 | 0 | TIAM2 | 2 |
| ARRDC3 | 0 | PRRX1 | 1 | BHLHB4 | 0 | CBX5 | 1 |
| | | GRIK2 | 1 | ZCSL2 | 0 | DKFZP434K1421 | 0 |
| | | POU4F1 | 0 | AAA1 | 0 | CRIM1 | 0 |
| | | SLC2A14 | 0 | OPRM1 | 0 | PCDH10 | 0 |
| | | BRF1 | 0 | CNTN1 | 0 | FLJ20014 | 0 |
| | | TMEM16G | 0 | | | TDP1 | 0 |
| | | ZNF552 | 0 | | | FBXW11 | 0 |
| | | LOC220869 | 0 | | | PPM1B | 0 |
| | | KIAA1102 | 0 | | | | |
| | | HMGA1L4 | 0 | | | | |
| | | NRP2 | 0 | | | | |
| | | C9orf112 | 0 | | | | |
| | | RBJ | 0 | | | | |
| | | PRAC | 0 | | | | |
| | | TNFAIP9 | 0 | | | | |

Further embodiments provide a method for simultaneous gene selection in, for example, B-cell lymphoma from methylation and expression microarrays. The approach is analogous to that described above in this example, except that rank fusion (rank averaging) is between a differentially methylated gene ranking (IMP, t-test) and a differentially expressed gene ranking (IEP, t-test), resulting in a fused rank list, from which genes are optimally selected by computing patient correlation matrix, and clustering of the patient similarity matrix using C-means to select for an optimal number of genes that best match the pathologically determined lymphoma diagnoses (see FIG. 32). Such embodiments provide a powerful approach to discovery of links between methylation and expression events that differ between major classes of, e.g., SBCL and provide for new diagnostic and/or prognostic, staging, etc. assays, and new insights into the biology of these diseases.

### REFERENCES CITED

### Examples 1-7

### Reference List for Example 1

1 Cheson, B. D. What is new in lymphoma?, CA Cancer J. Clin., 54: 260-272, 2004.
2 Jaffe E S Harris N L Stein H Vardiman J W eds. Pathology and genetics of tumours of haematopoietic and lymphoid tissue. WHO classification of tumors, IARC Press Lyon: France, 2001.
3 Bird, A. The essentials of DNA methylation, Cell, 70: 5-8, 1992.
4 Robertson, K. D. and Jones, P. A. DNA methylation: past, present and future directions, Carcinogenesis, 21: 461-467, 2000.
5 Craig, J. M. and Bickmore, W. A. The distribution of CpG islands in mammalian chromosomes, Nat. Genet., 7: 376-382, 1994.
6 Jones, P. A. and Baylin, S. B. The fundamental role of epigenetic events in cancer, Nat. Rev. Genet., 3: 415-428, 2002.
7 Esteller, M. Profiling aberrant DNA methylation in hematologic neoplasms: a view from the tip of the iceberg, Clin. Immunol., 109: 80-88, 2003.
8 Yang, H., Chen, C. M., Yan, P., Huang, T. H., Shi, H., Burger, M., Nimmrich, I., Maier, S., Berlin, K. and Caldwell, C. W. The androgen receptor gene is preferentially hypermethylated in follicular non-Hodgkin's lymphomas, Clin. Cancer Res., 9: 4034-4042, 2003.
9 Rossi, D., Capello, D., Gloghini, A., Franceschetti, S., Paulli, M., Bhatia, K., Saglio, G., Vitolo, U., Pileri, S. A., Esteller, M., Carbone, A. and Gaidano, G. Aberrant promoter methylation of multiple genes throughout the clinico-pathologic spectrum of B-cell neoplasia, Haematologica, 89: 154-164, 2004.
Takahashi, T., Shivapurkar, N., Reddy, J., Shigematsu, H., Miyajima, K., Suzuki, M., Toyooka, S., Zochbauer-Muller, S., Drach, J., Parikh, G., Zheng, Y., Feng, Z., Kroft, S. H., Timmons, C., McKenna, R. W. and Gazdar, A. F. DNA methylation profiles of lymphoid and hematopoietic malignancies, Clin. Cancer Res., 10: 2928-2935, 2004.
11 Shi, H., Yan, P. S., Chen, C. M., Rahmatpanah, F., Lofton-Day, C., Caldwell, C. W. and Huang, T. H. Expressed CpG island sequence tag microarray for dual screening of DNA hypermethylation and gene silencing in cancer cells, Cancer Res., 62: 3214-3220, 2002.
12 Kim, T. Y., Jong, H. S., Song, S. H., Dimtchev, A., Jeong, S. J., Lee, J. W., Kim, T. Y., Kim, N. K., Jung, M. and Bang, Y. J. Transcriptional silencing of the DLC-1 tumor suppressor gene by epigenetic mechanism in gastric cancer cells, Oncogene, 22: 3943-3951, 2003.
13 Yuan, B. Z., Durkin, M. E. and Popescu, N. C. Promoter hypermethylation of DLC-1, a candidate tumor suppressor gene, in several common human cancers, Cancer Genet. Cytogenet., 140: 113-117,2003.
14 Jeronimo, C., Henrique, R., Hoque, M. O., Mambo, E., Ribeiro, F. R., Varzim, G., Oliveira, J., Teixeira, M. R., Lopes, C. and Sidransky, D. A quantitative promoter methylation profile of prostate cancer, Clin. Cancer Res., 10: 8472-8478, 2004.
15 Lai, J. P., Douglas, S. D., Wang, Y. J. and Ho, W. Z. Real-time reverse transcription-PCR quantitation of substance P receptor (NK-1R) mRNA, Clin. Diagn. Lab Immunol., 12: 537-541, 2005.
16 Melo, M. R., Faria, C. D., Melo, K. C., Reboucas, N. A. and Longui, C. A. Real-time PCR quantitation of glucocorticoid receptor alpha isoform, BMC. Mol. Biol., 5: 19, 2004.
17 Lee, T. H., Montalvo, L., Chrebtow, V. and Busch, M. P. Quantitation of genomic DNA in plasma and serum samples: higher concentrations of genomic DNA found in serum than in plasma, Transfusion, 41: 276-282, 2001.
18 Thijssen, M. A., Swinkels, D. W., Ruers, T. J. and de Kok, J. B. Difference between free circulating plasma and serum DNA in patients with colorectal liver metastases, Anticancer Res., 22: 421-425, 2002.
19 Boddy, J. L., Gal, S., Malone, P. R., Harris, A. L. and Wainscoat, J. S. Prospective study of quantitation of plasma DNA levels in the diagnosis of malignant versus benign prostate disease, Clin. Cancer Res., 11: 1394-1399, 2005.
Grunau, C., Clark, S. J. and Rosenthal, A. Bisulfite genomic sequencing: systematic investigation of critical experimental parameters, Nucleic Acids Res., 29: E65, 2001.
21 Ng, I. O., Liang, Z. D., Cao, L. and Lee, T. K. DLC-1 is deleted in primary hepatocellular carcinoma and exerts inhibitory effects on the proliferation of hepatoma cell lines with deleted DLC-1, Cancer Res., 60: 6581-6584, 2000.
22 Yuan, B. Z., Miller, M. J., Keck, C. L., Zimonjic, D. B., Thorgeirsson, S. S. and Popescu, N. C. Cloning, characterization, and chromosomal localization of a gene frequently deleted in human liver cancer (DLC-1) homologous to rat RhoGAP, Cancer Res., 58: 2196-2199, 1998.
23 Yuan, B. Z., Jefferson, A. M., Baldwin, K. T., Thorgeirsson, S. S., Popescu, N. C. and Reynolds, S. H. DLC-1 operates as a tumor suppressor gene in human non-small cell lung carcinomas, Oncogene, 23: 1405-1411, 2004.
24 Wong, C. M., Lee, J. M., Ching, Y. P., Jin, D. Y. and Ng, I. O. Genetic and epigenetic alterations of DLC-1 gene in hepatocellular carcinoma, Cancer Res., 63: 7646-7651, 2003.
Saci, A. and Carpenter, C. L. RhoA GTPase regulates B cell receptor signaling, Mol. Cell, 17: 205-214, 2005.
26 Yuan, B. Z., Jefferson, A. M., Baldwin, K. T., Thorgeirsson, S. S., Popescu, N. C. and Reynolds, S. H. DLC-1 operates as a tumor suppressor gene in human non-small cell lung carcinomas, Oncogene, 23: 1405-1411, 2004.

### Reference List for Example 2

1 Jaffe, E. S., Harris, N. L., Stein, H. and Vardiman, J. E. World Health Organization Classification of Tumors. Pathology and Genetics of Tumours of Haematopoietic and Lymphoid Tissues., IARC Press: Lyon, 2001.
2 Dohner, H., Stilgenbauer, S., Benner, A., Leupolt, E., Krober, A., Bullinger, L., Dohner, K., Bentz, M. and Lichter, P. Genomic aberrations and survival in chronic lymphocytic leukemia, N. Engl. J. Med., 343: 1910-1916, 2000.
3 Egger, G., Liang, G., Aparicio, A. and Jones, P. A. Epigenetics in human disease and prospects for epigenetic therapy, Nature, 429: 457-463, 2004.
4 Costello, J. F., Fruhwald, M. C., Smiraglia, D. J., Rush, L. J., Robertson, G. P., Gao, X., Wright, F. A., Feramisco, J. D., Peltomaki, P., Lang, J. C., Schuller, D. E., Yu, L., Bloomfield, C. D., Caligiuri, M. A., Yates, A., Nishikawa, R., Su, H. H., Petrelli, N. J., Zhang, X., O'Dorisio, M. S., Held, W. A., Cavenee, W. K. and Plass, C. Aberrant CpG-island methylation has non-random and tumour-type-specific patterns, Nat. Genet., 24: 132-138, 2000.
5 Costello, J. F., Fruhwald, M. C., Smiraglia, D. J., Rush, L. J., Robertson, G. P., Gao, X., Wright, F. A., Feramisco, J. D., Peltomaki, P., Lang, J. C., Schuller, D. E., Yu, L., Bloomfield, C. D., Caligiuri, M. A., Yates, A., Nishikawa, R., Su, H. H., Petrelli, N. J., Zhang, X., O'Dorisio, M. S., Held, W. A., Cavenee, W. K. and Plass, C. Aberrant CpG-island methylation has non-random and tumour-type-specific patterns, Nat. Genet., 24: 132-138, 2000.
6 Li, Y., Nagai, H., Ohno, T., Yuge, M., Hatano, S., Ito, E., Mori, N., Saito, H. and Kinoshita, T. Aberrant DNA methylation of p57(KIP2) gene in the promoter region in lymphoid malignancies of B-cell phenotype, Blood, 100: 2572-2577, 2002.
7 Cameron, E. E., Baylin, S. B. and Herman, J. G. p15(INK4B) CpG island methylation in primary acute leukemia is heterogeneous and suggests density as a critical factor for transcriptional silencing, Blood, 94: 2445-2451, 1999.
8 Katzenellenbogen, R. A., Baylin, S. B. and Herman, J. G. Hypermethylation of the DAP-kinase CpG island is a common alteration in B-cell malignancies, Blood, 93: 4347-4353, 1999.
9 Kawano, S., Miller, C. W., Gombart, A. F., Bartram, C. R., Matsuo, Y., Asou, H., Sakashita, A., Said, J., Tatsumi, E. and Koeffler, H. P. Loss of p73 gene expression in leukemias/lymphomas due to hypermethylation, Blood, 94: 1113-1120, 1999.
10 Seto, M. Genetic and epigenetic factors involved in B-cell lymphomagenesis, Cancer Sci., 95: 704-710, 2004.
11 Byrd, J. C., Stilgenbauer, S. and Flinn, I. W. Chronic lymphocytic leukemia, Hematology.(Am. Soc. Hematol. Educ. Program.), 163-183, 2004.
12 Glas, A. M., Kersten, M. J., Delahaye, L. J., Witteveen, A. T., Kibbelaar, R. E., Velds, A., Wessels, L. F., Joosten, P., Kerkhoven, R. M., Bernards, R., van Krieken, J. H., Kluin, P. M., van't Veer, L. J. and de, J. D. Gene expression profiling in follicular lymphoma to assess clinical aggressiveness and to guide the choice of treatment, Blood, 105: 301-307, 2005.
13 Alizadeh, A., Eisen, M., Davis, R. E., Ma, C., Sabet, H., Tran, T., Powell, J. I., Yang, L., Marti, G. E., Moore, D. T., Hudson, J. R., Jr., Chan, W. C., Greiner, T., Weisenburger, D., Armitage, J. O., Lossos, I., Levy, R., Botstein, D., Brown, P. O. and Staudt, L. M. The lymphochip: a specialized cDNA microarray for the genomic-scale analysis of gene expression in normal and malignant lymphocytes, Cold Spring Harb. Symp. Quant. Biol., 64: 71-78, 1999.
14 Husson, H., Carideo, E. G., Neuberg, D., Schultze, J., Munoz, O., Marks, P. W., Donovan, J. W., Chillemi, A. C., O'Connell, P. and Freedman, A. S. Gene expression profiling of follicular lymphoma and normal germinal center B cells using cDNA arrays, Blood, 99: 282-289, 2002.
15 Adorjan, P., Distler, J., Lipscher, E., Model, F., Muller, J., Pelet, C., Braun, A., Florl, A. R., Gutig, D., Grabs, G., Howe, A., Kursar, M., Lesche, R., Leu, E., Lewin, A., Maier, S., Muller, V., Otto, T., Scholz, C., Schulz, W. A., Seifert, H. H., Schwope, I., Ziebarth, H., Berlin, K., Piepenbrock, C. and Olek, A. Tumour class prediction and discovery by microarray-based DNA methylation analysis, Nucleic A cids Res., 30: e21, 2002.
16 Beckwith, M., Longo, D. L., O'Connell, C. D., Moratz, C. M. and Urba, W. J. Phorbol ester-induced, cell-cycle-specific, growth inhibition of human B-lymphoma cell lines, J. Natl. Cancer Inst., 82: 501-509, 1990.
17 Klein, E., Klein, G., Nadkarni, J. S., Nadkarni, J. J., Wigzell, H. and Clifford, P. Surface IgM-kappa specificity on a Burkitt lymphoma cell in vivo and in derived culture lines, Cancer Res., 28: 1300-1310, 1968.
18 Pulvertaft, R. J. and Pulvertaft, I. Spontaneous “transformation” of lymphocytes from the umbilical-cord vein, Lancet, 2: 892-893, 1966.
19 Jadayel, D. M., Lukas, J., Nacheva, E., Bartkova, J., Stranks, G., De Schouwer, P. J., Lens, D., Bartek, J., Dyer, M. J., Kruger, A. R. and Catovsky, D. Potential role for concurrent abnormalities of the cyclin D1, p16CDKN2 and p15CDKN2B genes in certain B cell non-Hodgkin's lymphomas. Functional studies in a cell line (Granta 519), Leukemia, 11: 64-72, 1997.
20 Stacchini, A., Aragno, M., Vallario, A., Alfarano, A., Circosta, P., Gottardi, D., Faldella, A., Rege-Cambrin, G., Thunberg, U., Nilsson, K. and Caligaris-Cappio, F. MEC1 and MEC2: two new cell lines derived from B-chronic lymphocytic leukaemia in prolymphocytoid transformation, Leuk. Res., 23: 127-136, 1999.
21 Yan, P. S., Chen, C. M., Shi, H., Rahmatpanah, F., Wei, S. H., Caldwell, C. W. and Huang, T. H. Dissecting complex epigenetic alterations in breast cancer using CpG island microarrays, Cancer Res., 61: 8375-8380, 2001.
22 Heisler, L. E., Torti, D., Boutros, P. C., Watson, J., Chan, C., Winegarden, N., Takahashi, M., Yau, P., Huang, T. H., Farnham, P. J., Jurisica, I., Woodgett, J. R., Brenmer, R., Penn, L. Z. and Der, S. D. CpG Island microarray probe sequences derived from a physical library are representative of CpG Islands annotated on the human genome, Nucleic Acids Res., 33: 2952-2961, 2005.
23 Yan, P. S., Chen, C. M., Shi, H., Rahmatpanah, F., Wei, S. H., Caldwell, C. W. and Huang, T. H. Dissecting complex epigenetic alterations in breast cancer using CpG island microarrays, Cancer Res., 61: 8375-8380, 2001.
24 Shi, H., Wei, S. H., Leu, Y. W., Rahmatpanah, F., Liu, J. C., Yan, P. S., Nephew, K. P. and Huang, T. H. Triple analysis of the cancer epigenome: an integrated microarray system for assessing gene expression, DNA methylation, and histone acetylation, Cancer Res., 63: 2164-2171, 2003.
25 Shi, H., Yan, P. S., Chen, C. M., Rahmatpanah, F., Lofton-Day, C., Caldwell, C. W. and Huang, T. H. Expressed CpG island sequence tag microarray for dual screening of DNA hypermethylation and gene silencing in cancer cells, Cancer Res., 62: 3214-3220, 2002.
26 Yang, H., Chen, C. M., Yan, P., Huang, T. H., Shi, H., Burger, M., Nimmich, I., Maier, S., Berlin, K. and Caldwell, C. W. The androgen receptor gene is preferentially hypermethylated in follicular non-Hodgkin's lymphomas, Clin. Cancer Res., 9: 4034-4042, 2003.
27 Hochberg, Y. and Benjamini, Y. More powerful procedures for multiple significance testing, Stat. Med., 9: 811-818, 1990.
28 Smiraglia, D. J., Rush, L. J., Fruhwald, M. C., Dai, Z., Held, W. A., Costello, J. F., Lang, J. C., Eng, C. , Li, B. , Wright, F. A. , Caligiuri, M. A. and Plass, C. Excessive CpG island hypermethylation in cancer cell lines versus primary human malignancies, Hum. Mol. Genet. , 10: 1413-1419, 2001.
29 Laird, P. W. Cancer epigenetics, Hum. Mol. Genet. , 14 Spec No 1: R65-R76, 2005.
30 Jones, P. A. and Baylin, S. B. The fundamental role of epigenetic events in cancer, Nat. Rev. Genet. , 3: 415-428, 2002.
31 Egger, G. , Liang, G. , Aparicio, A. and Jones, P. A. Epigenetics in human disease and prospects for epigenetic therapy, Nature, 429: 457-463, 2004.
32 Laird, P. W. The power and the promise of DNA methylation markers, Nat. Rev. Cancer, 3: 253-266, 2003.
33 Shaker, S. , Bernstein, M. , Momparler, L. F. and Momparler, R. L. Preclinical evaluation of antineoplastic activity of inhibitors of DNA methylation (5-aza-2′-deoxycytidine) and histone deacetylation (trichostatin A, depsipeptide) in combination against myeloid leukemic cells, Leuk. Res. , 27: 437-444, 2003.
34 Stevenson, F. K. , Sahota, S. S. , Ottensmeier, C. H. , Zhu, D. , Forconi, F. and Hamblin, T. J. The occurrence and significance of V gene mutations in B cell-derived human malignancy, Adv. Cancer Res. , 83: 81-116, 2001.
35 Robetorye, R. S. , Bohling, S. D. , Morgan, J. W. , Fillmore, G. C. , Lim, M. S. and Elenitoba-Johnson, K. S. Microarray analysis of B-cell lymphoma cell lines with the t(14;18), J. Mol. Diagn. , 4: 123-136, 2002.
36 Xu, Y. , Baldassare, M. , Fisher, P. , Rathbun, G. , Oltz, E. M. , Yancopoulos, G. D. , Jessell, T. M. and Alt, F. W. LH-2: a LIM/homeodomain gene expressed in developing lymphocytes and neural cells, Proc. Natl. Acad. Sci. U. S. A, 90: 227-231, 1993.
37 Schreiber, J. , Enderich, J. , Sock, E. , Schmidt, C. , Richter-Landsberg, C. and Wegner, M. Redundancy of class III POU proteins in the oligodendrocyte lineage, J. Biol. Chem. , 272: 32286-32293, 1997.
38 Chen, H. , Chedotal, A. , He, Z. , Goodman, C. S. and Tessier-Lavigne, M. Neuropilin-2, a novel member of the neuropilin family, is a high affinity receptor for the semaphorins Sema E and Sema IV but not Sema III, Neuron, 19: 547-559, 1997.
39 Moreno-Garcia, M. E. , Lopez-Bojorques, L. N. , Zentella, A. , Humphries, L. A. , Rawlings, D. J. and Santos-Argumedo, L. CD38 signaling regulates B lymphocyte activation via a phospholipase C (PLC)-gamma 2-independent, protein kinase C, phosphatidylcholine-PLC, and phospholipase D-dependent signaling cascade, J. Immunol. , 174: 2687-2695, 2005.
40 Sonoda, I. , Imoto, I. , Inoue, J. , Shibata, T. , Shimada, Y. , Chin, K. , Imamura, M. , Amagasa, T. , Gray, J. W. , Hirohashi, S. and Inazawa, J. Frequent silencing of low density lipoprotein receptor-related protein 1B (LRP1B) expression by genetic and epigenetic mechanisms in esophageal squamous cell carcinoma, Cancer Res. , 64: 3741-3747, 2004.
41 Huang, T. H. , Perry, M. R. and Laux, D. E. Methylation profiling of CpG islands in human breast cancer cells, Hum. Mol. Genet. , 8. 459-470, 1999.

### Reference List for Example 3

1 Anonymous Cancer Facts & Figures, 2005, American Cancer Society: Atalanta, Geogia, 2005.
2 Jaffe E S Harris N L Stein H Vardiman J W eds. Pathology and genetics of tumours of haematopoietic and lymphoid tissue. WHO classification of tumors, IARC Press Lyon: France, 2001.
3 Shaffer, A. L. , Rosenwald, A. and Staudt, L. M. Lymphoid malignancies: the dark side of B-cell differentiation, Nat. Rev. Immunol. , 2: 920-932, 2002.
4 Jones, P. A. and Baylin, S. B. The fundamental role of epigenetic events in cancer, Nat. Rev. Genet. , 3: 415-428, 2002.
Esteller, M. , Corn, P. G. , Baylin, S. B. and Herman, J. G. A gene hypermethylation profile of human cancer, Cancer Res. , 61: 3225-3229, 2001.
6 Esteller, M. Profiling aberrant DNA methylation in hematologic neoplasms: a view from the tip of the iceberg, Clin. Immunol. , 109: 80-88, 2003.
7 Rossi, D. , Capello, D. , Gloghini, A. , Franceschetti, S. , Paulli, M. , Bhatia, K. , Saglio, G. , Vitolo, U. , Pileri, S. A. , Esteller, M. , Carbone, A. and Gaidano, G. Aberrant promoter methylation of multiple genes throughout the clinico-pathologic spectrum of B-cell neoplasia, Haematologica, 89: 154-164, 2004.
8 Li, Y. , Nagai, H. , Ohno, T. , Yuge, M. , Hatano, S. , Ito, E. , Mori, N. , Saito, H. and Kinoshita, T. Aberrant DNA methylation of p57(KIP2) gene in the promoter region in lymphoid malignancies of B-cell phenotype, Blood, 100: 2572-2577, 2002.
9 Yang, H. , Chen, C. M. , Yan, P. , Huang, T. H. , Shi, H. , Burger, M. , Nimmrich, I. , Maier, S. , Berlin, K. and Caldwell, C. W. The androgen receptor gene is preferentially hypermethylated in follicular non-Hodgkin's lymphomas, Clin. Cancer Res. , 9: 4034-4042, 2003.
10 Wei, S. H. , Chen, C. M. , Strathdee, G. , Harnsomburana, J. , Shyu, C. R. , Rahmatpanah, F. , Shi, H. , Ng, S. W. , Yan, P. S. , Nephew, K. P. , Brown, R. and Huang, T. H. Methylation microarray analysis of late-stage ovarian carcinomas distinguishes progression-free survival in patients and identifies candidate epigenetic markers, Clin. Cancer Res. , 8: 2246-2252, 2002.
11 Yan, P. S. , Chen, C. M. , Shi, H. , Rahmatpanah, F. , Wei, S. H. , Caldwell, C. W. and Huang, T. H. Dissecting complex epigenetic alterations in breast cancer using CpG island microarrays, Cancer Res. , 61: 8375-8380, 2001.
12 Beckwith, M. , Longo, D. L. , O'Connell, C. D. , Moratz, C. M. and Urba, W. J. Phorbol ester-induced, cell-cycle-specific, growth inhibition of human B-lymphoma cell lines, J. Natl. Cancer Inst. , 82: 501-509, 1990.
13 Amin, H. M. , McDonnell, T. J. , Medeiros, L. J. , Rassidakis, G. Z. , Leventaki, V. , O'Connor, S. L. , Keating, M. J. and Lai, R. Characterization of 4 mantle cell lymphoma cell lines, Arch. Pathol. Lab Med. , 127: 424-431, 2003.
14 Stacchini, A. , Aragno, M. , Vallario, A. , Alfarano, A. , Circosta, P. , Gottardi, D. , Faldella, A. , Rege-Cambrin, G. , Thunberg, U. , Nilsson, K. and Caligaris-Cappio, F. MEC1 and MEC2: two new cell lines derived from B-chronic lymphocytic leukaemia in prolymphocytoid transformation, Leuk. Res. , 23: 127-136, 1999.
15 Boddy, J. L. , Gal, S. , Malone, P. R. , Harris, A. L. and Wainscoat, J. S. Prospective study of quantitation of plasma DNA levels in the diagnosis of malignant versus benign prostate disease, Clin. Cancer Res. , 11: 1394-1399, 2005.
16 Shi, H. , Yan, P. S. , Chen, C. M. , Rahmatpanah, F. , Lofton-Day, C. , Caldwell, C. W. and Huang, T. H. Expressed CpG island sequence tag microarray for dual screening of DNA hypermethylation and gene silencing in cancer cells, Cancer Res. , 62: 3214-3220, 2002.
17 Shi, H. , Wei, S. H. , Leu, Y. W. , Rahmatpanah, F. , Liu, J. C. , Yan, P. S. , Nephew, K. P. and Huang, T. H. Triple analysis of the cancer epigenome: an integrated microarray system for assessing gene expression, DNA methylation, and histone acetylation, Cancer Res. , 63: 2164-2171, 2003.
18 Liu, H. , Wang, J. and Epner, E. M. Cyclin D1 activation in B-cell malignancy: association with changes in histone acetylation, DNA methylation, and RNA polymerase II binding to both promoter and distal sequences, Blood, 104: 2505-2513, 2004.
19 Takahashi, T. , Shivapurkar, N. , Reddy, J. , Shigematsu, H. , Miyajima, K. , Suzuki, M. , Toyooka, S. , Zochbauer-Muller, S. , Drach, J. , Parikh, G. , Zheng, Y. , Feng, Z. , Kroft, S. H. , Timmons, C. , McKenna, R. W. and Gazdar, A. F. DNA methylation profiles of lymphoid and hematopoietic malignancies, Clin. Cancer Res. , 10: 2928-2935, 2004.
20 Kim, T. Y. , Jong, H. S. , Song, S. H. , Dimtchev, A. , Jeong, S. J. , Lee, J. W. , Kim, T. Y. , Kim, N. K. , Jung, M. and Bang, Y. J. Transcriptional silencing of the DLC-1 tumor suppressor gene by epigenetic mechanism in gastric cancer cells, Oncogene, 22. 3943-3951, 2003.
21 Pang, J. C. , Chang, Q. , Chung, Y. F. , Teo, J. G. , Poon, W. S. , Zhou, L. F. , Kong, X. and Ng, H. K. Epigenetic inactivation of DLC-1 in supratentorial primitive neuroectodermal tumor, Hum. Pathol. , 36: 36-43, 2005.
22 Yuan, B. Z. , Durkin, M. E. and Popescu, N. C. Promoter hypermethylation of DLC-1, a candidate tumor suppressor gene, in several common human cancers, Cancer Genet. Cytogenet. , 140: 113-117, 2003.
23 Yuan, B. Z. , Jefferson, A. M. , Baldwin, K. T. , Thorgeirsson, S. S. , Popescu, N. C. and Reynolds, S. H. DLC-1 operates as a tumor suppressor gene in human non-small cell lung carcinomas, Oncogene, 23: 1405-1411, 2004.
24 Ng, I. O. , Liang, Z. D. , Cao, L. and Lee, T. K. DLC-1 is deleted in primary hepatocellular carcinoma and exerts inhibitory effects on the proliferation of hepatoma cell lines with deleted DLC-1, Cancer Res. , 60: 6581-6584, 2000.
25 Yuan, B. Z. , Miller, M. J. , Keck, C. L. , Zimonjic, D. B. , Thorgeirsson, S. S. and Popescu, N. C. Cloning, characterization, and chromosomal localization of a gene frequently deleted in human liver cancer (DLC-1) homologous to rat RhoGAP, Cancer Res. , 58: 2196-2199, 1998.
26 Wong, C. M. , Lee, J. M. , Ching, Y. P. , Jin, D. Y. and Ng, I. O. Genetic and epigenetic alterations of DLC-1 gene in hepatocellular carcinoma, Cancer Res. , 63: 7646-7651, 2003.
27 Saci, A. and Carpenter, C. L. RhoA GTPase regulates B cell receptor signaling, Mol. Cell, 17: 205-214, 2005.
28 Campo, E. , Raffeld, M. and Jaffe, E. S. Mantle-cell lymphoma, Semin. Hematol. , 36: 115-127, 1999.
29 Costello, J. F. , Fruhwald, M. C. , Smiraglia, D. J. , Rush, L. J. , Robertson, G. P. , Gao, X. , Wright, F. A. , Feramisco, J. D. , Peltomaki, P. , Lang, J. C. , Schuller, D. E. , Yu, L. , Bloomfield, C. D. , Caligiuri, M. A. , Yates, A. , Nishikawa, R. , Su, H. H. , Petrelli, N. J. , Zhang, X. , O'Dorisio, M. S. , Held, W. A. , Cavenee, W. K. and Plass, C. Aberrant CpG-island methylation has non-random and tumour-type-specific patterns, Nat. Genet. , 24: 132-138, 2000.
30 Welsh, J. Vitamin D and breast cancer: insights from animal models, Am. J. Clin. Nutr. , 80: 1721S-1724S, 2004.
31 Hsu, J. Y. , Feldman, D. , McNeal, J. E. and Peehl, D. M. Reduced 1alpha-hydroxylase activity in human prostate cancer cells correlates with decreased susceptibility to 25-hydroxyvitamin D3-induced growth inhibition, Cancer Res. , 61: 2852-2856, 2001.
32 Himanen, J. P. , Chumley, M. J. , Lackmann, M. , Li, C. , Barton, W. A. , Jeffrey, P. D. , Vearing, C. , Geleick, D. , Feldheim, D. A. , Boyd, A. W. , Henkemeyer, M. and Nikolov, D. B. Repelling class discrimination: ephrin-A5 binds to and activates EphB2 receptor signaling, Nat. Neurosci. , 7. : 501-509, 2004.
33 Huusko, P. , Ponciano-Jackson, D. , Wolf, M. , Kiefer, J. A. , Azorsa, D. O. , Tuzmen, S. , Weaver, D. , Robbins, C. , Moses, T. , Allinen, M. , Hautaniemi, S. , Chen, Y. , Elkahloun, A. , Basik, M. , Bova, G. S. , Bubendorf, L. , Lugli, A. , Sauter, G. , Schleutker, J. , Ozcelik, H. , Elowe, S. , Pawson, T. , Trent, J. M. , Carpten, J. D. , Kallioniemi, O. P. and Mousses, S. Nonsense-mediated decay microarray analysis identifies mutations of EPHB2 in human prostate cancer, Nat. Genet. , 36: 979-983, 2004.
34 Wu, Q. , Zhang, T. , Cheng, J. F. , Kim, Y. , Grimwood, J. , Schmutz, J. , Dickson, M. , Noonan, J. P. , Zhang, M. Q. , Myers, R. M. and Maniatis, T. Comparative DNA sequence analysis of mouse and human protocadherin gene clusters, Genome Res. , 11: 389-404, 2001.
35 Wang, X. , Su, H. and Bradley, A. Molecular mechanisms governing Pcdh-gamma gene expression: evidence for a multiple promoter and cis-alternative splicing model, Genes Dev. , 16: 1890-1905, 2002.
36 Zelent, A. , Petrie, K. , Chen, Z. , Lotan, R. , Lubbert, M. , Tallman, M. S. , Ohno, R. , Degos, L. and Waxman, S. Molecular target-based treatment of human cancer: summary of the 10th international conference on differentiation therapy, Cancer Res. , 65: 1117-1123, 2005.

### Reference List for Example 4

1 Heisler, L. E. , Torti, D. , Boutros, P. C. , Watson, J. , Chan, C. , Winegarden, N. , Takahashi, M. , Yau, P. , Huang, T. H. , Farnham, P. J. , Jurisica, I. , Woodgett, J. R. , Bremner, R. , Penn, L. Z. and Der, S. D. CpG Island microarray probe sequences derived from a physical library are representative of CpG Islands annotated on the human genome, Nucleic Acids Res. , 33: 2952-2961, 2005.
2 Jones, P. A. and Baylin, S. B. The fundamental role of epigenetic events in cancer, Nat. Rev. Genet. , 3: 415-428, 2002.
3 Ng, M. H. , Wong, I. H. and Lo, K. W. DNA methylation changes and multiple myeloma, Leuk. Lymphoma, 34: 463-472, 1999.
4 Guillerm, G. , Gyan, E. , Wolowiec, D. , Facon, T. , vet-Loiseau, H. , Kuliczkowski, K. , Bauters, F. , Fenaux, P. and Quesnel, B. p16(INK4a) and p15(INK4b) gene methylations in plasma cells from monoclonal gammopathy of undetermined significance, Blood, 98: 244-246, 2001.
5 Wong, I. H. , Ng, M. H. , Lee, J. C. , Lo, K. W. , Chung, Y. F. and Huang, D. P. Transcriptional silencing of the p16 gene in human myeloma-derived cell lines by hypermethylation, Br. J. Haematol. , 103: 168-175, 1998.
6 Ng, M. H. , To, K. W. , Lo, K. W. , Chan, S. , Tsang, K. S. , Cheng, S. H. and Ng, H. K. Frequent death-associated protein kinase promoter hypermethylation in multiple myeloma, Clin. Cancer Res. , 7: 1724-1729, 2001.
7 Chim, C. S. , Fung, T. K. , Cheung, W. C. , Liang, R. and Kwong, Y. L. SOCS1 and SHP1 hypermethylation in multiple myeloma: implications for epigenetic activation of the Jak/STAT pathway, Blood, 103: 4630-4635, 2004.
8 Galm, O. , Yoshikawa, H. , Esteller, M. , Osieka, R. and Herman, J. G. SOCS-1, a negative regulator of cytokine signaling, is frequently silenced by methylation in multiple myeloma, Blood, 101: 2784-2788, 2003.
9 Mateos, M. V. , Garcia-Sanz, R. , Lopez-Perez, R. , Moro, M. J. , Ocio, E. , Hernandez, J. , Megido, M. , Caballero, M. D. , Femandez-Calvo, J. , Barez, A. , Almeida, J. , Orfao, A. , Gonzalez, M. and San Miguel, J. F. Methylation is an inactivating mechanism of the p16 gene in multiple myeloma associated with high plasma cell proliferation and short survival, Br. J. Haematol. , 118: 1034-1040, 2002.
10 Garcia-Manero, G. Methylation, aging, and pediatric acute lymphocytic leukemia, Leukemia, 17: 2063-2064, 2003.
11 De, V. J. , Thykjaer, T. , Tarte, K. , Ensslen, M. , Raynaud, P. , Requirand, G. , Pellet, F. , Pantesco, V. , Reme, T. , Jourdan, M. , Rossi, J. F. , Orntoft, T. and Klein, B. Comparison of gene expression profiling between malignant and normal plasma cells with oligonucleotide arrays, Oncogene, 21: 6848-6857, 2002.
12 Zent, C. S. , Zhan, F. , Schichman, S. A. , Bumm, K. H. , Lin, P. , Chen, J. B. and Shaughnessy, J. D. The distinct gene expression profiles of chronic lymphocytic leukemia and multiple myeloma suggest different anti-apoptotic mechanisms but predict only some differences in phenotype, Leuk. Res. , 27. 765-774, 2003.
13 Zhan, F. , Hardin, J. , Kordsmeier, B. , Bumm, K. , Zheng, M. , Tian, E. , Sanderson, R. , Yang, Y. , Wilson, C. , Zangari, M. , Anaissie, E. , Morris, C. , Muwalla, F. , van, R. F. , Fassas, A. , Crowley, J. , Tricot, G. , Barlogie, B. and Shaughnessy, J. , Jr. Global gene expression profiling of multiple myeloma, monoclonal gammopathy of undetermined significance, and normal bone marrow plasma cells, Blood, 99: 1745-1757, 2002.
14 Shi, H. , Wei, S. H. , Leu, Y. W. , Rahmatpanah, F. , Liu, J. C. , Yan, P. S. , Nephew, K. P. and Huang, T. H. Triple analysis of the cancer epigenome: an integrated microarray system for assessing gene expression, DNA methylation, and histone acetylation, Cancer Res. , 63: 2164-2171, 2003.
15 Shi, H. , Yan, P. S. , Chen, C. M. , Rahmatpanah, F. , Lofton-Day, C. , Caldwell, C. W. and Huang, T. H. Expressed CpG island sequence tag microarray for dual screening of DNA hypernetliylation and gene silencing in cancer cells, Cancer Res. , 62. 3214-3220, 2002.
16 Nouzova, M. , Holtan, N. , Oshiro, M. M. , Isett, R. B. , Munoz-Rodriguez, J. L. , List, A. F. , Narro, M. L. , Miller, S. J. , Merchant, N. C. and Futscher, B. W. Epigenomic changes during leukemia cell differentiation: analysis of histone acetylation and cytosine methylation using CpG island microarrays, J. Pharmacol. Exp. Ther. , 311: 968-981, 2004.
17 Groot, G. S. and Kroon, A. M. Mitochondrial DNA from various organisms does not contain internally methylated cytosine in-, Biochim. Biophys. Acta, 564: 355-357, 1979.
18 Zhan g, Q. , Wang, H. Y. , Marzec, M. , Raghunath, P. N. , Nagasawa, T. and Wasik, M. A. S, Proc. Natl. Acad. Sci. U. S. A, 102: 6948-6953, 2005.
19 Yuan, B. Z. , Jefferson, A. M. , Baldwin, K. T. , Thorgeirsson, S. S. , Popescu, N. C. and Reynolds, S. H. DLC-1 operates as a tumor suppressor gene in human non-small cell lung carcinomas, Oncogene, 23: 1405-1411, 2004.

### Reference List for Example 7

1. Harris N L, Jaffe E S, Diebold J et al. World Health Organization classification of neoplastic diseases of the hematopoietic and lymphoid tissues: report of the Clinical Advisory Committee meeting-Airlie House, Virginia, November 1997. J Clin Oncol. 1999; 17:3835-3849.
2. Dybkaer K, Iqbal J, Zhou G et al. Molecular diagnosis and outcome prediction in diffuse large B-cell lymphoma and other subtypes of lymphoma. Clin Lymphoma. 2004; 5:19-28.
3. Egger G, Liang G, Aparicio A et al. Epigenetics in human disease and prospects for epigenetic therapy. Nature. 2004; 429:457-463.
4. Jones P A, Takai D. The role of DNA methylation in mammalian epigenetics. Science. 2001; 293:1068-1070.
5. Jones P A, Baylin S B. The fundamental role of epigenetic events in cancer. Nat Rev Genet. 2002; 3:415-428.
6. Gitan R S, Shi H, Chen C M et al. Methylation-specific oligonucleotide microarray: a new potential for high-throughput methylation analysis. Genome Res. 2002; 12:158-164.
7. Shi H, Maier S, Nimmrich I et al. Oligonucleotide-based microarray for DNA methylation analysis: principles and applications. J Cell Biochem. 2003; 88:138-143.
8. Yan P S, Wei S H, Huang T H. Methylation-specific oligonucleotide microarray. Methods Mol Biol. 2004; 287:251-260.
9. Adorjan P, Distler J, Lipscher E et al. Tumour class prediction and discovery by microarray-based DNA methylation analysis. Nucleic Acids Res. 2002; 30:e21.

### Normalization:

Yang, Y. H. , Dudoit S. , Luu P. , Lin D. M. , Peng V. , Ngai J. , Speed T. P. (2002), “Normalization for cDNA microarray data: a robust composite method addressing single and multiple slide systematic variation”, Nucleic Acids Res. , vol. 30, no. 4, e15.
Wolfinger R. D. , Gibson G. , Wolfinger E. D. , Bennett L. , Hamadeh H. , Bushel P. , Afshari C. , Paules R. S. (2001), “Assessing gene significance from cDNA microarray expression data via mixed models”, J of Computational Biology, 8:625-637.
Bolstand B. M. , Irizarry R. A. , Astrand M. , Speed T. P. (2003), “A comparison of normalization methods for high density oligonucleotide array data based on variance and bias”, Bioinfomratics, vol. 19, no. 2, pp. 185-193.
Huber W. , von Heydebreck A. , Sultmann H. , Pustka A. , Vingron M. (2002), “Variance stabilization applied to microarray data calibration and to the quantification of differential expression”, Bioinformatics, Vol. 18, Suppl. 1, pp. s96-s104.

### Feature Selection

Golub, T. R. , Slonim, D. K. , Tamayo, P. , Huard, C. , Gaasenbeek, M. , Mesirov, J. P. , Coller, H. , Loh, M. L. , Downing, J. R. , Caligiuri, M. A. , Bloomfield, C. D. and Lander, E. S. (1999), “Molecular classification of cancer: class discovery and class prediction by gene expression monitoring”, . Science, 286, 531-537.
Eng-Juh Yeoh, Mary E. Ross, Shelia A. Shurtleff, W. Kent Williams, Divyen Patel, Rami Mahfouz, Fred G. Behm, Susana C. Raimondi, Mary V. Relling, Anami Patel, Cheng Cheng, Dario Campana, Dawn Wilkins, Xiaodong Zhou, Jinyan Li, Huiqing Liu, Ching-Hon Pui, William E. Evans, Clayton NAeve, Limsoon Wong, James R. Downing, (2002). “Classification, subtype discovery, and prediction of outcome in pediatric acute lymphoblastic leukemia by gene expression profiling”, Cancer Cell, vol 1(2), pp 133-143.
Khan J. , Wei J. S. , Rigner M. , Saal L. H. , Ladanyi M. , Westermann F, Berthold F. , Schwab M. , Antonescu C. R. , Peterson C. , Meltzer P. S. (2001), “Classification and diagnostic prediction of cancers using gene expression profiling and artificial neural networks”, Nature Medicine, vol. 7, no. 6, pp. 673-679.
Lee M. T. (2004), “Analysis of Microarray gene expression data”, Kluwer Academic Publishers, Norwell, M A.
Cox T. F. , Cox M. A. A. (2001), Multidimensional Scaling, second edition, CRC Press, Boca Raton, Fla.

### Classification

Furey T. S. , Cristiani N. , Duffy N. , Bednarski D. W. , Schumm M. , Haussler D. , (2000) Support vector machines classification and validation of cancer tissue samples using microarray expressioin data”, Bioinformatics, 16, pp 906-914.
Asyali M. H. , Alci M. (2005), “Reliability analysis of microarray data using fuzzy c-means and normal mixture modeling based classification methods”, Bioinformatics, vol 21, no 5, pp. 644-649.
Clayerie, J-M. (1999), “Computational methods for the identification of differential and coordinated gene expression”, Human Molecular Genetics, 8, 1821-1832.
Bezdek, J. C. (1981), Pattern Recognition with Fuzzy Objective Function Algorithms, Plenum Press, NY.

### Co-Clustering

Cheng Y. , Church G. M. (2000). “Biclustering of expression data”, In Proceedings of the Eighth International Conference on Intelligent Systems for Molecular Biology (ISMB), pages 93-103.
Cho H. , Dhillon I. S. , Guan Y. , Sra S. (2004), “Minimum Sum-Squared Residue Co-clustering of Gene Expression Data”, Proceedings of SIAM Data Mining Conf. , pp 114-125.
Oh C. H. , Ichinashi H. (2001), “Fuzzy clustering for categorical multivariate data”, Proceedings of the IFSA World Congress, pp. 2154-2159, July 25-28, Vancouver, Canada.
Oyanagi S. , Kubota K. , Nakase A. (2001), “Application of matrix clustering to web log analysis and access prediction,” in WEBKDD, August 2001.
Getz G. , Levine E. , Domany E. (2000), “Coupled two-way clustering analysis of gene microarray data”, PNAS, vol 97, no 22, pp 12079-12084.
Kummamuru K. , Dhawale A. , Khrishnapuram R. (2003), Proceedings of the 12th IEEE The International Conference on Fuzzy Systems, St Louis, Mo. , pp 772-777.

1. A high-throughput method for distinguishing between non-Hodgkin's Lymphoma (NHL), and benign follicular hyperplasia (BFH) or normal lymph node tissue, comprising:
obtaining a test sample comprising genomic DNA;
contacting the genomic DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of a DLC-1 promoter CpG-island region, wherein distinguishing, based on the determined methylation state or level relative to a respective control or normalized control methylation state or level, non-Hodgkin's Lymphoma (NHL) from benign follicular hyperplasia (BFH) is, at least in part, afforded.

2. The method of claim 1, wherein, the DLC-1 promoter CpG-island region comprises a sequence selected from the group consisting of SEQ ID NO:128, portions thereof, and complements thereto.

3. The method of claim 1, wherein the test sample comprising genomic DNA is a serum sample from a subject to be tested.

4. The method of claim 1, wherein distinguishing is at 95 to 100%, or 100% specificity and at least 77% sensitivity, based on used methylation threshold values.

5. A high-throughput method for distinguishing between non-Hodgkin's Lymphoma NHL), and benign follicular hyperplasia (BFH) or normal lymph node tissue, comprising:
obtaining a test sample comprising expressed RNA; and
determining, using one or more suitable RNA measurement assays, a level or amount of expressed DLC-1 RNA in the test sample, wherein distinguishing, based on the determined level or amount relative to a control or normalized control level or amount of expressed DLC-1 RNA, non-Hodgkin's Lymphoma (NHL) from normal lymph node tissue, is at least in part, afforded.

6. The method of claim 5, wherein the test sample comprising genomic DNA is a serum sample from a subject to be tested.

7. A high-throughput method for identifying, or for distinguishing between and among subtypes of small B-cell lymphomas (SBCL), comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of at least one promoter CpG-island region selected from the promoter group consisting of LHX2, POU3F3, HOX10, NRP2, PRKCE, RAMP, MLLT2, NKX6-1, LPR1B, and ARF4, wherein distinguishing, based on the determined methylation state or level relative to a respective control or normalized control methylation state or level, germinal center-derived tumors from pre- and/or post-germinal center lymphomas is, at least in part, afforded.

8. The method of claim 7, wherein the at least one promoter CpG-island region selected from the promoter group consisting of LHY2, POU3F3, HOX10, NRP2, PRKCE, RAMP, NKX6-1, LPR1B, and ARF4 respectively comprises SEQ ID NO:101 (LHX2), SEQ ID NO:119 (POU3F3), SEQ ID NO:116 (HOX10), SEQ ID NO:122 (NRP2), SEQ ID NO:110 (PRKCE), SEQ ID NO:125 (RAMP), SEQ ID NO:155 (NKX6-1), SEQ ID NO:107 (LPR1B) and SEQ ID NO:104 (ARF4).

9. The method of claim 7, wherein distinguishing germinal center-derived tumors from pre- and/or post-germinal center lymphomas, comprises distinguishing between and/or among mantle cell lymphoma (MCL), follicular lymphoma (FL), and B-cell chronic lymphocytic leukemia/small lymphocytic lymphoma (B-CLL/SLL).

10. The method of claim 7, wherein the test sample comprising genomic DNA is a serum sample from a subject to be tested.

11. A high-throughput method for identifying, or for distinguishing between and among subtypes of non-Hodgkin's Lymphoma (NHL), comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of at least one promoter CpG-island region selected from the promoter group consisting of DLC-1, PCDHGB7, CYP27B1, EFNA5, CCND1 and RARβ2, wherein identifying or distinguishing between or among, based on the determined methylation state or level relative to a respective control or normalized control methylation state or level, subtypes of non-Hodgkin's Lymphoma (NHL) is, at least in part, afforded.

12. The method of claim 11, wherein the at least one promoter CpG-island region selected from the promoter group consisting of DLC-1, PCDHGB7, CYP27B1, EFNA5, CCND1 and RAR□ respectively comprises SEQ ID NO:128 (DLC-1), SEQ ID NO:136 (PCDHGB7), SEQ ID NO:133 (CYP27B1), SEQ ID NO:139 (EFNA5), SEQ ID NO:142 (CCND1), and SEQ ID NO: 130 (RARβ).

13. The method of claim 11, wherein identifying or distinguishing between or among subtypes of non-Hodgkin's Lymphoma (NHL), comprises distinguishing between and/or among mantle cell lymphoma (MCL), follicular lymphoma (FL), B-cell chronic lymphocytic leukemia/small lymphocytic lymphoma (B-CLL/SLL), and diffuse large B-cell lymphoma (DLBCL).

14. The method of claim 11, wherein identifying or distinguishing between or among subtypes of non-Hodgkin's Lymphoma (NHL), comprises identifying or distinguishing between and/or among germinal center-derived tumors, and pre- and/or post-germinal center lymphomas.

15. The method of claim 11, wherein the test sample comprising genomic DNA is a serum sample from a subject to be tested.

16. A high-throughput method for diagnosis, prognosis or monitoring multiple myeloma (MM), comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of at lease one promoter CpG-island region selected from the promoter group consisting of DL C-1, PCDHGB7, CYP27B1 and NOPE, wherein diagnosing, prognosing or monitoring multiple myeloma (MM), based on the determined methylation state or level relative to a respective control or normalized control methylation state or level is, at least in part, afforded.

17. The method of claim 16, wherein the at least one promoter CpG-island region selected from the promoter group consisting of DLC-1, PCDHGB7, CYP27B1, and NOPE respectively comprises SEQ ID NO:128 (DLC-1), SEQ ID NO:136 (PCDHGB7), SEQ ID NO:133 (CYP27B1), and SEQ ID NO:171: (NOPE).

18. The method of claim 16, wherein the test sample comprising genomic DNA is a serum sample from a subject to be tested.

19. A high-throughput method for identifying acute lymphoblastic leukemia (ALL), or for distinguishing ALL from normal bone marrow, comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of at least one promoter CpG-island region selected from the promoter group consisting of DCC, DLC-1, DDX51, KCNK2, LRP1B, NKX6-1, NOPE, PCDHGA12, RPIB9/ABCB1(MDR1) and SLC2A14, wherein identifying acute lymphoblastic leukemia (ALL) or distinguishing acute lymphoblastic leukemia (ALL) from normal bone marrow, based on the determined methylation state or level relative to a respective control or normalized control methylation state or level, is, at least in part, afforded.

20. The method of claim 19, wherein the at least one promoter CpG-island region selected from the promoter group consisting of DCC, DLC-1, DDX51, KCNK2, LRP1B, NKX6-1, NOPE, PCDHGA12, RPIB9/ABCB1(MDR1) and SLC2A14 respectively comprises SEQ ID NO:174 (DCC), SEQ ID NO:128 (DLC-1), SEQ ID NO:167 (DDX51), SEQ ID NO:151 (KCNK2), SEQ ID NO:107 (LRP1B), SEQ ID NO:113 (NKX6-1), SEQ ID NO:1171 (NOPE), SEQ ID NO:158 (PCDHGA12,) SEQ ID NO:161 (RPIB91ABCB1(MDR1)), and SEQ ID NO:164 (SLC2A14).

21. The method of claim 20, wherein the test sample comprising genomic DNA is a serum sample from a subject to be tested.

22. A high-throughput method for distinguishing B-ALL from T-ALL, comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of a DDX51 promoter CpG-island region, wherein distinguishing B-ALL from T-ALL, based on the determined methylation state or level relative to a respective control or normalized control methylation state or level, is, at least in part, afforded.

20. The method of claim 19, wherein the DDX51 promoter CpG-island region comprises a sequence selected from the group consisting of SEQ ID NO: 167, portions thereof, and complements thereto.

21. The method of claim 19, wherein the test sample comprising genomic DNA is a serum sample from a subject to be tested.

22. A high-throughput method for identifying acute lymphoblastic leukemia (ALL), or for distinguishing ALL from normal bone marrow, comprising:
obtaining a test sample comprising expressed RNA; and
determining, in the test sample and using one or more suitable RNA measurement assays, a level or amount of expressed RNA corresponding to at least one gene selected from the group consisting of ABCB1, DCC, DLC-1, PCDHGA12, RPIB9, KCNK2 and NOPE, wherein distinguishing, based on the determined level or amount relative to a control or normalized control level or amount of expressed DLC-1 RNA, non-Hodgkin's Lymphoma (NHL) from normal lymph node tissue, is at least in part, afforded.

23. The method of claim 22, wherein the at least one gene is selected from the group consisting of ABCB1, DCC, DLC-1, PCDHGA12, and RPIB9.

24. The method of claim 22, wherein the test sample comprising genomic DNA is a serum sample from a subject to be tested.

25. A high-throughput method for identifying subtypes of acute myelogenous leukemia (AML), or for distinguishing between acute myelogenous leukemia (AML) and acute lymphoblastic leukemia (ALL), comprising:
obtaining a test sample comprising genomic DNA;
contacting the DNA with a reagent or reagents that distinguish between cytosine and 5-methylcytosine to provide for a treated DNA; and
determining, using the treated DNA and at least one suitable methylation assay, a methylation state or level of at least one CpG dinucleotide sequence of at least one promoter CpG-island region selected from the promoter group consisting of DDX51, EXOSC8, NOPE, FBX036, SMAD9, and RP1B9, wherein distinguishing subtypes of acute myelogenous leukemia (AML), or distinguishing between acute myelogenous leukemia (AML) and acute lymphoblastic leukemia (ALL), based on the determined methylation state or level relative to a respective control or normalized control methylation state or level, is, at least in part, afforded.

26. The method of claim 25, wherein the at least one promoter CpG-island region selected from the promoter group consisting of DDX51, EXOSC8, NOPE, SMAD9, and RP1B9, respectively comprises SEQ ID NO: 167 (DDX51), SEQ ID NO: 177 (EXOSC8), SEQ ID NO: 171 (NOPE), SEQ ID NO:180 (SMAD9), and SEQ ID NO:161 (RP1B9).

27. The method of claim 25, wherein distinguishing subtypes of acute myelogenous leukemia (AML), comprises distinguishing between AML granulocyte FAB subtypes M0 to M3.

28. The method of claim 25 wherein the test sample comprising genomic DNA is a serum sample from a subject to be tested.

29. A method for identification of methylation markers for cancer, comprising:
obtaining a plurality of pathologically classified cancer tissue samples corresponding to at least one particular form, type or subtype of cancer, the samples comprising genomic DNA and corresponding to a plurality of different individuals or sources;
extracting and normalizing intensity data values corresponding to test nucleic acid samples hybridized to at least one nucleic acid-based probe array, wherein the intensity data values correspond to the methylation level of particular candidate marker DNA sequences, to provide for extracted features;
conducting a gene-finding step, comprising conducting a plurality of feature selection methods;
clustering, with respect to each of the feature selection methods, the pathologically classified cancer tissue samples or sources using a cross-correlation matrix;
assessing the clustering by using multidimensional scaling to provide for a selected gene marker set corresponding to each of the feature selection methods;
fusing the results of the plurality of feature selection methods to provide for at least one list of candidate differentially methylated gene markers, wherein said fusion comprises voting such that only candidate gene markers selected by all, or majority of the plurality of feature selection methods as being uniquely methylated in a given class are selected for further validation; and
validating of the listed candidate gene markers using at least one suitable methylation assay with cancer tissue or cells.

30. The method of claim 29, wherein conducting a gene-finding step, comprising conducting a plurality of feature selection methods comprises conducting at least two feature selection methods selected from the group consisting of: idealized methylation pattern; chi-square; T-test; correlation based feature selection; principal component analysis; and permutation tests.

31. The method of claim 30, wherein the at least two feature selection methods are an idealized methylation pattern, and a pair-wise T-test.

32. The method of claim 31, wherein the idealized methylation pattern feature test comprises establishing cross-correlation values, and ranking of the values.

33. The method of claim 31, wherein the pair-wise T-test feature test is suitable to determine if the mean level of methylation values in one class is higher than that of other classes.

34. The method of claim 29, wherein assessing the clustering by using multidimensional scaling is by Euclidean multidimensional scaling.

35. The method of claim 29, further comprising, prior to validation, ranking of the listed candidate gene markers based on their frequency of appearance in a comprehensive literature database, screened by searching each gene marker against the particular cancer form.

36. The method of claim 35, wherein the comprehensive literature database is Medline or Medline abstracts.

37. The method of claim 29, wherein clustering the cancer tissue samples or sources using a cross-correlation matrix, comprises use of fuzzy C-means on the cross-correlation matrix to select for a best match with the pathological classification.

37. The method of claim 29, wherein the at least one suitable methylation assay comprising at least one method selected from the group consisting of COBRA, MSP, MethyLight, and MS-SNuPE.

38. The method of claim 29, further comprising:
extracting and normalizing intensity data values corresponding to test nucleic acid samples hybridized to at least one nucleic acid-based probe array, wherein the intensity data values correspond to the expression level of particular candidate marker DNA sequences, to provide for extracted features, wherein rank fusion (rank averaging) is between a differentially methylated gene marker ranking (e.g., IMP, t-test) and a differentially expressed gene marker ranking (e.g., IEP, t-test), resulting in a fused rank list from which candidate gene markers are optimally selected by computing a patient correlation matrix and clustering of the patient similarity matrix using C-means to select for an optimal number of gene that best match the pathologically-determined diagnosis/classification.

39. The method of claim 38, wherein the methylation array and the expression array are different arrays.

40. The method of claim 38, wherein the methylation array and the expression array are the same array.


SEQUENCE LISTING (181 sequences)

SEQ ID NO: 1 - DNA, 24 bp, Artificial Sequence
  DLC-1 promoter region primer methylated (+)
cccaacgaaaaaacccgactaacg

SEQ ID NO: 2 - DNA, 24 bp, Artificial Sequence
  DLC-1 promoter region primer methylated (-)
tttaaagatcgaaacgagggagcg

SEQ ID NO: 3 - DNA, 27 bp, Artificial Sequence
  DLC-1 promoter region primer unmethylated (+)
aaacccaacaaaaaaacccaactaaca

SEQ ID NO: 4 - DNA, 27 bp, Artificial Sequence
  DLC-1 promoter region primer unmethylated (-)
ttttttaaagattgaaatgagggagtg

SEQ ID NO: 5 - DNA, 24 bp, Artificial Sequence
  DLC-1 promoter region probe
aagttcgtgagtcggcgtttttga

SEQ ID NO: 6 - DNA, 24 bp, Artificial Sequence
  H24 PCR linker
aggcaactgtgctatccgagggat

SEQ ID NO: 7 - DNA, 12 bp, Artificial Sequence
  H24 PCR linker
taatccctcgga

SEQ ID NO: 8 - DNA, 22 bp, Artificial Sequence
  HOX10 antisense methylated primer
ttttaaagttacggtttgtcgg

SEQ ID NO: 9 - DNA, 23 bp, Artificial Sequence
  HOX10 sense methylated primer
ctcaaaaccactaaaactccgaa

SEQ ID NO: 10 - DNA, 20 bp, Artificial Sequence
  HOX10 antisense unmethylated primer
ttaaagttatggtttgttgg

SEQ ID NO: 11 - DNA, 20 bp, Artificial Sequence
  HOX10 sense unmethylated primer
aaaaccactaaaactccaaa

SEQ ID NO: 12 - DNA, 25 bp, Artificial Sequence
  ARF4 antisense methylated primer
tcggaactaacctttattatttcga

SEQ ID NO: 13 - DNA, 25 bp, Artificial Sequence
  ARF4 sense methylated primer
aaaattaaccaatttcgctaacgta

SEQ ID NO: 14 - DNA, 24 bp, Artificial Sequence
  ARF4 antisense unmethylated primer
tggaagtaaggtttattattttga

SEQ ID NO: 15 - DNA, 25 bp, Artificial Sequence
  ARF4 sense unmethylated primer
aaaattaaccaatttcactaacata

SEQ ID NO: 16 - DNA, 22 bp, Artificial Sequence
  BLK antisense methylated primer
gtttattttagcggaaaaaggc

SEQ ID NO: 17 - DNA, 25 bp, Artificial Sequence
  BLK sense methylated primer
aacctataaaacacacacgtacgta

SEQ ID NO: 18 - DNA, 24 bp, Artificial Sequence
  BLK antisense unmethylated primer
gtttattttagtggaaaaaggtgt

SEQ ID NO: 19 - DNA, 27 bp, Artificial Sequence
  BLK sense unmethylated primer
caacctataaaacacacacatatcata

SEQ ID NO: 20 - DNA, 25 bp, Artificial Sequence
  LHX2 antisense methylated primer
tttagtttatttcgttggggtaaac

SEQ ID NO: 21 - DNA, 24 bp, Artificial Sequence
  LHX2 sense methylated primer
caaataattcaacttccactcgaa

SEQ ID NO: 22 - DNA, 25 bp, Artificial Sequence
  LHX2 antisense unmethylated primer
tagtttattttgttggggtaaatgg

SEQ ID NO: 23 - DNA, 25 bp, Artificial Sequence
  LHX2 sense unmethylated primer
tcaaataattcaacttccactcaaa

SEQ ID NO: 24 - DNA, 21 bp, Artificial Sequence
  LRP1B antisense methylated primer
agtttgcgttggagattgttc

SEQ ID NO: 25 - DNA, 25 bp, Artificial Sequence
  LRP1B sense methylated primer
aataacatttataaataccgccgtt

SEQ ID NO: 26 - DNA, 23 bp, Artificial Sequence
  LRP1B antisense unmethylated primer
aagtttgtgttggagattgtttg

SEQ ID NO: 27 - DNA, 27 bp, Artificial Sequence
  LRP1B sense unmethylated primer
ccaataacatttataaataccaccatt

SEQ ID NO: 28 - DNA, 25 bp, Artificial Sequence
  MLLT2 antisense methylated primer
agagtaggtagtttcgtaatatcgg

SEQ ID NO: 29 - DNA, 20 bp, Artificial Sequence
  MLLT2 sense methylated primer
aatcttccgtccataaacgc

SEQ ID NO: 30 - DNA, 26 bp, Artificial Sequence
  MLLT2 antisense unmethylated primer
gagagtaggtagttttgtaatattgg

SEQ ID NO: 31 - DNA, 23 bp, Artificial Sequence
  MLLT2 sense unmethylated primer
aaaatcttccatccataaacacc

SEQ ID NO: 32 - DNA, 24 bp, Artificial Sequence
  NKX6-1 antisense methylated primer
ttttagagtggtcgtttgtagtcg

SEQ ID NO: 33 - DNA, 26 bp, Artificial Sequence
  NKX6-1 sense methylated primer
aaatctcgtatattttctctttccgt

SEQ ID NO: 34 - DNA, 25 bp, Artificial Sequence
  NKX6-1 antisense unmethylated primer
ttttagagtggttgtttgtagttga

SEQ ID NO: 35 - DNA, 26 bp, Artificial Sequence
  NKX6-1 sense unmethylated primer
aatctcatatattttctctttccatc

SEQ ID NO: 36 - DNA, 25 bp, Artificial Sequence
  RAMP antisense methylated primer
atgaatttcgttagtttcgagtagc

SEQ ID NO: 37 - DNA, 24 bp, Artificial Sequence
  RAMP sense methylated primer
ctcaactaaaacttttcctccgac

SEQ ID NO: 38 - DNA, 25 bp, Artificial Sequence
  RAMP antisense unmethylated primer
gaattttgttagttttgagtagtgg

SEQ ID NO: 39 - DNA, 25 bp, Artificial Sequence
  RAMP sense unmethylated primer
tctcaactaaaacttttcctccaac

SEQ ID NO: 40 - DNA, 27 bp, Artificial Sequence
  POU3F3 antisense methylated primer
tgtatatatatatatacgaggaagcgg

SEQ ID NO: 41 - DNA, 20 bp, Artificial Sequence
  POU3F3 sense methylated primer
gatcaacgaaaccgtacgat

SEQ ID NO: 42 - DNA, 27 bp, Artificial Sequence
  POU3F3 antisense unmethylated primer
tgtatatatatatatatgaggaagtgg

SEQ ID NO: 43 - DNA, 26 bp, Artificial Sequence
  POU3F3 sense unmethylated primer
aaaataccaatcaacaaaaccataca

SEQ ID NO: 44 - DNA, 25 bp, Artificial Sequence
  NRP2 antisense methylated primer
ttttagagattagcgttgtagtcga

SEQ ID NO: 45 - DNA, 21 bp, Artificial Sequence
  NRP2 sense methylated primer
aaaccgaaactaaaacctccg

SEQ ID NO: 46 - DNA, 25 bp, Artificial Sequence
  NRP2 antisense unmethylated primer
ttttagagattagtgttgtagttga

SEQ ID NO: 47 - DNA, 23 bp, Artificial Sequence
  NRP2 sense unmethylated primer
aaaaccaaaactaaaacctccac

SEQ ID NO: 48 - DNA, 25 bp, Artificial Sequence
  PRKCE antisense methylated primer
tcggtaagtttgtagtgataaagtc

SEQ ID NO: 49 - DNA, 21 bp, Artificial Sequence
  PRKCE sense methylated primer
ctcgaaaaccactaaaacgaa

SEQ ID NO: 50 - DNA, 27 bp, Artificial Sequence
  PRKCE antisense unmethylated primer
ttggtaagtttgtagtgataaagttgt

SEQ ID NO: 51 - DNA, 25 bp, Artificial Sequence
  PRKCE sense unmethylated primer
aaacctcaaaaaccactaaaacaaa

SEQ ID NO: 52 - DNA, 24 bp, Artificial Sequence
  CCND1 sense primer for COBRA analysis
ggtttgggtaataagttgtaggga

SEQ ID NO: 53 - DNA, 26 bp, Artificial Sequence
  CCND1 antisense primer for COBRA analysis
caaccataaaacaccaactcctatac

SEQ ID NO: 54 - DNA, 25 bp, Artificial Sequence
  EFNA5 sense primer for COBRA analysis
tttaaggagggaaagaggagtagtt

SEQ ID NO: 55 - DNA, 25 bp, Artificial Sequence
  EFNA5 antisense primer for COBRA analysis
aaatccctccaactcctaaataaac

SEQ ID NO: 56 - DNA, 30 bp, Artificial Sequence
  PCDHGB7 sense primer for COBRA analysis
tggggtagaataaaggtagtagtaaaggaa

SEQ ID NO: 57 - DNA, 25 bp, Artificial Sequence
  PCDHGB7 antisense primer for COBRA analysis
acaatcccacacaaaacctctaaac

SEQ ID NO: 58 - DNA, 30 bp, Artificial Sequence
  NOPE sense primer for COBRA analysis
ttttttgttttatttattttagttttagtt

SEQ ID NO: 59 - DNA, 24 bp, Artificial Sequence
  NOPE antisense primer for COBRA analysis
aaaacccatctccacaaatatcat

SEQ ID NO: 60 - DNA, 27 bp, Artificial Sequence
  RPIB9 sense primer for COBRA analysis
attggaattgatataaagtttagggtt

SEQ ID NO: 61 - DNA, 25 bp, Artificial Sequence
  RPIB9 antisense primer for COBRA analysis
acccccttaaacaaatataaaaaac

SEQ ID NO: 62 - DNA, 25 bp, Artificial Sequence
  PON3 sense primer for COBRA analysis
tttttgggtagaggttaaggtttaa

SEQ ID NO: 63 - DNA, 26 bp, Artificial Sequence
  PON3 antisense primer for COBRA analysis
ccccaaatcctaaaaaaaataaatta

SEQ ID NO: 64 - DNA, 25 bp, Artificial Sequence
  FLJ39155 sense primer for COBRA analysis
ggtttttgtttttggtttttagttt

SEQ ID NO: 65 - DNA, 30 bp, Artificial Sequence
  FLJ39155 antisense primer for COBRA analysis
atctaaaaaattaatcattcttttaataaa

SEQ ID NO: 66 - DNA, 22 bp, Artificial Sequence
  DCC sense primer for COBRA analysis
ggatattttagaaaagtgagag

SEQ ID NO: 67 - DNA, 26 bp, Artificial Sequence
  DCC antisense primer for COBRA analysis
caaatcatcaataaaccacatccaaa

SEQ ID NO: 68 - DNA, 27 bp, Artificial Sequence
  DDX51 sense primer for COBRA analysis
ttttttatttgttttatttaaggtgtt

SEQ ID NO: 69 - DNA, 25 bp, Artificial Sequence
  DDX51 antisense primer for COBRA analysis
tctactaaacttacccctatcctcc

SEQ ID NO: 70 - DNA, 25 bp, Artificial Sequence
  KCNK2 sense primer for COBRA analysis
tttagtaaaggggttttgttttgag

SEQ ID NO: 71 - DNA, 25 bp, Artificial Sequence
  KCNK2 antisense primer for COBRA analysis
aaccctaacttcttccaatctacac

SEQ ID NO: 72 - DNA, 26 bp, Artificial Sequence
  NXX6-1 antisense primer for COBRA analysis
ttttgtatatttggagggataggtat

SEQ ID NO: 73 - DNA, 25 bp, Artificial Sequence
  NXX6-1 antisense primer for COBRA analysis
ccttttattcatcaaaaatttaccc

SEQ ID NO: 74 - DNA, 30 bp, Artificial Sequence
  PCDHGA12 sense primer for COBRA analysis
aatgtttagatttaatgtatatttgatggt

SEQ ID NO: 75 - DNA, 25 bp, Artificial Sequence
  PCDHGA12 antisense primer for COBRA analysis
ctccaaaaacctaaaactaaaaccc

SEQ ID NO: 76 - DNA, 25 bp, Artificial Sequence
  SLC2A14 sense primer for COBRA analysis
ggttttaaggttagttttttagagt

SEQ ID NO: 77 - DNA, 21 bp, Artificial Sequence
  SLC2A14 antisense primer for COBRA analysis
aaacaattaataaatcccaac

SEQ ID NO: 78 - DNA, 22 bp, Artificial Sequence
  ABCB1 sense primer for real-time SYBR green analysis
tgtatgctcagagtttgcaggt

SEQ ID NO: 79 - DNA, 21 bp, Artificial Sequence
  ABCB1 antisense primer for real-time SYBR green analysis
ttccaaagatgtgtgctttcc

SEQ ID NO: 80 - DNA, 20 bp, Artificial Sequence
  DCC sense primer for real-time SYBR green analysis
ccgaaagtcccttacacacc

SEQ ID NO: 81 - DNA, 21 bp, Artificial Sequence
  DCC antisense primer for real-time SYBR green analysis
catgggtcttaggaagagtgg

SEQ ID NO: 82 - DNA, 20 bp, Artificial Sequence
  DDX51 sense primer for real-time SYBR green analysis
cacactgctcctgaaagtgc

SEQ ID NO: 83 - DNA, 21 bp, Artificial Sequence
  DDX51 antisense primer for real-time SYBR green analysis
ttcagttagcattcggaggaa

SEQ ID NO: 84 - DNA, 21 bp, Artificial Sequence
  HPRT1 sense primer for real-time SYBR green analysis
tgacactggcaaaacaatgca

SEQ ID NO: 85 - DNA, 21 bp, Artificial Sequence
  HPRT1 antisense primer for real-time SYBR green analysis
ggtccttttcaccagcaagct

SEQ ID NO: 86 - DNA, 26 bp, Artificial Sequence
  KCNK2 sense primer for real-time SYBR green analysis
taacaactattggatttggtgactac

SEQ ID NO: 87 - DNA, 20 bp, Artificial Sequence
  KCNK2 antisense primer for real-time SYBR green analysis
gccctacaaggatccagaac

SEQ ID NO: 88 - DNA, 21 bp, Artificial Sequence
  LRP1B sense primer for real-time SYBR green analysis
catgatcacaacgatggaggt

SEQ ID NO: 89 - DNA, 20 bp, Artificial Sequence
  LRP1B antisense primer for real-time SYBR green analysis
cttgaaagcactgggtcctc

SEQ ID NO: 90 - DNA, 18 bp, Artificial Sequence
  NKX6-1 sense primer for real-time SYBR green analysis
cttctggcccggagtgat

SEQ ID NO: 91 - DNA, 20 bp, Artificial Sequence
  NKX6-1 antisense primer for real-time SYBR green analysis
tcttcccgtctttgtccaac

SEQ ID NO: 92 - DNA, 19 bp, Artificial Sequence
  NOPE sense primer for real-time SYBR green analysis
acagggctgaagtgcacag

SEQ ID NO: 93 - DNA, 19 bp, Artificial Sequence
  NOPE antisense primer for real-time SYBR green analysis
cttggttgagcccaggaga

SEQ ID NO: 94 - DNA, 20 bp, Artificial Sequence
  PCDHGA12 sense primer for real-time SYBR green analysis
tgctgtcaggtgattcggta

SEQ ID NO: 95 - DNA, 19 bp, Artificial Sequence
  PCDHGA12 antisense primer for real-time SYBR green analysis
agaaacgccagtccgtgtt

SEQ ID NO: 96 - DNA, 20 bp, Artificial Sequence
  RP1B9 sense primer for real-time SYBR green analysis
ggccagtcacaagaaggaga

SEQ ID NO: 97 - DNA, 20 bp, Artificial Sequence
  RP1B9 antisense primer for real-time SYBR green analysis
gagatccacagaggccaagt

SEQ ID NO: 98 - DNA, 20 bp, Artificial Sequence
  SLC2A14 sense primer for real-time SYBR green analysis
tccacgctcatgactgtttc

SEQ ID NO: 99 - DNA, 20 bp, Artificial Sequence
  SLC2A14 antisense primer for real-time SYBR green analysis
caggccacaaagaccaagat

SEQ ID NO: 100 - DNA, 343 bp, Artificial Sequence
  FJ46G1 clone sequence
ctgaaggcactaccacagatgtagctctctggaacttccatccctcctctcctaccaccccccaaaaaaagacaaaaccgagttcagaccggctcccccaacaccaagccgcttctatttatcaagtgggtcaacttccactcggaagcacctcgcggggctcggctccagggcacctggtggctggggagctgtattgttttcctgggcacggaggttcggcgccggttttaggattgtgcaaaaagagagtagaaggtacagagatttatttctgctttttgctgttcagccgccgtttgccccagcgaggtgggctggaggctgaatttcaagccttgtt

SEQ ID NO: 101 - DNA, 7707 bp, Homo sapiens
cgggaagagcgagaagctacacgctgggctgcagattgggccctagcgggcttggagcgtggatatgctggctggcccccctccccgggagtcacagctctcgccggtctcgccactcaggctctgccgggtacccaggaggcttgcacggccgcctgcagcccgctgtgcagagcccgggccgaaggcggagctcgatgggaaacggccggccgaaggctcttgcaactctgccacaggcctgccttcccgggcctcccaggcgggtgcctgaggccgcggctccaggccgaggggagaccgcagtgagacgagcatccccttgctgcgccttcttaggatagagggtttaattttcctttctgaagatatcgcaggaagctgttcgtatcttaaaaactccaaaccccgcgctctccctcctccctgcctcccccccacccccgccctccagcctcgcccaccagctcccaccatctcgactctcctctgctcctcttgcctctcccctccctcttgggtctcccgccttcccggagcacgcgctgccagggcctggggcgccgagcggccaatggcacggcggcaggacgtgatgtcaggcgcggctgtagaaaaggcgcggaggcttgcgctggcgcggactgcagagccggggctgggctaggcgcgcgcttggagagcattgcgcgcggctgggcccgcggccggcggctcctcctcccactctgctcctcctcttttttctcctcctccacctcctcctccgcctcctcctcctcctcttcctcctcctcttcaattctcccggtggctcgactcggctcgcaggcttcggagaaacccctactccagtcgccgactcagcgcccaagagggtcgccttgggctgggggcgcaccccagggaggggaggggtccaggcagctgggccgccgcggacacctagcggcttcagggtgaaccccgaccgcagccgtcgccgcctcgggcagagtttgcgcccttgctttgcgccccgggcgctgaagccgggcgggcgatgcccgcggcgtgaaagcgcccgcggcgggcgccgacctctgtcctagtctcctgctccccccgccccgcttgtcccgtgcccttgtgaccctggctttggcgccgtcgcccaggcgccccgcaatgtagctgcccctgcgcctcggcgggaggcgtcctgccccgcgagcgcccggggcccggagcccggcctgggggctcagccgagctcgggcggggccggggccgcggtggcgatgcaccgggcccgttagcgccaggagcgccaggcagctgaggcggggggcaagccctccctcggaggagccgcgcccccggccccgccggtcccgccgcgatgctgttccacagtctgtcgggccccgaggtgcacggggtcatcgacgagatggaccgcagggccaagagcgaggctcccgccatcagctccgccatcgaccgcggcgacaccgagacggtaggcgcgcggctgtggggtcggggctgagagctgggatggggccgggccagtcagcgcctctgctccccgaagtttggggagcgtccttcgtgccgcacgggactgggtgctggggatcctcggtcagaatgcaaggccggtggctcccggttcgggggaaacccggctgctgggacgcagaagggaaacaaggttgaaaccgaaatctcggccctgggggtagaggagagcgtttcttccgaactggaagcgaagtcccatccgcggcccggggcggctcccttctcaccttgcccggtgccggggtcgacagccccgcgctctcctccacctctcggctccggttgctggcggcgccgcgagcggcgccagggaagggcgaaccagctgggagcattggggctccagccggcttgggccgctcccagctttccggcaatcggggatcctcctcaacccccagcgcagtttcagaggccgaagtcttcggggccaacatttgtcgttgatcgcgtccccagacccttgactggtcagacttagccaggccagggctgggagttcaggctccggcctggccctcgccgaaggagactccatttggatctctacacctggctccgcgggcccagccccaaatagccagttcctcgcctcaggcctccctgggggccagacgagcagacactgcccgaccagcgggcccagaagtgacctttaggaggccgcggaggtggggagcacgggagaagcttctctgctccgggagcaggagcagcggcgccagtgtcctcccggcctctgagcgcttcttcggttagaccttctctgctggtcagtttggatagggaagtatttgggttgaacctgtccttcacccacggactttgagggtgtccctgcaccccacttacctcatccccggacccaagagggccccagcccgtgtggcagaggagccagaagttggctgacttgtcctggccttaacctctggtctaaggatccagggatcactggagctggggcccaggaactccgctgtctctccaaagaggattctgtgtggagggtgacttaatggtcaccttatcccccgggtggctcatttaagaagcagtttagggaaagctcttggagggcttgactggagtagctgtcctggtccctaaacacagcccgagcattttgggggaaaggacagggaggactggaaggaagagaggtaagcaccagagccatttaggccaggagcccggcctgggcccgtggctggcgagggctgcgcaggcaggcctgggttctgaaccgcccagaaatggaaatgggccttttggggtggggggaagcgcgccgcatgtcctggcagccccctccgcgttcagggtagccaaggccacagagggagttgtgggtgccggtttcccggcggcggaggggccgctggctgacgcaggcgctgctgtcttccgcctccctcccttcgcagaccatgccgtccatcagcagtgaccgcgccgcgctgtgcgccggctgcgggggcaagatctcggaccgctactacctgctggcggtggacaagcagtggcacatgcgctgcctcaagtgctgcgagtgcaagctcaacctggagtcggagctcacctgtttcagcaaggacggtagcatctactgcaaggaagactactacaggtagcccccccacccaactgcccctcaggacccctccccccaatctcaggcacagtcttacagtttggccctctcctttccgtttagtcccaggagagggttcactactcaggactcccccgctccccccccaagttctccaagccaccacaagttgggtgataaccttttaaagcagcaatttggggagctcttggaaaggtctacgaagtaggagaaccagaaaaaaagcagaagctgccctcctgctcggagcttagaccacaaaaaagcttgagttgggatccttgctcccctctctctttgaagtttcttgagttaatccgaggttatagaaacaggcacccccaaacctaggcagcccaagctggagtgaaacacagctggaaagagagctgtgggagtgggtgcatttccaggtcttttgagaaaatgggaatgaaaggtggccaagatcaaagaaccagaatcactagtagactccaagttctctgtttctccttctccccagttttaggattagggtctatgtatattctctctgtctctgtctctacgtctgtgtctctctctctttccctgtctctgtgtttcttccaaattataaaagtcagtaggattcccaggcgctggtttggagggaggagtaaaggttgaggagggggtaagtggtaagtgtctccctccactcccaggtaaaggctttcctagggcttgcggagactctgggtgaagtagaagtctctgtaggcataagtgtgttaagggaaactattttaggacaggaccaggcctgggtcaaaatctagttctctctcccccccatcctccaaataaaggccgggttgttcgtcttgaggaggggattgccccccgcagcagcagcggcacctggaggaggaaaaggggggtacccaaccgtgtgttcccacagcccctccctccatggtccctacaggcgcttctctgtgcagcgctgcgcccgctgccacctgggcatctcggcctcggagatggtgatgcgcgctcgggacttggtttatcacctcaactgcttcacgtgcaccacgtgtaacaagatgctgaccacgggcgaccacttcggcatgaaggacagcctggtctactgccgcttgcacttcgaggcgctgctgcagggcgagtaccccgcacacttcaaccatgccgacgtggcagcggcggccgctgcagccgcggcggccaagagcgcggggctgggcgcagcaggggccaaccctctgggtcttccctactacaatggcgtgggcactgtgcagaaggggcggccgaggaaacgtaagagcccgggccccggtgcggatctggcggcctacaacgctggtgagtgcgcggcgcacgaagcgcccccatagggttgggggaaagtgtgcggcctcgacggccgggagctggattgaatctctgtgtgctgggcaaatagcgagccttaagcaccggacggcctcgcagaagggacattagccccctgggcttccagactgtgcgtcctcggctggagcgggaggagagggtgcagtggtcccttgctgctccgggtgcagggccttgtctctgataaattgtttttttggagatggctttttggtttgggcctttgccccactttgctaggcaggaagtggcagggatggagaaagcaaggcggcgctgacgccaaacaggttttgggttggcgcggctgagggccgggaactggggcagcgaaggaacgaggcagggcggcgagggtcccaagagaaagggctggctgtggcccggggcgccgagctcggcctggagtgcggcctgacctcgtgaaatgtcccaagggcggcaggcttggggaactcgggcttggggaactcaggaaagcaaaggctgcggttccttttgctcggcccgatcctcctttaaagacaggtctcagttttcccggactttttcctccgagtttcctggcgcctgctggggtgagggccgtgaccctcggaagcgagccccccgggcggggacgagaccggagcaggcctggcctcgcgccggggtggggtggggtggggtgaggtggggggcttggttcggatttccggcatctttgaaccccaggccattcccggagaagctctgccccctcccgcgcccctccctgctcaggacagctgcagaggttctgagttccggcaaatgagccgtcaacatctgcccgaagtctgcaaggcccggaaaggtttatgactctccgggcttccgaactagagtttatgtgcaattattttctttctttcgtttgcaacagaattagatttggagattttgtgttcttcttccttttccctttagtctaatgcacaagcagaaaaaagcaaaaacaaaaacaaacccaagactgtgcagagggtgctacggcgggaagaagtcagttattttcatcttaaagaatctgagttgaatagagagggaaatgaggggcgggtgttcgctccaacgaaatcgcttggaggatcatggggcgtgtgtccctgtgtgcggaactgggaggaaaacgcagcccccagtttggtaaatggtgaagcagcggtaggccggtcggtggcgcggatttaagatttgctgaaggcactaccacagatgtagctctctggaacttccatccctcctctcctaccaccccccaaaaaaagacaaaaccgagttcagaccggctcccccaacaccaagccgcttctatttatcaagtgggtcaacttccactcggaagcacctcgcggggctcggctccagggcacctggtggctggggagctgtattgttttcctgggcacggaggttcggcgccggttttaggattgtgcaaaaagagagtagaaggtacagagatttatttctgctttttgctgttcagccgccgtttgccccagcgaggtgggctggaggctgaatttcaagccttgtttaacctctacaagagacaccctccattcagccatctcactttctctctggcctccctctctctttttttcctttccgttctctccgtcctttctctctatctctgtctctgtgtgtgtcgtgtttgttcccgtgccctcctctccgaccttggccggggctcctagtcctgagagaaacggcgttcggtgcgccggcggtggctatgcggctggctctttcggggctcccgggactaggttggggaaagagggcatctccccggcctctcggggcccagcccagtcttcctagatctggcgtccgcccttccctcccctcccgcactggcaggagagaaatggccgcagtgtgggccgcggggcagctaggactggaaagcggggaccctggagggtgcgatcgcggacggggtgtgcgggcgcgggtcgtgtgcgtgtgcgtgcagggttccgaccacggggacacgagcttgtttgtggcagtgtcccacatcctgtggcccagccacgacgaccccttgcaaagcctcttgctctggggacagtccctccgaggcgcggcggcaccttactgaagggcggcgagctgggggccgagtggggagggggcgccgtcggggcgccgggcgctgggcttacagcagagccgcgggccgcggggtcggaaagtccttccggggcggggccgcagcggcctcttcccgcagcccctcgggcccgggccccggtggaacggaaacctccccctaccccgggaggggctgccagcgggctgggggtgcgaaaacggcggcaggagcgggcgaggggcccgggccgcgcactttgcgcctgggtttgcgcgccgcggccgcgggagtcccgcgcggaccggccggacgcccggcctcccccagccccagctttttgtgtgtgtgtgcctggcggcgtaattactgatttgattccaatccattatttagacaattgaacctacaatctcgtctttagtaaaatgaggcgaagtcagatttgattacaggttcagtcccagcgacaagagctcgaaacccgatgggttaataacagatcacgagtaaattattcatgattttacgagctctttagctccattgaatcggcctaattgagaggaaaaaaaaaaaaaaggagagagaaagcccgggtcctccccctcccctcggcccctcgctcctccccggatccgatcctggggaatctcgacccgcccccgggcactgggggcgggagtgagggggttcggggcgccggccaaacctgggccccacggctgcctcccccgccgccgccccctgcccttgcctctggccggcctcggcctcgctacttagggccggctcctttccctttttctccactccccttctttccccttttctctactcccccgccaataacggcttcggaaaaggcctcccccgcagggaccgggtctcccggagccccgggattcagctcggccaccggaccctcgccacaagctgcgcctgtttccgggactcgctttcccctccg

SEQ ID NO: 102 - DNA, 201 bp, Artificial Sequence
  FJ46G1 amplicon sequence
tccagcccacctcgctggggcaaacggcggctgaacagcaaaaagcagaaataaatctctgtaccttctactctctttttgcacaatcctaaaaccggcgccgaacctccgtgcccaggaaaacaatacagctccccagccaccaggtgccctggagccgagccccgcgaggtgcttccgagtggaagttgaaccacttga

SEQ ID NO: 103 - DNA, 961 bp, Artificial Sequence
  FJ45F11 clone sequence
aagaattccttccgacccccgacccggcgcccctcccccaccacagcgggcggaggaaaaaaacaggcccagaggccaccccaattgtggagaccctgcctttcccaggtcccgcctgactcgcagcccctcactcacccatcaaaatgcgcatctgcttcttgccaaatagtcgggagaagagggaggagatagtgaggcccatggcggtagtggcacttgtgatgggcagaagcagaaggggtttggggcgaccccgtgctttctcctttcaagctcccaggcaaactaaacgagagggaagagaaagagcggaggaagaaagagggaggcagaaacgtctcagtggcccctgtgccgatgaagatccggcacaggaataagccggtagaggacctgctaggcgactggcgcggcagctccggctctagcctttaggctctggctgtgccacgtcacgcggcggccgcggcgactccagcagcggcccggcccctccaacgcggacagaatcgcgtcaccgccgccccatccccctgcgcttccggtgcgcccgagccactgcgcttgcgcctgcaggggattggccagtttcgctgacgagcatctctcgacggcgccaccgcccggacgtggccccaggagccgggagaggccggctgaggcacatgcgtactgggagacggacaaagtcccacaatacctcgcttcgtcactgccaaatcagactcagggtgagacgcttccgcccggatgaaggtattgtcggggtgatagtccttgcttccggaaaggcgagctgagcattatgggttaggtgaggaacctcgccctcttctattacggtacgcgcgatgggtattccctgatgccatgaacttacacgtttcacacacgggaccagacgcttgctttagttgacgcatgaagaccggtccggtcttttgcggagaaaagtggt

SEQ ID NO: 104 - DNA, 591 bp, Homo sapiens
cgatgaagatccggcacaggaataagccggtagaggacctgctaggcgactggcgcggcagctccggctctagcctttaggctctggctgtgccacgtcacgcggcggccgcggcgactccagcagcggcccggcccctccaacgcggacagaatcgcgtcaccgccgccccatccccctgcgcttccggtgcgcccgagccactgcgcttgcgcctgcaggggattggccagtttcgctgacgagcatctctcgacggcgccaccgcccggacgtggccccaggagccgggagaggccggctgaggcacatgcgtactgggagacggacaaagtcccacaatacctcgcttcgtcactgccaaatcagactcagggtgagacgcttccgcccggatgaaggtattgtcggggtgatagtccttgcttccggaaaggcgagctgagcattatgggttaggtgaggaacctcgccctcttctattacggtacgcgcgatgggtattccctgatgccatgaacttacacgtttcacacacgggaccagacgcttgctttagttgacgcatgaagaccggtccggtcttttgcg

SEQ ID NO: 105 - DNA, 212 bp, Artificial Sequence
  FJ45F11 amplicon sequence
tccggaagcaaggtctatcaccccgacaataccttcatccgggcggaagcgtctcaccctgagtctgatttggcagtgacgaagcgaggtattgtgggactttgtccgtctcccagtacgcatgtgcctcagccggcctctcccggctcctggggccacgtccgggcggtggcgccgtcgagagatgcacgtcagcgaaactggccaatccc

SEQ ID NO: 106 - DNA, 485 bp, Artificial Sequence
  FJ25G8 CGI sequence
aagtttcacactcacttatctgcaagcatcgcccagcaggaaagccaaggaagtcaggggaggggagcggggaggtgttctccttacctcggtcggctcccacggtcagcaccctggcaatcggcaataatcccgagagagtgagtaaggcgaggagaaactcggacattgtggtcgcccggtaaggaagcctgcgctggagactgctcggcggcaccttcggcccggcggcggcggcggcggcaggggccgcttggagcctggaatcgagcggcgtcatttacaaatgtcactggaaattcttcagctcaatgagtccagccagtcagccttctcctgcctggagcaggatgtggaaggtggagggatgcgcgcgtgcgggagagaggaggcagagcgtgtgtgagcgcgagcgagacgcccgtgtgtcttgacttttcaatgcagggtacgtctgatcttacatcacgcaagaggggcagtga

SEQ ID NO: 107 - DNA, 829 bp, Homo sapiens
cgaaaggggcctgaatcggcgctttcgccgctccacggacctcgaatgcagcaggtaaggaagttaacctgggagctaagttgagggcggacacgcttaacgcggagaacctggagctcgcaaaagctcgcagttgagggccatcgcggagggaagtctttcggaaatgcgaattctcattccaacatctgcagcgcttagaattcgcggacagaagcgcacagccaagtgcacacactcccggaggggacaaaatggggaccgggaaacggccaaagggaaaagctgtgggggtgccgaggactcaggcacttggttcagaaatcccgcaccgcacctctcacccccgcacagggccagcgcacggtggtcacccggtcccggggagcggagctgcaaggacttaagtttcacactcacttatctgcaagcatcgcccagcaggaaagccaaggaagtcaggggaggggagcggggaggtgttctccttacctcggtcggctcccacggtcagcaccctggcaatcggcaataatcccgagagagtgagtaaggcgaggagaaactcggacattgtggtcgcccggtaaggaagcctgcgctggagactgctcggcggcaccttcggcccggcggcggcggcggcggcaggggccgcttggagcctggaatcgagcggcgtcatttacaaatgtcactggaaattcttcagctcaatgagtccagccagtcagccttctcctgcctggagcaggatgtggaaggtggagggatgcgcgcgtgcgggagagaggaggcagagcgtgtgtgagcgcgagcgagacgcccg

SEQ ID NO: 108 - DNA, 107 bp, Artificial Sequence
  FJ25G8 clone sequence
agcctgcgctggagactgctcggcggcaccttcggcccggcggaggcggcggcggcagcagccgcttggagcctgaaatcgaacggcggcatttacaaatgtcactg

SEQ ID NO: 109 - DNA, 712 bp, Artificial Sequence
  FJ46A4 clone sequence
cctccctcccagttgctttctgttctgctgttcttgtgggtctctgttctcctggctccaggcctaacggccgccccctccaaccttggccttgggaagtctcttggggcggagaagagaggggctgcctcctggcccccaccctgcgacccttcgtcccccgctaagccgggatggcaggcggactcgccggacccctttttagacccaaggctctcttcgcctctccatccggctccggccggggtggggtgacctgagggacccggtagccgacgacagagaaactagaccggccaggacgtagaagggtcgcttcccccaggctggtccggggctgagcggggcaccacagggagcggagacagcggaggcggtgagagcctcgggagccactggggcgagcgcgccagcgtccaccagagggcgctgtcggctcgcggagtggggcgcgggcggcctggagccccccggtgcggcccggcatcagagcgcgcagcgactttgtcactgcaggcttgccgggtgggttgcagaagagagaagagacccacccatcctcaaggcactcatttgggcaagaagggaagagacccacaagaagactatttcacactcttctcatttcaaatgtgtatagagcacaaagttgcatttaccatgtccaagataccgagcagcaccaacatgagtcatcctgggtctccaaatt

SEQ ID NO: 110 - DNA, 2507 bp, Homo sapiens
cggcagcggagccgacctcttcaccgccgccgtcttcgcgggtgtcccagctgggccgcgccccggggcccagcgctccggccctgccctcccaggttcccagtctcgggcgcggggcagtccctcgccggccgccggcccggtcccggctcccagccgaggaaatttttccccagcagcccagggctctcctcacgtgaccagccgcggcggtagcttccagcgagagtttaaaggttacggaggaaattagcaaggacacagccactcgccccccggagggcgcgccccattttcccgcagcccggacggcggctgtcagcgcgctgatcagtgccgaagtcccccaaaggggcaccgataaggaccagcgcaaacaggaacagtcactttgcaaaaaataagcagaccacaaaccaagccggggctctaccgagctttcacttgtcaacaagttctcgcagcctgcgaagtccaggacagatcccaaaggggagaatgtccccaaagaaaggcaagagagaagaacagagtttaaaacttcacccgagccatgcttgtccctcccccttccccctcccccaccgccgtggatcacagcgtcccgggacagagtgacagatgggccagaactccgcagactgctcctgcccgccgcggcccgagtgggggtgggcaagggaagggtcccgaggcctctagggcaacagggccccctccactcccccatccccggctctaaaagacagtgttggccgcagggtccccgagagatacgcggcccggcggggcccatccatggccatccacggcccccggcacgcgcatccagcccagcgccgccgcaccccggggtcccgcgcggggtccccgccccccggctgacccgcccgccgctgcgaccccgggcagcctcgccagctctccaccgttgtctcctcggctcactttttccgtattgctcccccaaccggcaaaactttctatttccccaaacactgccgtgcgcgcgcgcgcgtgcacacacacacactcacacacacacactcacacacactgacgtcttttgcgcatttcctgcattagagggagggagactcgctcgcacaccgacagagggagaggagaccgcgggggagcgcgggctggcgggcggcgagcgagcggcagccgagagcggggacgcggggagcgcggagggcgggcgccggcgcccaggaggtggggccgcgctgagtcgcagtccctgccccgccgccgcgctgcgcaccgcccggtgctggcctgcgcccccgagccggatcggcggccgcccggactccgagcccgcgcggatgtgagattccgggctcctggcgcctcccgatcgccggctccggcgcggcgctgcttcgcccaggaggcggcggccccggctggccgtggggcttgtggatttttaaaaatttggctccgaggaggaccatttcctctcgacatgcatccctccggatagactgaacatccctccgttcacccccccgccccgcgcggtcagaggcaggcgctgagtgtgcgaagaggatccgggttgaaatctgcgcccccggttctcgtccccgccccgtcccaccgccccatccccctcccggagtcgaaatttcccgggattatgtttcggaaaggtaggtaagcgccgggcgcggcgcccgctttcccgcagtccggagccggagagccagcgaggcggcgaggcagcccccgcggcttgcagcggagccgacagctcgtcttctcttctggaggtgcagctggtggtcggggggagagacttgctccaaacacggacatcccccagctctcccccctccctgttttccgttaggaacccggcgaggaaatacatgcactggctgagaatcgcccgcgccagggcgcaacgccacaaggtgtagggagtgtgcggggtggggcgaaaggggacccaagagtccctgtggctcggagtgccgggccgtcggttcttcattcctgccctcggggcagacggagtgaccccggcccccactccccgccccgaccatggtagtgttcaatggccttcttaagatcaaaatctgcgaggccgtgagcttgaagcccacagcctggtcgctgcgccatgcggtgggaccccggccgcagactttccttctcgacccctacattgccctcaatgtggacgactcgcgcatcggccaaacggccaccaagcagaagaccaacagcccggcctggcacgacgagttcgtcaccgatgtgtgcaacggacgcaagatcgagctggctgtctttcacgatgcccccataggctacgacgacttcgtggccaactgcaccatccagtttgaggagctgctgcagaacgggagccgccacttcgaggactgggtgagtgcggcgcctccccgtcattccgggaacccggttgtggggtcccggggaaagactcgctggtcttgatcgtagggctccgggacttattgacg

SEQ ID NO: 111 - DNA, 143 bp, Artificial Sequence
  FJ46A4 amplicon sequence
ccggcaagcctgcagtgacaaagtcgctgcgcgctgatgccgggccgcaccgcggggctccaggccgcccgcgccccactccgcgagccgacagcgccctctggtggacgctggcgcgctcgccccagtggctcccgaggctc

SEQ ID NO: 112 - DNA, 613 bp, Artificial Sequence
  FJ32F2 clone sequence
aacctaatttcaaaggcggggacttgattccttttgttcatcaagagtttgcccgcctgcaagaccccagcgcctttgtagtccccaccctccttcactgctctcaacttctccccaggctccttggattcgcacctcccaggcgactgtttgttagtttggggtgtgtgtgtgtgtgtgtgtgcccatgtgtgcacgcgcgcgcgacaggggcggtacctatccctccaggtatgcaaggtccactcaccttgacctgactctctgtcatccccaacgaataggccaaacgagccctctcgggccccgccaagtattttgtttgttcgaaagtcttctccagggcgaagatctgctgtccggaaaaagtgggtctcgtgtgttttctcttcccgtctttgtccaacaaaatggatccttgatctgtgagaaccaataaacaacgagagagggggaaaaacaatcggttacaagcggctgcactagggggaaaaaacgaactcgaaaaacaatgttttgtggggaaagaaagctcagtctgtggcggacaccagaagtgtagtggcgcttccagactaacggcacgtctccctttgcttttttacatccacct

SEQ ID NO: 113 - DNA, 3140 bp, Homo sapiens
cgcaacttcccgcagttactgccgctcagccgcactaaggaggtccggagacttggaagaaacttcggagactcgctgaaaggcgaagaactgagcgagaaaaggaccccaggctgggccctgaagtccacctggaaggctgggagcctccgcggcgctgcggcctactaccccgaagactagcgtgatcttggggcaactcagtttgcctcaagcaaggtctttgcgggatggtacattttccccaggccgtgtcccttcatctcactcttctcctggctcgctcttgtgttagggtgttgagctccacgatgtccccaaatgtgcccggacgcagcgtgtgccagcccccagagtggaggcaggtgggaaagcgcgggcccagcgctggtgagcggctggaagcgctcgccctgccctggggacgctggactgtcttcaatgggctcacccaggccgagtgtactcgggcggcgagggcgaagaggtgaggtggggacaccccaggcggagacgtgcatgcaaatacactcacttcccgggcggtggagggctcaccgctgccgggcctcactgcttcgaagagaggcagcgggttaggctggcgaaggcggaatggggcgctggacgacagggaggagccgggaaagcagagatgcagtggcctctcctcccctctcctccccatctttctaatccaggccaggcctgcggtcctggccaggccgtttcaggaacagccagggcctcggacgagacacacgtccctcaccgcgttgtggtccacacgaacacacacacaccggctcagcccaggcgtactcaagacttagagagagggaggcgcttggatacagccacgcgagctcagaaaagcgagaatccctttctggaagccctggcccaacctaactggtgtgattccggcgctgtcaaactactggggcgggccacaggatggactgagcggcatgcacaccaggggccgcgacccgggagtgggcagaacaggcacggcaggcaggcatcggggcgcgggtggtagtactcacgaggggtacaggccaggcgtgcgtccctccagggcgggctctgcatcactccgggccagaagatgggcgtccggccaggcagctcagccagcggcttggggtaccggcccacggcggccacggccgcggcgctggggctgaagtagagcccgggcggcggcggcggcgggctcaggctgctaaagcgtggcagtccggccagcagccccgccggggatgaggcggcggctgcggccgcggcagcagccgcggcggcggcagaggcggaggaggcagaggcggacgaggaagaggaggaggaggaaccggagggcgaggcggagggcagggcggcccccgaggccacgggcatggagggccggctcaggatatcgttgatgccgtgtggggtggcggccgagagctgctgcggggggctgccgagggatgagagcccccccgtggccgggggcttcaggccgcctgggttgtgggtgcccagaggcggggagggcgacgaggaggacgacgacgaggacgaggaggagggggggccggcaggcagcgggggatacgcggcagggtacagcggggtcttcatctcggccatgctgtgcagggcggccaggggagggctgctgagcaggaatgcgctctgccgggtgccctccattgcccccaccgctaacatcccacggccacgccggagaccgtagccttgcagcgagggcgctggctggtgccccccgcggggctcagaggagccggaagcgccgagggcgcgagcggagaggcactcggcgcgcccggaggcgagctgccaactgaaccaaaaatgccgctgccgggagttgctcgcctagctgcgcagcagagatgtccaaaccctccacgcgggaggcggcagctcgccgagaaaagcaggcgtcccggcgggctaggcagtcctttcgttccgcgagtcctagattcgatccctggctattctctcttcctcacttttcctagctgttcaaagtgcgctacttctcatcttctgcccccgggaaataagcaaaacaaaacccaggctggctccggagagtttgtagcaaagttagttgccgaatctccactttgaagttggagggtgggggtggcttgctttctttggggaggttcaaaggacgccttgtgcagcccgtggcgctcctctgatcttactgggatgctctgctctttcggtcgcgcggctgattcgcattcgacgctcactgtgcccggggagaaagcgcatccagcccgcgggagatctagcctctgtgcgggcttcctccgcccacgctgccccgggctgctgcgccagaaagggcagtgccacggatccccgcggctcccaggtcctcacctccacccgccttgccctctggcccacggggcactggagttgcaatattgaaaagaaaaagggggaggaaaaacacagaaaaacaaaagactaagtgtgaaaagtctgacggctgggtttcggcgccgctcgtcagtccacttctgcaaacgggcccggcgacccccgccccacccccgctccctctctccctctcactctcagcctttgactctcctctctggcattttcttcgcggcttcccaggcttcggtcttctaagtcctgtcctctggccaaactgaccctctccgcggctcctccgctctttctctcctttcctctctccctccgggcctgacagttcagatgaagctctcaaaggtaataaaacatttgcatcactccctacccctctttagctgggggacgaaagggagggggaagtgggggaccaaaatgagaactttgagggacgtcactccgaggctgcgggggccggcgagccgggcgcaggccgggggcgtaaccaccgtgtccaatagctcgccgcctcgatccttgcgtcgcccctggggctcgcccactcgggggcgccgcaccctctgattggctgaggcgggccaggcgtagggccgcgcccgcctagtcccctcccgctccctacttctttctcctgtgggcggggccaggaggggcgggagctggtgagtggtgcggttccgcgctggggccagtggccg

SEQ ID NO: 114 - DNA, 186 bp, Artificial Sequence
  FJ32F2 amplicon sequence
tctcaaagccacggcctgccgggcctgcattcccgccggcgcccccgccctgacgctccgcagcaagcggggctatttccggctcccccaacgcggtctggttttccagcctgggagtggctgagggggagccgccccagagctagagccgctccctgccctgcccggagccccagtggccctgag

SEQ ID NO: 115 - DNA, 738 bp, Artificial Sequence
  FJ27D1 clone sequence
acaggctttctccggtgtccccagcgggccatcccggctgccgcaggcagggagcccgctcccggctcgtcagcacccgccctgcgcggagaaggccgtactccatttgctttcattttccttctccccctgaaatgggctcatttcacctctccactgccctcttcccctccccgccccctccctggttcccctctactttccccccttctctctcctgatccctctttgttgggaaaaacacacccacacacaactcctcctagctaaggcctgcgctggaagcagaaactgagttctcttggcctgccgcgaggaaacccgcgtcctgcccccacccaaggtgggtatctgggatccctacacaccaggagtcaggtcccgaggcccagcaggagtgaaatccccagggcctgtgaacgtggctctctcaaagccacggcctgccgggcctgcattcccgccggcgcccccgccctgacgctccgcagcaagcggggctatttccggctcccccaacgcggtccggttttccagcctgggagtggctggagggggagccgccccagagctagagccgctccctgccctgcccggagccccagtggccctgagcgccttgttacctgatcttttgtgtgatgtatgtgtcgcaaggaaatttgcagtgactttggagcccagactgaggaagcggaggtggcgaaaggagagacaatacgagccccgaagtacaat

SEQ ID NO: 116 - DNA, 407 bp, Homo sapiens
cgggctgagtttccggctccaggttcgcgtgtcgccctgaggtttgaggccagacagctcgcagtcgggcagggagggcgggggagagacgagcggctctggccccttaattgtacttcgggctcgtattgtctctcctttcgccacctccgcttcctcagtctgggctccaaagtcactgcaaatttccttgcgacacatacatcacacaaaagatcaggtaacaaggcgctcagggccactggggctccgggcagggcagggagcggctctagctctggggcggctccccctccagccactcccaggctggaaaaccagaccgcgttgggggagccggaaatagccccgcttgctgcggagcgtcagggcgggggcgccggcgggaatgcaggcccggcaggccg

SEQ ID NO: 117 - DNA, 186 bp, Artificial Sequence; identical to SEQ ID NO: 114
  FJ27D1 amplicon sequence

SEQ ID NO: 118 - DNA, 471 bp, Artificial Sequence
  FJ63F2 clone sequence
tccggccgccatggccgcgggatatcactagtgcggccgcctgcaggtcgaccattactctttcagctgggttagagctgagaaagcatttgtcgccgccagccccatccaccacgcaaatccatctgagacagaaaggaaagaaaaaaaagcaccaccatgcctaagaatagagagcgagcaaacccccccaccgctaatcacacacacacacacgcacacacacacacacgaggaagcggtggagcagagaagggcgcggctagccgatcccggttctctcgcccggctcctgctgccacgggaattcctaaagccattggggtcgaatacacttacgatgaatcaatcgtggaaggtcggactgattgcttttcaaatacatcgcacggctccgctgaccggcaccctccaaactcacaagggcacgcacgctacttgcacgaatcccagaggagggaggagggagga

SEQ ID NO: 119 - DNA, 4637 bp, Homo sapiens
cgctcggcagaggcacgatacagcgggagagaagggcaggcccgttcacattttaatcggagcgcaccggcggccgctcctcggctgcgtcctgggctgccgctcgggctcgggactgccagatgcaggctctggctgggggcggcgggcgcaagcgggcgcacccgcagctaggggtgcggggtgcacgcacacgcacgctcattaagagccatgtatttattgaatgtccgagttgggttagttcattggaaatccccgaggagggctcaatttgcccttgttttcgttgccactttctcttttttcttggttcgctgaggttcctctgtgcagcgtttccgcttggccgcgtccccccaccccaccccaccccacccccgcttctctcgcctaccgggtgcactccccctcccatcccccttaactctttcagctgggttagagctgagaaagcatttgtcgccgccagccccatccaccacgcaaatccatctgagacagaaaggaaagaaaaaaaagcaccaccatgcctaagaatagagagcgagcaaacccccccaccgctaatcacacacacacacacacacacacacacacacacacacgaggaagcggtggagcagagaagggcgcggctagccgatcccggttctttcgcccggctcctgctgccacagggaattcctaaagccattggggtcgaatacacttacgatgaatctatgggggaaggtcggactgattgcttttcaaatacatcgcacggctccgctgaccggcaccctccaaactcacaagggcacgcacgctacttgcacgaatcccagaggagggaggagggaggaagggagggagagcgaaggagggagagagggggtggaggagccagggagcggcggcagcgagcggtccgtctcgcacgcgcgggcaccgcgctggtcctgggctgcaggtttcccagatgatggcatccgagaacttaaacaaagggggctgccgccggcgcgcaacggctgcggaaagttgcggtggcggatttccaaggagcgtggccacgaccagagctcttggcgatgcgagcccccgcttcccacccccgccgatcagagaagggggccggctggtgaagggaagaggaaactttgaaaccactggggacacatctgtctataggtattagcttgaatggtacatccgtgccgcgcgctttacaaatctctggcggggcgggggtaagggggtgagcaacccgacgcgtactaggtggggtgggcaggttgctatttttcatgtttcccccttcgttttccttctgattctcttcccccctctcgtccctccctcccttttatctcccctttcttccccccgcccagacctattgcgaacgcccagagccgccgagggaacgccaacgtctgggccggacactaagagttaagatgtggcggagggggcggcgggggaggggcggggaggggaaagtggtgagggggagggaggctggaggacgacagatttccagcttctacgacgctctgcctaaattaaaaagcaaccaatcggaacggccggaaggggggcctcgcgtcctgagccagtcattccgagcctgccaatcacccagcgggtagccaatcagcgggggccctggtgctcgacttccttgtatttgggaaagtgtggtggtgggtgcgcgctcgcggcggagggtaaacattcgacagtccccgctctgagagggagggacagagagcgaactgtcagatcggagcgagagcgggcgcccgagagagggagagagagagagggagggagaggaaaagtgagagagggaaagagagcgcgaacgagggcgcagagcgagctcctgctgcaactctgctccagcacggccagcgccagcgcccgccgtcggtgcactctacgagccgtgcagcgtgcccactggagttgttgtgtatcaaggatcgatcccctatatgcacacacacacctccacctccaccaatgcactcttcttcctcctccttctccagacaactgctgggaaaaaaataaaacaccaaccccaaccgtcagcaacaaggtaacagagcgattcgacatcattttttttcctgttcaattttttccttgttatatttgtttcctaatttctgcccaaaaggaaagatgtcgcatcagactgtgactgttgcgaggagaatgaaaaaggactcttgtttcagaggcaaccaagagctccggcaatagcaacttcagagaaatgcaccatcgcaagaagttttcctaggacagaacaaaacttgaaacgagaggaccagagggggagagcaggagccagcctcccctctccgcactcgcgagcagccagcagcaccacgccttcaaggacgaaaaagttttactactctaagggaaaacgagtgaaatgtgttcctgaggaggagaggagaggagaggagagagcggacaagagaaggagcgggccggttgctggtcatccgtaatttggctaaggaagaaaggagcagcttctttctttgttatctcccgtgaaaccttcacttagcaggtggacggagccccgcgaccgggcagagtccgggctcgcccgaggacaggaggaggagcgggagcccgcgcgtcccgggagagcgccccgagtgcaggtccccgccccgcccggcgagccccgctggagcgagcccagcgcgccggggctggggggcggccacgaccccccctgaagggggtggccacggagcgcaccccgagaagcgagcccccctccccagagcgctgctcctgcggctgctgctgctgctggtgaccaaggccggccggcgacccccgcgccctgccgagcggccttgcagctgcagccgggggccgcggcggcggcggcggcggcgggggcggaggcggcggcggaggaggaggcggcgaaggcggcggggccggcgggggcccggggcgggggcggggaaggagggggggaggaggcgggaggcggggggcgcggcggcggcggcggcggcggcggccgcggctgctgctgcggcggcggcggcggtggtggcggcggtggggtggcgggagcggagcggcatggccacggcggcttctaacccctacctgccggggaacagcctgctcgcggccggctctattgtgcactcggacgcggcaggggctggcggcggcgggggtggcggcggcggcggcggcgggggcggcgcagggggcgggggcggcggcatgcagccgggcagcgccgccgtgacctcgggcgcctaccggggggacccgtcctctgtcaagatggtccagagcgacttcatgcagggggccatggccgccagcaacggcggccatatgctgagccacgcgcaccagtgggtcacagccctgccccacgccgccgccgccgccgccgctgccgccgccgccgccgtggaggcgagctcgccgtggtcgggcagcgccgtgggcatggctggcagcccccagcagccaccgcagccgccgccgccaccgccgcagggccccgacgtgaagggcggcgccgggcgcgacgacctgcacgcgggcacagcgctgcaccaccgcgggccgccgcacctcggacccccgccgccgcccccacaccagggccaccctgggggctggggggcggccgccgctgccgcagccgcagccgccgccgccgccgccgccgcgcacctcccgtccatggccgggggccagcagccgccgccgcagagtctgctctactcgcagcccggaggcttcacggtgaacggcatgctgagcgcgccaccggggcccggcggcggcggcggcggcgcgggcggtggagcccagagcttggtgcacccggggctggtgcgcggggacacgccagagctggccgagcaccaccaccaccaccaccaccacgcgcatcctcacccgccgcacccgcaccacgcgcagggacccccgcaccacggcggcggcggcggcggcgcggggcctggactcaacagccacgacccgcactcggacgaggacacgccgacgtcggacgacctggagcagttcgccaagcagttcaagcagcggcgcatcaagctgggcttcacgcaggccgacgtggggttggcgctgggcacactctacggcaacgtgttctcgcagaccaccatctgccgcttcgaggccctgcagctgagcttcaagaacatgtgcaagctcaagccgctgctgaacaagtggctggaggaggcggactcaagcaccggcagccccacaagcatcgacaagatcgcggcgcagggccgcaagcgcaagaagcggacctctatcgaggtgagcgtcaagggcgcgctggagagccacttcctcaagtgccccaagccctccgcgcaggagatcaccaacctggccgacagcctgcagctcgagaaggaggtggtgcgggtctggttctgcaatcggcgccaaaaggagaagcgcatgacgccgcccgggatccaacagcagacgcccgacgacgtctactcgcaggtgggcaccgtgagcgccgacacgccgccgcctcaccacgggctgcagacgagcgttcagtgaagccagggcgcagagcg

SEQ ID NO: 120 - DNA, 189 bp, Artificial Sequence
  FJ63F2 amplicon sequence
cgcacacacacacacacgaggaagcggtggagcagagaagggcgcggctagccgatcccggttctctcgcccggctcctgctgccacgggaattcctaaagccattggggtcgaatacacttacgatgaatcaatcgtggaaggtcggactgattgcttttcaaatacatcgcacggctccgctgaccg

SEQ ID NO: 121 - DNA, 274 bp, Artificial Sequence
  FJ46C3 clone sequence
gaagttggccgcctccaactactccatgcttgtgccctcctcctcctcctcctcctaaggacacccccagggaaaagcctgcggcatttcttcaacgctgccctatgccggaaagttaggtctcggctgcagcgctggtctctggagaagcctctgcctgcagccgccggcgagttcccgcctcccctccccagccgcctcgctctttgcttttccacgtgagaaaaagaaatgttcacggccgatgcttcattttcactgattgatttctttt

SEQ ID NO: 122 - DNA, 1717 bp, Homo sapiens
cgcgcgcgcgccgtgaggatgcccacagtatattatgtattcagacacggactccagtaggccccagcgcgcgagcgcgcacacactggcactcacactggcgcgcacacacatgcaacttcaggttccgtgtgctgtgcggagcgggaggggtcgtgaagactcaggggccatctcctaccccagatgttcgcacatctgtccccgagcgctgcttggagtgtgtgtacgtgtgtgcgcgacacccgcttggacccactcccgctctgccacctgcctgccgcgctctctgcagaaccccctcccatcccaatccccagcagtgctgcgtcacttccagagaagcccagaggcgcggagttcggggcaacctcggacccggggtgggatctcagggtgggattcgaagctgggacctgccagggacctccagctccccaaaatgcgatctcaagccgtgagctgctcaggctttcaggcggagcaaagcactggcaccctgggagccggcagcgggtggccagggcgaggccaagccacatacctctgactcacccggtgtgcgccggccgggaagggggcctggttcagccgccgccgccggcgacgagaaccaattctgggaaggagcaagatggggcagaaggtgcgcgcgggggagcaactgagcaaataacacggcggtcgcggcgcgcggcaccttgggctggcgcgtggctgggaggccgcatgggcgacgcgcaccaggctgcccctctcctcactgcgccgcctcgccagaccgcagtgcagccgctagcctgcctccctccctgccccgggcgcccccgccagtcccctgcgctccgctctgcgcggcaggctcgccgcgacccctagagagacggctttgagtggcgggaggccgaggccggctggcttgggaatccaagcgctggcccaactaggtgagaggaaactggggtgggatttcggagaggagggcggagagaggcgggaaggaaccagcttgagccccaggaatgcgcgcaacttgcacagcctctagaagcgccgcgcgcccagcgcccgccggtgcgcaggaacaggtgaagagcacgcggcgtgcggccatccacgtggtggggacgtgatagcccctcagctctcctcctagccctccctccccttccccgccccccagcgccttctctaacgccgcatcgatttgtttgggctacgcagcagaaacagagcccagcgatccataaacattctattaagcaaaaatattcccagcgaaagcaaaaccgaggctgaagcctccgcgaagttggccgcctccaactactccatgcttgtgccctcctcctcctcctcctcctaaggacacccccagggaaaagcctgcggcatttcttcaacgctgccctatgccggaaagttaggtctcggctgcagcgctggtctctggagaagcctctgcctgcagccgccggcgagttcccgcctcccctccccagccgcctcgctctttgcttttccacgtgagaaaaagaaatgttcacggccgatgcttcattttcactgattgatttctttttaatatcaacacaagttaatggaggcaaggcgcgctctgattgaagggctgcccccgcccttcgactcgggctggctgtgccgggggtctttcccaccgggtcgcaggcgtccagcggctgggtggcgggcgccg

SEQ ID NO: 123 - DNA, 170 bp, Artificial Sequence
  FJ46C3 amplicon sequence
ctccagagaccagcgctgcagccgagacctaactttccggcatagggcagcgttgaagaaatgccgcaggctttttggagtagttggaggcggccaactttgcggaggcttcagcctcggttttccctgggggtgtccttaggaggaggaggaggaggagggcacaagca

SEQ ID NO: 124 - DNA, 1357 bp, Artificial Sequence
  FJ47G6 clone sequence
gctccctctacaccaagctgctccctgtacggcaacagaactcctggaacttgtccgcagagaatgagcagggcccttgcttttttatggtcttggccaggtttagcctgccttgtaacccgcaggtaacagagctatggaaatcaaatgaatcgctgtcaagtcggtgcaagattactattcagcacatttcatgaagacctggtctcaactgaacagactgaaaaccaatttcacaatttctttgcagtgtctcttagaaactagccactgtcactgcttagaaaatcgggggactgctccctcaagtggtcaaaacgggcctctcactagagctcgctacttgtttagccggtataaggccagtcacacaatcctgcaaatttcccgcaacctcccgaaggggtcaaagcccacgccccagcaaaagcacaggcgctccccgcccagcgaagacatgcgcatgcgcctatccgtcttctcccaaagcaaccaccacctggtggcgccacttccccccacgcctgtttcacccattcagccctctctttcgaatacctttgtccaattccataagggcagagttggcatccagttcctgttcgccatagccggcatctgccaggaaagacttagttgagtttgacgccatgacccgaatagttactcgactagcctagtcagaaagcttgcaaactctaccccaggaccgccatcttcccccgccgccttcttgctggtttttcttccgcgcgctgtcaagccctgttacgcatgcgccctggtcaccccgcggtttgtccgcgcctctgctaccccctgcgcaggcgctcaaggagctcttggactccaggttcccgcggctgggagaaaaggaggcggggatccgaagggggaaatgactctgaggcgcccggacgtcgctcggaagccaatcagagagcgtgacgtcagtttggcgcggagtttggcggccggggcttacagtggcgggagttggaggcgataacgatttgtgttgtgagaggcgcaagctgcgatttctgctgaacttggaggcatttctacgacttttctctcagctgaggcttttcctccgaccctgatgctcttcaattcggtgctccgccagccccagcttggcgtcctgagaaatggtgagtaacggtcccaaccgctgctcggagctggcggaattcatttcccccgaaacacacgccacctccgaccagggccgactccaattctgaactcagcttctgagttctcccatggcaagggtaaattagtgttagcagggactactaaagaaagctgtactttcatcccctcgggacacttgtaatcgtaatcgggc

SEQ ID NO: 125 - DNA, 527 bp, Homo sapiens
cgccatcttcccccgccgccttcttgctggtttttcttccgcgcgctgtcaagccctgttacgcatgcgccctggtcaccccgcggtttgtccgcgcctctgctaccccctgcgcaggcgctcaaggagctcttggactccaggttcccgcggctgggagaaaaggaggcggggatccgaagggggaaatgactctgaggcgcccggacgtcgctcggaagccaatcagagagcgtgacgtcagtttggcgcggagtttggcggccggggcttacagtggcgggagttggaggcgataacgatttgtgttgtgagaggcgcaagctgcgatttctgctgaacttggaggcatttctacgacttttctctcagctgaggcttttcctccgaccctgatgctcttcaattcggtgctccgccagccccagcttggcgtcctgagaaatggtgagtaacggtcccaaccgctgctcggagctggcggaattcatttcccccgaaacacacgccacctccgaccagggccg

SEQ ID NO: 126 - DNA, 124 bp, Artificial Sequence
  FJ47G6 amplicon sequence
atgaattccgccagctccgagcagcggttgggaccgttactcaccatttctcaggacgccaagctgggtctggcggagcaccgaattgaagagcatcagggtcggaggaaaagcctcagctgag

SEQ ID NO: 127 - DNA, 467 bp, Artificial Sequence
  FJ8F8 clone sequence
ttaaagtatcttacccaaaaatggttgaaaaggaaaacaagtttttggtgaactttcaggcagtgatgaaaaaaaaaaatttccaagcgccactagggtctaaaatgttcccaaacagtaaactctcccggagttcactttgattttggactaagctggtgaatgacaggagatcagtacgagcgcagagcagtccccagaggaaaggaaggtagaaggcggtgtcgccgcgcccctcgagccagagccgcgagcccccgcccggctcaaggaggaaagtgaaccagggcttcccttcacgggttgcgaccgatccggagcccgcctggtgcgctggcccgcggtccccaggcaaaaggtaatcaagagtcactcctccaaaattcaaactccctccccaaactgcgagtcctgctatccccacaccacctccaagaaaatccggagactctgcagaaagcgtttaa

SEQ ID NO: 128 - DNA, 824 bp, Homo sapiens
cggtgtcgccgcgcccctcgagccagagccgcgagcccccgcccggctcaaggaggaaagtgaaccagggcttcccttcacgggttgcgaccgatccggagcccgcctggtgcgctggcccgcggtccccaggcaaaaggtaatcaagagtcactcctccaaaattcaaactccctccccaaactgcgagtcctgctatccccacaccacctccaagaaaatccggagactctgcagaaagcgtttaaagagcacagaacaggcaccgacttgacaaggcggggtgacactttctcgcggcgggtcccctccgcagcccgctcccgcggccagcccgacggcaagacgcaagtctagcttacgtgttaggatcatggtgtccggcttctttctgcacatcaagcacggcaggcggcggcggaagcgctgtggggaagtcgaggcaggcggaggcggctcggcttccgcgtcgggacccacggcggcacccgagacgcgcgccctcgcggtcctcaacgcatccttgctcgccgctccctgcccctcgtcacggccccagaaagaaagcggggttttctaaagatcgaaacgagggagcgctcagggagttgggcgagaagtccgtgagccggcgctcctgatgcggagaggtgcggccatgtcctggctgggagcgaagcgccctcgctcgggcagtcggagcgaactgtctcccgcgcgctccgccagccgggccctcccgctgggcccaccccccgaggggcggggccagagcgggcggcaccgcctcctccccgctgtctgggtcgcaggccttagcgacg

SEQ ID NO: 129 - DNA, 149 bp, Artificial Sequence
  FJ8F8 amplicon sequence
tctaaagatcgaaacgagggagcgctcagggagttgggcgagaagtccgtgagccggcgctcctgatgcggagaggtgcggccatgtcctggctgggagcgaagcgccctcgctcgggcagtcggagcgaactgtctcccgcgcgctcc

SEQ ID NO: 130 - DNA, 775 bp, Artificial Sequence
  Sanger26F2 clone sequence
aagctctgtgagaatcctgggagttggtgatgtcagactagttgggtcatttgaaggttagcagcccgggtagggttcaccgaaagttcactcgcatatattaggcaattcaatctttcattctgtgtgacagaagtagtaggaagtgagctgttcagaggcaggagggtctattctttgccaaaggggggaccagaattcccccatgcgagctgtttgaggactgggatgccgagaacgcgagcgatccgagcagggtttgtctgggcaccgtcggggtaggatccggaacgcattcggaaggctttttgcaagcatttacttggaaggagaacttgggatctttctgggaaccccccgccccggctggattggccgagcaagcctggaaaatggtaaatgatcatttggatcaattacaggcttttagctggcttgtctgtcataattcatgattcggggctgggaaaaagaccaacagcctacgtgccaaaaaaggggcagagtttgatggagttgggtggacttttctatgccatttgcctccacacctagaggataagcacttttgcagacattcagtgcaagggagatcatgtttgactgtatggatgttctgtcagtgagtcctgggcaaatcctggatttctacactgcgagtccgtcttcctgcatgctccaggagaaagctctcaaagcatgcttcagtggattgacccaaaccgaatggcagcatcggcacactgctcaatgtaggtttatttttttcccttct

SEQ ID NO: 131 - DNA, 130 bp, Artificial Sequence
  Sanger26F2 amplicon sequence
gatgccgagaacgcgagcgatccgagcagggtttgtctgggcaccgtcggggtaggatccggaacgcattcggaaggctttttgcaagcatttacttggaaggagaacttgggatctttctgggaacccc

SEQ ID NO: 132 - DNA, 568 bp, Artificial Sequence
  FR1A6 clone sequence
aagccgcaagcaggcggagggtactgcccactccggcctccccctcccgcgcacttgtactttatctgcaggatctccacacctcaggacagcgagaaggcgctttcgcagcgccctctgctaagagtcgggacgggctctgcccgagtgtagccacaccgcccccagcctcaactcgccttttccttatttcatgggcatccgttctctctggctgtcccacatttgctctgcactagtcagtcctttctgacgctgtcaaaaccagtttccccagcactctgtctcgggaaaggcgtcccttcctacctgcagctcgtgtagcctcgacagcccccccttgcagaaaagttcggccagaaagctgggcgtagaggggcctgggatgtctgccaagctccggcgtgctgagtggtactctcggtagcctagggaggcgcccaactcgggcgcccagcggacgcgatggaacactctggaggcgtacttgagggtctgggtcatggtctggttcagggtgctcgcgaaagaaagcgcttctcctgagcatcatatctcaacccctatt

SEQ ID NO: 133 - DNA, 1145 bp, Homo sapiens
cgcagccagtggggcatcgccatggtcaacagcgtggacacaaacaccgagcccacagcgcggatgaaggtctccgtgtcgggtggcacttgagcctccaggcagcccaagcgcgagccgagcagaaccgcggcgatgccttgtcgggagggggcgccgtcagggttccgggaggctctggtagggcgcccccgacgcctgcccagctctgtcctgggactcaccttccagtccgaacttgtaaaattcccccgccacgtcccgaaccagggcgggcggccccgtgccacgtccccgctggcgcctcagacgccgcacaaggtcgcagactacgttgttcagggttccggcgtagcgggcggccgcttgaggccggaggaggagcggggccaggagactgcggagcctttgccattcttcgccttccctgcagggttgaggagagagtgcgcatcgggaaggtggggacggctcgacgggactggctgcagtgaaggagcgcgttcggctgcggtggccaggagagggattgcgtctgcccccttggggtacggaaacctgcaccggcctgcgcctgtcttccagcccccttcctctttactcatttcctgccctcaggggccggggttcccgggtaacttgagtcacagaaccttacattcattccatccagatccttgtaccctagcccaattcctccggttcccccagttcccaggattcgggcctcaaaggataaggaggtaggcggggagtgaggttggggctcaacctgagtgtgggtgaagcagactgggatgggaaccccaagatgcccaatggtagagtgggacagccgacctcccaccatgcccccagattgatagtttcgggacccgcagcaggagagggccgctgcagggcgtctgggcttctgggggcagagaagactcacgcagtgagcagtccgcaagcccgctggcggcagcggcggtgctccgtccagggcgagaagctgcagcgctcgggccggggtccctcctgtcgcagcagctcctcgacgagtgcaggggcagccacgtacacggtgcgcactgtcccaaagctggctagccacaccggcccgaagtgcgcggcgccctgcacctgggggagcggacacagcg

SEQ ID NO: 134 - DNA, 304 bp, Artificial Sequence
  FR1A6 amplicon sequence
ccagtttccccagcactctgtctcgggaaaggcgtcccttcctacctgcagctcgtgtagcctcgacagcccccccttgcagaaaagttcggccagaaagctgggcgtagaggggcctgggatgtctgccaagctccggcgtgctgagtggtactctcggtagcctagggaggcgcccaactcgggcgcccagcggacgcgatggaacactctggaggcgtacttgagggtctgggtcatggtctggttcagggtgctcgcgaaagaaagcgcttctcctgagcatcatatctcaacccctatt

SEQ ID NO: 135 - DNA, 459 bp, Artificial Sequence
  FJ3F12 clone sequence
ggattgccagctccgagacccgggactcctcctgtcctgggccgaatgctcttttagcgcggtagagtgcactttctccaactggaaaagcggggacccagcgagaacccgagcgaacgatgggagggagctgcgcgcagaggcgccgggccggcccgcggcaggtgctatttcctttgctgctgcctttgttctaccccaccctgagtgagccgatccgctactcgattccggaggagctggccaagggctcggtggtggggaacctcgctaaggatctagggctcagtgtcctggatgtgtcggctcgcaagctgcgagtgagcgcggagaagctgcacttcagcgtagacgcggagagcggggacttacttgtgaagaaccgaatagaccgtgagcaaatatgcaaagagagaagaagatgtgagttgcaattggaagctgtggtggaaaatcctt

SEQ ID NO: 136 - DNA, 539 bp, Homo sapiens
cgccgccgtcggccagtgcagagcaagcgctgacgccggggatccctcagcctctagcctgggattccctgcgcagccaacaacagaaaagaaaaccagctcccacacagaggctcccggctgcgcagaccttgcccagcacaccagattgccagctccgagacccgggactcctcctgtcctgggccgaatgctcttttagcgcggtagagtgcactttctccaactggaaaagcggggacccagcgagaacccgagcgaacgatgggagggagctgcgcgcagaggcgccgggccggcccgcggcaggtactatttcctttgctgctgcctttgttctaccccacgctgtgtgagccgatccgctactcgattccggaggagctggccaagggctcggtggtggggaacctcgctaaggatctagggcttagtgtcctggatgtgtcggctcgcgagctgcgagtgagcgcggagaagctgcacttcagcgtagacgcgcagagcggggacttacttgtgaaggaccgaatagaccg

SEQ ID NO: 137 - DNA, 371 bp, Artificial Sequence
  FJ3F12 amplicon sequence
cgagaacccgagcgaacgatgggagggagctgcgcgcagaggcgccgggccggcccgcggcaggtactatttcctttgctgctgcctttgttctaccccacgctgtgtgagccgatccgctactcgattccggaggagctggccaagggctcggtggtggggaacctcgctaaggatctagggcttagtgtcctggatgtgtcggctcgcgagctgcgagtgagcgcggagaagctgcacttcagcgtagacgcgcagagcggggacttacttgtgaaggaccgaatagaccgtgagcaaatatgcaaagagagaagaagatgtgagttgcaattggaagctgtggtggaaaatcctttaaatatttttca

SEQ ID NO: 138 - DNA, 402 bp, Artificial Sequence
  FJ31B11 clone sequence
aaagtgattacaattcagcgcgaaccttccacagagaggaaatgaatgcggcagacgagcccttcagaaataacctcacacctcttccatttcaaccttgagggtgatttgctgcccactccccaaagaataaacgcctttatcgaacccggtgcggaaagaaatgagggccgggggcgggggctgtgggaagaaaagcagttcctttattgttggaaaaggcccctcggtcctggacttcaagggcaggttgcaagcctggtcttcagccgccactgaagagcgatctcctggcagcgagactgtcccagcgcctcgggacgcgtccccactgtcgtccagcgcccaggaagtgagcccgacgcctgctctgcggggccacaggcccccgagacccgccct

SEQ ID NO: 139 - DNA, 3061 bp, Homo sapiens
cggtatgggcctagcggggccgacttctgtgcacaggccgggaagcaataaaggttcggaactgctccagacccggaggaccccgccgggcactctggataacctccccactccacttccagggtccgaaaacacgggacgtctgcgactggccggtgtccagcacgcgcactaggaccgcccccggcgcccacctctagtctcccgcatacccggcgggcttcgggacgcctacaaatcggttccaggtaatcgcgcgcgttcctccggcagcacatcgccaggccgcgggcgaagggctcccacctcctggctcggccaactctggcagcttgttctgcacacatgacgtaacccggcattagatcaaactttctggcaccttgaacccactggggtgggtggagggatggagaagatgggagaatcctctccactgccaggcgcgccgggcccagactgactcaggggccgaatgatttcctcgcagttgaagaggaaaataaaaacctaaagcttccgcttcccagcccccctcacttcccccagcccgtgcgctctctctgcgcgcaccgggaaaagagaagggaaaattaaaattaaaaaaaaaaaaaaaaaaaaaaaaactttgggggatgtttggcgggttctctccgcagtccctgcggacccacgctccaggaaggagctcccggctgcgcacactgggggctccccgcgccgggaaccggatgccagccagatgccggggctgggaagcagagaagtccctactccccggctcccgcccttttccgagtctcccggcgccccgcagaagcagttggtctaggcagccccggggactcccaggggtacctggcctttccgcggcggcttaacaaagaggggcgcgacgcgagcgcttcccgccgacgcctcaagccatcagcgcccgggcgacgcaggctccgagggtgcgcgccgccagcggttggtgcgcgccgatccccgccgccgctccttccgcagggccccgaggcccgggtagccctggctctccgcctgttatcggcttacctggggttgctgctgttccagtagacagcgtagcggtcggcgacggccttggagcccgggtcctggctgaacacacacatccagagcaccagaaacaccagcgtcaacatctccacgtgcaacatcacgcctggccagcggcggagcccccgacgcgccactccggggagagagcggggatccggagggagggaggcaggcaaagggacagagagagagcgggcgccaaataaatatgaataaataaaaatgaaagtgggcgagaaaggaaagaggcgcccaccaagctggggaggggtaggagagcgagaagaaaagaaggcggtgggatggggggtgataaagacaaactcgcacccccactggagggttcaggaggaaaaaggaatcacaagatggagagaagcgtgcgtgtgtgtggtggcggcggcgagggcgggggaagaggtgccaagctgtgtctcgggcggcggcggcaggccggtcaccccgggagagggaggtgcgcgccgggccgggcggctgcagcgcgggtcggggcaggcggcggcgcgcgctgcacagtcaccgggagttgcgcgccgcagccgcggggagacatacaccggcccgccgggcccggctcagcgtcggcgcagcaggctacgcacggctccgccaccgcccggggccctcgcactgcgccacgcttcgctccaagttactttggcggccggtgggaaactgcaggagaagggcgacgtgctgatagagggcttcgcgcttgtggtcccgggagtctgagaacccctttcctcagcctcctctgtgccttagcggggcgacccccagaaatctcagaggagtcgtaagtgctgcggtaccgggcggtgcgccgagaagggtggggatccggagccgcaacccagccttgtcggtgtcctggaactggttaagcggcagctgcgccggctttcgaagcggtcttgcctcgaaaacctcaaagaaaagtttggctcttcgcgtcttccggcaggtcccccagcagagtcggaacgcggcggtagcggaagagggcaccccgggggctggccgagcctcggggatttctcgcgcgtgctttccttggctgggtgtgggtgcaccacagtgaatgaagagcagaggttaagaccagccggcctggcagagctgggattcctaaacctgcaagacgacgccaacccaactgttcggctggagagcgggaggggaaacaatcactttcaaatggatccctccaactcctggatgggccgatgtcagtctccgatcgccttccgagagggagactgagcccccacacgcggtgccaagagcccggggtgagctctcagaagtgtccctgcgactccagcgggtccaagcctagcaacttgtagagatgcatcaatcagtttgaaatgggaaaagaggaaaaaaaaaaagcgcacgagcgagtggcacctccccgatgactacatcagaaaaccttcccgatcctcccgcagctgctcctctttccctccttaggcacagcagcaacagaggtgaaaaccacaaaccgaatcctaaaacccctacccaggcgggaaaataaataaacttgagtgcagcagcagcagcacgagcagcccaggaggaattgaaacgactggcaagaagtggaaagggttttgtaaaagtcagttttgcaaaagagggggaaaaaaagagagcgagagaaaaaatgctttatgggaaaaaaaatctcctcgggcacgccccctcgttaatgacttgcgtggaaagccgacagcggcggcgacccagtggcagcagcctcagcagagcgcgaggccgcacgcgtgaacgtggacgtgggcggggggatggggctccgcccgggcgtgttgcgcgggacgcgggacgggcgggcggcgcgagcgcgggtgtgagctccggacgaagcagggggaggtgccgccagcccgcgcccagccagcgctgctgccgctgcggcccg

SEQ ID NO: 140 - DNA, 184 bp, Artificial Sequence
  FJ31B11 amplicon sequence
cccccacacgcggtgccaagagcccggggtgagctctcagaagtgtccctgcgactccagcgggtccaagcctagcaacttgtagagatgcatcaatcagtttgaaatgggaaaagaggaaaaaaaaaaagcgcacgagcgagtggcacctccccgatgactacatcagaaaaccttcccgatc

SEQ ID NO: 141 - DNA, 359 bp, Artificial Sequence
  FJ43G12 clone sequence
cgagctggagaggcgcggggatgcggggtcctccccgcagtcttccggaaagggcgggggagggcgcggcaagttccggagtggggcatgccgtgggagcccacgagggcctcagcgcggatcctccgccggaaaaccggctcccgcgagccgccgccgcaggtttcctaggccccgcgagtcccgcagcgaagccctgcgtctccgtccgacgcgggggtctgctcagcctcgggtgggccgcggccaggcctgactgcgggggagagggccgaacgtgacctccgaggtcacccccagccagctttctctcctgtggtcggaagtggttttcttctcgatctgggcgcctactcccc

SEQ ID NO: 142 - DNA, 7460 bp, Homo sapiens
cggcccggttctgtcactttcttcagccggacagtcgccttattacggattccagtagggccgagcacactccttcctgcccgtctttacagatgaacatgctatcgctccagcagtacaacccgccttattacgtaaaaaagcagcccctatctacccgcagggaaggagtatgttcggcaccacagctggactggtgctcgagttaagcgtcctgggaggtccgcatgcgctccggaaccgtaatgcgcgctttttctaagccttacggtaaacgcggacgcagggcaaccacgtggcggtggaaccgaggcccggcgggaatgcgcggaatgcgcggcgcggcctcgcgcggttcccgagccacggcccagggtccggcggcgcgcgctctcgcctcctcccctcacctctcccagccgcaccccggccctggccctgccgcccagaactcgctgggcaagtcgtgccccgcgtgaacacacagaaggggcttggggaccgagcgcggcccatcagtccctcagaccctgaggacccagaattccctaaggggtccgaatccgagtcctgcccccagcccttaaggcacgggctccagggaccccaggggaagggcgcggggcattaggtacgcaacccgtttccccgcacctggaaaaaaactccctttccctcccctcccctgcttgttgagtgtccggataaccagaactctaaggcgccccgtaataacgaccccgctgtccctccacccacccccaagtgccaaagcgagggatggaagcgctttcaagcgttccaagggcattgaggagcgagctggagaggcgcggggatgcggggtcctccccgcagtcttccggaaagggcgggggagggcgcggcaagttccggagtggggcatgccgtgggagcccacgagggcctcagcgcggatcctccgccggaaaaccggctcccgcgagccgccgccgcaggtttcctaggccccgcgagtcccgcagcgaagccctgcgtctccgtccgacgcgggggtctgctcagcctcgggtgggccgcggccaggcctgactgcgggggagagggccgaacgtgacctccgaggtcacccccagccagctttctctcctgtggtcggaagtggttttcttctcgatctgggcgcctactccccaccacttggtctgagaggggctggggccggaaggccagggaatctctggtggatttgggggttcatattgctcagggtaccagccgatgcgttttgaggggcgggagtcgaggaattagaatcgcctttaaccctcaagagttgcgccttcagcctcgggatcccagatgcgtcgttggagccagggccgcccccctacctgttgggtttgcgttttaactccagcgcacaccttgccggcagccctcggagctaggggaggggtctcgtttccccgcagcccgccggacagacgactggggcacgggaggggcggtggcagggtggtctgtgtgtggctgaaactaattgatctggagcggaaacgcacgtctgcggttggggcgatggggggggcggtgcggctgtccatgtgccgagcgtgtggctgtctcgggtgggcactggggccggagttcgccccggcccacctcgcagttttggggcgcctgggatcggcgctacgtaagcgaagcagagctgccatagcacgtgggccgccacgcgcaccccaaaagcaagcagtgtggggggaaggggagctcgagcgccttcggagcccaggggccggctttcggaagcgttttcccgggcgacttaagggcttaacaatggaaaactcgcggagcctgagccaagtcctttcaagtcgccgccaggtatgcggctgcaggtgaccccacctgggtgcgcccgcccgccagccgccctggtggaaaagcgggtgcgggaggtcgctggcgaaaggtcgggactggtccctgcaccacccgcccccaacccaagccccgagccccgcggcgcgcagccgcgctgagtcccggggtctgcgtcgcggcgcgccggttcctgaatgaacgcgctcccttcccccgcctgaatgaaggttcccacagccagggacggtggcgaacacgcgcctgcagcggaattcgctttctcctgaccgaccatccgcccaggccgcggtcaccggggcgggggccagggggcgaggaaagcgtgaaggtgatttcagttaattttggattttctttcaaacaacgtggttaccctcccgactgggccacttgccctttgtctccaaatggtcaccaagaaataagaacagagcactttaaatgagcccagaatccgcagttcctgcttcgtggtgggttttaagaagacagtgtaaagtaaaactgcaaccgaaaagttttttaaagttgcttttctctttggaaaaaataaaatcaaaatgctttctctgcgcttcttgaagcaatgaccctcaaaagcccagaggtattggccccctcgggggacccgggggccgccaagcagggttcccccaggtgggggctgggcagctggcgctccccgccgggccccaaattccagcgccgggccccaaattccagcgcctcccccgcgggttcctggacggctctttacgctcgctaaccgggcttgcaattttgcgctcgtccctgagccgggaaatcaacgaagttcctagtcgagatctgcccggtccgcctagtaacagcgccgcgcccccattggctcatgctaattccagtttcctctgtcttgcgcccgggatgggggggtgaagctccctcctggacccagagccggttgtgccggagtgggcgagcctctttatgccctgctgcccctagccgacttcggcccgcttcgcgcctcgggctgggccagggcgcacgcggggctcggggcccctcgccccacgggatgggagaggccgggtgatagctccgggccccataaatcatccaggcggccgccgggtcgggattttatgaatgaaaaagcagctgggccgcccttgtgcgcgggctgatgctctgaggcttggctatgcgggggccaacgcgattgtgggtgctcggggagtgggggggggcacgaccgtaggtgctccctgctggggcaacccatcgctccccatgcggaatccgggggtaattacccccccaggacccggaatattagtaatcctaattcccggcgggggagggggcgcgggaggaattcaccctgaaaggtgggggtggggggggtcgcatcttgctgtgagcaccctggcgaaggggagagggctttttctatcagttttctttgagcttttactgttaagagggtacggtggtttgatgacactgaactatattcaaaaggaagtaaatgaacagttttcttaatttggggcaggtactgtaaaaataaaaacaaaagttaagacagtaaaatgtccttttattttttaatgcaccaaagagacagaacctgtaattttaaaaactgtgtattttaatttacatctgcttaagtttgcgataatattggggaccctctcatgtaaccacgaacacctatcgattttgctaaaaatcagatcagtacactcgtttgtttaattgataattgttctgaattatgccggctcctgccagccccctcacgctcacgaattcagtcccagggcaaattctaaaggtgaagggacgtctacacccccaacaaaaccaattaggaaccttcggtggtcttgtcccaggcagaggggactaatatttccagcaatttaatttcttttttaattaaaaaaaatgagtcagaatggagatcactgtttctcagctttccattcagaggtgtgtttctcccggttaaattgccggcacgggaagggagggggtgcagttggggacccccgcaaggaccgactggtcaaggtaggaaggcagcccgaagagtctccaggctagaaggacaagatgaaggaaatgctggccaccatcttgggctgctgctggaattttcgggcatttattttattttattttttgagcgagcgcatgctaagctgaaatccctttaacttttagggttacccccttgggcatttgcaacgacgcccctgtgcgccggaatgaaacttgcacaggggttgtgtgcccggtcctccccgtccttgcatgctaaattagttcttgcaatttacacgtgttaatgaaaatgaaagaagatgcagtcgctgagattctttggccgtctgtccgcccgtgggtgccctcgtggcgttcttggaaatgcgcccattctgccggcttggatatggggtgtcgccgcgccccagtcaccccttctcgtggtctccccaggctgcgtgtggcctgccggccttcctagttgtcccctactgcagagccacctccacctcaccccctaaatcccgggggacccactcgaggcggacggggccccctgcacccctcttccctggcggggagaaaggctgcagcggggcgatttgcatttctatgaaaaccggactacaggggcaactccgccgcagggcaggcgcggcgcctcagggatggcttttgggctctgcccctcgctgctcccggcgtttggcgcccgcgccccctccccctgcgcccgcccccgcccccctcccgctcccattctctgccgggctttgatctttgcttaacaacagtaacgtcacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtgcggggcttcgggcggctctcttaagacttccctgcaacttgttgcccagacccacgtttctttgctactcacccccctcccttctctcccgctagaactttgaagtttgccgtggtgtttctagggatccgtattttcaaaataaaaattgcgggtattttctgaaggaggaaggggtgggggtgggggtgctagaagtagcgtttcgtgggaggggagaagggggtccgggaggggtgccttcgggagaagccagtgccaggggcaccccaatgggcccgagggtgcgggctggcaggctgggtgcgctttgtgtcccccgcctgcgccccagcccggctgcgcctcagcggccgggagccgccaactccggggggagggggcatagatttgatttttaaattaatatccatggacacgtatgcaagggccgctcgtgccagtattatgcgccatctttgctcttttattgcaaagcaaaagtgtttattaataattgggggcagggtgggggcggggagcggccgccgggcgctggggccgcagctaagggccgcgcggctgccgggagcccgcgggaggggcgcagggacgcggcatgggtagttttggggggacgccgctagggaagggggggcctttgttcaagcagcgagtcccggggcgccccgaacgggcagcctgggccggagagcacggcgagctgcaaggtcgcgtggcccccaagacgccagggcttgatccccgtctgcagggatatcggcttggaggaccttctccgagcgagccgggggcctgggagcacattttcagaccttcggtgggcgcctgaggggcccgcaagtattttaaaataatttttgaaagtgcggcgtggtgcccttgcgagagggaaacgccgcccgcgcccagggggaagggggggccccggagtttgaattcctggggctccccccggagcctgtaacgaactcccaacccccggcctgggtaaagggtcgcccgagggtcattttcagggtttttttatgcacttagttatttttttaatatttttaaatattttttgaaaagatgacgtctggggaaatgcggcgcggcggcctgggacgccacctttgtgtctcgcaggcgcggcgcccaaccccgcggcccgttccgcggccccgcaccccagttggtgtcgacccccagtcagagggaccacggagctccagggcgggccagggtcccgggggccggcagcccgcgccgccgcgcacgccgcccagctgtgcccgctcccgcccccaccgtgccagcctcgcggggactttccctttcagtttcggggagggtgggtactggggacgcgcgggggagggggcgcatcacgggaagctcctgccgcccccagccccgacccctcggcgccctccagacctggcggccctgccaagcgcgatggggggtgcgggggcgtgcgggggggcggcgcgacctggcggcggcggtcacgggccccgtgcctccgtaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctggtaaccactggaccccgccgccccccgccccccgcgagccgcacgcaggaccacggggccggggaaggtgcaggcggtggcggccggcccgcctctgacatatctgctcctccgagggagggcggccccgccgccgggcgtccctgtccggggagcgggcgggatcctagccgccctcgtcccgccgccctgtgtgcgcttgcctgcgactcccaccgcgttcgcgccccgcggtgtggccgaaaagtgggcggcgcgcgccctccagcggctgcacgaggagcgccgcgctcggcgctgagcctccagttccaggtggtgggaggtctttttgtttccacttgcagagtcttttcacgcggcgggcgccttttctgttttgatctgggattgcgtgttgccccagctcccttgagtccccagcattcgccagccctcccctccaacatccaggaccgcacgagacgcaggggccagtgctctgagccggaggtgcggcgtggcccggcccccgtgctgccggcttccccgcgcccccgggctggcccgcacctcccctgatggccgctcaccctgtgttcg

SEQ ID NO: 143 - DNA, 261 bp, Artificial Sequence
  FJ43G12 amplicon sequence
ggatgggggggtgaagctccctcctggacccagagccggttgtgccggagtgggcgagcctctttatgccctgctgcccctagccgacttcggcccgcttcgcgcctcgggctgggccagggcgcacgcggggctcggggcccctcgccccacgggatgggagaggccgggtgatagctccgggccccataaatcatccaggcggccgccgggtcgggattttatgaatgaaaaagcagctgggccgcccttgtgcgcggg

SEQ ID NO: 144 - DNA, 1006 bp, Artificial Sequence
  FJ60C11 clone sequence
aaatctaggataggttccataacccagtacttctgcatggttagccagacctgggcaatgtctcagtcagccgtaaacatccaactggtagcctgatcctctctcccaagtccggcgtttccaatggacgtccttttttcccaagcagaatgttgagggcgagctgggaaaggggtcatcacgttcagaaagtaaaagggggtcatgaccttcagaaagcaaagtgggggatcataaccttcaaaaaataaatgggactaattcggctcggcagtctttggaaacgcaagtagctcactttccccataagcgagtctcaaggggcggtttgctgggtgagatggaggagtgaggttccaaagctgctctttcctacccgctaggaagggggcgccaggcagcccctgacctcacttggaagaggagagaaagagagtcgaagacgccctgtcaagatgccactcacctaaacgccaggaacatctccccgactaaggacaggccgacccccagcaggaccagcgccacgagcttccccatggtctcggggtgcccagcggcgactgcgcggcgccgagagctctcgggggcgcggcgggcggttcctgcctcgcgtacggattggggcccgctcggccccgcccgcacacgcctcctacttacctgagcggcccgggtgccgcagcagggcgctgacgagtcccgccgagccccgccgcccgggttcaaggccgccttcacgcccacgattccttgttttttgggcagaggtcaaggctcaacggagagggagcccgggaccgagttgtggtgggagtttgctcctaacacccgcgaaatcctacctcaattcctcagatggcctgagggagaaaccggagggcccgggaatggcggggagctccagttcagagccggaactccggacttctgcaaggccagacgtgaggcgacactgaattgctcccgagacgcgggaagggcgaaggcaatcgaagcgaagagc

SEQ ID NO: 145 - DNA, 563 bp, Homo sapiens
cgaagacgccctgtcaagatgccactcacctaaacgccaggaacatctccccgactaaggacaggccgacccccagcaggaccagcgccacgagcttccccatggtctcggggtgcccagcggcgactgcgcggcgccgagagctctcgggggcgcggcgggcggttcctgcctcgcgtacggattggggcccgctcggccccgcccgcacacgcctcctacttacctgagcggcccgggtgccgcagcagggcgctgacgagtcccgccgagccccgccgcccgggttcaaggccgccttcacgcccacgattccttgttttttgggcagaggtcaaggctcaacggagagggagcccgggaccgagttgtggtgggagtttgctcctaacacccgcgaaatcctacctcaattcctcagatggcctgagggagaaaccggagggcccgggaatggcggggagctccagttcagagccggaactccggacttctgcaaggccagacgtgaggcgacactgaattgctcccgagacgcgggaagggcgaaggcaatcgaagcg

SEQ ID NO: 146 - DNA, 278 bp, Artificial Sequence
  FJ60C11 amplicon sequence
tttttgggcagaggtcaaggctcaacggagagggagcccgggaccgagttgtggtgggagtttgctcctaacacccgcgaaatcctacctcaattcctcagatggcctgagggagaaaccggagggcccgggaatggcggggagctccagttcagagccggaactccggacttctgcaaggccagacgtgaggcgacactgaattgctcccgagacgcgggaagggcgaaggcaatcgaagcgaagagctttaactcatcttctccaggatttggggc

SEQ ID NO: 147 - DNA, 596 bp, Artificial Sequence
  FJ30A12 clone sequence
tgtgtgtgtgtgtttggggggctggggaggggagaagaatatatcagtcaacattgtttcctcccgcgctcccaaacataggctaactgtgaagagccaggacgcccaccgagtcctggcagcgttgcaaggtggggagggggcgggcgcggtcctgtcgctagtgactgcagatttcctctgcggggcacttcctacggcctttgatctttccccaggaactgccgagggccgcgtttgcctctaagtttattccctcttggtttatttattgtatctgctttctcttctcgctggtttggaggtcagaacgccagtatgcaacgggacttgtgtcccaggcagggtctcccctctccaggcactaccgcgaaatctggcaaaaggtacattttactgagtcctagggcagcaaattcagcggcggagaaagagtctttctccattcaggagacaacccaggaagcccgatcccgggatcagaggccaggaccccgccccggccccctgccccaggcccagctgggcgccgcccaggggcttttctcggtccgcctgggggctcgagctcgcgtccctccctaagacgccaggct

SEQ ID NO: 148 - DNA, 1311 bp, Homo sapiens
cgatcccgggatcagaggccaggaccccgccccggccccctgccccaggcccagctgggcgccgcccaggggcttttctcggtccgcctgggggctcgagctcgcgtccctccctaagacgccaggcttaacttggagtttcatctcctccccaggagggtctcaagttgccttcctctgccgggaagggcccccactcccccgctccaggccctaatctccacccggcgtcccagtctgagcggcctggcctcagctctccatctgcgagccttaaatcccatttatcttcttagcgcctcagacccggacggtcccgcctctcttcctctcacccccaccccacttgcaaatcacagacccccgtctacccctcgcatcccccaagtgcgctggaccaactacagcaacctttgaggtgttagaaaaagtcgcctagaggggcgcggcaggaggagggcccaggtggggtggagggggggcgccccaagatttcagggtgaggaccgcggagggtcctggacccgcagtcgcctaactcctgccctgcgtctccagtgcttgaagtcggtccctcccaggtccccaatctaggtcaccagcggggaagggctgcgctgggggtcgctagagagggtgaggggcgggctgtctgcttccgaggtgggcgaggagtgaaggtttcagggcctgtccctctcacttccactcgattgtaatttcatccccgggccggtcgagcctccctccctcccgcggccagcccttccctcccagtcggcctccttcgtcgcccccgcccccgcgaaaagccctgcagcttgcagccggcttcactcgcgcacgccgacctcccggctgcagtcctacctcttggaactacccgtgtttccgggcccagccctcgcagccccccacctcctcgccccggcccggggatccgttggggccgcgtcccccacgcgcccccggagacgccctttccgtgtgcgcccgggacttggtgaaactttgcaggcgccggctgcgaaatggatttaatccgaggcgtcttgctccggctcctgctcctggcttccagcctcggacccggcgcggtgtcgctccgagcggccatccgaaaaccaggtaatgcgctcctccgcccagagccaccacgccccgagcgcccctgctgggctccggggcgaggacacagagcgggcagcgcaccgcctccctctccccgaccaacgtctgcttaactcgcttcagctctgccaggagctgagaaaagacgcaaacctcgccctcccccgagcccgggcggcg

SEQ ID NO: 149 - DNA, 296 bp, Artificial Sequence
  FJ30A12 amplicon sequence
ggctcctgctcctggcttccagcctcggacccggcgcggtgtcgctccgagcggccatccgaaaaccaggtaatgcgctcctccgcccagagccaccacgccccgagcgcccctgctgggctccggggcgaggacacagagcgggcagcgcaccgcctccctctccccgaccaacgtctgcttaactcgcttcagctctgccaggagctgagaaaagacgcaaacctcgccctcccccgagcccgggcggcgctagggatatttatttattaaaagaatgatcaattttccagaca

SEQ ID NO: 150 - DNA, 754 bp, Artificial Sequence
  FJ12A3 clone sequence
aataggggaaagaggtgctgaatgtattctggaatctagaaaaaattgcatttttatcattgacggtaacagttttgcaaaagatttacaagctttttttcccctacaattacgtctatatttttcactaagaagttcttgtaactgggaaaatactccaatctggccgagcagcagttatggaagaaaaactctagcaaacagcagttgggatgggagggtggaggtcatcaggggattcccgctgagaaagtgggaaggaagagagtttagaaacggattgaagttccactacatcacgtgtgttgttagagaattcgcctattacgacgacagcaacgtctgatctcctggctttcttcccctccttggtgggttccgttccgtccccctgtcgctgcgagtttggctgtccctacgccggggtcttggagtttgggcggggtctggtacctgccaaatacggatcctgatggggaggaggtcggggagcgctaagttcaggctggagtctgagttctcccaccctgctgctcccacccaccgcgcttagaaacctatgtttccagcaaaggggtcttgctctgagagaacctaaccgggttttcttccattccctttgcatctctagatgtcttcgccctggctccaggtgcgttccccatccttttcggtcccggacgttctgcagattatagcgcccagctcatgtccagcaccgccggaggtgaagtggggcgagctggtgactcct

SEQ ID NO: 151 - DNA, 585 bp, Homo sapiens
cgaagaggagggagttccgaaaggagggaaacgagccggcaaggggcgacaggagcgagccgggaagggagagcagcggagggctgcagagctgcgggcgcccggaccgtgccacacaccccccgcggggcacggagggcattgcggggggagacacacaaagacatgcgaagaggggctgaacgaggctcgcgcacaaagacgccggggcgcacggcagctggggctgcacccaccgcccccgcgccgcgccgtgccggggccgggccagcgagcagcccggggctgagcgcgtggcggcggcggcggcggcggcggcggcgggcaggcgcgggacccgggtcgcccgcgctctccgggtgacccgggcccggcagcaggcgcgcgcgggggcggcggcgcccaagcccaacttggcctccgcctcgccctctgcccagcccgccggtgtcccctccttcccgcgatttcgtttcttctcacgctcccccccccgccccctcccgcgtccagccccgctctccccaccttgtaaaacaaagccggggaaaatgcctgcccgtgcagctcggagcgcgcagcccg

SEQ ID NO: 152 - DNA, 230 bp, Artificial Sequence
  FJ12A3 amplicon sequence (+strand)
tccagcaaaggggtcttgctctgagagaacctaaccgggttttcttccattccctttgcatctctagatgtcttcgccctggctccaggtgcgttccccatccttttcggtcccggacgttctgcagattatagcgcccagctcatgtccagcaccgccggaggtgaagtggggcgagctggtgactccttaacccttcccgaaggtgtagactggaagaagccagggtc

SEQ ID NO: 153 - DNA, 613 bp, Artificial Sequence; identical to SEQ ID NO: 112
  FJ32F2 clone sequence

SEQ ID NO: 154 - DNA, 877 bp, Artificial Sequence
  FJ32F2 CGI sequence 1
cgcccgcccgcttcgctccgaggtcccccatgcggtgagcgtgaacccttccacagcacctctcggcgccccctaccccgccgcccgcgggggcaaaatctgggggcgagggggacccgggcgctgaggccgctgcccgcctatggggacgcggctgggaccgtggcccggctgcgggtttcagtagcgacatttacgcagtgcatttggtggtctttcttgccttatcaaccccggagtctctctcccctacatcccctcccacaacttcaaggttaaaaaaatagatatgtacatctcaaaaatagcaaagggtccccgcaggcagggccgggtcctccgggccccgaggagcgggcaggcgcggcgtgcacgcggtccccgcgcccctcgcggccccagaggtggaggccggagccgggaaggtgcggcgggcggcggcgttcaggatgagctctccggctcggacgcgtgcagtaggaggccgccgccgccgccgctgctggacttgtgcttcttcaacagctgcgtgattttctcgtcgtccgagttgggatccagaggcttattgtagtcgtcgtcctcttcctcgttctccgaggcccccttgaggcgctctgtctccgagtcctgcttcttcttggccgtggccatctcggcagcgtgcttcttcctccacttggtccggcggttctggaaccagacctgagggcggagaaaagggaggagaggggaggcaagggcgaggaattaaacgagcagatccaggccatgctaccactcccgcatctcgatggccctcccgcggccccccgaaggcctgctgccctccctcgcagccctcccttttctcggccgtcaaagtcagtctccgtcg

SEQ ID NO: 155 - DNA, 3140 bp, Homo sapiens; identical to SEQ ID NO: 113

SEQ ID NO: 156 - DNA, 212 bp, Artificial Sequence
  FJ32F2 amplicon sequence (-strand)
ccttgcatacctggagggataggtaccgcccctggcgcgcgcgcgtgcacacatggacacacacacacacacacacaccccaaactaacaaacagacgcctgggaggtgcgaatcctaggagcctggggagaagttgagagcatggaaggatggtggggactacaaaggcgctggggacttgcaggcgggcaaactcttgatgaacaaaagg

SEQ ID NO: 157 - DNA, 351 bp, Artificial Sequence
  FJ7H3 clone sequence
taagggcagaattgaaaataattctggaggaagataagaatgattcctgcgcgactgcaccgggactacaaagggcttgtcctgctgggaatcctcctggggactctgtgggagaccggatgcacccagatacgctattcagttccggaagagctggagaaaggctctagggtgggcgacatctccagggacctggggctggagccccgggagctcgcggagcgcggagtccgcatcatccccagaggtaggacgcagcttttcgccctgaatccgcgcagcggcagcttggtcacggcgggcaggatagaccgggaggagctctgtatgggggccatcaagtgtcaatta

SEQ ID NO: 158 - DNA, 2123 bp, Homo sapiens
cgggagctcgcggagcgcggagtccgcatcatccccagaggtaggacgcagcttttcgccctgaatccgcgcagcggcagcttggtcacggcgggcaggatagaccgggaggagctctgtatgggggccatcaagtgtcaattaaatctagacattctgatggaggataaagtgaaaatatatggagtagaagtagaagtaagggacattaacgacaatgcgccttactttcgtgaaagtgaattagaaataaaaattagtgaaaatgcagccactgagatgcggttccctctaccccacgcctgggatccggatatcgggaagaactctctgcagagctacgagctcagcccgaacactcacttctccctcatcgtgcaaaatggagccgacggtagtaagtaccccgaattggtgctgaaacgcgccctggaccgcgaagaaaaggctgctcaccacctggtccttacggcctccgacgggggcgacccggtgcgcacaggcaccgcgcgcatccgcgtgatggttctggatgcgaacgacaacgcaccagcgtttgctcagcccgagtaccgcgcgagcgttccggagaatctggccttgggcacgcagctgcttgtagtcaacgctaccgaccctgacgaaggagtcaatgcggaagtgaggtattccttccggtatgtggacgacaaggcggcccaagttttcaaactagattgtaattcagggacaatatcaacaataggggagttggaccacgaggagtcaggattctaccagatggaagtgcaagcaatggataatgcaggatattctgcgcgagccaaagtcctgatcactgttctggacgtgaacgacaatgccccagaagtggtcctcacctctctcgccagctcggttcccgaaaactctcccagagggacattaattgcccttttaaatgtaaatgaccaagattctgaggaaaacggacaggtgatctgtttcatccaaggaaatctgccctttaaattagaaaaatcttacggaaattactatagtttagtcacagacatagtcttggatagggaacaggttcctagctacaacatcacagtgaccgccactgaccggggaaccccgcccctatccacggaaactcatatctcgctgaacgtggcagacaccaacgacaacccgccggtcttccctcaggcctcctattccgcttatatcccagagaacaatcccagaggagtttccctcgtctctgtgaccgcccacgaccccgactgtgaagagaacgcccagatcacttattccctggctgagaacaccatccaaggggcaagcctatcgtcctacgtgtccatcaactccgacactggggtactgtatgcgctgagctccttcgactacgagcagttccgagacttgcaagtgaaagtgatggcgcgggacaacgggcacccgcccctcagcagcaacgtgtcgttgagcctgttcgtgctggaccagaacgacaatgcgcccgagatcctgtaccccgccctccccacggacggttccactggcgtggagctggctccccgctccgcagagcccggctacctggtgaccaaggtggtggcggtggacagagactccggccagaacgcctggctgtcctaccgtctgctcaaggccagcgagccgggactcttctcggtgggtctgcacacgggcgaggtgcgcacggcgcgagccctgctggacagagacgcgctcaagcagagcctcgtagtggccgtccaggaccacggccagccccctctctccgccactgtcacgctcaccgtggccgtggccgacagcatcccccaagtcctggcggacctcggcagcctcgagtctccagctaactctgaaacctcagacctcactctgtacctggtggtagcggtggccgcggtctcctgcgtcttcctggccttcgtcatcttgctgctggcgctcaggctgcggcgctggcacaagtcacgcctgctgcaggcttcaggaggcggcttgacaggagcgccggcgtcgcactttgtgggcgtggacggggtgcaggctttcctgcagacctattcccacgaggtttccctcaccacggactcgcg

SEQ ID NO: 159 - DNA, 181 bp, Artificial Sequence
  FJ7H3 amplicon sequence (-strand)
aatgtctagatttaattgacacttgatggcccccatacagagctcctcccggtctatcctgcccgccgtgaccaagctgccgctgcgcggattcagggcgaaaagctgcgtcctacctctggggatgatgcggactccgcgctccgcgagctcccggggctccagccccaggtccctggag

SEQ ID NO: 160 - DNA, 508 bp, Artificial Sequence
  FJ30F9 clone sequence
aagcaggtgtggggggcgtgcggggtggcacgagacaaaaggggcacgggggtaagcccgccatggcctcccggagcctggggggcctgagcgggatccgcggcggtggcggcggaggcggcaagaaaagcctgagcgcccgcaatgctgcggtggagaggaggaacctgatcaccgtgtgcaggtacggcagcgcagggcgaggggaaccagcctcccgccggggctgagagctctgggcttccgcgcgggtccttgggggtcccgggcatgatgggctgccgcccagtgcccccgcctatgttgcgccagccaaatctgtgagcgcgcagctccttggacaggggcccgggtctggacaccgtcgcagccctggactttgtgtcagttccagtgctgaaggtactggagggtagagcttggggcggggctggaggaggatttgttttgaatgtgcaatctagcgtcagagtgaaagaggagggcaaggaagaagagctctttcatt

SEQ ID NO: 161 - DNA, 1486 bp, Homo sapiens
cgcttccgaacacgcgcgtcgaggagggcgttccaggactctgagggagcagcccagctggaccgaggccgcgtcgttcctgggcttactattcccagacccggactcccgattccggagtcacggcccaggacgcgaaaagactctacactggcaccacgctcctccttaggcgggccgtcagtcccgggtgcgggctgcgctggaggctgaggtgggagcgacatggtgtggaggggcaagaaatgtcggcactagacgcgccaagaaggagattctacgagcaattcccccctcgggccattgtgttgctgtttattagcccctgggagggcgtcaggacaaaaggaaccctcctcccttcttagtacttaggcccaaggtcgggtgtgggagccggcgcgctgctttctaggcaggcactgaagctacggcagccacgcaaataggtatcagccgttaaagcttggctacaggcaaggggggggcaataggcccctggcgctgtggggccccgcatcccacaatccccgcggctagcctgtgtggctactggcggcagctagcgggctgcgaaagcgagcccagcgtccttgacagcagcccacgcgtcggggcggggcttgagcccgctgctttaaaaggtccgcgcggccggccccgcccctctggtgccgcgattggatccggcgggggtagcgttgatttgataggcgcagagagggtggggctgcgcacgcgaggccgggggccttgccgctgcctcccgggctggggcacgagtggctgcggagtgtgggtggttgggcgtgaggggccgacgggctcgcgcgcgcgccgtctgctgaggtccctcgggaaggaggagagcgcctgacgccgacccgcaggcgcagcccggcagtcggcggcgcgccgagggcggaggtggtgcgtgcgtgcgtgtgtgtgtgtgtgtgtgtgtgtgtgtgtgtgtgtgtgtggagctcgggtgccaagggcgagccgtcagtccccgggtgcgagtccctgctgtcttccacacccttcctccctccaggctcctttcctacatccttcccgcgcccccacggttgcggaccgagcgagaacccccttaagcaggtgtggggggcgtgcggggtggcacgagacaaaaggggcacgggggtaagcccgccatggcctcccggagcctggggggcctgagcgggatccgcggcggtggcggcggaggcggcaagaaaagcctgagcgcccgcaatgctgcggtggagaggaggaacctgatcaccgtgtgcaggtacggcagcgcagggcgaggggaaccagcctcccgccggggctgagagctctgggcttccgcgcgggtccttgggggtcccgggcatgatgggctgccgcccagtgcccccgcctatgttgcgccagccaaatctgtgagcgcgcagctccttggacaggggcccgggtctggacaccgtcg

SEQ ID NO: 162 - DNA, 403 bp, Artificial Sequence
  FJ30F9 amplicon sequence (-strand)
actggaactgacacaaagtccagggctgcgacggtgtccagacccgggcccctgtccaaggagctgcgcgctcacagatttggctggcgcaacataggcgggggcactgggcggcagcccatcatgcccgggacccccaaggacccgcgcggaagcccagagctctcagccccggcgggaggctggttcccctcgccctgcgctgccgtacctgcacacggtgatcaggttcctcctctccaccgcagcattgcgggcgctcaggcttttcttgccgcctccgccgccaccgccgcggatcccgctcaggccccccaggctccgggaggccatggcgggcttacccccgtgccccttttgtctcgtgccaccccgcacgccccccacacctgcttaagggggt

SEQ ID NO: 163 - DNA, 875 bp, Artificial Sequence
  FJ23G11 clone sequence
aaactgggacaaaactaataacagtattttaccaaggacacttggccttactccccttctgtcacacttcagacataaatcactgaatgaatccagcactgaataaatgaatgatccataatttcctaagacaattccaattttcatcttgcacaaatctacaaagctgatgcacccgcacagcacttgaactcctggtaggtatctttcaaggccaaggcagttcctcctcccgaatccagttccctccgcctggatcatgctcccaaggctccctcatttcattcaggtctcctgccaaaagacacttcctcacaaaggcctctcagaccgaccccttccccactcccaacttcacagtccactcccctagccctactacatttttattcatgaagatttacggcattttgttacgtatctatacattttcttgtctgtctcttccctagaatatacagtccagcaaggcaagggctggggtggcttcgtttgctgtgggatcccagcacctagagcggagcccaggacatagtaggtgctcagtaaatcacaaatctcttctccaaggctccaaggctagtttcccagagccgcagctctcttctttcttagcccggcccacctccgagtagccgcccacctctactctagctcggccacctctaccccagctcaggtcctcattcccgcaccccccggccgcagggacggcgcggcgcacccacctcccagaccccgcgactgcggttgggccccgcggcttcgctcaaccacgcacctcccgggccgctgcgcccccgccggccccgcctgcagccgttgggacccactaactgcctcgaaaagcctaggattcgactttgaatggtccg

SEQ ID NO: 164 - DNA, 544 bp, Homo sapiens
cgcaccccccggccgcagggacggcgcggcgcacccacctcccagaccccgcgactgcggttgggccccgcggcttcgctcaaccacgcacctcccgggccgctgcgcccccgccggccccgcctgcagccgttgggacccactaactgcctcgaaaagcctaggattcgactttgaatggtccgttaatgtggttacaaaacgtgactcggttcatcgggagccctccgtaagcaagacaagcacccacctgcggtcagagcaggggtccggctcgcggtcggggttgtccggcccctcccggcttctcacctgcgcaccgcacggtccagccccggcggctgctggggatccctccgagcgccccctcaggcgtcttcctgcgaccgggcgggggaactctttacggggaaacagttttgggacccaccacccttggcgccgccgttaggagggtgtgaacgtatctattttcaaagatacccgagcccctcaccgcctgcagcagggagatagtgcccgggctatccccgcgggcgggccg

SEQ ID NO: 165 - DNA, 273 bp, Artificial Sequence
  FJ23G11 amplicon sequence (+strand)
ggctccaaggctagtttcccagagccgcagctctcttctttcttagcccggcccacctccgagtagccgcccacctctactctagctcggccacctctaccccagctcaggtcctcattcccgcaccccccggccgcagggacggcgcggcgcacccacctcccagaccccgcgactgcggttgggccccgcggcttcgctcaaccacgcacctcccgggccgctgcgcccccgccggccccgcctgcagccgttgggacccactaactgcct

SEQ ID NO: 166 - DNA, 537 bp, Artificial Sequence
  FJ55C3 clone sequence
cagtggcacccaccctcccgcagccaggaggtcctgtgacctggcgctcgcctgccccagccacgccggcgtccctgcctcgaggcctctgcctggcacccccaacccccaaccttgacctggcttcaagtctttgcatcccaactcaaagagaacctcctccacctgccctacctaaggtgcccgagcactctgtcaaacttctcgctttcttcacctgtctcgccggaaatctctttaccgaaaaccacacgtgctaagcaaaaacaccaacggtcaggagcttgcagaacggcctctgacttactcctgaggcggtgagcgcacagcaggaaaccccgcacgttcacgcaaacacatcttgcagaactggcgcggcgcggcgggaggacaggggcaagcctagcagaggaaacgggagctctgcacgtgcaggatccagacccctaggcgatgaaactgcagacctggcagcgaggcgcggctgtgacccgggaacattcgctgaacgaaatcttccggggcgaccgccgcact

SEQ ID NO: 167 - DNA, 1603 bp, Homo sapiens
cgccggaaatctctttaccgaaaaccacacgtgctaagcaaaaacaccaacggtcaggagcttgcagaacggcctctgacttactcctgaggcggtgagcgcacagcaggaaaccccgcacgttcacgcaaacacatcttgcagaactggcgcggcgcggcgggaggacaggggcaagcctagcagaggaaacgggagctctgcacgtgcaggatccagacccctaggcgatgaaactgcagacctggcagcgaggcgcggctgtgacccgggaacattcgctgaacgaaatcttccggggcgaccgccgcacttaagaatccgcgcagcctaccctctcacgccgaccaccctcccgcccgccgaggctcaccttcggcgccttcctcttcccgaacccccccagcaccaggccggggaccaggggtccggccgcctcctccagggccggtccatctggggccgcctcggcgctggcgctggtgctgcgctcccccggcgcctcctcgctgctccctgcgctgggctcccctggcgcctcctcgctgctccctgcgctgggctcccctggcgcctcctcgttgctttctgcgaggcagacacccacccggcagcgcgtcagcaccgagtcgccggcgccccagagggagcccgctcgccccgcggcccacctgcgcccgcgtcctcgccgtccgccttccgtcgctttccctgcggcgcctccgggctccccggctccgcgtcgttcacccgccgccgccgccggggccgccgtcgcctcctggtcgccggctcggtcgatgcagccgcctcggtctgcgcgggctcccgctgctgctgccgttcgcgggcccggctctgcagccgctcgagcagcgcgcgggccctgccgtgcgccccggcctccgcgccctccggccccgccgcagctgccgcatcggggcccgggtaccgcgcgacgtagaacagcgccatggccagccgcacgcctgggactcgggcgtggcgcgctgcgatgacgtcggcggcacgcctgcgactcgggctccgcgcaaaagatggggttggggtacggcgcgtagagatgacgtcgggttctacgcgcagtggtgacgtcacgggagcgccggcggctgagaatccgcgttgttccgtgttgggggcggcatggagcgggagccgggcgccgcgggagttcgccgggctctgggccgccggctggaggcggtgctggcgagccgcagtgaggccaacgccgtgttcgacatcctggccgtgctgcaggtgggcctggcggcgtcgcagggccggagtcgcggcacgggagcgggacttgaatggggggctgcggcggcaggtccccaggaggttccgagacggcgttggggggtcagggtgggaggcgtgtgggtcacgggtcgggggtgacggggctggcgtcccgagggggaagggaacgggttgggggcagcctaggcaggggcgaaggtgacagttggcggccgggcaccctgccgccgcctctcctgcagtctgaggaccaggaggagatccaggaagcagtccgcacgtgcagccgtcttttcggggccttgctggagcg

SEQ ID NO: 168 - DNA, 253 bp, Artificial Sequence
  FJ55C3 amplicon sequence (+strand)
tcctccacctgccctacctaaggtgcccgagcactctgtcaaacttctcgctttcttcacctgtctcgccggaaatctctttaccgaaaaccacacgtgctaagcaaaaacaccaacggtcaggagcttgcagaacggcctctgacttactcctgaggcggtgagcgcacagcaggaaaccccgcacgttcacgcaaacacatcttgcagaactggcgcggcgcggcgggaggacaggggcaagcctagcaga

SEQ ID NO: 169 - DNA, 564 bp, Artificial Sequence
  FJ71F3 clone sequence
tcacccctctcgctccatcccagactcaacgccccgcactccgtcctccagttaccaagtccccatccctcgtcccacctccgccttactagggactcctccctgagttcgtcctccaggagagcgcctgagtgcccccagcacacccacccgtccacctccccctgctctattcaccccagccccagctaacccgctccctccgcccttcggcccgcctcacccagcacacggagctcagcggctgcagtggcgaagtcgcgcgtgcggggcttgttggcgcggcagacatagacgccggagtgccagggctgcgcgttggcaattagtaggttggtgcggcccaggacgatgacatctgtggagatgggcttcccgtctggggaaggagagggagacgcgctggaggggacgctagggactgcccttcccttcagtctccgaacctcaggaacactctgcacccggataaagcaaacacaaccgtccctggtccgaatctcatttcctcctcttgctgggactctctgagcccggctgtctccatctgtaagggggaattag

SEQ ID NO: 170 - DNA, 220 bp, Artificial Sequence
  FJ71F3 CGI sequence 1
cgctccctccgcccttcggcccgcctcacccagcacacggagctcagcggctgcagtggcgaagtcgcgcgtgcggggcttgttggcgcggcagacatagacgccggagtgccagggctgcgcgttggcaattagtaggttggtgcggcccaggacgatgacatctgtggagatgggcttcccgtctggggaaggagagggagacgcgctggaggggacg

SEQ ID NO: 171 - DNA, 500 bp, Homo sapiens
cgccccgcccgggcctgcgctggtgcatcccgccccaggccgacgtaccccgtgccttctggtagtggagagagaagccgatgatctgctcgctgtgcatctcgggccgctcccaggccaccaacacagcggagctgctcagtggcgtagcagtgacccgcgtgggggcgctgggcagcccctcgcgcaccaccacggccagcgacgcggcagcgcacgccattcccgcgctgttctcagccacgcactggtagtagccggcgtcctgcaggccgatctgtgtgatgaccaggctgccaccgccgccctggaccttgacgcgcccgttgggccgcagcggcgccccgttgtgcagccagcgcagcgctggccgcggctcccccgacgcgcggcacacgaagcgcgctgtgctcgcccgcgtccgcgacagcgcctcgggcgcctgagtgatggcgggagccgctaggggcgcgaggggcgacgctgagcgcgggatcccg

SEQ ID NO: 172 - DNA, 402 bp, Artificial Sequence
  FJ71F3 amplicon sequence (+strand)
tccccctgctctattcaccccagccccagctaacccgctccctccgcccttcggcccgcctcacccagcacacggagctcagcggctgcagtggcgaagtcgcgcgtgcggggcttgttggcgcggcagacatagacgccggagtgccagggctgcgcgttggcaattagtaggttggtgcggcccaggacgatgacatctgtggagatgggcttcccgtctggggaaggagagggagacgcgctggaggggacgctagggactgcccttcccttcagtctccgaacctcaggaacactctgcacccggataaagcaaacacaaccgtccctggtccgaatctcatttcctcctcttgctgggactctctgagcccggctgtctccatctgtaagggggaat

SEQ ID NO: 173 - DNA, 675 bp, Artificial Sequence
  FJ78C8 clone sequence
actttttccagtactctgcatggttacaaggaaactatttttcattctctgccacagccttgttttgttctgcatgttcaattgagaatagagccaccatctcaagtcatacatacctaaactctcactcttacaccccaagaccaactctttgaagtgctctaatttctgtccacaattttccccgtttctcctctcttttttttttttttttttttgagacagagtctcactctctcacccgggctggagtgcagtggcatgatcttggctcactgcaatctctgcctcctgggttcaagcgattctcctgccccagcctcccgagtagatgggattacagtcccctgccccactatgcccggctaaatcttgtatttttagtagcggtggggtttcaccatgttggccaggttggtctcgaactcctgacctcaggtgacctgcccgcctcggcctcccaaagtgctgggattacaggaatgagccactgctcctggccccctattccttcttttagttatttattcacctataatgctaaaatcatcatcatcagagtcatacagtatcagccacttcaaatgaaaccctgtgtttattcatctttctgagctatagttttctcagctataaaatggggttagtaatgtctgcctcagagatgcataatta

SEQ ID NO: 174 - DNA, 382 bp, Homo sapiens
cgcggccaagcccccagcctccccggagtccgcagctccggctttctcttctctgctaagtgctgcatggccaagggttgcgaacgcgagcagaaaatgcgcctctcactgtcgcgagggattcagacagtcaagcgccaaggcagcccgaggctccccaaagcctcgctcggccgcacgcgggcaggaatctgcgcttgcactcgggctcagctcctcatcttcctttggccagagacagagagagcaggcaggaggggtgtgtgtgtgtgcgtgtgtgtgtgtgtgtgtgtgcacgcgcgctgtcctgtgatgagcgcgtacccgcatccccgcgcttagccgctggtgctccctccctccgtctgtccctcccgggccg

SEQ ID NO: 175 - DNA, 241 bp, Artificial Sequence
  FJ78C8 amplicon sequence
aacttcaaagcagccattgtctgtaggacttgattagagagtacgaccaaggaattggctacagtcatgtgtttgagaatcaaatctgtggatcttaatgtgcacctggtgaaataaaggaaactataacgttaaagaagaaagacatttcctaggaattcaactataatctgagataagaagatcattccttttgccaaataccccagggcatttgtcattttttcagtgacttttattt

SEQ ID NO: 176 - DNA, 272 bp, Homo sapiens
tacgtccaaggcaaggaaacctagaaaggcgtctgggcaggggaaagtcgatgcgagggcgggccagggacctttcgtcgcgtccccaccttggcatttcccgtggcgtgagcggccccggcatccgtgtcgaaagtgcggcggcggaacaggcgcgcaggagaggagcggcgcaggcgcagacgcgcgggcgggaagatggcggctgggttcaagtgagtgttggcgggtggcgggtagagttctgtaccctggcggacggcagcttcctt

SEQ ID NO: 177 - DNA, 431 bp, Homo sapiens
cgatgcgagggcgggccagggacctttcgtcgcgtccccaccttggcatttcccgtggcgtgagcggccccggcatccgtgtcgaaagtgcggcggcggaacaggcgcgcaggagaggagcggcgcaggcgcagacgcgcgggcgggaagatggcggctgggttcaagtgagtgttggcgggtggcgggtagagttctgtaccctggcggacggcagcttcctttaactcttagctgggattctctcacctggaggccgaccccgttggggtgccatttccttcctcgtcgaggcagacgatgggcgggaggacctggaggttgtcacagtgaaaagaaagggcttcttcctcagtgagacgggagatgaaaggactctgtagagttccatacccgctgcgagcgctcgtgagcgtggtgtgtagtgaacg

SEQ ID NO: 178 - DNA, 282 bp, Homo sapiens
tccaaggcaaggaaacctagaaaggcgtctgggcaggggaaagtcgatgcgagggcgggccagggacctttcgtcgcgtccccaccttggcatttcccgtggcgtgagcggccccggcatccgtgtcgaaagtgcggcggcggaacaggcgcgcaggagaggagcggcgcaggcgcagacgcgcgggcgggaagatggcggctgggttcaagtgagtgttggcgggtggcgggtagagttctgtaccctggcggacggcagcttcctttaactcttagctgg

SEQ ID NO: 179 - DNA, 609 bp, Homo Sapiens
agcctccatgcttcttgctaacctacatttccaaaagatcatccgagaaatctggcaagccaccgaaatggtactagtaaactggatccatccaaagcactccacagtgccctgtgggtgtgtttctcactgtaaactactgggtaaagaatttacatacaattttatctccaaactcgatttgtatcctttgtaggttagttctcaggagtacccactccagcccagaaataagtcaacgtagtatttccaaactatcacttcaaatgtccacggtcaagggcaggaggaggaaggaatagtaacgggaaaacttgtcagcgattagccatccataaaaatgttctcccagcgctcctccttccgatcacaaagctggcgtcaggcagtctcctgtatctgcttcccacctcgccccctcccctctgcggagagctggcaccgaggggcgggccaaggaaaagacaaacaccccggctccgacacagtccaagcgcgagcccggagacacttcaacaacattcctaactttttttctcagttcgcatgcctcaaaggtaaacaaagcaaaaagcctacgaaacagaaggcaagtgaggccaattcggc

SEQ ID NO: 180 - DNA, 856 bp, Homo sapiens
cgcgaggaaggcgagctgcgccgcggccaactccccagagcagtcccctccggccccgcgcccaccccaccccaccccactccaccccagacaggctccaccaaaaaacccgggaacaacgaccgccaaaagccaagcctcacgccgaggagcctcccaggctgccccgaggacgaacaacttcgccctccactcaatgcgctcacacgcacgcacacaagcagcgcgcggccgaggagggcacgtcttacctgtccccgccgtgccactcatccctcccccacccaggtccggcgcccccaaccccgcccccgccgtggtcccccggccgcccgctccgagcgccgcgcactcactgcctggctgcgcgcgcccagcgcctgcagggcccggcggcggcggcggggaccgagacagcggctgcagcagcggcggcggcggcggcggcggcggcggccccagccggcgtcagtcagactggagccgcgaagcctcatcgcccgtattagtgcgccgacctggaaagcggccagggagccctgcttgcggcccgcccccgcccgccgccgcgcgctctggccgctccgggagccgcagaggagggggctgcgcgggcccggcggggggcgggagcgagaccggccccacggccacgtgcgcggaaactcgcggcgcgggggtggcggggtagtactcctcgcagcggcctgcggctcggttcccgcctcttccccacccccagccccgcgctgccctctcggtccccctgcgcgaccccaggctcggcccctgcccggcctgccggggtggcccgggggtggggtgggagccctttgtctgcgtgggtcgcctcgcg

SEQ ID NO: 181 - DNA, 182 bp, Homo sapiens
ggcagtctcctgtatctgcttcccacctcgccccctcccctctgcggagagctggcaccgaggggcgggccaaggaaaagacaaacaccccggctccgacacagtccaagcgcgagcccggagacacttcaacaacattcctaactttttttctcagttcgcatgcctcaaaggtaaacaaa